(19) United States
(12) Patent Application Publication
(10) Pub. No.: US 2025/0354138 A1
(43) Pub. Date: Nov. 20, 2025

(54) PRIME EDITING OF SINGLE BASE MUTATIONS IN ALPHA-1 ANTITRYPSIN DEFICIENCY

(71) Applicant: BEAM THERAPEUTICS INC., Cambridge, MA (US)

(72) Inventors: Maria Montiel, Cambridge, MA (US); Ayan Banerjee, Cambridge, MA (US); Brian Cafferty, Cambridge, MA (US); James Tam, Cambridge, MA (US); David Bryson, Cambridge, MA (US); Markrete Krikorian, Cambridge, MA (US)

(21) Appl. No.: 19/080,343

(22) Filed: Mar. 14, 2025

Related U.S. Application Data
(60) Provisional application No. 63/565,839, filed on Mar. 15, 2024.

(51) Int. Cl. C12N 15/11; A61K 9/51; A61K 31/7125; C12N 9/12; C12N 9/22; C12N 15/88; C12N 15/90

(52) U.S. Cl. CPC C12N 15/11; A61K 9/5123; A61K 31/7125; C12N 9/1276; C12N 9/226; C12N 15/88; C12N 15/907; C12Y 207/07049; C07K 2319/09; C12N 2310/20; C12N 2310/321; C12N 2310/3231

(57) ABSTRACT

This disclosure provides compositions and methods for the editing of a SERPINA1 gene with prime editing.

### CROSS REFERENCE TO RELATED APPLICATION

This application claims the benefit of U.S. Provisional Patent Application Ser. No. 63/565,839, filed Mar. 15, 2024. The entire content of the above-referenced patent application is incorporated by reference in its entirety herein.

### SEQUENCE LISTING

The instant application contains a Sequence Listing that has been submitted in a XML format and is hereby incorporated by reference in its entirety. Said XML file, created on 08/08/2025 is named 762368_BEAM9-011_ST26.xml and is 652,084 bytes in size.

### BACKGROUND

Prime editing, a recently described genome-editing tool, uses an engineered Moloney Murine Leukemia Virus reverse transcriptase (M-MLV RT) fused to a catalytically disabled Cas nickase enzyme and a 3′-extended prime editor gRNA (pegRNA) (denoted “PE2”) (Anzalone et al. Nature 576, 149-157. 2019). PE2 locally re-writes small (typically <50 nt) regions of the genome to generate any combination of insertion, deletion, transversion, or transition mutations (Anzalone, supra). Follow-on prime editing systems often include a second nicking gRNA (termed nRNA) (denoted “PE3”) (Anzalone, supra; Anzalone et al. Nat Biotechnol 38, 824-844. 2020). Prime editors have been used in a wide range of cell types and organisms, including mice (Jang et al. Nat Biomed Eng. 1-14. 2021; Liu et al. Nat Commun. 12, 2121. 2021), zebrafish (Petri et al. Nat Biotechnol. 1-5. 2021), human primary T cells (Petri, supra; Chen et al. Cell. 184(22):5635-5652. 2021) and patient-derived organoids (Schene et al. Nat Commun. 11, 5352. 2020). The inherent flexibility of prime editing distinguishes it as a promising complementary approach to existing genome-editing tools, such as nucleases and base editors (Anzalone 2020, supra; Rees et al. Nat Rev Genet. 19, 770-788. 2018).

To date, there have been no reports of prime editing in human primary fibroblast and in A1AT E342K cells for the correction of the E342K G to A SNP in the SERPINA1 gene. Described herein is the application of prime editing towards correction of human genetic variants causal for Alpha-1 Antitrypsin Deficiency (A1AD).

### SUMMARY

Provided herein are prime editors, prime editing guide RNAs (PEgRNAs), and nickRNAs (ngRNA) useful for the editing of the E342K mutation in Alpha-1 Antitrypsin Deficiency (A1AD) for the treatment of A1AD.

In one aspect, this disclosure provides a prime editing guide RNA (PEgRNA) comprising: a spacer that is complementary to a search target sequence on a first strand of a human SERPINA1 gene, a primer binding site sequence (PBS) at least partially complementary to the spacer, an editing template that comprises a region of complementarity to an editing target sequence on a second strand of the SERPINA1 gene, and a gRNA core that associates with a prime editor comprising a DNA binding domain and a DNA polymerase domain, wherein the PBS comprises or consists of the sequence GCACGGC (SEQ ID NO: 2) and the editing template comprises or consists of the sequence

(SEQ ID NO: 25)
UUUCUCGUCGAUGGUCAGCACAGCCUUAU.

In another aspect, the spacer sequence comprises or consists of the sequence UCCCCUCCAGGCCGUGCAUA (SEQ ID NO: 1). In another aspect, the gRNA core is between the spacer and the editing template. In another aspect, the editing template comprises an intended nucleotide edit compared to the SERPINA1 gene. In another aspect, the PEgRNA guides the prime editor to incorporate the intended nucleotide edit into the SERPINA1 gene when contacted with the SERPINA1 gene. In another aspect, the prime editor synthesizes a single stranded DNA encoded by the editing template, wherein the single stranded DNA replaces the editing target sequence and results in incorporation of the intended nucleotide edit into a region corresponding to the editing target in the SERPINA1 gene. In another aspect, the search target sequence is complementary to a protospacer sequence in the SERPINA1 gene, and wherein the protospacer sequence is adjacent to a search target adjacent motif (PAM) in the SERPINA1 gene. In another aspect, the PAM comprises NGG. In another aspect, the PEgRNA results in incorporation of the intended nucleotide edit in the PAM when contacted with the SERPINA1 gene. In another aspect, the PBS is about 2 to about 20 base pairs in length. In another aspect, the PBS is about 8 to about 16 base pairs in length.

In one aspect, the PEgRNA disclosed herein comprising the PBS comprises or consists of the sequence GCACGGCC (SEQ ID NO: 3), GCACGGCCU (SEQ ID NO: 4), GCACGGCCUG (SEQ ID NO: 5), GCACGGCCUGG (SEQ ID NO: 6), GCACGGCCUGGA (SEQ ID NO: 7), GCACGGCCUGGAG (SEQ ID NO: 8), GCACGGCCUGGAGG (SEQ ID NO: 9), or GCACGGCCUGGAGGG (SEQ ID NO: 10).

In another aspect, the PEgRNA the editing template is about 4 to 30 base pairs in length. In another aspect, the editing template is about 10 to 30 base pairs in length. In another aspect, the editing template comprises or consists of the sequence

(SEQ ID NO: 11)
GCAGCUUCAGUCCCUUUCUCGUCGAUGGUCAGCACAGCCUUAU,
(SEQ ID NO: 12)
CAGCUUCAGUCCCUUUCUCGUCGAUGGUCAGCACAGCCUUAU,
(SEQ ID NO: 13)
AGCUUCAGUCCCUUUCUCGUCGAUGGUCAGCACAGCCUUAU,
(SEQ ID NO: 14)
GCUUCAGUCCCUUUCUCGUCGAUGGUCAGCACAGCCUUAU,
(SEQ ID NO: 15)
CUUCAGUCCCUUUCUCGUCGAUGGUCAGCACAGCCUUAU,
(SEQ ID NO: 16)
UUCAGUCCCUUUCUCGUCGAUGGUCAGCACAGCCUUAU,
(SEQ ID NO: 17)
UCAGUCCCUUUCUCGUCGAUGGUCAGCACAGCCUUAU,
(SEQ ID NO: 18)
CAGUCCCUUUCUCGUCGAUGGUCAGCACAGCCUUAU,
(SEQ ID NO: 19)
AGUCCCUUUCUCGUCGAUGGUCAGCACAGCCUUAU,
(SEQ ID NO: 20)
GUCCCUUUCUCGUCGAUGGUCAGCACAGCCUUAU,
(SEQ ID NO: 21)
UCCCUUUCUCGUCGAUGGUCAGCACAGCCUUAU,
(SEQ ID NO: 22)
CCCUUUCUCGUCGAUGGUCAGCACAGCCUUAU,
(SEQ ID NO: 23)
CCUUUCUCGUCGAUGGUCAGCACAGCCUUAU,
(SEQ ID NO: 24)
CUUUCUCGUCGAUGGUCAGCACAGCCUUAU,
or
(SEQ ID NO: 25)
UUUCUCGUCGAUGGUCAGCACAGCCUUAU.

In one aspect, the PEgRNA disclosed herein results in incorporation of intended nucleotide edit about 0 to 27 base pairs downstream of the 5′ end of the PAM when contacted with the SERPINA1 gene. In another aspect, the intended nucleotide edit comprises a single nucleotide substitution compared to the region corresponding to the editing target in the SERPINA1 gene. In another aspect, the intended nucleotide edit comprise an insertion compared to the region corresponding to the editing target in the SERPINA1 gene. In another aspect, the intended nucleotide edit comprises a deletion compared to the region corresponding to the editing target in the SERPINA1 gene.

In one aspect, the PEgRNA disclosed herein comprises the editing target sequence comprises a mutation associated with alpha-1 antitrypsin deficiency (A1AD). In one aspect, the editing template comprises a wild type SERPINA1 gene sequence. In another aspect, the PEgRNA results in correction of the mutation when contacted with the SERPINA1 gene. In another aspect, the PEgRNA comprises or consists of any one of the sequences recited in Table 1.

In one aspect, the PEgRNA disclosed herein comprises at least one chemical modification. In another aspect, the at least one chemical modification is selected from the group consisting of a 2′-O-methyl (2′-OMe) modification, a 2′-deoxy (2′-H) modification, a 2′-fluoro (2′-F) modification, a 2′-methoxyethyl (2′-MOE) modification, a 2′-amino (2′-NH2) modification, a 2′-arabinosyl (2′-arabino) modification, a 2′-F-arabinosyl (2′-F-arabino) modification, and a locked nucleic acid (LNA) modification. In another aspect, the at least one chemical modification comprises an internucleotide linkage modification. In another aspect, the at least one internucleotide linkage modification comprises a phosphonoacetate (PACE) modification. In another aspect, the PEgRNA comprises or consists of any one of the sequences recited in Table 1.

In one aspect, the disclosure provides a PEgRNA system comprising the PEgRNA disclosed herein and further comprises a nick guide RNA (ngRNA), wherein the ngRNA comprises an ng spacer that is complementary to a second search target sequence in the SERPINA1 gene. In another aspect, the second search target sequence is on the second strand of the SERPINA1 gene. In another aspect, the ngRNA comprises a spacer sequence selected from the group consisting of: GAAGCAGAGACACGUUGUA (SEQ ID NO: 26), GUCAGCACAGCCUUAUGCA (SEQ ID NO: 27), GAAAGGGACUGAAGCUGCU (SEQ ID NO: 28), CCUCGGGGGGGAUAGACAU (SEQ ID NO: 29), UGAUCCCAGGCCUCGAGCA (SEQ ID NO: 30), ACGUUGUAAGGCUGAUCCC (SEQ ID NO: 31), AAAGGGACUGAAGCUGCUG (SEQ ID NO: 32), GGUAUGGCCUCUAAAAACA (SEQ ID NO: 33), CCCAUGUCUAUCCCCCCCG (SEQ ID NO: 34), GCCUCGAGCAAGGCUCACG (SEQ ID NO: 35), GGUUUGUUGAACUUGACCU (SEQ ID NO: 36), CCUUAUGCACGGCCUGGAG (SEQ ID NO: 37), AGAAAGGGACUGAAGCUGC (SEQ ID NO: 38), CACAGCCUUAUGCACGGCC (SEQ ID NO: 39), GGGGGGAUAGACAUGGGUA (SEQ ID NO: 40), GUUUGUUGAACUUGACCUC (SEQ ID NO: 41), UGCUGACCAUCGACAAGAA (SEQ ID NO: 42), UUGUUGAACUUGACCUCGG (SEQ ID NO: 43), GCCUUAUGCACGGCCUGGA (SEQ ID NO: 44), UUUGUUGAACUUGACCUCG (SEQ ID NO: 45), GUUGAACUUGACCUCGGGG (SEQ ID NO: 46), CUCUGCUUCUCUCCCCUCC (SEQ ID NO: 47), UGAGCCUUGCUCGAGGCCU (SEQ ID NO: 48), AGCCUUAUGCACGGCCUGG (SEQ ID NO: 49), ACCUCGGGGGGGAUAGACA (SEQ ID NO: 50), UCAGUCCCUUUCUUGUCGA (SEQ ID NO: 51), UGUUGAACUUGACCUCGGG (SEQ ID NO: 52), CCCCUCCAGGCCGUGCAUA (SEQ ID NO: 53), GUGAGCCUUGCUCGAGGCC (SEQ ID NO: 54), GCUGACCAUCGACAAGAAA (SEQ ID NO: 55), GCUGGGGCCAUGUUUUUAG (SEQ ID NO: 56), UGCUGACCAUCGACGAGAA (SEQ ID NO: 57), UCAGUCCCUUUCUCGUCGA (SEQ ID NO: 58), and/or GCUGACCAUCGACGAGAAA (SEQ ID NO: 59).

In one aspect, the disclosure provides a PEgRNA system comprising the PEgRNA according to the disclosure as well the ngRNA disclosed herein. In another aspect, a prime editing complex comprises: (i) the PEgRNA disclosed herein or the PEgRNA system disclosed herein; and (ii) a prime editor comprising a DNA binding domain and a DNA polymerase domain. In another aspect, the prime editing complex described herein comprises the DNA binding domain is a CRISPR associated (Cas) protein domain. In another aspect, the prime editing complex described herein comprises the Cas protein domain comprising nickase activity. In another aspect, the Cas protein domain is a Cas9. In another aspect, the Cas9 comprises a mutation in an HNH domain. In another aspect, the Cas9 comprises a H840A mutation in the HNH domain. In another aspect, the Cas9 comprises the sequence of SEQ ID NO: 60. In another aspect, the Cas9 comprises a mutation at one or more amino acids positions of R765, K848, K855, K959, K961, K968, K974, or R976 relative to SEQ ID NO: 60. In another aspect, the Cas9 comprises one or more mutations of R765A, K848A, K855A, K959A, K961A, K968A, K974A, or R976A relative to SEQ ID NO: 60. In one aspect, the Cas9 comprises or consists of any one of the sequences of SEQ ID NO: 61 to 72.

In one aspect, the disclosure provides a prime editing complex wherein the DNA polymerase domain is a reverse transcriptase. In another aspect, the reverse transcriptase is a retrovirus reverse transcriptase. In another aspect, the reverse transcriptase is a Moloney murine leukemia virus (M-MLV) reverse transcriptase. In another aspect, the reverse transcriptase comprises the sequence of SEQ ID NO: 75

(TLNIEDEYRLHETSKEPDVSLGSTWLSDFPQAWAETGGMGLAVRQAPL
IIPLKATSTPVSIKQYPMSQEARLGIKPHIQRLLDQGILVPCQSPWNTP
LLPVKKPGTNDYRPVQDLREVNKRVEDIHPTVPNPYNLLSGLPPSHQWY
TVLDLKDAFFCLRLHPTSQPLFAFEWRDPEMGISGQLTWTRLPQGFKNS
PTLFNEALHRDLADFRIQHPDLILLQYVDDLLLAATSELDCQQGTRALL
QTLGNLGYRASAKKAQICQKQVKYLGYLLKEGQRWLTEARKETVMGQPT
PKTPRQLREFLGKAGFCRLFIPGFAEMAAPLYPLTKPGTLFNWGPDQQK
AYQEIKQALLTAPALGLPDLTKPFELFVDEKQGYAKGVLTQKLGPWRRP
VAYLSKKLDPVAAGWPPCLRMVAAIAVLTKDAGKLTMGQPLVILAPHAV
EALVKQPPDRWLSNARMTHYQALLLDTDRVQFGPVVALNPATLLPLPEE
GLQHNCLDILAEAHGGGSKRTADGSEFE).

In one aspect of the disclosure, the prime editing complex described herein comprises the DNA polymerase and the DNA binding domain that are fused or linked to form a fusion protein. In one aspect, the DNA polymerase and the programmable DNA binding domain are linked by a linker comprising an amino acid sequence of SGGSEAAAKEAAAKEAAAKEAAAKSGGS (SEQ ID NO: 277). In one aspect, the fusion protein comprises the sequence of SEQ ID NO: 77

(MKRTADGSEFESPKKKRKVDKKYSIGLDIGTNSVGWAVITDEYKVPSK
KFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPD
NSDVDKLFIQLVQTYNQLFEENPINASGVDAKAILSARLSKSRRLENLI
AQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILED
IVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMAR
ENQTTQKGQKNSRERMKRIEEGIKELGSQILKEHPVENTQLQNEKLYLY
YLQNGRDMYVDQELDINRLSDYDVDAIVPQSFLKDDSIDNKVLTRSDKN
RGKSDNVPSEEVVKKMKNYWRQLLNAKLITQRKFDNLTKAERGGLSELD
KAGFIKRQLVETRQITKHVAQILDSRMNTKYDENDKLIREVKVITLKSK
LVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFEL
ENGRKRMLASAGELQKGNELALPSKYVNFLYLASHYEKLKGSPEDNEQK
QLFVEQHKHYLDEIIEQISEFSKRVILADANLDKVLSAYNKHRDKPIRE
QAENIIHLFTLTNLGAPAAFKYFDTTIDRKRYTSTKEVLDATLIHQSIT
GLYETRIDLSQLGGDSGGSEAAAKEAAAKEAAAKEAAAKSGGSTLNIED
EYRLHETSKEPDVSLGSTWLSDFPQAWAETGGMGLAVRQAPLIIPLKAT
STPVSIKQYPMSQEARLGIKPHIQRLLDQGILVPCQSPWNTPLLPVKKP
GTNDYRPVQDLREVNKRVEDIHPTVPNPYNLLSGLPPSHQWYTVLDLKD
AFFCLRLHPTSQPLFAFEWRDPEMGISGQLTWTRLPQGFKNSPTLFNEA
LHRDLADFRIQHPDLILLQYVDDLLLAATSELDCQQGTRALLQTLGNLG
YRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQ
ALLTAPALGLPDLTKPFELFVDEKQGYAKGVLTQKLGPWRRPVAYLSKK
LDPVAAGWPPCLRMVAAIAVLTKDAGKLTMGQPLVILAPHAVEALVKQP
PDRWLSNARMTHYQALLLDTDRVQFGPVVALNPATLLPLPEEGLQHNCL
DILAEAHGGGSKRTADGSEFE).

In one aspect, the prime editing complex disclosed herein comprises the fusion protein comprising a nuclear localization signal (NLS). In another aspect, the NLS comprises an amino acid sequence of PKKKRKV (SEQ ID NO: 282).

In another aspect, the prime editing complex disclosed herein comprises the fusion protein is encoded by the polynucleotide sequence comprising:

(SEQ ID NO: 78)
ATGAAACGGACAGCCGACGGAAGCGAGTTCGAGTCACCAAAGAAGAAGCGGAAAGTCGACAAGAAGTA
CAGCATCGGCCTGGACATCGGCACCAACTCTGTGGGCTGGGCCGTGATCACCGACGAGTACAAGGTGC
CCAGCAAGAAATTCAAGGTGCTGGGCAACACCGACCGGCACAGCATCAAGAAGAACCTGATCGGAGCC
CTGCTGTTCGACAGCGGCGAAACAGCCGAGGCCACCCGGCTGAAGAGAACCGCCAGAAGAAGATACAC
CAGACGGAAGAACCGGATCTGCTATCTGCAAGAGATCTTCAGCAACGAGATGGCCAAGGTGGACGACA
GCTTCTTCCACAGACTGGAAGAGTCCTTCCTGGTGGAAGAGGATAAGAAGCACGAGCGGCACCCCATC
TTCGGCAACATCGTGGACGAGGTGGCCTACCACGAGAAGTACCCCACCATCTACCACCTGAGAAAGAA
ACTGGTGGACAGCACCGACAAGGCCGACCTGCGGCTGATCTATCTGGCCCTGGCCCACATGATCAAGT
TCCGGGGCCACTTCCTGATCGAGGGCGACCTGAACCCCGACAACAGCGACGTGGACAAGCTGTTCATC
CAGCTGGTGCAGACCTACAACCAGCTGTTCGAGGAAAACCCCATCAACGCCAGCGGCGTGGACGCCAA
GGCCATCCTGTCTGCCAGACTGAGCAAGAGCAGACGGCTGGAAAATCTGATCGCCCAGCTGCCCGGCG
AGAAGAAGAATGGCCTGTTCGGAAACCTGATTGCCCTGAGCCTGGGCCTGACCCCCAACTTCAAGAGC
AACTTCGACCTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTCCGACGCCA
TCCTGCTGAGCGACATCCTGAGAGTGAACACCGAGATCACCAAGGCCCCCCTGAGCGCCTCTATGATC
AAGAGATACGACGAGCACCACCAGGACCTGACCCTGCTGAAAGCTCTCGTGCGGCAGCAGCTGCCTGA
GAAGTACAAAGAGATTTTCTTCGACCAGAGCAAGAACGGCTACGCCGGCTACATTGACGGCGGAGCCA
GCCAGGAAGAGTTCTACAAGTTCATCAAGCCCATCCTGGAAAAGATGGACGGCACCGAGGAACTGCTC
GTGAAGCTGAACAGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCATTCCTGAAGGACA
ACCGGGAAAAGATCGAGAAGATCCTGACCTTCCGCATCCCCTACTACGTGGGCCCTCTGGCCAGGGGA
AACAGCAGATTCGCCTGGATGACCAGAAAGAGCGAGGAAACCATCACCCCCTGGAACTTCGAGGAAGT
GGTGGACAAGGGCGCTTCCGCCCAGAGCTTCATCGAGCGGATGACCAACTTCGATAAGAACCTGCCCA
ACGAGAAGGTGCTGCCCAAGCACAGCCTGCTGTACGAGTACTTCACCGTGTATAACGAGCTGACCAAA
GTGAAATACGTGACCGAGGGAATGAGAAAGCCCGCCTTCCTGAGCGGCGAGCAGAAAAAGGCCATCGT
GGACCTGCTGTTCAAGACCAACCGGAAAGTGACCGTGAAGCAGCTGAAAGAGGACTACTTCAAGAAAA
TCGAGTGCTTCGACTCCGTGGAAATCTCCGGCGTGGAAGATCGGTTCAACGCCTCCCTGGGCACATAC
CACGATCTGCTGAAAATTATCAAGGACAAGGACTTCCTGGACAATGAGGAAAACGAGGACATTCTGGA
AGATATCGTGCTGACCCTGACACTGTTTGAGGACAGAGAGATGATCGAGGAACGGCTGAAAACCTATG
CCCACCTGTTCGACGACAAAGTGATGAAGCAGCTGAAGCGGCGGAGATACACCGGCTGGGGCAGGCTG
AGCCGGAAGCTGATCAACGGCATCCGGGACAAGCAGTCCGGCAAGACAATCCTGGATTTCCTGAAGTC
CGACGGCTTCGCCAACAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCCAATCTGGCCGGCAGC
CCCGCCATTAAGAAGGGCATCCTGCAGACAGTGAAGGTGGTGGACGAGCTCGTGAAAGTGATGGGCCG
GCACAAGCCCGAGAACATCGTGATCGAAATGGCCAGAGAGAACCAGACCACCCAGAAGGGACAGAAGA
ACAGCCGCGAGAGAATGAAGCGGATCGAAGAGGGCATCAAAGAGCTGGGCAGCCAGATCCTGAAAGAA
CACCCCGTGGAAAACACCCAGCTGCAGAACGAGAAGCTGTACCTGTACTACCTGCAGAATGGGCGGGA
TATGTACGTGGACCAGGAACTGGACATCAACCGGCTGTCCGACTACGATGTGGACGCTATCGTGCCTC
AGAGCTTTCTGAAGGACGACTCCATCGACAACAAGGTGCTGACCAGAAGCGACAAGAACCGGGGCAAG
AGCGACAACGTGCCCTCCGAAGAGGTCGTGAAGAAGATGAAGAACTACTGGCGGCAGCTGCTGAACGC
CAAGCTGATTACCCAGAGAAAGTTCGACAATCTGACCAAGGCCGAGAGAGGCGGCCTGAGCGAACTGG
ATAAGGCCGGCTTCATCAAGAGACAGCTGGTGGAAACCCGGCAGATCACAAAGCACGTGGCACAGATC
CTGGACTCCCGGATGAACACTAAGTACGACGAGAATGACAAGCTGATCCGGGAAGTGAAAGTGATCAC
CCTGAAGTCCAAGCTGGTGTCCGATTTCCGGAAGGATTTCCAGTTTTACAAAGTGCGCGAGATCAACA
ACTACCACCACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCATGAACTTTTTCAAGA
CCGAGATTACCCTGGCCAACGGCGAGATCCGGAAGCGGCCTCTGATCGAGACAAACGGCGAAACCGGG
GAGATCGTGTGGGATAAGGGCCGGGATTTTGCCACCGTGCGGAAAGTGCTGAGCATGCCCCAAGTGAA
TATCGTGAAAAAGACCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCTTCGACAGCCCCACC
GTGGCCTATTCTGTGCTGGTGGTGGCCAAAGTGGAAAAGGGCAAGTCCAAGAAACTGAAGAGTGTGAA
AGAGCTGCTGGGGATCACCATCATGGAAAGAAGCAGCTTCGAGAAGAATCCCATCGACTTTCTGGAAG
CCAAGGGCTACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACTGGCCCTGCC
CTCCAAATATGTGAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATCGAGCAGATCAGC
GAGTTCTCCAAGAGAGTGATCCTGGCCGACGCTAATCTGGACAAAGTGCTGTCCGCCTACAACAAGCA
CCGGGATAAGCCCATCAGAGAGCAGGCCGAGAATATCATCCACCTGTTTACCCTGACCAATCTGGGAG
CCCCTGCCGCCTTCAAGTACTTTGACACCACCATCGACCGGAAGAGGTACACCAGCACCAAAGAGGTG
CTGGACGCCACCCTGATCCACCAGAGCATCACCGGCCTGTACGAGACACGGATCGACCTGTCTCAGCT
GGGAGGTGACTCCGGCGGCAGCGAGGCCGCCGCCAAGGAAGCCGCCGCCAAGGAAGCCGCTGCCAAGG
AGGCCGCTGCTAAAAGCGGCGGATCTACCCTGAACATCGAGGACGAGTACAGGCTGCACGAGACCAGC
AAGGAGCCCGACGTGAGCCTGGGCAGCACCTGGCTGAGCGATTTCCCTCAGGCTTGGGCCGAGACCGG
CGGCATGGGCCTGGCCGTGCGGCAGGCCCCCCTGATTATCCCCCTGAAGGCCACCAGCACCCCCGTGA
GCATCAAGCAGTACCCAATGTCCCAGGAGGCCAGGCTGGGCATCAAGCCTCACATCCAGAGGCTGCTG
GACCAGGGCATCCTGGTGCCATGCCAGTCCCCCTGGAACACCCCTCTGCTGCCCGTGAAGAAGCCTGG
CACCAACGACTACCGGCCCGTGCAGGACCTGAGAGAAGTGAACAAGCGGGTGGAGGACATCCACCCAA
CCGTGCCCAACCCTTACAACCTGCTGTCCGGCCTGCCCCCCAGCCACCAGTGGTACACCGTGCTGGAC
CTGAAGGACGCCTTCTTCTGCCTGAGACTGCACCCCACCTCTCAGCCCCTGTTCGCCTTCGAGTGGCG
CGACCCCGAGATGGGCATCAGCGGCCAGCTGACCTGGACCAGACTGCCACAGGGCTTTAAGAATAGCC
CAACCCTGTTTAACGAGGCCCTGCACAGGGACCTGGCCGACTTCAGGATCCAGCACCCCGACCTGATT
CTGCTGCAGTACGTGGACGACCTGCTGCTGGCCGCTACCAGCGAGCTGGACTGCCAGCAGGGCACCAG
AGCCCTGCTGCAGACCCTGGGCAACCTGGGCTACAGAGCCAGCGCCAAGAAGGCCCAGATCTGTCAGA
AGCAGGTGAAGTATCTGGGCTACCTGCTGAAGGAAGGCCAGAGATGGCTGACCGAGGCCAGAAAGGAG
ACTGTGATGGGCCAGCCCACCCCCAAGACCCCCAGGCAGCTGCGGGAGTTCCTGGGCAAGGCCGGCTT
TTGCAGACTGTTTATCCCTGGCTTCGCCGAGATGGCCGCCCCACTGTACCCTCTGACCAAGCCTGGCA
CCCTGTTTAACTGGGGCCCCGACCAGCAGAAGGCCTACCAGGAGATCAAGCAGGCCCTGCTGACCGCC
CCCGCCCTGGGCCTGCCCGACCTGACCAAGCCTTTCGAGCTGTTCGTGGACGAGAAGCAGGGATACGC
CAAAGGCGTGCTGACCCAGAAGCTGGGCCCCTGGCGGAGGCCCGTGGCCTACCTGAGCAAAAAACTGG
ACCCTGTGGCCGCCGGCTGGCCCCCATGCCTGCGGATGGTGGCCGCCATCGCTGTGCTGACCAAGGAC
GCCGGCAAGCTGACCATGGGCCAGCCCCTGGTGATCCTGGCCCCTCACGCCGTGGAGGCTCTGGTGAA
GCAGCCTCCAGACAGGTGGCTGTCCAACGCCAGGATGACCCACTACCAGGCCCTGCTGCTGGACACCG
ACCGGGTGCAGTTCGGCCCTGTGGTGGCCCTGAACCCCGCCACCCTGCTGCCTCTGCCAGAGGAGGGC
CTGCAGCACAACTGCCTGGACATCCTGGCCGAGGCCCACGGCGGCGGCTCCAAACGCACCGCCGACGG
GAGCGAGTTCGAGCCCAAGAAGAAGAGGAAAGTCTAA.

In another aspect, the prime editing complex disclosed herein comprises a polynucleotide sequence comprising an mRNA.

In one aspect, a PEgRNA system disclosed herein comprises: i) a prime editing guide RNA (PEgRNA) comprising: a spacer that is complementary to a search target sequence on a first strand of a human SERPINA1 gene, a primer binding site sequence (PBS) at least partially complementary to the spacer, an editing template that comprises a region of complementarity to an editing target sequence on a second strand of the SERPINA1 gene, and a gRNA core that associates with a prime editor comprising a DNA binding domain and a DNA polymerase domain; and ii) a nick guide RNA (ngRNA), wherein the ngRNA comprises an ng spacer that is complementary to a second search target sequence in the SERPINA1 gene, wherein the ngRNA comprises a spacer sequence selected from the group consisting of: GAAGCAGAGACACGUUGUA (SEQ ID NO: 26), GUCAGCACAGCCUUAUGCA (SEQ ID NO: 27), GAAAGGGACUGAAGCUGCU (SEQ ID NO: 28), CCUCGGGGGGGAUAGACAU (SEQ ID NO: 29), UGAUCCCAGGCCUCGAGCA (SEQ ID NO: 30), ACGUUGUAAGGCUGAUCCC (SEQ ID NO: 31), AAAGGGACUGAAGCUGCUG (SEQ ID NO: 32), GGUAUGGCCUCUAAAAACA (SEQ ID NO: 33), CCCAUGUCUAUCCCCCCCG (SEQ ID NO: 34), GCCUCGAGCAAGGCUCACG (SEQ ID NO: 35), GGUUUGUUGAACUUGACCU (SEQ ID NO: 36), CCUUAUGCACGGCCUGGAG (SEQ ID NO: 37), AGAAAGGGACUGAAGCUGC (SEQ ID NO: 38), CACAGCCUUAUGCACGGCC (SEQ ID NO: 39), GGGGGGAUAGACAUGGGUA (SEQ ID NO: 40), GUUUGUUGAACUUGACCUC (SEQ ID NO: 41), UGCUGACCAUCGACAAGAA (SEQ ID NO: 42), UUGUUGAACUUGACCUCGG (SEQ ID NO: 43), GCCUUAUGCACGGCCUGGA (SEQ ID NO: 44), UUUGUUGAACUUGACCUCG (SEQ ID NO: 45), GUUGAACUUGACCUCGGGG (SEQ ID NO: 46), CUCUGCUUCUCUCCCCUCC (SEQ ID NO: 47), UGAGCCUUGCUCGAGGCCU (SEQ ID NO: 48), AGCCUUAUGCACGGCCUGG (SEQ ID NO: 49), ACCUCGGGGGGGAUAGACA (SEQ ID NO: 50), UCAGUCCCUUUCUUGUCGA (SEQ ID NO: 51), UGUUGAACUUGACCUCGGG (SEQ ID NO: 52), CCCCUCCAGGCCGUGCAUA (SEQ ID NO: 53), GUGAGCCUUGCUCGAGGCC (SEQ ID NO: 54), GCUGACCAUCGACAAGAAA (SEQ ID NO: 55), GCUGGGGCCAUGUUUUUAG (SEQ ID NO: 56), UGCUGACCAUCGACGAGAA (SEQ ID NO: 57), UCAGUCCCUUUCUCGUCGA (SEQ ID NO: 58), and/or GCUGACCAUCGACGAGAAA (SEQ ID NO: 59).

In one aspect, the PEgRNA system disclosed herein comprising the PBS comprises or consists of the sequence GCACGGC (SEQ ID NO: 2) and the editing template comprises or consists of the sequence UUUCUCGUCGAUGGUCAGCACAGCCUUAU (SEQ ID NO: 25).

In one aspect, the PEgRNA system disclosed herein comprising the spacer sequence comprises or consists of the sequence UCCCCUCCAGGCCGUGCAUA (SEQ ID NO: 1).

In one aspect, a prime editing guide RNA (PEgRNA) comprises: a spacer that is complementary to a search target sequence on a first strand of a human SERPINA1 gene, a primer binding site sequence (PBS) at least partially complementary to the spacer, an editing template that comprises a region of complementarity to an editing target sequence on a second strand of the SERPINA1 gene, and a gRNA core that associates with a prime editor comprising a DNA binding domain and a DNA polymerase domain, wherein the editing template comprises or consists of the sequence

(SEQ ID NO: 283)
UUUCUCAUCGAUGGUCAGCACAGCCUUAU
or
(SEQ ID NO: 284)
CUUUCUCAUCGAUGGUCAGCACAGCCUUAU.\

In one aspect, the PEgRNA of the disclosure comprising the PBS comprises or consists of the sequence GCACGGC (SEQ ID NO: 2). In another aspect, the spacer sequence comprises or consists of the sequence UCCCCUCCAGGCCGUGCAUA (SEQ ID NO: 1).

In one aspect, the disclosure provides a lipid nanoparticle (LNP) or ribonucleoprotein (RNP) comprising the prime editing complex disclosed herein, or a component thereof. In another aspect, a lipid nanoparticle (LNP) composition comprises: i) the PEgRNA disclosed herein, ii) the ngRNA disclosed herein, and iii) a polynucleotide encoding the prime editor disclosed herein. In one aspect, the PEgRNA, the ngRNA, and the polynucleotide encoding the prime editor are each encapsulated in separate LNPs. In another aspect, the PEgRNA, the ngRNA, and the polynucleotide encoding the prime editor are encapsulated in a single LNP. In another aspect, the mass ratio of the polynucleotide encoding the prime editor to the combination of the PEgRNA and the ngRNA (total gRNA content) is about 0.1:1 to about 3.0:1. In another aspect, the mass ratio of the polynucleotide encoding the prime editor to the combination of the PEgRNA and the ngRNA (total gRNA content) is about 0.5:1. In another aspect, the mass ratio of the PEgRNA to the ngRNA is about 1:1 to about 25:1. In another aspect, the mass ratio of the PEgRNA to the ngRNA is about 19:1.

In one aspect, the disclosure provides a polynucleotide encoding the PEgRNA disclosed herein, the PEgRNA system disclosed herein, or the fusion protein disclosed herein. In another aspect, the polynucleotide is a mRNA. In another aspect, the polynucleotide is operably linked to a regulatory element. In another aspect, the regulatory element is an inducible regulatory element. In another aspect, a vector comprises the polynucleotide disclosed herein. In another aspect, the vector is an AAV vector.

In one aspect, an isolated cell comprises the PEgRNA disclosed herein, the PEgRNA system, the prime editing complex, the LNP or RNP, the polynucleotide, or the vector disclosed herein. In another aspect, the cell is a human cell. In another aspect, the cell is a hepatocyte, a hepatic stellate cell, a Kupffer cell, or a liver sinusoidal endothelial cell. In another aspect, the cell is a hepatocyte or a hepatic stellate cell.

In one aspect, a pharmaceutical composition comprises (i) the PEgRNA, the PEgRNA system, the prime editing complex, the LNP or RNP, the polynucleotide, the vector, or the cell disclosed herein; and (ii) a pharmaceutically acceptable carrier.

In one aspect, the disclosure provides a method for editing a SERPINA1 gene, the method comprising contacting the SERPINA1 gene with (i) the PEgRNA or the PEgRNA system disclosed herein and (ii) a prime editor comprising a DNA binding domain and a DNA polymerase domain, wherein the PEgRNA or the PEgRNA system directs the prime editor to incorporate the intended nucleotide edit in the SERPINA1 gene, thereby editing the SERPINA1 gene.

In one aspect, the disclosure provides a method for editing an SERPINA1 gene, the method comprising contacting the SERPINA1 gene with the prime editing complex disclosed herein, wherein the PEgRNA directs the prime editor to incorporate the intended nucleotide edit in the SERPINA1 gene, thereby editing the SERPINA1 gene. In one aspect, the disclosure provides a method for editing a SERPINA1 gene, the method comprising contacting a cell with the LNP composition disclosed herein. In another aspect, the prime editor synthesizes a single stranded DNA encoded by the editing template, wherein the single stranded DNA replaces the editing target sequence and results in incorporation of the intended nucleotide edit into a region corresponding to the editing target in the SERPINA1 gene. In another aspect, SERPINA1 gene is in a cell. In another aspect, the cell is a mammalian cell. In another aspect, the cell is a human cell. In another aspect, the cell is a hepatocyte or a hepatic stellate cell. In another aspect, the cell is in a subject. In another aspect, the subject is a human. In another aspect, the cell is from a subject having A1AD. In another aspect, the method disclosed herein further comprises administering the cell to the subject after incorporation of the intended nucleotide edit. In another aspect, the disclosure provides a cell generated by the method disclosed herein. In another aspect, the disclosure provides a population of cells generated by the method described herein.

In one aspect, the disclosure provides a method for treating A1AD in a subject in need thereof, the method comprising administering to the subject (i) the PEgRNA or the PEgRNA system disclosed herein and (ii) a prime editor comprising a DNA binding domain and a DNA polymerase domain, wherein the PEgRNA directs the prime editor to incorporate the intended nucleotide edit in the SERPINA1 gene in the subject, thereby treating A1AD in the subject.

In one aspect, the disclosure provides a method for treating A1AD in a subject in need thereof, the method comprising administering to the subject: the prime editing complex, the LNP or RNP, or the pharmaceutical composition disclosed herein, wherein the PEgRNA directs the prime editor to incorporate the intended nucleotide edit in the SERPINA1 gene in the subject, thereby treating A1AD in the subject. In another aspect, the disclosure provides a method for treating A1AD in a subject in need thereof, the method comprising administering to the subject the LNP composition disclosed herein, thereby treating A1AD in the subject. In another aspect, the subject is a human.

### BRIEF DESCRIPTION OF THE DRAWINGS

Aspects, features, benefits, and advantages of the embodiments described herein will be apparent with regard to the following description, examples, claims, and accompanying drawings where:

FIG. 1 depicts a schematic diagram showing prime editing (PE) components.

FIG. 2 (SEQ ID Nos: 277, 281 and 303) depicts prime editor constructs employing alternative linkers and/or modified (G504X) or full-length RT domains.

FIG. 3A-FIG. 3B depict the results of a prime editing screening experiment on the A1AT E342K genetic target in human primary fibroblast with PE2 editing using the pHRB-314 prime editor employing the SGGS-(EAAAK)₄-SGGS (SEQ ID NO: 277) linker and the G504X RT with the optimized C3 codon. FIG. 3A depicts the results of the prime editing screening on A1AT E342K in human primary fibroblast using all tested pegRNAs. FIG. 3B depicts prime editing results of pegRNAs that yielded about 1% editing efficiency on A1AT E342K in human primary fibroblast. 1 μg of mRNA encoding HRB-314 and 750 ng of pegRNA were transfected into cells for each experiment.

FIG. 4 depicts the results of the prime editing screening using the pHRB-314 prime editor with 10 select pegRNAs with the highest prime editing efficiency (˜1%). Each pegRNA employs a PBS and RTT of varying length.

FIG. 5 depicts pegRNA screening with an alternative prime editor in A1AT E342K cells. Prime editing efficiencies of different pegRNAs were assessed at 750, 950, and 1150 ng and using 1 and 2 μg (0.5 and 1 μmol, respectively) of the pHRB-311 prime editor employing the SGGS-(EAAAK)₄-SGGS (SEQ ID NO: 277) linker and the full length RT. Left bars for each tested pegRNA correspond to 1 μg primer editor and right bars for each tested pegRNA correspond to 2 μg primer editor.

FIG. 6 depicts pegRNA screening with an alternative prime editor in A1AT E342K cells. Prime editing efficiencies of different pegRNAs were assessed at 750, 950, and 1150 ng and using 1 and 2 μg (0.5 and 1 μmol, respectively) of the pHRB-303 employing the SGGS-(EAAAK)₈-SGGS (SEQ ID NO: 281) linker and the full length RT. Left bars for each tested pegRNA correspond to 1 μg primer editor and right bars for each tested pegRNA correspond to 2 μg primer editor.

FIG. 7 depicts pegRNA screening with an alternative prime editor in A1AT E342K cells. Prime editing efficiencies of different pegRNAs were assessed at 950 and 1150 ng and using 1 and 2 μg (0.5 and 1 μmol, respectively) of the pHRB-245 prime editor employing the XTEN linker and the G504X RT with a C3 codon optimization. Left bars for each tested pegRNA correspond to 1 μg primer editor and right bars for each tested pegRNA correspond to 2 μg primer editor.

FIG. 8 depicts pegRNA screening with an alternative prime editor in A1AT E342K cells. Prime editing efficiencies of different pegRNAs were assessed at 750, 950, and 1150 ng and using 1 and 2 μg (0.5 and 1 μmol, respectively) of the pHRB-314 prime editor employing the SGGS-(EAAAK)₄-SGGS (SEQ ID NO: 277) linker and the G504X RT with a C3 codon optimization. Left bars for each tested pegRNA correspond to 1 μg primer editor and right bars for each tested pegRNA correspond to 2 μg primer editor.

FIG. 9 depicts the editing efficiencies of the pHRB-314 prime editor with 34 different nicking guides in A1AT E342K cells with pegRNA5278.

FIG. 10 depicts a comparison of the prime editing efficiencies of prime editor variants with a pegRNA containing a silent mutation of D341D in addition to the E342K mutation. This was compared against a pegRNA containing the same silent mutation, but also a PAM disruption. Hek293T cells with the A1AT E342K mutation were employed using the PE3 prime editing strategy. Nicking guide 7 was used.

FIG. 11 depicts a comparison of the prime editing efficiencies of prime editor variants with a pegRNA containing a silent mutation of D341D in addition to the E342K mutation. This was compared against a pegRNA containing the same silent mutation, but also a PAM disruption. Human primary fibroblast cells with the A1AT E342K mutation were employed using the PE3 prime editing strategy. Nicking guide 7 was used.

FIG. 12 depicts the prime editing efficiency of several optimized prime editors with Cas9 variants.

FIG. 13A-FIG. 13B depict the indel frequencies of pegRNA and ngRNA using optimized prime editors with Cas9 variants. FIG. 13A depicts the indel frequencies with pegRNA 5278 and three different prime editors (HRB-314 and variant 5 and 6). FIG. 13B depicts the indel frequencies ngRNA 7 and three different prime editors (HRB-314 and variant 5 and 6).

FIG. 14A-FIG. 14B depicts the prime editing efficiency improvement when a nicking guide (ngRNA) is added to the prime editing system described herein. FIG. 14A shows the prime editing efficiency of correcting the A1AT E342K mutation in human primary fibroblasts (HPF E342K) cells via an A-to-G correction with two distinct chemically modified ngRNAs (labeled in “ngRNA 19 nt” corresponding to Table 3 ID as ngRNA8722 nick7; and “ngRNA 20 nt” corresponding to Table 3 ID as gRNA8722 nick7) when adding 1 ug prime editor (0.5 μmol). FIG. 14B is the same experimental set-up as FIG. 14A but compares the A-to-G correction efficiency when two different concentrations of prime editor are added (either 1 ug prime editor (0.5 μmol) (left two columns) or 2 ug prime editor (1 μmol) (right two columns)).

FIG. 15 depicts a graph of the dose-response of the A-to-G editing efficiency of two different prime editors (prime editor V5 (PEv5) vs. parental control—prime editor V0 (PEv0)) utilizing an optimized pegRNA (pegRNA 5278) described herein (top of figure) in a HepG2 E342K cell line. The top percent editing efficiency and the EC50 of the payload prime editor total mass (ng) vs. the pegRNA of the dose-response curve are presented in a corresponding table (bottom of figure).

FIG. 16A-FIG. 16B tests optimal ngRNAs and chemical synthesized LNA (LNA —locked nucleic acid) pegRNA are added to a prime editing system in HepG2 cells harboring the E34K mutation (HepG2 E34K). FIG. 16A depicts a graph of the percent of A-to-G editing efficiency in HepG2 E34K cells utilizing a prime editor (prime editor V6 (PEv6 mRNA6431)) with several optimized nicking guides (ng7, 32, 21, and 28) in addition to several optimized chemically modified LNA pegRNAs (pegRNAs tested depicted on the X-axis). FIG. 16B is schematic map of the designed LNA pegRNAs indicating the location of the LNA modification and the PBS complementary region over the length of the 20-nucleotide (20 nt) spacer region.

FIG. 17 depicts a graph of the dose-response curve of the percent of A-to-G editing efficiency for various prime editing system utilizing different combinations of two different prime editors (prime editor V5 (PEv5) and prime editor V6 (PEv6)), an optimized pegRNA (pegRNA 5278), and either ngRNA7 or ngRNA18 in a HepG2 E34K cell line (top of figure). The top percent editing efficiency and the EC50 values of the dose-response curve for each prime editing system experimental group is reported in tabular form (bottom of figure).

FIG. 18 is a schematic of one embodiment of the LNP delivery system for the prime editor system described herein wherein the components of the prime editing system (prime editor, pegRNA, ngRNA) are split and encapsulated into three LNPs.

FIG. 19 is a graph depicting the stability of the formulation for prime editing components (PE mRNA, pegRNA, and ngRNA) after LNP encapsulation described in FIG. 18 and a suspension in a sucrose buffer (post-sucrose group, x-axis left) and a subsequent −80 C freeze/thaw (one freeze thaw (1×FT) at −80° C. group, x-axis right). The left y-axis displays the RNA concentration (ug/mL) and the right y-axis displays the percent of LNP encapsulation (top of figure). The concentration (ug/mL), endotoxin (EU/mL), LNP diameter (d.nm), and polydispersity index (PdI) values for each experimental group is reported in tabular form (bottom of figure).

FIG. 20 shows ten distinct conditions of LNP mass ratios (PE mRNA: pegRNA; ngRNA; middle columns) and dosing (100, 550, and 1000 ng; last right column) tested for the LNP encapsulated prime editing components described in FIG. 18.

FIG. 21 is a graph showing the prime editing efficiency of correcting the A1AT E342K mutation in HepG2 E342K cells via an A-to-G correction with ten distinct experimental groups (conditions 1-10) of LNP encapsulated prime editing components shown in FIG. 20. Experimental group 11 is a control group with 2 ug of editor LNP, 1 ug pegRNA 5278 LNP and 225 ng ngRNA LNP. The bars above paired conditions (conditions 1 and 10; 3 and 4; and 7 and 8) indicate that those particular experimental group had the same mass ratio but different dose considerations as a second experimental group.

FIG. 22 is a graph showing the prime editing efficiency of correcting the A1AT E342K mutation in HepG2 E342K cells via an A-to-G correction (A to G % correction; y-axis) with twenty distinct conditions (1-20 conditions labeled on the x-axis) of the LNP encapsulated prime editing components (PEv6 6431+pegRNA5258+ngRNA7=PE3) which expand the testing of mass ratios of the LNP encapsulated prime editing components (PE mRNA: pegRNA; ngRNA) (top of figure). The controls are non-LNP encapsulated master mixes (“MessMax”) utilizing two distinct editor mRNAs (Prime Editor V6 “PE3” or an adenosine-base editor “ABE”) along with pegRNA and ngRNA tested in the experimental groups. The expanded mass ratio conditions of the twenty experimental groups are displayed in the corresponding table (bottom of figure).

FIG. 23A-FIG. 23B are data graphs showing the prime editing efficiency of correcting the A1AT E342K mutation in HepG2 E342K cells via an A-to-G correction (A to G % correction; y-axis) as in FIG. 22 but the x-axis plots escalating mass ratio amounts. FIG. 23A x-axis plots escalating the mass ratio of PE mRNA versus total gRNA (“mRNA: total gRNA (mass)”). FIG. 23B x-axis plots escalating the mass ratio of pegRNA: ngRNA (“pegRNA:ngRNA (mass)”).

FIG. 24 is a table showing the experimental set-up for in vivo administration in of LNP encapsulated prime editing components (mRNA PEv6; pegRNA5278; ngRNA7) at mass ratios of 0.5 PE mRNA: 0.95 pegRNA5278: 0.05 ngRNA7 at a 0.5 or 2 milligrams per kilogram (mpk) dose in NSG-PiZ mice (mice express mutant SERPINA1 (E342K) mutation).

FIG. 25A-FIG. 25B are data showing the prime editing efficiency of correcting the A1AT E342K mutation in NSG-PiZ mice via an A-to-G correction (A to G % correction; y-axis). The right portion of the graph in FIG. 25A shows the percent of detectable E342K correction in livers of NSG-PiZ administered LNP encapsulated prime editing components in mass ratios and dosages outlined in FIG. 24 as well as displayed on the graph's x-axis. The left portion of the graph in FIG. 25A shows the percent indel rate in the same experiment. FIG. 25B is the corresponding table displaying the percent mean (mean (%)), standard deviation (SD), and number of animals in each group (n) for the A-to-G correction and indel percent rates shown in FIG. 25A.

FIG. 26A-FIG. 26B are data showing the editing efficiency of correcting the A1AT E342K mutation in HepG2 E342K cells via an A-to-G correction (A to G % correction; y-axis). FIG. 26A is a graph showing the percentage of editing efficiency (A7G) along with the indel rate after transfection with a prime editor variant (“PE2 V5”; “PE3 V5”; “PE2 V6”; “PE3 V6”) and a designed nuclear localization signal (NLS) pegRNA 5278 (also referred to as “gRNA9120” or “NLS-pegRNA9120”) when added at saturation. The FIG. 26B graph shows the dose response curve for the A-to-G correction versus the dosing (ng) for the addition of escalating concentrations of prime editor and pegRNA. The corresponding FIG. 26B table tabulates the hillslope, top A-to-G percent efficiency, and EC50 values for each experimental group.

FIG. 27 is a heat map displaying a scale of 0-15% editing efficiency of a A-to-G correction upon transfecting a HepG2 E342K cell line harboring the G>A SNP with prime editor and pegRNA variants. The heat map displays a panel of prime editor variants matrixed with a panel of pegRNA variants where one prime editor (labeled on the y-axis; see also Table 9) was added along with one pegRNA (labeled across the x-axis; see also Table 10) in one well at a dose of about 111 ng in a 96-well format.

FIG. 28 (SEQ ID Nos: 303 & 393-394) is a graph displaying the editing efficiency of an A-to-G correction (A to G % correction; y-axis) in the HPF E342K cell line for transfections with a panel of prime editor variants optimized with a variety of linkers (labeled on x-axis). Along with a prime editor variant the transfection master mix also included a pegRNA 5278 and nicking guide #7.

FIG. 29A (SEQ ID Nos: 305-306) is a graph displaying the editing efficiency of an A-to-G correction (A to G % correction; y-axis) in the HPF E342K cell line for a panel of prime editor variants optimized with a variety of linkers (labeled on x-axis). The legend refers to the ID number of the pegRNA variant included. The assay was performed at a saturation dose of 3.225 ug. FIG. 29B (SEQ ID Nos: 305-306) is the indel rate for the experiment described in FIG. 29A.

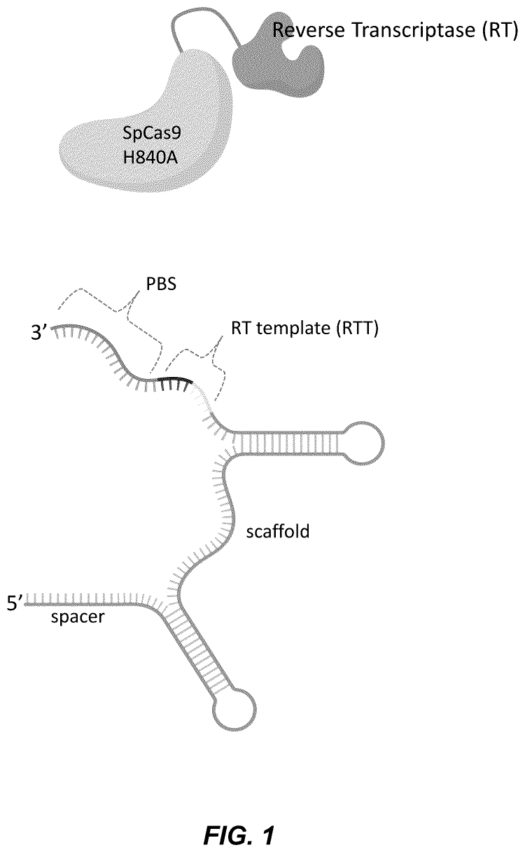

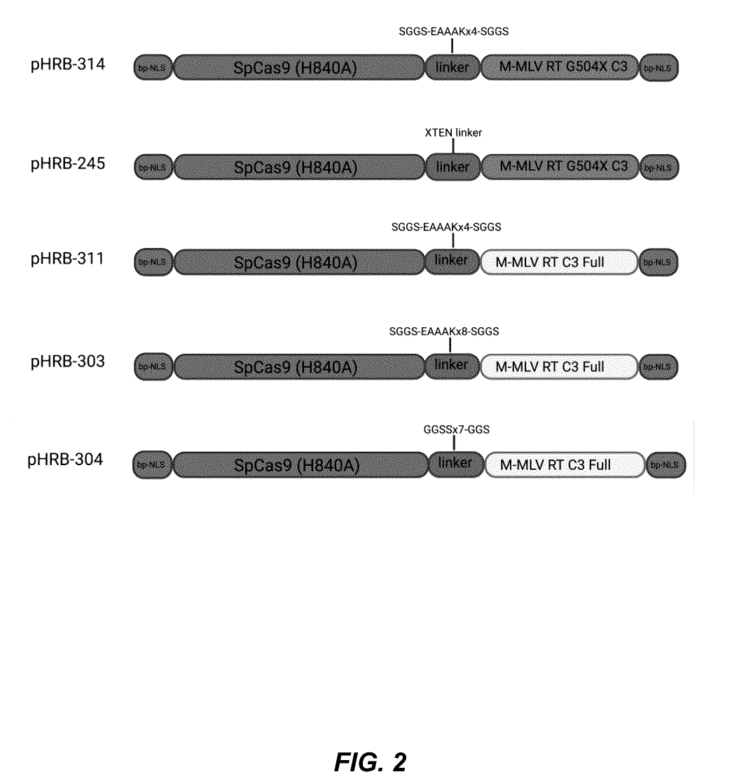

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

### Definitions

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a mammalian cell. In some embodiments, the cell is a human cell. A cell can be of or derived from different tissues, organs, and/or cell types. In some embodiments, the cell is a primary cell. As used herein, the term “primary cell”, means a cell isolated from an organism, e.g., a mammal, which is grown in tissue culture (i.e., in vitro) for the first time before subdivision and transfer to a subculture. In some embodiments, the cell is a stem cell. In some non-limiting examples, mammalian cells including primary cells and stem cells, can be modified through introduction of one or more polynucleotides, polypeptides, and/or prime editing compositions (e.g., through transfections, transduction, electroporation, and the like) and further passaged. Such modified cells may include hematopoietic stem cells (HSCs), hematopoietic progenitor cells, (HSPCs), hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells, hematopoietic stem progenitor cells), muscle cells and precursors of these somatic cell types. In some embodiments, the cell is a primary hepatocyte. In some embodiments, the cell is a primary human hepatocyte. In some embodiments, the cell is a stem cell. In some embodiments, the cell is a progenitor cell. In some embodiments, the cell is a pluripotent cell (e.g., a pluripotent stem cell) In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is an embryonic stem cell (ESC). In some embodiments, the cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a human subject. In some embodiments, the cell is an epithelial cell from lung, liver, stomach, or intestine. In some embodiments, the cell is an epithelial cell from lung, liver, stomach, or intestine of a human subject. In some embodiments, the cell is a retinal cell. In some embodiments, the cell is a retinal cell from a human subject.

In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a mesenchymal stem cell. In some embodiments, the cell is a human embryonic stem cell. In some embodiments, the cell is an human embryonic kidney cell. In some embodiments, the cell is a HEK293 cell. In some embodiments, the cell is a HEK293T cell.

In some embodiments, the cell is a CD34+ cell. In some embodiments, the cell is a hematopoietic stem cell (HSC). In some embodiments, the cell is a hematopoietic progenitor cell (HPC). In some embodiments, hematopoietic stem cells and hematopoietic progenitor cells are referred to as hematopoietic stem or progenitor cells (HSPCs). In some embodiments, the cell is a human HSC. In some embodiments, the cell is a human HPC. In some embodiments, the cell is a human HSPC. In some embodiments, the cell is a long term (LT)-HSC. In some embodiments, the cell is a short-term (ST)-HSC. In some embodiments, the cell is a myeloid progenitor cell. In some embodiments, the cell is a lymphoid progenitor cell. In some embodiments, the cell is a granulocyte monocyte progenitor cell. In some embodiments, the cell is a megakaryocyte erythroid progenitor cell. In some embodiments, the cell is a multipotent progenitor cell (MPP).

In some embodiments, the cell is a stem cell. In some embodiments, the cell is a human stem cell. In some embodiments, the cell is a hematopoietic stem cell (HSC) or a hematopoietic stem and progenitor cell. In some embodiments, the HSC is from bone marrow or mobilized peripheral blood. In some embodiments the human stem cell is an induced pluripotent stem cell (iPSC). In some embodiments, the cell is a human HSC. In some embodiments, the cell is a human CD34+ cell. In some embodiments, the cell is a hematopoietic stem and progenitor cell (HSPC). In some embodiments, the cell is a human hematopoietic stem and progenitor cell (HSPC). In some embodiments, the cell is a hematopoietic progenitor cell, multipotent progenitor cell, lymphoid progenitor cell, a myeloid progenitor cell, a megakaryocyte —erythroid progenitor cell, a granulocyte-megakaryocyte progenitor cell, a granulocyte, a promyelocyte, a neutrophil, an eosinophil, a basophil, an erythrocyte, a reticulocyte, a thrombocyte, a megakaryoblast, a platelet-producing megakaryocyte, a monocyte, a macrophage, a dendritic cell, a microglia, an osteoclast, a lymphocyte, a NK cell, a B-cell, or a T-cell. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into or give rise to recovery of a population of cells, e.g., neutrophils, platelets, red blood cells, monocytes, macrophages, antigen-presenting cells, microglia, osteoclasts, dendritic cells, inner ear cell, inner ear support cell, cochlear cell and/or lymphocytes. In some embodiments, the cell is in a subject, e.g., a human subject.

In some embodiments, a cell is not isolated from an organism but forms part of a tissue or organ of an organism, e.g., a mammal. In some non-limiting examples, mammalian cells include formed elements of the blood (e.g., lymphocytes, bone marrow cells), precursors of any of these somatic cell types, and stem cells.

In some embodiments, a cell is isolated from an organism. In some embodiments, a cell is derived from an organism. In some embodiments, a cell is a differentiated cell. In some embodiments, the cell is a fibroblast. In some embodiments, the cell is differentiated from an induced pluripotent stem cell. In some embodiments, the cell is differentiated from an HSC or an HPSC. In some embodiments, the cell is differentiated from an induced pluripotent stem cell (iPSC). In some embodiments, the cell is differentiated from an embryonic stem cell (ESC).

In some embodiments, the cell is a differentiated human cell. In some embodiments, cell is a human fibroblast. In some embodiments, the cell is differentiated from an induced human pluripotent stem cell. In some embodiments, the cell is differentiated from a human iPSC or a human ESC.

In some embodiments, the cell comprises a prime editor, a PEgRNA, or a prime editing composition disclosed herein. In some embodiments, the cell is from a human subject. In some embodiments, the human subject has a disease or condition, or is at a risk of developing a disease or a condition associated with a mutation to be corrected by prime editing. In some embodiments, the cell is from a human subject, and comprises a prime editor or a prime editing composition for correction of the mutation. In some embodiment, the cell comprises a mutation in a double stranded target DNA. In some embodiments, the cell comprises a mutation in a target gene. In some embodiments, the cell comprises a mutation that is associated with a disease, disorder, or a condition. In some embodiments, the cell is in a human subject. In some embodiments, the cell comprises a prime editor or a prime editing composition for correction of the mutation. In some embodiments, the cell is in a human subject, and comprises a prime editor, a PEgRNA, or a prime editing composition disclosed herein for correction of the mutation.

In some embodiments, the cell is from a human subject. In some embodiments, the cell is from a human subject and the mutation has been edited or corrected by prime editing.

The term “substantially” as used herein can refer to a value approaching 1000% of a given value. In some embodiments, the term can refer to an amount that can be at least about 70%, 80%, 90%, 91%, 92%, 93%, 94%, 95%, 96%, 97%, 98%, 99%, 99.9%, or 99.99% of a total amount. In some embodiments, the term can refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises at least 10 amide bonds, 15 amide bonds, 20 amide bonds, 30 amide bonds, or 50 amide bonds. In some embodiments, a protein comprises an enzyme, enzyme precursor protein, regulatory protein, structural protein, cytokine, chemokine, growth factor, receptor, nucleic acid binding protein, a biomarker, a member of a specific binding pair (e.g., a ligand or aptamer), or an antibody. In some embodiments, a protein can be a full-length protein (e.g., a fully processed protein having certain biological function). In some embodiments, a protein can be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor can be a fusion protein comprising a Cas9 protein domain of S. pyogenes or a fragment, mutant, or variant thereof and a reverse transcriptase protein domain of a retrovirus (e.g., Moloney murine leukemia virus) or a mutant, fragment, or variant of the retrovirus. A protein that comprises amino acid sequences from different origins or naturally occurring proteins can be referred to as a fusion, or a chimeric protein.

In some embodiments, a protein comprises a functional variant or functional fragment of a full-length wild-type protein. A “functional fragment” or “functional portion”, as used herein, refers to any portion of a reference protein (e.g., a wild-type protein) that encompasses less than the entire amino acid sequence of the reference protein while retaining one or more of the functions, e.g., catalytic or binding functions. For example, a functional fragment of a reverse transcriptase can encompass less than the entire amino acid sequence of a wild-type reverse transcriptase but retains the ability under at least one set of conditions to catalyze the polymerization of a polynucleotide. When the reference protein is a fusion of multiple functional domains, a functional fragment thereof can retain one or more of the functions of at least one of the functional domains. For example, a functional fragment of a Cas9 can encompass less than the entire amino acid sequence of a wild-type Cas9 but retains its DNA binding ability and lack its nuclease activity partially or completely.

A “functional variant” or “functional mutant”, as used herein, refers to any variant or mutant of a reference protein (e.g., a wild-type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase can comprise one or more amino acid substitutions compared to the amino acid sequence of a wild-type reverse transcriptase but retains the ability under at least one set of conditions to catalyze the polymerization of a polynucleotide. When the reference protein is a fusion of multiple functional domains, a functional variant thereof can retain one or more of the functions of at least one of the functional domains. For example, in some embodiments, a functional fragment of a Cas9 can comprise one or more amino acid substitutions in a nuclease domain, e.g., an H840A amino acid substitution, compared to the amino acid sequence of a wild-type Cas9, but retains the DNA binding ability and lacks the nuclease activity partially or completely.

The term “function” and its grammatical equivalents as used herein may refer to a capability of operating, having, or serving an intended purpose. Functional can comprise any percent from baseline to 100% of an intended purpose. For example, functional can comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional can mean over or over about 100% of normal function, for example, 125%, 150%, 175%, 200/o, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence, or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences can exhibit at least 93%, 95%, 98% or 99% sequence identity to the reference nucleic acid sequence. For example, a “region of homology to a genomic region” can be a region of DNA that has a similar sequence to a given genomic region in the genome. A region of homology can be of any length that is sufficient to promote binding of, e.g., a spacer or a primer binding site sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, an alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another corresponding position in a Cas9 homolog when the Cas9 homolog is aligned against the reference Cas9 sequence. The term “homolog” as used herein refers to a gene or a protein that is related to another gene or protein by a common ancestral DNA sequence. A homolog can be an ortholog or a paralog. An ortholog refers to a gene or protein that is related to another gene or protein by a speciation event. A paralog refers to a gene or protein that is related to another gene or protein by a duplication event within a genome. A paralog may be within the same species of the gene or protein it is related to. A paralog may also be in a different species of the gene or protein it is related to. In some embodiments, an ortholog may retain the same function. In some embodiments, a paralog may evolve a new function.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide can comprise one or more other nucleotide bases, such as inosine (I), which is read by the translation machinery as guanine (G).

In some embodiments, a polynucleotide can be modified. As used herein, the terms “modified” or “modification” refers to chemical modification with respect to the A, C, G, T and U nucleotides. In some embodiments, modifications can be on the nucleoside base and/or sugar portion of the nucleosides that comprise the polynucleotide. In some embodiments, the modification can be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which can be Watson Crick, Hoogsteen or reversed Hoogsteen hydrogen bonding. For example, an adenine on one polynucleotide molecule will base pair to a thymine or uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3\ A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be at least 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantially complementary” can also refer to a 100% complementarity over a portion or region of two polynucleotide molecules. In some embodiments, the portion or region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides, e.g., DNA, are transcribed into mRNA and/or the process by which polynucleotides, e.g., the transcribed mRNA, translated into peptides, polypeptides, or proteins. If the polynucleotide is derived from genomic DNA, expression can include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, can comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations can comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state. A “reference sequence” is a defined sequence used as a basis for sequence comparison. A reference sequence can be a subset of or the entirety of a specified sequence; for example, a segment of a full-length cDNA sequence, RNA sequence, DNA sequence, gene sequence or polypeptide sequence, or the complete cDNA sequence, RNA sequence, DNA sequence, gene sequence or polypeptide sequence. In some embodiments, a reference sequence is a wild-type sequence of a protein of interest or a variant thereof. In other embodiments, a reference sequence is a polynucleotide sequence encoding a wild-type protein or a variant thereof.

The term “subject” and its grammatical equivalents as used herein may refer to a human or a non-human. A subject can be a mammal. A human subject can be male or female. A human subject can be of any age. A subject can be a human embryo. A human subject can be a newborn, an infant, a child, an adolescent, or an adult. A human subject can be up to about 100 years of age. A human subject can be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include supportive treatment, that is, treatment employed to supplement another specific therapy directed toward the improvement of the disease, condition, or disorder. In some embodiments, a condition can be pathological. In some embodiments, a treatment can not completely cure or prevent a disease, condition, or disorder. In some embodiments, a treatment ameliorates, but does not completely cure or prevent a disease, condition, or disorder. In some embodiments, a subject can be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, reverse, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g. a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example, a prime editing composition comprising a construct, that can be sufficient to result in a desired activity upon introduction into a subject as disclosed herein. An effective amount of the prime editing compositions can be provided to a target gene or cell, whether the cell is ex vivo or in vivo. An effective amount can be the amount to induce, for example, at least about a 2-fold change (increase or decrease) or more in the amount of target nucleic acid modulation observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target gene to produce a functional protein). The amount of target gene modulation can be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

An effective amount can be the amount to induce, when administered to a population of cells, a certain percentage of the population of cells to have a correction of a mutation. For example, in some embodiments, an effective amount can be the amount to induce, when administered to or introduced to a population of cells, installation of one or more intended nucleotide edits that correct a mutation in the target gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

The term “reverse transcriptase” or “RT” as used herein refers to a class of enzymes that synthesize a DNA molecule from an RNA template. An RT may require the primer molecule with an exposed 3′ hydroxyl group. In some embodiments, the primer molecule of an RT is a DNA molecule. In some embodiments, the primer molecule of an RT is an RNA molecule. In some embodiments, an RT comprises both DNA polymerase activity and RNase H activity. The two activities can reside in two separate domains in an RT.

The term “linker” as used herein refers to a bond, a chemical group, or a molecule linking two molecules or moieties, e.g., two protein domains to form a fusion protein. In some embodiments, a linker is a peptide linker. In some embodiments, a linker is a polynucleotide or a oligonucleotide linker. For example, a RNA-binding protein recruitment sequence, such as a MS2 polynucleotide sequence, can be used to connect a Cas9 domain and a DNA polymerase domain of a prime editor, wherein one of the Cas9 domain and the DNA polymerase domain is fused to a MS2 coat protein. In some embodiments, a peptide linker can have various lengths, depending on the application of a linker or the sequences or molecules being linked by a linker.

The term “fusion protein” refers to a protein comprised of domains from more than one naturally occurring or recombinantly produced protein, where generally each domain serves a different function. A domain may comprise a particular makeup of amino acids. A domain may also comprise a structure of proteins as described herein.

Disclosed herein in some embodiments, are compositions comprising polynucleotides and constructs that comprises a nucleic acid that codes for a PEgRNA as described above, a nick guide sequence as describe above, a primer editor, a prime editing composition or any combination thereof. In certain embodiments, provided herein are prime editors for programmable prime editing of target polynucleotides, e.g., target genes.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target DNA polynucleotide, e.g., a target gene of prime editing can comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand can also be referred to as the “non-Protospacer Adjacent Motif (non-PAM strand).” In some embodiments, the non-target strand can also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence can be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease, e.g., a Cas9 nickase or a Cas9 nuclease. A PAM sequence can be modified by introducing one or more mutations to alter the PAM specificity. A protospacer sequence refers to a specific sequence in the PAM strand of the double stranded target DNA (e.g., target gene) that is complementary to the search target sequence. In a PEgRNA, a spacer sequence can have a substantially identical sequence as the protospacer sequence on the edit strand of the double stranded target DNA (e.g., target gene) except that the spacer sequence can comprise Uracil (U) and the protospacer sequence can comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a C. lari Cas9 nickase that comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

In some embodiments, a PEgRNA complexes with and directs a prime editor to bind to the search target sequence of the target gene. In some embodiments, the bound prime editor generates a nick on the edit strand (PAM strand) of the target gene. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to an endogenous target gene sequence. Accordingly, in some embodiments, the editing template of a PEgRNA is complementary to a sequence in the edit strand except for one or more mismatches at the intended nucleotide edit positions in the editing template. In some embodiments, the newly synthesized single stranded DNA has identity or substantial identity to a sequence in the editing target sequence, except for one or more insertions, deletions, or substitutions at the intended nucleotide edit positions. The endogenous, e.g., genomic, sequence that is partially complementary to the editing template may be referred to as an “editing target sequence”.

In some embodiments, the newly synthesized single-stranded DNA equilibrates with the editing target on the edit strand of the double stranded target DNA (e.g., the target gene) for pairing with the target strand of the targe gene. In some embodiments, the editing target sequence of the double stranded target DNA (e.g., target gene) is excised by a flap endonuclease (FEN), for example, FEN1. In some embodiments, the FEN is an endogenous FEN, for example, in a cell comprising the double stranded target DNA, e.g., a target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the double stranded target DNA (e.g., target gene). In some embodiments, the newly synthesized single stranded DNA and the endogenous DNA on the target strand form a heteroduplex DNA structure at the region corresponding to the editing target sequence of the double stranded target DNA (e.g., target gene). In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the double stranded target DNA (e.g., the target gene).

### Prime Editor

The term “prime editor (PE)” refers to the polypeptide or polypeptide components involved in prime editing. In various embodiments, a prime editor includes a polypeptide domain having DNA binding activity (e.g., a DNA binding domain) and a polypeptide domain (e.g., a DNA polymerase domain) having DNA polymerase activity. In some embodiments, a prime editor comprises a polypeptide domain (e.g., a DNA binding domain) having DNA binding activity. In some embodiments, a prime editor comprises a polypeptide that comprises a DNA binding domain. In some embodiments, a prime editor comprises a DNA binding domain. In some embodiments, a prime editor comprises a polypeptide domain having DNA polymerase activity (e.g., a DNA polymerase domain). In some embodiments, a prime editor comprises a polypeptide that comprises a DNA polymerase domain. In some embodiments, a prime editor comprises a DNA polymerase domain. In some embodiments, a prime editor comprises a polypeptide that comprises a DNA binding domain and a polypeptide that comprises a DNA polymerase domain. In some embodiments, a prime editor comprises a DNA binding domain and a DNA polymerase domain. In some embodiments, the prime editor comprises a DNA binding domain and DNA polymerase domain that is linked by a linker, e.g., a peptide linker, e.g., a GS rich peptide linker. In some embodiments, the prime editor comprises a fusion polypeptide that comprises a DNA binding domain and a DNA polymerase domain linked by a linker, e.g., a peptide linker, e.g., a GS rich peptide linker.

In some embodiments, the prime editor comprises a polypeptide domain having a nuclease activity. In some embodiments, the polypeptide domain having DNA binding activity comprises a nuclease domain or nuclease activity. In some embodiments, the DNA binding domain comprises a nuclease domain or nuclease activity. In some embodiments, the polypeptide domain having the nuclease activity comprises a nickase, or a fully active nuclease. In some embodiments, the DNA binding domain comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the DNA binding domain comprises a nuclease domain that is an inactive nuclease; e.g., dCas9. In some embodiments, the DNA binding domain comprises a comprises a nucleic acid guided DNA binding domain, for example, a CRISPR-Cas protein, for example, a Cas9 nickase, a Cpf1 nickase, or another CRISPR-Cas nuclease. In some embodiments, the DNA binding domain (e.g., a nucleic acid guided DNA binding domain) is a Cas protein domain. In some embodiments, the Cas protein is a Cas9; e.g., Cas9 nuclease; e.g., dCas9, Cas9 nickase. In some embodiments, the Cas protein domain comprises a nickase or a nickase activity. In some embodiments, the DNA binding domain is a Cas9 or a variant thereof (e.g., a nickase variant). In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease.

In some embodiments, the polypeptide domain having DNA polymerase activity comprises a template-dependent DNA polymerase, for example, a DNA-dependent DNA polymerase or an RNA-dependent DNA polymerase. In some embodiments, the DNA binding domain comprises a template-dependent DNA polymerase for example, a DNA-dependent DNA polymerase or an RNA-dependent DNA polymerase. In some embodiments, the DNA polymerase domain comprises a reverse transcriptase domain (RT domain) or a reverse transcriptase (RT). In some embodiments, the DNA polymerase domain is a RT domain or a RT. In some embodiments, a prime editor comprises a reverse transcriptase (RT) activity. For example, the first polypeptide of the prime editor may have activity for target primed reverse transcription. In some embodiments, the polypeptide domain having DNA polymerase activity comprises a reverse transcriptase activity (e.g., activity for target primed reverse transcription).

In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA —protein recruitment polypeptide, for example, a MS2 coat protein.

In some embodiments, a prime editor comprises a Cas polypeptide (i.e., a DNA binding domain) and a reverse transcriptase polypeptide (i.e., a DNA polymerase domain) that are derived from different species. For example, a prime editor may comprise a S. pyogenes Cas9 polypeptide and a Moloney murine leukemia virus (M-MLV) reverse transcriptase polypeptide. In some embodiments, the prime editor comprises a fusion polypeptide that comprises a comprises a Cas polypeptide (i.e., a DNA binding domain) and a reverse transcriptase polypeptide (i.e., a DNA polymerase domain) that are derived from different species. For example, a prime editor may comprise a S. pyogenes Cas9 polypeptide and a Moloney murine leukemia virus (M-MLV) reverse transcriptase (RT) polypeptide.

In some embodiments, polypeptide domains of a prime editor (e.g., a DNA binding domain and a DNA polymerase domain) are fused or linked by a peptide linker to form a fusion protein. In other embodiments, a prime editor comprises one or more polypeptide domains (e.g., a DNA binding domain and a DNA polymerase domain) provided in trans as separate proteins, which are capable of being associated to each other through non-peptide linkages or through aptamers or recruitment sequences. In some embodiments, a prime editor comprises a DNA binding domain and a DNA polymerase domain (e.g., a reverse transcriptase domain or RT) fused or linked with each other by a peptide linker.

In some embodiments, the prime editor comprises a DNA binding domain and a DNA polymerase domain (e.g., a reverse transcriptase domain or RT) fused or linked with each other by an RNA-protein recruitment aptamer, e.g., a MS2 aptamer, which can, in some embodiments, be linked to a PEgRNA.

In some embodiments, a prime editor further comprises one or more nuclear localization sequence (NLS). In some embodiments, one or more polypeptides of the prime editor are fused to or linked to (e.g., via a peptide linker) one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

Prime editor polypeptide components can be encoded by one or more polynucleotides in whole or in part. The present disclosure contemplates polynucleotides encoding the prime editor components, for example, a polynucleotide encoding a DNA binding domain, and a polynucleotide encoding a DNA polymerase domain. The present disclosure also contemplates a single polynucleotide comprising a polynucleotide encoding a DNA binding domain, and a polynucleotide encoding a DNA polymerase domain. In some embodiments, a prime editing composition comprises a polynucleotide encoding a DNA polymerase domain. In some embodiments, the polynucleotide encoding a DNA polymerase domain is a DNA. In some embodiments, the polynucleotide encoding a DNA polymerase domain is an RNA (e.g., a mRNA). In some embodiments, a prime editing composition comprises a polynucleotide encoding a DNA binding domain. In some embodiments, the polynucleotide encoding the DNA binding domain is a DNA. In some embodiments, the polynucleotide encoding the DNA binding domain is an RNA (e.g., a mRNA). In some embodiments, the polynucleotide encoding a DNA binding domain, and the polynucleotide encoding a DNA polymerase domain are linked by a linker polynucleotide (e.g., that encodes a peptide linker) to result in a fusion protein (e.g., a prime editor) that comprises the DNA polymerase domain and DNA binding domain linked by a peptide linker. In some embodiments, the linker polynucleotide is a DNA. In some embodiments, the linker polynucleotide is an RNA (e.g., mRNA). In some embodiments, the polynucleotide sequence encoding a DNA binding domain, and the polynucleotide encoding a DNA polymerase domain are linked by a linker polynucleotide (e.g., that encodes a peptide linker) further comprises one or more polynucleotide sequences encoding one or more NLS to result in a fusion protein (e.g., a prime editor) that comprises the DNA polymerase domain and DNA binding domain linked by a peptide linker and further fused to or linked to one or more NLS.

In some embodiments, a single polynucleotide (e.g., a single mRNA) construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein can comprise an N-terminal portion fused to an intein-N and a C-terminal portion fused to an intein-C, each of which is individually encoded by an AAV vector. In some embodiments, components of a prime editor disclosed herein (e.g., a polypeptide comprising a DNA binding domain and/or a polypeptide comprising a DNA polymerase domain) can be brought together post-translationally via a split-intein.

In some embodiments, a prime editor polypeptide may comprise an amino acid sequence, wherein the initial methionine (at position 1) is optionally not present. In some embodiments, a prime editor polypeptide sequence may comprise a N-terminal methionine residue. In some embodiments, a prime editor polypeptide sequence may lack a N-terminus methionine. In some embodiments, the N-terminal methionine encoded by the translation initiation codon, e.g., ATG, may be removed from the prime editor polypeptide after translation. In some embodiments, the N-terminal methionine encoded by the translation initiation codon, e.g., ATG, may remain present in the prime editor polypeptide sequence. In some embodiments, the amino acid sequence of a prime editor polypeptide can be N-terminally modified by one or more processing enzymes, e.g., by Methionine aminopeptidases (MAP).

In some embodiments, a prime editor comprises a DNA polymerase domain and a DNA binding domain, wherein the amino acid sequences of the DNA polymerase domain and/or the DNA binding domain comprise aN terminus methionine. In some embodiments, a prime editor comprises a DNA polymerase domain that comprises an amino acid sequence that lacks a N-terminus methionine relative to a reference DNA polymerase amino acid sequence. In some embodiments, a prime editor comprises a DNA binding domain that comprises an amino acid sequence that lacks a N-terminus methionine relative to a reference DNA binding domain amino acid sequence.

In some embodiments, a prime editor and/or a component thereof (e.g., a DNA binding domain or a polypeptide comprising a DNA binding domain and/or a DNA polymerase domain or a polypeptide comprising a DNA polymerase domain) can be engineered. In some embodiments, the polypeptide components of a prime editor do not naturally occur in the same organism or cellular environment. In some embodiments, the polypeptide components of a prime editor can be of different origins or from different organisms. In some embodiments, a prime editor comprises a DNA binding domain and a DNA polymerase domain that are derived from different species.

In some embodiments, a prime editor comprises a RT or an RT domain (e.g., a M-MLV RT) that is rationally engineered. Such an engineered RT or RT domain can comprise, for example, sequences or amino acid changes different from a naturally occurring RT or RT domain. In some embodiments, the engineered RT or RT domain comprises improved RT activity relative to a corresponding naturally occurring RT or RT domain. In some embodiments, the engineered RT or RT domain comprises improved prime editing efficiency relative to a corresponding naturally occurring RT or RT domain, when used in a prime editor. In some embodiments, the pegRNA contains a

### Prime Editor Nucleotide Polymerase Domain

In some embodiments, a prime editor comprises a polypeptide domain (e.g., a DNA polymerase domain) comprising a DNA polymerase activity. In some embodiments, the prime editor comprises a polypeptide that comprises a DNA polymerase domain. In some embodiments, a prime editing composition comprises a polynucleotide that encodes a polymerase domain, e.g., a DNA polymerase domain. In some embodiments, a prime editor comprises a nucleotide polymerase domain, e.g., a DNA polymerase domain. In some embodiments, the DNA polymerase domain can be a wild-type DNA polymerase domain, a full-length DNA polymerase protein domain, or can be a functional mutant, a functional variant, or a functional fragment thereof. In some embodiments, the DNA polymerase domain is a template dependent DNA polymerase domain. For example, the DNA polymerase can rely on a template polynucleotide strand, e.g., the editing template sequence, for new strand DNA synthesis. In some embodiments, the prime editor comprises a DNA polymerase domain that is a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. In some embodiments, the chimeric or hybrid PEgRNA can comprise an RNA portion (including the spacer and the gRNA core) and a DNA portion (the extension arm comprising the editing template that includes a strand of DNA).

In some embodiments, the prime editor comprises a DNA polymerase domain that is a RNA-dependent DNA polymerase. In some embodiments, the DNA polymerase domain can be a wild type polymerase, for example, from eukaryotic, prokaryotic, archaeal, or viral organisms. In some embodiments, the DNA polymerase domain is a modified DNA polymerase, for example, a wild-type DNA polymerase that is modified by genetic engineering, mutagenesis, or directed evolution-based processes.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase comprises is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase is an eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Revl DNA polymerase. In some embodiments, the DNA polymerase is a human Revl DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DPI/DP22-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase is a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity. Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). In some embodiments, the DNA polymerase domain is an RNA-dependent DNA polymerase domain, for example, a reverse transcriptase (RT). In some embodiments, the DNA polymerase domain is a reverse transcriptase (RT) domain, for example, a reverse transcriptase (RT). In some embodiments, the reverse transcriptase (RT), or a RT domain is a M-MLV RT (e.g., a wild-type M-MLV RT, a reference M-MLV RT, a functional mutant, a functional variant, or a functional fragment thereof). An RT or an RT domain can be a wild-type RT domain, a full-length RT domain, or may be a functional mutant, a functional variant, or a functional fragment thereof. An RT or an RT domain of a prime editor can comprise a wild-type RT a full length RT, a functional mutant, a functional variant, or a functional fragment thereof or can be engineered or evolved to contain specific amino acid substitutions, truncations, or variants. An engineered RT can comprise sequences or amino acid changes different from a naturally occurring RT or a corresponding reference RT. In some embodiments, the engineered RT can have improved reverse transcription activity over a naturally occurring RT or RT domain. In some embodiments, the engineered RT can have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, the reverse transcriptase domain or RT can be between 200 and 800 amino acids in length, between 300 and 700 amino acids in length, or at least 400 and 600 amino acids in length. In some embodiments, the reverse transcriptase domain or RT can be at least 200 amino acids in length, at least 300 amino acids in length, at least 400 amino acids in length, at least 500 amino acids in length, or at least 600 amino acids in length. In some embodiments, the reverse transcriptase domain or RT is 250 amino acids in length. In some embodiments, the reverse transcriptase domain or RT is 350 amino acids in length. In some embodiments, the reverse transcriptase domain or RT is 450 amino acids in length. In some embodiments, the reverse transcriptase domain or RT is 550 amino acids in length. In some embodiments, the reverse transcriptase domain or RT is 650 amino acids in length.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retro RT.

In some embodiments, a prime editor comprises a virus RT, for example, a retrovirus RT. Non limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild-type M-MLV RT, a reference M-MLV RT, a functional mutant, a functional variant, or a functional fragment thereof. In some embodiments, the RT domain or a RT is a M-MLV RT (e.g., wild-type M-MLV RT, a reference M-MLV RT, a functional mutant, a functional variant, or a functional fragment thereof). In some embodiments, a reference M-MLV RT is a wild-type M-MLV RT. An exemplary sequence of a wild-type M-MLV RT is provided in SEQ ID NO: 73 (TLNIEDEHRLHETSKEPDVSLGSTWLSDFPQAWAETGGMGLAVRQAPLIIPLKATST PVSIKQYPMSQEARLGIKPHIQRLLDQGILVPCQSPWNTPLLPVKKPGTNDYRPVQDL REVNKRVEDIHPTVPNPYNLLSGLPPSHQWYTVLDLKDAFFCLRLHPTSQPLFAFEW RDPEMGISGQLTWTRLPQGFKNSPTLFDEALHRDLADFRIQHPDLILLQYVDDLLLA ATSELDCQQGTRALLQTLGNLGYRASAKKAQICQKQVKYLGYLLKEGQRWLTEAR KETVMGQPTPKTPRQLREFLGTAGFCRLWIPGFAEMAAPLYPLTKTGTLFNWGPDQ QKAYQEIKQALLTAPALGLPDLTKPFELFVDEKQGYAKGVLTQKLGPWRRPVAYLS KKLDPVAAGWPPCLRMVAAIAVLTKDAGKLTMGQPLV ILAPHAVEALVKQPPDRW LSNARMTHYQALLLDTDRVQFGPVVALNPATLLPLPEEGLQHNCLDILAEAHGTRPD LTDQPLPDADHTWYTDGSSLLQEGQRKAGAAVTTETEVIWAKALPAGTSAQRAELI ALTQALKMAEGKKLNVYTDSRYAFATAHIHGEIYRRRGLLTSEGKEIKNKDEILALL KALFLPKRLSIIHCPGHQKGHSAEARGNRMADQAARKAAITETPDTSTLLIENSSP).

An exemplary sequence of a reference M-MLV RT is provided in SEQ ID NO: 74 (TLNIEDEYRLHETSKEPDVSLGSTWLSDFPQAWAETGGMGLAVRQAPLIIPLKATST PVSIKQYPMSQEARLGIKPHIQRLLDQGILVPCQSPWNTPLLPVKKPGTNDYRPVQDL REVNKRVEDIHPTVPNPYNLLSGLPPSHQWYTVLDLKDAFFCLRLHPTSQPLFAFEW RDPEMGISGQLTWTRLPQGFKNSPTLFDEALHRDLADFRIQHPDLILLQYVDDLLLA ATSELDCQQGTRALLQTLGNLGYRASAKKAQICQKQVKYLGYLLKEGQRWLTEAR KETVMGQPTPKTPRQLREFLGTAGFCRLWIPGFAEMAAPLYPLTKTGTLFNWGPDQ QKAYQEIKQALLTAPALGLPDLTKPFELFVDEKQGYAKGVLTQKLGPWRRPVAYLS KKLDPVAAGWPPCLRMVAAIAVLTKDAGKLTMGQPLVILAPHAVEALVKQPPDRW LSNARMTHYQALLLDTDRVQFGPVVALNPATLLPLPEEGLQHNCLDILAEAHGTRPD LTDQPLPDADHTWYTDGSSLLQEGQRKAGAAVTTETEVIWAKALPAGTSAQRAELI ALTQALKMAEGKKLNVYTDSRYAFATAHIHGEIYRRRGLLTSEGKEIKNKDEILALL KALFLPKRLSIIHCPGHQKGHSAEARGNRMADQAARKAAITETPDTSTLLIENSSP).

An exemplary sequence of a M-MLV RT is provided in SEQ ID NO: 383 (TLNIEDEYRLHETSKEPDVSLGSTWLSDFPQAWAETGGMGLAVRQAPLIIPLKATST PVSIKQYPMSQEARLGIKPHIQRLLDQGILVPCQSPWNTPLLPVKKPGTNDYRPVQDL REVNKRVEDIHPTVPNPYNLLSGLPPSHQWYTVLDLKDAFFCLRLHPTSQPLFAFEW RDPEMGISGQLTWTRLPQGFKNSPTLFNEALHRDLADFRIQHPDLILLQYVDDLLLA ATSELDCQQGTRALLQTLGNLGYRASAKKAQICQKQVKYLGYLLKEGQRWLTEAR KETVMGQPTPKTPRQLREFLGKAGFCRLFIPGFAEMAAPLYPLTKPGTLFNWGPDQQ KAYQEIKQALLTAPALGLPDLTKPFELFVDEKQGYAKGVLTQKLGPWRRPVAYLSK KLDPVAAGWPPCLRMVAAIAVLTKDAGKLTMGQPLVILAPHAVEALVKQPPDRWL SNARMTHYQALLLDTDRVQFGPVVALNPATLLPLPEEGLQHNCLDILAEAHG).

In some embodiments, the M-MLV RT variant comprises D200N, T306K, W313F, T330P, and L603W amino acid substitutions as compared to reference M-MLV RT sequence SEQ ID NO: 73.

In some embodiments, a prime editor comprises an RT that comprises an engineered RNase domain compared to a corresponding reference RT (e.g., a reference M-MLV RT or a wild-type M-MLV RT). In some embodiments, the RT of the prime editor comprises one or more amino acid substitutions, insertions, or deletions compared to a reference RT. In some embodiments, the RT of the prime editor is truncated compared to a corresponding reference RT (e.g., a reference M-MLV RT or a wild-type M-MLV RT). A polypeptide is “truncated” when, compared to a reference polypeptide sequence, the polypeptide lacks an end portion, for example, a N-terminal portion or a C-terminal portion. A polypeptide is truncated after amino acid position n means that the polypeptide, compared to a reference polypeptide sequence, lacks amino acids that are C-terminal to amino acid n or corresponding amino acids thereof, but retains amino acid n. In other words, “truncated after amino acid at position n” or “truncated at C terminus between positions n and n+1” refers to a truncation of a polypeptide between positions n and n+1, wherein amino acids that are C-terminal to amino acid n are deleted compared to a reference polypeptide sequence. In some embodiments, a polypeptide truncated after amino acid n, when compared to a reference polypeptide sequence, comprises amino acid n and all amino acids N terminal to amino acid n and lacks amino acids C terminal to amino acid n, or corresponding amino acids thereof.

In some embodiments, a polypeptide truncated before amino acid n, or a polypeptide truncated at N terminus between positions n−1 and n, when compared to a reference polypeptide sequence, comprises amino acid n and all amino acids C terminal to amino acid n and lacks amino acids N terminal to amino acid n, or corresponding amino acids thereof. In some embodiments, a truncated polypeptide is truncated at the N terminus, at the C terminus, or both the N terminus and the C terminus. A C terminal truncated polypeptide may also be truncated at its N terminus. An N terminal truncated polypeptide may also be truncated at its C terminus. In some embodiments, the RT of the prime editor consists of 95%, 90%, 85%, 80%, 75%, 70%, 65%, 60%, 55%, 50%, 45%, 40%, 35%, 30%, 25%, 20%, 15% or 10% of amino acids of a corresponding reference RT. In some embodiments, the prime editor comprises a truncated RT compared to a corresponding reference RT, wherein the truncation is at the N-terminus of the RT. In some embodiments, the prime editor comprises a truncated RT compared to a corresponding reference RT, wherein the truncation is at the C-terminus of RT. In some embodiments, the prime editor comprises a truncated RT compared to a corresponding reference RT, wherein the truncation is within the middle of corresponding reference RT. In some embodiments, the prime editor comprises a truncated RT compared to a corresponding reference RT, wherein the RT domain is truncated at both the N-terminus and the C-terminus. In some embodiments, the prime editor comprises a truncated RT compared to a corresponding reference RT, wherein the RT is truncated at the N-terminus, the C-terminus, and/or the middle of the RT referenced by the corresponding RT. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390,400, 410, 420, 430, 440,450, 460, 470, 480, 490, 500, 510, 520, 530, 540, 550 or more amino acids are truncated at the N-terminus of the RT in a prime editor compared to a corresponding reference RT. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550 or more amino acids are truncated at the C-terminus of the RT in a prime editor compared a corresponding reference RT. In some embodiments, a reference RT sequence has the sequence of SEQ ID NO: 73.

In some embodiments, a prime editor comprises an RT that is a Moloney murine leukemia virus (M-MLV) reverse transcriptase (M-MLV RT). In some embodiments, the M-MLV RT of the prime editor comprises one or more amino acid substitutions, insertions, or deletions compared to a wild-type M-MLV RT or a reference M-MLV RT. In some embodiments, a prime editor comprises a truncated M-MLV RT compared to a wild-type M-MLV RT or a reference M-MLV RT. In some embodiments, the M-MLV RT of the prime editor consists of 95%, 90%, 85%, 80%, 75%, 70%, 65%, 60%, 55%, 50%, 45%, 40%, 35%, 30%, 25%, 20%, 15% or 10% of amino acids of a wild-type M-MLV RT or a reference M-MLV RT. In some embodiments, the M-MLV RT of the prime editor is truncated at the N-terminus compared to a wild-type M-MLV RT or a reference M-MLV RT. In some embodiments, the M-MLV RT of the prime editor is truncated at the C-terminus compared to a wild-type M-MLV RT or a reference M-MLV RT. In some embodiments, the M-MLV RT of the prime editor is truncated compared to a wild-type M-MLV RT or a reference M-MLV RT, wherein the truncation is within the middle of the RT referenced by a wild-type M-MLV RT or a reference M-MLV RT. In some embodiments, the M-MLV RT of the prime editor comprises a truncated M-MLV RT compared to a wild-type M-MLV RT or a reference M-MLV RT, wherein RT is truncated at both the N-terminus and the C-terminus. In some embodiments, the M-MLV RT of the prime editor comprises a truncated M-MLV RT compared to a wild-type M-MLV RT or a reference M-MLV RT, wherein the RT is truncated at the N-terminus, the C-terminus, and/or the middle of the RT as reference by a wild-type M-MLVRT or a reference M-MLV RT.

In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more amino acids are truncated at the N-terminus of the M-MLV RT in a prime editor compared to a wild-type M-MLV RT or a reference M-MLV RT. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410,420, 430, 440, 450, 460,470, 480, 490, 500, 510, 520, 530, 540, 550 or more amino acids are truncated at the C-terminus of the M-MLV RT in a prime editor compared a wild-type M-MLV RT or a reference M-MLV RT.

In some embodiments, a prime editor comprises a reverse transcriptase (RT) that comprises a RNase domain. For example, in some embodiments, the RT of the prime editor is a virus RT domain that comprises a RNase domain. In some embodiments, the RT of the prime editor is a virus RT domain that comprises a RNase H domain. In some embodiments, the RT of the prime editor comprises a RNase H domain having 5′ and/or 3′ ribonuclease activity. In some embodiments, the RT of the prime editor comprises a RNase H domain having 3′ and/or 5′ nuclease activity toward the RNA strand when contacted with a DNA-RNA hybrid double strand.

In some embodiments, a prime editor comprises an RT that comprises an engineered RNase domain compared to a corresponding reference RT. In some embodiments, a prime editor comprises a RT that comprises an engineered RNase H domain compared to a corresponding reference RT. In some embodiments, the RT of the prime editor comprises one or more amino acid substitutions, insertions, or deletions in the RNase H domain compared to a corresponding. In some embodiments, the one or more amino acid substitutions, insertions, or deletions in the RNase H domain reduces or abolishes RNase activity of the RNase H domain. In some embodiments, the RT of the prime editor comprises a RNase H domain that has decreased or abolished RNase activity. In some embodiments, the RT of the prime editor comprises an inactivated RNase H domain. In some embodiments, the RT of the prime editor comprises one or more amino acid substitutions in a RNase H domain that decrease or abolish activity of the RNase H domain as compared to a corresponding reference RT. In some embodiments, the RT of the prime editor comprises a truncated RNase H domain compared to a corresponding reference RT. In some embodiments, the truncation in the RNase H domain decreases or abolishes RNase activity of the RNase H domain. In some embodiments, the RT of the prime editor comprises a RNase H domain that consists of 95%, 90%, 85%, 80%, 75%, 70%, 65%, 60%, 55%, 50%, 45%, 40%, 35%, 30%, 25%, 20%, 15% or 10% of amino acids of a corresponding wild-type RNase H domain (e.g., a wild-type RNase H domain from a reference M-MLV RT or a wild-type M-MLV RT).

In some embodiments, a reference RT sequence has the sequence of SEQ ID NO: 73.

In some embodiments, the RT of the prime editor comprises a truncated RNase H domain compared to a corresponding reference RT, wherein the truncation is at the N-terminus of the RNase H domain. In some embodiments, the RT of the prime editor comprises a truncated RNase H domain compared to a corresponding reference RT, wherein the truncation is at the C-terminus of the RNase H domain. In some embodiments, the RT of the prime editor comprises a truncated RNase H domain compared to a corresponding reference RT, wherein the truncation is within the middle of the RNase H domain referenced by the RNase H domain of the corresponding reference RT. In some embodiments, the RT of the prime editor comprises a truncated RNase H domain compared to a corresponding reference RT, wherein the truncated RNase H domain is truncated at both the N-terminus and the C-terminus of the RNase H domain. In some embodiments, the RT of the prime editor comprises a truncated RNase H domain compared to a corresponding reference RT, wherein the truncated RNase H domain is truncated at the N-terminus, the C-terminus, and/or the middle of the RNase H domain referenced by the RNase H domain of the corresponding reference RT. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550 or more amino acids are truncated at the N-terminus of the RNase H domain of the RT in a prime editor compared to the RNase H domain of a corresponding reference RT. In some embodiments, about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400,410, 420, 430, 440, 450, 460, 470, 480, 490, 500, 510, 520, 530, 540, 550 or more amino acids are truncated at the C-terminus of the RNase H domain of the RT in a prime editor compared to the RNase H domain of a corresponding reference RT. In some embodiments, the RT of the prime editor lacks a RNase H domain. In some embodiments, a reference RT sequence has the sequence of SEQ ID NO: 73.

In some embodiments, a prime editor comprises an RT that is a Moloney murine leukemia virus (M-MLV) reverse transcriptase (M-MLV RT) that comprises an RNase H domain. In some embodiments, the M-MLV RT of the prime editor comprises one or more amino acid substitutions, insertions, or deletions in the RNase H domain compared to the RNase H domain of a wild-type M-MLV RT. In some embodiments, the one or more amino acid substitutions, insertions, or deletions in the RNase H domain reduces or abolishes RNase activity of the RNase H domain. In some embodiments, the M-MLV RT of the prime editor comprises a RNase H domain that has decreased or abolished RNase activity compared to a RNase H domain in a wild-type M-MLV RT. In some embodiments, the M-MLV RT of the prime editor comprises an inactivated RNase H domain.

In some embodiments, a prime editor comprises a M-MMLV RT comprising one or more of amino acid substitutions P51$, S67$, E69$, L139$, T197$, D200$, H204$, F209$, E302$, T306$, F309$, W313$, T330$, L345$, L435$, N454$, D524$, E562$, D583$, H594$, L603$, E607$, or D653$ as compared to a reference M-MMLV RT as set forth in SEQ ID NO: 73, where $ is any amino acid other than the wild-type amino acid. In some embodiments, the prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to a reference M-MMLV RT as set forth in SEQ ID NO: 73. In some embodiments, the prime editor comprises a M-MLV RT comprising one or more amino acid substitutions D200N, T330P, L603W, T306K, and W313F as compared to a reference M-MMLV as set forth in SEQ ID NO: ##. In some embodiments, the prime editor comprises a M-MLV RT comprising amino acid substitutions D200N, T330P, L603W, T306K, and W313F as compared to a reference M-MMLV RT as set forth in SEQ ID NO: 73.

In some embodiments, a prime editor comprising a reverse transcriptase harboring the D200N, T330P, L603W, T306K, and W313F as compared to the reference M-MMLV RT set forth in SEQ ID NO: 73, maybe referred to as a “PE2” prime editor, and the corresponding prime editing system a PE2 prime editing system. In some embodiments, a prime editor comprises a M-MMLV RT comprising one or more of amino acid substitutions D200N, T306K, W313F, T330P, L603W, or any combination thereof as compared to the reference M-MMLV RT as set forth in SEQ ID NO: 73, where X is any amino acid other than the wild-type amino acid. In some embodiments, a prime editor comprises a M-MMLV RT comprising one or more of amino acid substitutions Y134X, Y272X, L435X, D524X, or any combination thereof as compared to the reference M-MMLV RT as set forth in SEQ ID NO: 73, where X is any amino acid other than the wild-type amino acid. In some embodiments, a prime editor comprises a M-MMLV RT comprising one or more of amino acid substitutions Y134R, Y272R, L435K, D524N, or any combination thereof as compared to the reference M-MMLV RT as set forth in SEQ ID NO: 73, where X is any amino acid other than the wild-type amino acid.

In some embodiments, the M-MLV RT variant comprises one or more of D200N, T306K, W313F, T330P, and L603W amino acid substitutions as compared to reference M-MLV RT sequence SEQ ID NO: 73. In some embodiments, the M-MLV RT variant comprises D200N, T306K, W313F, T330P, and L603W amino acid substitutions as compared to reference M-MLV RT sequence SEQ ID NO: 73.

In some embodiments, a DNA polymerase domain, e.g., a reverse transcriptase domain, for example a M-MLV RT can comprise one or more mutations (e.g., one or more amino acid substitution, amino acid deletion, and/or amino acid insertion). Mutant reverse transcriptase can, for example, be obtained by mutating the gene or genes encoding the reverse transcriptase of interest by site-directed or random mutagenesis. In some embodiments, the mutation increases the efficiency of the DNA polymerase domain, e.g., a reverse transcriptase domain, e.g., by increasing editing efficiency, by increasing reverse transcriptase activity, and/or by increasing stability (e.g., thermostability). In some embodiments, a prime editor comprising the DNA polymerase domain comprising one or more mutations disclosed herein, can exhibit at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a prime editor comprising a corresponding non-mutated DNA polymerase. In some embodiments, a DNA polymerase domain that is a M-MLV RT comprises one or more mutations selected from the group consisting of a P51$, a S67$, an E69$, an L139$, a T197$, a D200$, a H204$, a F209$, an E302$, a T306$, a F309$, a W313$, a T330$, an L435$, a P448$, a D449$, an N454$, a D524$, an E562$, a D583$, an H594$, an L603$, an E607$, a G615$, an H634$, a G637$, an H638$, a D653$, or an L671$ mutation relative to the reference M-MLV RT as set forth in SEQ ID NO: 73, where $ is any amino acid other than the wild-type amino acid. In some embodiments, a DNA polymerase domain, for example, a M-MLV RT can comprise one or more amino acid substitution selected from the group consisting of a P51L, a S67K, an E69K, an L139P, a T197A, a D200N, a H204R, a F209N, an E302K, a T306K, a F309N, a W313F, a T330P, an L435G, a P448A, a D449G, an N454K, a D524G, an E562Q, a D583N, an H594Q, an L603W, an E607K, a G615, an H634Y, a G637R, an H638G, a D653N, or an L671P relative to the reference M-MLV RT as set forth in SEQ ID NO: 73.

In some embodiments, the engineered RT may have improved stability, reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

A prime editor comprising any of the engineered RTs described herein can have altered functional features compared to a reference prime editor having the corresponding reference RT (e.g., a reference RT such as set forth in SEQ ID NO: 73). In some embodiments, a prime editor comprising an engineered RT described herein has improved stability compared to a reference prime editor having the corresponding reference RT (e.g., a reference RT such as set forth in SEQ ID NO: 73). In some embodiments, a prime editor comprising an engineered RT described herein has improved thermostability compared to a reference prime editor having the corresponding reference RT (e.g., a reference RT such as set forth in SEQ ID NO: ##). In some embodiments, a prime editor comprising an engineered RT described herein has improved solubility or reduced aggregation compared to a reference prime editor having the corresponding reference RT (e.g., a reference RT such as set forth in SEQ ID NO: 73). In some embodiments, the prime editor comprising the engineered RT has improved prime editing efficiency compared to a reference prime editor having the corresponding reference RT (e.g., a reference RT such as set forth in SEQ ID NO: 73). In some embodiments, the prime editor comprising the engineered RT has increased prime editing efficiency by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300% or more compared to the reference prime editor having the corresponding reference RT (e.g., or a reference RT as set forth in SEQ ID NO: 73). In some embodiments, the prime editor comprising the engineered RT has increased prime editing efficiency by at least 1.1 fold, 1.2 fold, 1.3 fold, 1.4 fold, 1.5 fold, 1.6 fold, 1.7 fold, 1.8 fold, 1.9 fold, 2 fold, 2.1 fold, 2.2 fold, 2.3 fold, 2.4 fold, 2.5 fold, 2.6 fold, 2.7 fold, 2.8 fold, 2.9 fold, 3 fold, 3.1 fold, 3.2 fold, 3.3 fold, 3.4 fold, 3.5 fold, 3.6 fold, 3.7 fold, 3.8 fold, 3.9 fold, 4 fold, 4.1 fold, 4.2 fold, 4.3 fold, 4.4 fold, 4.5 fold, 4.6 fold, 4.7 fold, 4.8 fold, 4.9 fold, 5 fold or more compared to the reference prime editor having the corresponding reference RT (e.g., a reference RT such as set forth in SEQ ID NO: 73).

### Programmable DNA Binding Domain

In some embodiments, a prime editor comprises a polypeptide domain having DNA binding activity (e.g., a DNA binding domain). In some embodiments, a prime editor comprises a polypeptide domain having DNA binding activity (e.g., a DNA binding domain). In some embodiments, a prime editor comprises a DNA binding domain. In some embodiments, the DNA binding domain comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or 100% identical to any one of amino acid sequences set forth in SEQ ID NO: 60(MDKKYSIGLDIGTNSVGWAVITDEYKVPSKKFKVLGNTDRHSIKKNLIGALLFDS GETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHFLI EGDLNPDNSDVDKLFIQLVQTYNQLFEENPINASGVDAKAILSARLSKSRRLENLIAQ LPGEKKNGLFGNLIALSLGLTPNFKSNFDLAEDAKLQLSKDTYDDDLDNLLAQIGDQ YADLFLAAKNLSDAILLSDILRVNTEITKAPLSASMIKRYDEHHQDLTLLKALVRQQL PEKYKEIFFDQSKNGYAGYIDGGASQEEFYKFIKPILEKMDGTEELLVKLNREDLLRK QRTFDNGSIPHQIHLGELHAILRRQEDFYPFLKDNREKIEKILTFRIPYYVGPLARGNS RFAWMTRKSEETITPWNFEEVVDKGASAQSFIERMTNFDKNLPNEKVLPKHSLLYEY FTVYNELTKVKYVTEGMRKPAFLSGEQKKAIVDLLFKTNRKVTVKQLKEDYFKKIE CFDSVEISGVEDRFNASLGTYHDLLKIIKDKDFLDNEENEDILEDIVLTLTLFEDREMIE ERLKTYAHLFDDKVMKQLKRRRYTGWGRLSRKLINGIRDKQSGKTILDFLKSDGFA NRNFMQLIHDDSLTFKEDIQKAQVSGQGDSLHEHIANLAGSPAIKKGILQTVKVVDE LVKVMGRHKPENIVIEMARENQTTQKGQKNSRERMKRIEEGIKELGSQILKEHPVEN TQLQNEKLYLYYLQNGRDMYVDQELDINRLSDYDVDHIVPQSFLKDDSIDNKVLTR SDKNRGKSDNVPSEEVVKKMKNYWRQLLNAKLITQRKFDNLTKAERGGLSELDKA GFIKRQLVETRQITKHVAQILDSRMNTKYDENDKLIREVKVITLKSKLVSDFRKDFQF YKVREINNYHHAHDAYLNAVVGTALIKKYPKLESEFVYGDYKVYDVRKMIAKSEQ EIGKATAKYFFYSNIMNFFKTEITLANGEIRKRPLIETNGETGEIVWDKGRDFATVRK VLSMPQVNIVKKTEVQTGGFSKESILPKRNSDKLIARKKDWDPKKYGGFDSPTVAYS VLVVAKVEKGKSKKLKSVKELLGITIMERSSFEKNPIDFLEAKGYKEVKKDLIIKLPK YSLFELENGRKRMLASAGELQKGNELALPSKYVNFLYLASHYEKLKGSPEDNEQKQ LFVEQHKHYLDEIIEQISEFSKRVILADANLDKVLSAYNKHRDKPIREQAENIIHLFTL TNLGAPAAFKYFDTTIDRKRYTSTKEVLDATLIHQSITGLYETRIDLSQLGGD).

In some embodiments, the DNA binding domain comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or 100% identical to any one of amino acid sequences set forth in SEQ ID NO:

SEQ ID NO: 285
(MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKD
TYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTNEDK
NLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYETR
IDLSQLGGD).

In some embodiments, the DNA-binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions or substitutions compared to any one of the amino acid sequences set forth in SEQ ID NOs: 60-72. In some embodiments, the DNA binding domain comprises an amino acid sequence that lacks a N-terminus methionine compared to a corresponding DNA binding domain (e.g., a DNA binding domain set forth in any one of SEQ ID NOs: 60-72.

In some embodiments, the amino acid sequence of a DNA binding domain can be N-terminally modified by one or more processing enzymes, e.g., by Methionine aminopeptidases (MAP).

In some embodiments, the DNA binding domain comprises a nuclease activity, for example, an RNA-guided DNA endonuclease activity of a Cas polypeptide. In some embodiments, the DNA binding domain comprises a nuclease domain or nuclease activity. In some embodiments, the DNA binding domain comprises a nickase, or a fully active nuclease. As used herein, the term “nickase” refers to a nuclease capable of cleaving only one strand of a double-stranded DNA target. In some embodiments, the DNA binding domain is an inactive nuclease.

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a double stranded target DNA (e.g., the target gene). In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single strand DNA cleavage activity. For example, the endonuclease domain may comprise a Fokl nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild-type endonuclease domain. For example, the endonuclease domain may comprise one or more amino acid substitutions as compared to a wild-type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild-type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, CasF, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild-type or a modified form of a Cas protein. A Cas protein, e.g., Cas9, can be a nuclease active variant, nuclease inactive variant, a nickase, or a functional variant or functional fragment of a wild-type Cas protein. In some embodiments, a Cas protein, e.g., Cas9, can comprise an amino acid change such as a deletion, insertion, substitution, fusion, chimera, or any combination thereof relative to a wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the double stranded target DNA in a sequence-specific manner and generate a single-strand break at a protospacer within double-stranded DNA in the double stranded target DNA, but not a double-strand break. For example, the Cas nickase can cleave the edit strand or the non-edit strand of the double stranded target DNA but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a D10$ amino acid substitution compared to a wild-type S. pyogenes Cas9, wherein $ is any amino acid other than D. In some embodiments, a prime editor comprises a Cas9 nickase having an amino acid substitution in the HNH domain. In some embodiments, the Cas9 nickase comprises a H840$ amino acid substitution compared to a wild-type S. pyogenes Cas9, wherein $ is any amino acid other than H.

In some embodiments, a prime editor comprises a Cas protein that can bind to the double stranded target DNA in a sequence-specific manner but lacks or has abolished nuclease activity and may not cleave either strand of a double stranded DNA in a double stranded target DNA. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

A Cas protein may be provided in any form. For example, a Cas protein may be provided in the form of a protein, such as a Cas protein alone or complexed with a guide nucleic acid. A Cas protein may be provided in the form of a nucleic acid encoding the Cas protein, such as an RNA (e.g., messenger RNA (mRNA)) or DNA. The nucleic acid encoding the Cas protein may be codon optimized for efficient translation into protein in a particular cell or organism.

Nucleic acids encoding Cas proteins may be stably integrated in the genome of the cell. Nucleic acids encoding Cas proteins may be operably linked to a promoter active in the cell. Nucleic acids encoding Cas proteins may be operably linked to a promoter in an expression construct. Expression constructs may include any nucleic acid constructs capable of directing expression of a gene or other nucleic acid sequence of interest (e.g., a Cas gene) and which may transfer such a nucleic acid sequence of interest to a target cell.

In some embodiments, a Cas protein may comprise a modified form of a wild type Cas protein. In some embodiments, the modified form of the wild type Cas protein may comprise one or more mutations (e.g., amino acid deletion, insertion, and/or substitution) that reduces the nucleic acid-cleaving activity of the Cas protein. For example, the modified form of the Cas protein may have less than 90%, less than 80%, less than 70%, less than 60%, less than 50%, less than 40%, less than 30%, less than 20%, less than 10%, less than 5%, or less than 1% of the nucleic acid-cleaving activity compared to the corresponding protein (e.g., Cas9 from S. pyogenes). In some embodiments, the modified form of Cas protein may have no substantial nucleic acid-cleaving activity. When a Cas protein is a modified form that has no substantial nucleic acid-cleaving activity, it may be referred to as enzymatically inactive and/or “dead” (abbreviated by “d”). A dead Cas protein (e.g., dCas, dCas9) may bind to a target polynucleotide but may not cleave the target polynucleotide. In some embodiments, a dead Cas protein is a dead Cas9 protein.

Enzymatically inactive can refer to a polypeptide that can bind to a nucleic acid sequence in a polynucleotide in a sequence-specific manner but may not cleave a target polynucleotide. An enzymatically inactive site-directed polypeptide may comprise an enzymatically inactive domain (e.g., nuclease domain). Enzymatically inactive can refer to no activity. Enzymatically inactive may refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a corresponding wild-type exemplary activity (e.g., nucleic acid cleaving activity, wild-type Cas9 activity).

In some embodiments, one or a plurality of the nuclease domains (e.g., RuvC, HNH) of a Cas protein may be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, may generate a single-strand break at a CRISPR RNA (crRNA) recognition sequence within a double-stranded DNA but not a double-strand break. Such a nickase can cleave the complementary strand or the non-complementary strand but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein may have a reduced or no ability to cleave both strands of a double-stranded target DNA. An example of a mutation that may convert a Cas9 protein into a nickase is a D10A amino acid substitution (aspartate to alanine at position 10 of Cas9 as set forth in SEQ ID NO: 2) mutation in the RuvC domain of Cas9 from S. pyogenes. A mutation corresponding to the H840A amino acid substitution (histidine to alanine at amino acid position 840 as set forth in SEQ ID NO: ##) in the HNH domain of Cas9 from S. pyogenes may convert the Cas9 into a nickase. An example of a mutation that may convert a Cas9 protein into a dead Cas9 is a D10A (aspartate to alanine at position 10 of Cas9) mutation in the RuvC domain and H840A (histidine to alanine at amino acid position 840) in the HNH domain of Cas9 from S. pyogenes.

In some embodiments, a dead Cas protein may comprise one or more mutations relative to a wild-type version of the protein. The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type Cas protein. The mutation may result in one or more of the plurality of nucleic acid-cleaving domains retaining the ability to cleave the complementary strand of the target nucleic acid but reducing its ability to cleave the non-complementary strand of the target nucleic acid. The mutation may result in one or more of the plurality of nucleic acid-cleaving domains retaining the ability to cleave the non-complementary strand of the target nucleic acid but reducing its ability to cleave the complementary strand of the target nucleic acid. The mutation may result in one or more of the plurality of nucleic acid-cleaving domains lacking the ability to cleave the complementary strand and the non-complementary strand of the target nucleic acid. The residues to be mutated in a nuclease domain may correspond to one or more catalytic residues of the nuclease. For example, residues in the wild type exemplary S. pyogenes Cas9 polypeptide such as Asp10, His840, Asn854 and Asn856 may be mutated to inactivate one or more of the plurality of nucleic acid-cleaving domains (e.g., nuclease domains). The residues to be mutated in a nuclease domain of a Cas protein may correspond to residues Asp10, His840, Asn854 and Asn856 in the wild type S. pyogenes Cas9 polypeptide, for example, as determined by sequence and/or structural alignment.

As non-limiting examples, one or more of amino acid residues D10, G12, G17, E762, H840, N854, N863, H982, H983, A984, D986, and/or A987 in a SpCas9 as set forth in SEQ ID NO: 60, or corresponding amino acid residues in another Cas9 protein may be mutated. For example, a Cas9 protein variant may comprise one or more of D10A, G12A, G17A, E762A, H840A, N854A, N863A, H982A, H983A, A984A, and/or D986A amino acid substitutions as set forth in SEQ ID NO: 60 or corresponding mutations. In some embodiments, mutations other than alanine substitutions can be suitable.

In some embodiments, the DNA-binding domain comprises a Cas protein domain that is a nickase. In some embodiments, the Cas nickase comprises one or more amino acid substitutions in a nuclease domain compared to a corresponding Cas protein. In some embodiments, the one or more amino acid substitutions in a nuclease domain reduces or abolishes its double strand nuclease activity but retains DNA binding activity. In some embodiments, the Cas nickase comprises an amino acid substitution in a HNH domain compared to a corresponding Cas protein. In some embodiments, the Cas nickase comprises an amino acid substitution in a RuvC domain compared to a corresponding Cas protein. In some embodiments, the Cas nickase is a Cas9 nickase. In some embodiments, the Cas9 nickase comprises one or more mutation in the HNH domain compared to a corresponding Cas9 protein. In some embodiments, one or more mutation in the HNH domain that reduces or abolishes nuclease activity of the HNH domain. In some embodiments, a Cas protein domain is a nuclease active variant, nuclease inactive variant, a nickase, or a functional variant or functional fragment of a wild type Cas protein.

In some embodiments, the Cas protein domain can be between 800 and 1500 amino acids in length, between 1400 and 900 amino acids in length, or at least 1000 and 1300 amino acids in length. In some embodiments, the Cas9 protein domain may be at least 800 amino acids in length, at least 900 amino acids in length, at least 1000 amino acids in length, at least 1100 amino acids in length, or at least 1200 amino acids in length. In some embodiments, the Cas9 protein domain is 1057 amino acids in length. In some embodiments, the Cas protein domain is 1069 amino acids in length. In some embodiments, the Cas protein domain is 1369 amino acids in length.

In some embodiments, the Cas protein domain recognizes the PAM sequence “NGA,” wherein N is any nucleotide. In some embodiments, the Cas protein domain recognizes the PAM sequence “NGN,” wherein N is any nucleotide. In some embodiments, the Cas protein domain recognizes the PAM sequence “NRN,” wherein N is any nucleotide. In some embodiments, the Cas protein domain recognizes the PAM sequence “NNGRRT,” wherein N is any nucleotide. In some embodiments, the Cas protein domain recognizes the PAM sequence “NNGG,” wherein N is any nucleotide.

In some embodiments, a prime editor provided herein comprises a Cas protein domain that contains modifications that allow altered PAM recognition. In prime editing using a Cas-protein-based prime editor, a “protospacer adjacent motif (PAM)”, PAM sequence, or PAM-like motif, may be used to refer to a short DNA sequence immediately adjacent to the protospacer sequence on the PAM strand of the target gene. In some embodiments, the PAM is recognized by the Cas nuclease in the prime editor during prime editing. In certain embodiments, the PAM is required for target binding of the Cas protein domain. The specific PAM sequence required for Cas protein domain recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild-type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild-type reference Cas9 protein (e.g., Cas9 from S. pyogenes). In some embodiments, the Cas9 comprises an amino acid change such as a deletion, insertion, substitution, fusion, chimera, or any combination thereof as compared to a wild-type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide, e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. AOA3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is aNmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP_116882154.1, WP_116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. A0Q5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9).

An exemplary Streptococcus pyogenes Cas9 (SpCas9) amino acid sequence is provided in any one of SEQ ID NOs: 60-72 and 75.

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: ##, or a corresponding mutation thereof. In some embodiments, the Cas9 comprises a D10A mutation as compared to a wild type SpCas9 as set forth in SEQ ID NO: 60, or a corresponding mutation thereof. In some embodiments, the Cas9 polypeptide comprises a mutation at amino acid D10, G12, and/or G17 as compared to a wild-type SpCas9 as set forth in SEQ ID NO: 60, or a corresponding mutation thereof. In some embodiments, the Cas9 polypeptide comprises a D10A mutation, a G12A mutation, and/or a G17A mutation as compared to a wild-type SpCas9 as set forth in SEQ ID NO: 60, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild-type SpCas9 as set forth in SEQ ID NO: 60, or a corresponding mutation thereof. In some embodiments, the Cas9 polypeptide comprises a H840A mutation as compared to a wild-type SpCas9 as set forth in SEQ ID NO: 60, or a corresponding mutation thereof. In some embodiments, the Cas9 polypeptide comprises a mutation at amino acid E762, D839, H840, N854, N856, N863, H982, H983, A984, D986, and/or a A987 as compared to a wild-type SpCas9 as set forth in SEQ ID NO: 60, or a corresponding mutation thereof. In some embodiments, the Cas9 polypeptide comprises a E762A, D839A, H840A, N854A, N856A, N863A, H982A, H983A, A984A, and/or a D986A mutation as compared to a wild-type SpCas9 as set forth in SEQ ID NO: 60, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840$ substitution and a D10X mutation compared to a wild-type SpCas9 as set forth in SEQ ID NO: 60 or corresponding mutations thereof, wherein $ is any amino acid other than H for the H840$ substitution and any amino acid other than D for the D10$ substitution. In some embodiments, the dead Cas9 comprises a H840A and a D10A mutation as compared to a wild-type SpCas9 as set forth in SEQ ID NO: 60, or corresponding mutations thereof.

In some embodiments, the N-terminal methionine is removed from a Cas9 nickase, or from any Cas9 variant, ortholog, or equivalent disclosed or contemplated herein.

Besides dead Cas9 and Cas9 nickase variants, the Cas9 proteins used herein may also include other Cas9 variants having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9. In some embodiments, a reference Cas9 comprises an amino acid sequence selected from the group consisting of SEQ ID NO: ## or ##. In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the double stranded target DNA (e.g., target gene). In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. It should be appreciated that for each of the variants provided, the Cas protein comprises one or more of the amino acid substitutions as indicated compared to a wild-type Cas protein sequence, for example, the Cas9 as set forth in SEQ ID NO: 60.

### Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the double stranded target DNA (e.g., the target gene) and assists incorporation of the intended nucleotide edit into the double stranded target DNA (e.g., the target gene). In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs.

In addition, the NLSs can be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 286), KRTADGSEFESPKKKRKV (SEQ ID NO: 287), KRTADGSEFEPKKKRKV (SEQ ID NO: 288), or MKRTADGSEFESPKKKRKV (SEQ ID NO:304).

In some embodiments, a nuclear localization signal (NLS) is truncated. In some embodiments, the NLS truncated at the N-terminus. In some embodiments, the NLS truncated at the C-terminus. In some embodiments, the NLS truncated at the N-terminus and at the C-terminus.

In some embodiments, a NLS is a monopartite NLS. For example, in some embodiments, a NLS is a SV40 large T antigen NLS; PKKKRKV (SEQ ID NO: 282). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of

(SEQ ID NO: 286)
MDSLLMNRRKFLYQFKNVRWAKGRRETYLC,
(SEQ ID NO: 287)
KRTADGSEFESPKKKRKV,
(SEQ ID NO: 288)
KRTADGSEFEPKKKRKV,
(SEQ ID NO: 304)
MKRTADGSEFESPKKKRKV,
or
(SEQ ID NO: 282)
PKKKRKV.

### Linkers

Polypeptides comprising components of a prime editor, e.g., the DNA binding domain and the DNA polymerase domain, may be fused via linkers, e.g., peptide or non-peptide linkers or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence.

Non-limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a DNA polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In some embodiments, the linker is a covalent bond (e.g., a carbon-carbon bond, disulfide bond, carbon-heteroatom bond, etc.).

In certain embodiments, the linker is a carbon-nitrogen bond of an amide linkage. In some embodiments, the linker is a polymeric linker many atoms in length, for example, a polypeptide sequence.

In some embodiments, a linker joins two domains of a prime editor, for example, a DNA binding domain and a DNA polymerase domain. In some embodiments, linkers join each of, or at least two of, two or more domains of a prime editor, for example, a DNA binding domain, a DNA polymerase domain, a RNA-binding protein domain (e.g., a MS2 coat protein that binds to MS2 recruitment aptamer RNA sequence), and/or a flap nuclease domain. In some embodiments, linkers join each of, or at least two of, two or more domains of a prime editor, for example, a DNA binding domain, a DNA polymerase domain, an RNA-binding protein domain (e.g., a MS2 coat protein that binds to MS2 recruitment aptamer RNA sequence), a flap nuclease domain, and/or one or more nuclear localization sequences.

In some embodiments, the linker is an amino acid or is a peptide comprising a plurality of amino acids. In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-120, 120-130, 130-140, 140-150, or 150-200 amino acids in length. In some embodiments, the peptide linker is 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140,150, 160, 175, 180, 190, or 200 amino acids in length. In some embodiments, the peptide linker is 5-100 amino acids in length. In some embodiments, the peptide linker is 10-80 amino acids in length. In some embodiments, the peptide linker is 15-70 amino acids in length. In some embodiments, the peptide linker is 16 amino acids in length, 24 amino acids in length, 64 amino acids in length, or 96 amino acids in length. In some embodiments, the peptide linker is at least 50 amino acids in length. In some embodiments, the peptide linker is at least 40 amino acids in length. In some embodiments, the peptide linker is at least 30 amino acids in length. In some embodiments, the peptide linker is 46 amino acids in length. In some embodiments, the peptide linker is 92 amino acids in length.

For example, the DNA binding domain and the DNA polymerase domain of a prime editor may be joined by a peptide or protein linker. In some embodiments, a prime editor comprises a fusion protein comprising one or more peptide linkers that join a DNA binding domain, e.g., a Cas9 nickase domain, and a DNA polymerase domain, e.g., a M-MLV reverse transcriptase domain.

In some other embodiments, the peptide linker comprises the amino acid motif GGGS (SEQ ID NO: 289), GGSS (SEQ ID NO: 290), GGS, GGGGS (SEQ ID NO: 291), SGGS (SEQ ID NO: 280), EAAAK (SEQ ID NO: 292), or any combination thereof. In some embodiments, the peptide linker comprises amino acid sequence (GGGGS)n (SEQ ID NO: 310), (G)n (SEQ ID NO: 311), (EAAAK)n (SEQ ID NO: 312), (GGS)n (SEQ ID NO: 313), (SGGS)n (SEQ ID NO: 314), (GGSS)n (SEQ ID NO: 315), (XP)n (SEQ ID NO: 316), or any combination thereof, wherein n is independently an integer between 1 and 30, and wherein X is any amino acid. In some embodiments, the peptide linker comprises the amino acid sequence (GGS)n (SEQ ID NO: 317), wherein n is 1, 3, or 7. In some embodiments, the peptide linker comprises the amino acid sequence SGSETPGTSESATPES (SEQ ID NO: 293), which may be referred to as an XTEN motif. In some embodiments, the peptide linker comprises 2, 3, 4, 5, or 6 contiguous XTEN motifs. In some embodiments, the peptide linker comprises the amino acid sequence SGGSSGGSSGSETPGTSESATPESSGGSSGGS (SEQ ID NO: 294). In some embodiments, the peptide linker comprises the amino acid sequence SGGSGGSGGS (SEQ ID NO: 295). In some embodiments, the peptide linker comprises the amino acid sequence SGGS (SEQ ID NO: 280). In other embodiments, the peptide linker comprises the amino acid sequence

(SEQ ID NO: 296)
SGGSSGGSSGSETPGTSESATPESAGSYPYDVPDYAGSAAPAAKKKKLDGS
GSGGSSGGS.

In some embodiments, the peptide linker comprises at least 2 GGSS (SEQ ID NO: 362) motifs. In some embodiments, the peptide linker comprises at least 3 GGSS (SEQ ID NO: 363) motifs. In some embodiments, the peptide linker comprises at least 4 GGSS (SEQ ID NO: 364) motifs. In some embodiments, the peptide linker comprises at least 5 GGSS (SEQ ID NO: 365) motifs. In some embodiments, the peptide linker comprises at least 6 GGSS (SEQ ID NO: 366) motifs. In some embodiments, the peptide linker comprises at least 7 GGSS (SEQ ID NO: 367) motifs. In some embodiments, the peptide linker comprises at least 8 GGSS (SEQ ID NO: 368) motifs. In some embodiments, the peptide linker comprises at least 9 GGSS (SEQ ID NO: 369) motifs. In some embodiments, the peptide linker comprises 7, 8, 9, 10, 11, 12, 13, 14, 15, 16, 17, 18, 19, or 20 GGSS (SEQ ID NO: 370) motifs. In some embodiments, the peptide linker comprises at least 2 contiguous GGSS (SEQ ID NO: 362) motifs. In some embodiments, the peptide linker comprises at least 3 contiguous GGSS (SEQ ID NO: 363) motifs. In some embodiments, the peptide linker comprises at least 4 contiguous GGSS (SEQ ID NO: 364) motifs. In some embodiments, the peptide linker comprises at least 5 contiguous GGSS (SEQ ID NO: 365) motifs. In some embodiments, the peptide linker comprises at least 6 contiguous GGSS (SEQ ID NO: 366) motifs. In some embodiments, the peptide linker comprises at least 7 contiguous GGSS (SEQ ID NO: 367) motifs. In some embodiments, the peptide linker comprises at least 8 contiguous GGSS (SEQ ID NO: 368) motifs. In some embodiments, the peptide linker comprises at least 9 contiguous GGSS (SEQ ID NO: 369) motifs. In some embodiments, the peptide linker comprises 7, 8, 9, 10, 11, 12, 13, 14, 15, 16, 17, 18, 19, or 20 contiguous GGSS (SEQ ID NO: 370) motifs. In some embodiments, the peptide linker further comprises at least one GGS motif. In some embodiments, the peptide linker comprises at least one GGS motif and 3, 4, 5, 6, 7, 8, 9, 10, 11, 12, 13, 14, 15, 16, 17, 18, 19, or 20 GGSS (SEQ ID NO: 371) motifs. In some embodiments, the peptide linker comprises at least one GGS motif and 3, 4, 5, 6, 7, 8, 9, 10, 11, 12, 13, 14, 15, 16, 17, 18, 19, or 20 contiguous GGSS (SEQ ID NO: 371) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 GGSS (SEQ ID NO: 371) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 contiguous GGSS (SEQ ID NO: 371) motifs.

In some embodiments, the peptide linker comprises at least 2 SGGS (SEQ ID NO: 373) motifs. In some embodiments, the peptide linker comprises at least 3 SGGS (SEQ ID NO: 374) motifs. In some embodiments, the peptide linker comprises at least 4 SGGS (SEQ ID NO: 375) motifs. In some embodiments, the peptide linker comprises at least 5 SGGS (SEQ ID NO: 376) motifs. In some embodiments, the peptide linker comprises at least 6 SGGS (SEQ ID NO: 377) motifs. In some embodiments, the peptide linker comprises at least 7 SGGS (SEQ ID NO: 378) motifs. In some embodiments, the peptide linker comprises at least 8 SGGS (SEQ ID NO: 379) motifs. In some embodiments, the peptide linker comprises at least 9 SGGS (SEQ ID NO: 380) motifs. In some embodiments, the peptide linker comprises 7, 8, 9, 10, 11, 12, 13, 14, 15, 16, 17, 18, 19, or 20 SGGS (SEQ ID NO: 381) motifs. In some embodiments, the peptide linker comprises at least 2 contiguous SGGS (SEQ ID NO: 373) motifs. In some embodiments, the peptide linker comprises at least 3 contiguous SGGS (SEQ ID NO: 374) motifs. In some embodiments, the peptide linker comprises at least 4 contiguous SGGS (SEQ ID NO: 375) motifs. In some embodiments, the peptide linker comprises at least 5 contiguous SGGS (SEQ ID NO: 376) motifs. In some embodiments, the peptide linker comprises at least 6 contiguous SGGS (SEQ ID NO: 377) motifs. In some embodiments, the peptide linker comprises at least 7 contiguous SGGS (SEQ ID NO: 378) motifs. In some embodiments, the peptide linker comprises at least 8 contiguous SGGS (SEQ ID NO: 379) motifs. In some embodiments, the peptide linker comprises at least 9 contiguous SGGS (SEQ ID NO: 380) motifs. In some embodiments, the peptide linker comprises 7, 8, 9, 10, 11, 12, 13, 14, 15, 16, 17, 18, 19, or 20 contiguous SGGS (SEQ ID NO: 381) motifs. In some embodiments, the peptide linker further comprises at least one GGS motif. In some embodiments, the peptide linker comprises at least one GGS motif and 3, 4, 5, 6, 7, 8, 9, 10, 11, 12, 13, 14, 15, 16, 17, 18, 19, or 20 SGGS motifs (SEQ ID NO: 382). In some embodiments, the peptide linker comprises at least one GGS motif and 3, 4, 5, 6, 7, 8, 9, 10, 11, 12, 13, 14, 15, 16, 17, 18, 19, or 20 contiguous SGGS (SEQ ID NO: 382) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 SGGS (SEQ ID NO: 382) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 contiguous SGGS (SEQ ID NO: 382) motifs.

In some embodiments, the peptide linker comprises at least 3 EAAAK (SEQ ID NO: 384) motifs. In some embodiments, the peptide linker comprises at least 4 EAAAK (SEQ ID NO: 385) motifs. In some embodiments, the peptide linker comprises at least 5 EAAAK (SEQ ID NO: 386) motifs. In some embodiments, the peptide linker comprises at least 6 EAAAK (SEQ ID NO: 387) motifs. In some embodiments, the peptide linker comprises at least 7 EAAAK (SEQ ID NO: 388) motifs. In some embodiments, the peptide linker comprises at least 8 EAAAK (SEQ ID NO: 389) motifs. In some embodiments, the peptide linker comprises at least 9 EAAAK (SEQ ID NO: 390) motifs. In some embodiments, the peptide linker comprises 7, 8, 9, 10, 11, 12, 13, 14, 15, 16, 17, 18, 19, or 20 EAAAK (SEQ ID NO: 391) motifs. In some embodiments, the peptide linker comprises at least 3 contiguous EAAAK (SEQ ID NO: 384) motifs. In some embodiments, the peptide linker comprises at least 4 contiguous EAAAK (SEQ ID NO: 385) motifs. In some embodiments, the peptide linker comprises at least 5 contiguous EAAAK (SEQ ID NO: 386) motifs. In some embodiments, the peptide linker comprises at least 6 contiguous EAAAK (SEQ ID NO: 387) motifs. In some embodiments, the peptide linker comprises at least 7 contiguous EAAAK (SEQ ID NO: 388) motifs. In some embodiments, the peptide linker comprises at least 8 contiguous EAAAK (SEQ ID NO: 389) motifs. In some embodiments, the peptide linker comprises at least 9 contiguous EAAAK (SEQ ID NO: 390) motifs. In some embodiments, the peptide linker comprises 7, 8, 9, 10, 11, 12, 13, 14, 15, 16, 17, 18, 19, or 20 contiguous EAAAK (SEQ ID NO: 391) motifs. In some embodiments, the peptide linker further comprises at least one GGS motif. In some embodiments, the peptide linker comprises at least one GGS motif and 3, 4, 5, 6, 7, 8, 9, 10, 11, 12, 13, 14, 15, 16, 17, 18, 19, or 20 EAAAK (SEQ ID NO: 392) motifs. In some embodiments, the peptide linker comprises at least one GGS motif and 3, 4, 5, 6, 7, 8, 9, 10, 11, 12, 13, 14, 15, 16, 17, 18, 19, or 20 contiguous EAAAK (SEQ ID NO: 392) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 EAAAK (SEQ ID NO: 392) motifs. In some embodiments, the peptide linker comprises 2, 3, 4, 5, 6, 7, 8, 9, 10, 11, 12, 13, 14, 15, 16, 17, 18, 19, or 20 GGS (SEQ ID NO: 372) motifs and 3, 4, 5, 6, 7, 8, 9, 10, 11, 12, 13, 14, 15, 16, 17, 18, 19, or 20 contiguous EAAAK (SEQ ID NO: 392) motifs.

In some embodiments, a prime editor comprises a fusion protein comprising the structure, from N-terminus to C-terminus:
[NLS1]-[DNA binding domain]-[peptide linker]-[Reverse transcriptase];
[DNA binding domain]-[peptide linker]-[Reverse transcriptase]-[NLS1];
[NLS1]-[DNA binding domain]-[peptide linker]-[Reverse transcriptase]-[NLS2];
[NLS1]-[NLS2]-[DNA binding domain]-[peptide linker]-[Reverse transcriptase]-[NLS];
[NLS1]-[NLS2]-[DNA binding domain]-[peptide linker]-[Reverse transcriptase]-[NLS3]-[NLS4];
[NLS1]-[DNA binding domain]-[peptide linker]-[Reverse transcriptase]-[NLS2]-[NLS3];
[NLS1]-[NLS2]-[NLS3]-[DNA binding domain]-[peptide linker]-[Reverse transcriptase]-[NLS4];
[NLS1]-[NLS2]-[NLS3]-[DNA binding domain]-[peptide linker]-[Reverse transcriptase]-[NLS4]-[NLS5];
[NLS1]-[NLS2]-[NLS3]-[DNA binding domain]-[peptide linker]-[Reverse transcriptase]-[NLS4]-[NLS5]-[NLS6];
[NLS1]-[DNA binding domain]-[peptide linker]-[Reverse transcriptase]-[NLS2]-[NLS3]-[NLS4];
[NLS1]-[NLS2]-[DNA binding domain]-[peptide linker]-[Reverse transcriptase]-[NLS3]-[NLS4]-[NLS5];
[NLS1]-[Reverse transcriptase]-[peptide linker]-[DNA binding domain];
[Reverse transcriptase]-[peptide linker]-[DNA binding domain]-[NLS1];
[NLS1]-[Reverse transcriptase]-[peptide linker]-[DNA binding domain]-[NLS2];
[NLS1]-[NLS2]-[Reverse transcriptase]-[peptide linker]-[DNA binding domain]-[NLS];
[NLS1]-[NLS2]-[Reverse transcriptase]-[peptide linker]-[DNA binding domain]-[NLS3]-[NLS4];
[NLS1]-[Reverse transcriptase]-[peptide linker]-[DNA binding domain]-[NLS2]-[NLS3];
[NLS1]-[NLS2]-[NLS3]-[Reverse transcriptase]-[peptide linker]-[DNA binding domain]-[NLS4];
[NLS1]-[NLS2]-[NLS3]-[Reverse transcriptase]-[peptide linker]-[DNA binding domain]-[NLS4]-[NLS5];
[NLS1]-[NLS2]-[NLS3]-[Reverse transcriptase]-[peptide linker]-[DNA binding domain]-[NLS4]-[NLS5]-[NLS6];
[NLS1]-[Reverse transcriptase]-[peptide linker]-[DNA binding domain]-[NLS2]-[NLS3]-[NLS4]; or
[NLS1]-[NLS2]-[Reverse transcriptase]-[peptide linker]-[DNA binding domain]-[NLS3]-[NLS4]-[NLS5].

In some embodiments, a prime editor comprises a fusion protein comprising the structure, from N-terminus to C-terminus:
[NLS]n-[DNA binding domain]-[peptide linker]-[Reverse transcriptase]-[NLS]m, or [NLS]n-[Reverse transcriptase]-[peptide linker]-[DNA binding domain]-[NLS]m, wherein n and m are any integer between 0 and 50, wherein [NLS]n refers to n NLS motif sequences, and wherein [NLS]m refers to m NLS motif sequences. The n NLS motif sequences may or may not be the same. In some embodiments, m and n are the same. In some embodiments, n and m are different.

The DNA polymerase can be any of the DNA polymerase described herein or known in the art. In some embodiments, the DNA polymerase is a Cas9 nickase (nCas9). In some embodiments, the DNA polymerase is a nCas9 comprising a nuclease inactivating amino acid substitution in a HNH domain. In some embodiments, the DNA polymerase is a nCas9 comprising a H840A amino acid substitution as compared to a wild type SpCas9.

The Reverse transcriptase can be any of the reverse transcriptase described herein or known in the art. In some embodiments, the reverse transcriptase is a M-MLV RT. In some embodiments, the reverse transcriptase is a M-MLV RT functional variant with any one of the amino acid substitutions or truncations as described herein.

In some embodiments, any one of NLS1, NLS2, NLS3, NLS4, NLS5, NLS6 is independently a NLS known in the art or described herein. In some embodiments, any one of NLS1, NLS2, NLS3, NLS4, NLS5, NLS6 is a bipartite NLS. In some embodiments, any one of NLS1, NLS2, NLS3, NLS4, NLS5, NLS6 is a c-Myc NLS comprising the amino acid sequence PAAKRVKLD (SEQ ID NO: 297). In some embodiments, any one of NLS1, NLS2, NLS3, NLS4, NLS5, NLS6 is a monopartite NLS. In some embodiments, any one of NLS1, NLS2, NLS3, NLS4, NLS5, NLS6 is a SV40 NLS.

In some embodiments, two or more of the NLSsl-6 are the same. In some embodiments, the NLSs 1-6 are different from each other.

In any of the prime editor structures, the peptide linker may be any peptide linker described herein or known in the art. In some embodiments, the peptide linker comprises the amino acid sequence, from N terminus to C-terminus: (GGSS)m-(GGS)n (SEQ ID NO: 318), wherein m and n are each any integer between 0 and 50. In some embodiments, the peptide linker comprises the amino acid sequence, from N terminus to C-terminus: (GGS)n-(GGSS)m (SEQ ID NO: 319), wherein m and n are each any integer between 0 and 50. In some embodiments, m and n are the same. In some embodiments, m and n are different. In some embodiments, the peptide linker comprises the amino acid sequence from N terminus to C-terminus: (GGSS)-(GGS) (SEQ ID NO: 320). In some embodiments, the peptide linker comprises the amino acid sequence from N terminus to C-terminus: (GGSS)2-(GGS) (SEQ ID NO: 321). In some embodiments, the peptide linker comprises the amino acid sequence from N terminus to C-terminus: (GGSS)3-(GGS) (SEQ ID NO: 322). In some embodiments, the peptide linker comprises the amino acid sequence from N terminus to C-terminus: (GGSS)4-(GGS) (SEQ ID NO: 323). In some embodiments, the peptide linker comprises the amino acid sequence from N terminus to C-terminus: (GGSS)5-(GGS) (SEQ ID NO: 324). In some embodiments, the peptide linker comprises the amino acid sequence from N terminus to C-terminus: (GGSS)6-(GGS) (SEQ ID NO: 325). In some embodiments, the peptide linker comprises the amino acid sequence from N terminus to C-terminus: (GGSS)7-(GGS) (SEQ ID NO: 303). In some embodiments, the peptide linker comprises the amino acid sequence from N terminus to C-terminus: (GGSS)8-(GGS) (SEQ ID NO: 327). In some embodiments, the peptide linker comprises the amino acid sequence from N terminus to C-terminus: (GGSS)9-(GGS) (SEQ ID NO: 328). In some embodiments, the peptide linker comprises the amino acid sequence from N terminus to C-terminus: (GGSS)IO-(GGS) (SEQ ID NO: 329). In some embodiments, the peptide linker comprises the amino acid sequence from N terminus to C-terminus: (GGSS)11-(GGS) (SEQ ID NO: 330). In some embodiments, the peptide linker comprises the amino acid sequence from N terminus to C-terminus: (GGSS)12-(GGS) (SEQ ID NO: 331). In some embodiments, the peptide linker comprises the amino acid sequence from N terminus to C-terminus: (GGSS)13-(GGS) (SEQ ID NO: 332). In some embodiments, the peptide linker comprises the amino acid sequence from N terminus to C-terminus: (GGSS)14-(GGS) (SEQ ID NO: 333). In some embodiments, the peptide linker comprises the amino acid sequence from N terminus to C-terminus: (GGSS)15-(GGS) (SEQ ID NO: 334). In some embodiments, the peptide linker comprises the amino acid sequence from N terminus to C-terminus: (GGSS)-(GGS)2 (SEQ ID NO: 335). In some embodiments, the peptide linker comprises the amino acid sequence from N terminus to C-terminus: (GGSS)-(GGS)3 (SEQ ID NO: 336). In some embodiments, the peptide linker comprises the amino acid sequence from N terminus to C-terminus: (GGSS)-(GGS)4 (SEQ ID NO: 337). In some embodiments, the peptide linker comprises the amino acid sequence from N terminus to C-terminus: (GGSS)-(GGS)5 (SEQ ID NO: 338). In some embodiments, the peptide linker comprises the amino acid sequence from N terminus to C-terminus: (GGSS)-(GGS)6 (SEQ ID NO: 339). In some embodiments, the peptide linker comprises the amino acid sequence (GGSS)-(GGS)7 (SEQ ID NO: 340). In some embodiments, the peptide linker comprises the amino acid sequence from N terminus to C-terminus: (GGSS)-(GGS)8 (SEQ ID NO: 341). In some embodiments, the peptide linker comprises the amino acid sequence (GGSS)-(GGS)9 (SEQ ID NO: 342). In some embodiments, the peptide linker comprises the amino acid sequence from N terminus to C-terminus: (GGSS)-(GGS)K (SEQ ID NO: 343)). In some embodiments, the peptide linker comprises the amino acid sequence from N terminus to C-terminus: (GGSS)-(GGS)11(SEQ ID NO: 344). In some embodiments, the peptide linker comprises the amino acid sequence from N terminus to C-terminus: (GGSS)-(GGS)12(SEQ ID NO: 345). In some embodiments, the peptide linker comprises the amino acid sequence from N terminus to C-terminus: (GGSS)-(GGS)13(SEQ ID NO: 346). In some embodiments, the peptide linker comprises the amino acid sequence from N terminus to C-terminus: (GGSS)-(GGS)14 (SEQ ID NO: 347). In some embodiments, the peptide linker comprises the amino acid sequence from N terminus to C-terminus: (GGSS)-(GGS)15(SEQ ID NO: 348).

In some embodiments, the peptide linker comprises the amino acid sequence from N terminus to C-terminus: (GGSS)2-(GGS)2(SEQ ID NO: 349). In some embodiments, the peptide linker comprises the amino acid sequence from N terminus to C-terminus: (GGSS)3-(GGS)3(SEQ ID NO: 326). In some embodiments, the peptide linker comprises the amino acid sequence from N terminus to C-terminus: (GGSS)4-(GGS)4 (SEQ ID NO: 350). In some embodiments, the peptide linker comprises the amino acid sequence from N terminus to C-terminus: (GGSS)5-(GGS)5 (SEQ ID NO: 351). In some embodiments, the peptide linker comprises the amino acid sequence from N terminus to C-terminus: (GGSS)6-(GGS)6 (SEQ ID NO: 352). In some embodiments, the peptide linker comprises the amino acid sequence from N terminus to C-terminus: (GGSS)7-(GGS)7 (SEQ ID NO: 353). In some embodiments, the peptide linker comprises the amino acid sequence from N terminus to C-terminus: (GGSS)8-(GGS)8(SEQ ID NO: 354). In some embodiments, the peptide linker comprises the amino acid sequence from N terminus to C-terminus: (GGSS)9-(GGS)9(SEQ ID NO: 355). In some embodiments, the peptide linker comprises the amino acid sequence from N terminus to C-terminus: (GGSS)IO-(GGS)K)(SEQ ID NO: 356). In some embodiments, the peptide linker comprises the amino acid sequence from N terminus to C-terminus: (GGSS)11-(GGS)11 (SEQ ID NO: 357). In some embodiments, the peptide linker comprises the amino acid sequence from N terminus to C-terminus: (GGSS)12-(GGS)12(SEQ ID NO: 358).

In some embodiments, the peptide linker comprises the amino acid sequence from N terminus to C-terminus: (GGSS)13-(GGS)13(SEQ ID NO: 359). In some embodiments, the peptide linker comprises the amino acid sequence from N terminus to C-terminus: (GGSS)14-(GGS)14(SEQ ID NO: 360). In some embodiments, the peptide linker comprises the amino acid sequence from N terminus to C-terminus: (GGSS)15-(GGS)15(SEQ ID NO: 361).

Prime editing guide RNAs (PEgRNAs)

The term “prime editing guide RNA”, or “PEgRNA”, refers to a guide polynucleotide that comprises one or more intended nucleotide edits for incorporation into a double stranded target polynucleotide, e.g., double stranded target DNA. In some embodiments, the PEgRNA associates with and directs a prime editor to incorporate the one or more intended nucleotide edits into the double stranded target DNA, e.g., a target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the double stranded target DNA, e.g., a target gene. Intended nucleotide edit may refer to the edit on the editing template as compared to the sequence on the target strand of the double stranded target DNA, e.g., a target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the double stranded target DNA, e.g., a target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the double stranded target DNA, e.g., a target gene, wherein the extended nucleotide sequence may be referred to as an extension arm.

In certain embodiments, the extension arm comprises a primer binding site sequence (PBS) that can initiate target-primed DNA synthesis. In some embodiments, the PBS is complementary or substantially complementary to a free 3′ end on the edit strand of the double stranded target DNA, e.g., a target gene at a nick site generated by the prime editor. In some embodiments, the extension arm further comprises an editing template that comprises one or more intended nucleotide edits to be incorporated in the double stranded target DNA, e.g., a target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the double stranded target DNA, e.g., a target gene. In some embodiments, the editing template comprises substantial or partial complementarity to the editing target sequence except at the position of the intended nucleotide edits to be incorporated into the double stranded target DNA, e.g., a target gene. In some embodiments, the editing template comprises a silent mutation. A silent mutation is a change in the DNA sequence that does not alter the amino acid sequence of the protein encoded by the gene. In some embodiments, the editing template comprises a silent mutation that alters the pegRNA PAM (PAM disruption mutationg). In some embodiments, the editing template comprises a 2′-O-methyl-3′-phosphonoacetate (2′-O-methyl-3′-PACE, or MP) at the 3′ end.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template.

An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing target, a PBS, a spacer, and a gRNA core.

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem.

In some embodiments, a spacer sequence comprises a region that has substantial complementarity to a search target sequence on the target strand of a double stranded target DNA, e.g., a SERPINA1 gene. In some embodiments, the spacer sequence of a PEgRNA is identical or substantially identical to a protospacer sequence on the edit strand of the double stranded target DNA, e.g., a target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the double stranded target DNA, e.g., a target gene. In some embodiments, the spacer comprises is substantially complementary to the search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to an uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that hybridizes with a free 3′ end of a single stranded DNA in the double stranded target DNA, e.g., a target gene generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS may be complementary or substantially complementary to a DNA sequence in the edit strand of the double stranded target DNA, e.g., a target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the double stranded target DNA, e.g., a target gene. In some embodiments, the PBS is perfectly complementary, or 100% complementary, to a region of the edit strand of the double stranded target DNA, e.g., a target gene.

An extension arm of a PEgRNA may comprise an editing template that serves as a DNA synthesis template for the DNA polymerase in a prime editor during prime editing. The length of an editing template may vary depending on, e.g., the prime editor components, the search target sequence, and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the editing template (e.g., RTT) sequence is about 70%, 75%, 80%, 85%, 90%, 95%, or 99% complementary to the editing target sequence on the edit strand of the double stranded target DNA, e.g., a target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated int the double stranded target DNA, e.g., a target gene. In some embodiments, the editing template comprises a nucleotide sequence comprising about 85% to about 95% complementarity to an editing target sequence in the edit strand in the double stranded target DNA, e.g., a target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the double stranded target DNA, e.g., a target gene.

An intended nucleotide edit in an editing template of a PEgRNA may comprise various types of alterations as compared to the double stranded target DNA, e.g., a target gene sequence. In some embodiments, the nucleotide edit is a single nucleotide substitution as compared to the double stranded target DNA, e.g., a target gene sequence. In some embodiments, the nucleotide edit is a deletion as compared to the double stranded target DNA, e.g., a target gene sequence. In some embodiments, the nucleotide edit is an insertion as compared to the double stranded target DNA, e.g., a target gene sequence. In some embodiments, the editing template comprises one to ten intended nucleotide edits as compared to the double stranded target DNA, e.g., a target gene sequence. In some embodiments, the editing template comprises one or more intended nucleotide edits as compared to the double stranded target DNA, e.g., a target gene sequence. In some embodiments, the editing template comprises two or more intended nucleotide edits as compared to the double stranded target DNA, e.g., a target gene sequence. In some embodiments, the editing template comprises three or more intended nucleotide edits as compared to the double stranded target DNA, e.g., a target gene sequence. In some embodiments, the editing template comprises four or more, five or more, or six or more intended nucleotide edits as compared to the double stranded target DNA, e.g., a target gene sequence. In some embodiments, the editing template comprises two single nucleotide substitutions, insertions, deletions, or any combination thereof, as compared to the double stranded target DNA, e.g., a target gene sequence. In some embodiments, the editing template comprises three single nucleotide substitutions, insertions, deletions, or any combination thereof, as compared to the double stranded target DNA, e.g., a target gene sequence. In some embodiments, the editing template comprises four, five, or six single nucleotide substitutions, insertions, deletions, or any combination thereof, as compared to the double stranded target DNA, e.g., a target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the double stranded target DNA, e.g., a target gene, to be edited. Position of the intended nucleotide edit(s) relevant to other components of the PEgRNA, or to particular nucleotides (e.g., mutations) in the double stranded target DNA, e.g., a target gene, may vary. In some embodiments, the nucleotide edit is in a region of the PEgRNA corresponding to or homologous to the protospacer sequence. In some embodiments, the nucleotide edit is in a region of the PEgRNA corresponding to a region of the double stranded target DNA outside of the protospacer sequence.

In some embodiments, the position of a nucleotide edit incorporation in the double stranded target DNA, e.g., a target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the double stranded target DNA, e.g., a target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5′ most nucleotide of the PAM sequence in the edit strand of the double stranded target DNA, e.g., a target gene. By 0 nucleotide upstream or downstream of a reference position, it is meant that the intended nucleotide is immediately upstream or downstream of the reference position. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the editing template is at a position corresponding to 6 nucleotides upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5′ most nucleotide of the PAM sequence in the edit strand of the double stranded target DNA, e.g., a target gene. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence.

In some embodiments, a nucleotide edit is incorporated at a position corresponding to 6 nucleotides downstream of the 5′ most nucleotide of the PAM sequence. By “upstream” and “downstream” it is intended to define relevant positions at least two regions or sequences in a nucleic acid molecule orientated in a 5′-to-Y direction. For example, a first sequence is upstream of a second sequence in a DNA molecule where the first sequence is positioned 5′ to the second sequence. Accordingly, the second sequence is downstream of the first sequence.

When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the double stranded target DNA, e.g., a target gene may also be referred to based on the nicking position generated by a prime editor based on sequence homology and complementarity. For example, in embodiments, the distance between the nucleotide edit to be incorporated into the double stranded target DNA, e.g., a target gene, and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated to the double stranded target DNA, e.g., a target gene, sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 base pairs 3′ to the nucleotide edit to be incorporated to the double stranded target DNA, e.g., a target gene, sequence. In some embodiments, the editing template comprises at least 4 to 30 base pairs 3′ to the nucleotide edit to be incorporated to the double stranded target DNA, e.g., a target gene, sequence. In some embodiments, the editing template comprises at least 4 to 25 base pairs 3′ to the nucleotide edit to be incorporated to the double stranded target DNA, e.g., a target gene, sequence. In some embodiments, the editing template comprises at least 4 to 20 base pairs 3′ to the nucleotide edit to be incorporated to the double stranded target DNA, e.g., a target gene, sequence. In some embodiments, the editing template comprises at least 4 to 30 base pairs 5′ to the nucleotide edit to be incorporated to the double stranded target DNA, e.g., a target gene, sequence. In some embodiments, the editing template comprises at least 4 to 25 base pairs 5′ to the nucleotide edit to be incorporated to the double stranded target DNA, e.g., a target gene, sequence. In some embodiments, the editing template comprises at least 4 to 20 base pairs 5′ to the nucleotide edit to be incorporated to the double stranded target DNA, e.g., a target gene, sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace a target sequence in the double stranded target DNA, e.g., a target gene. In some embodiments, the editing target sequence in the edit strand of the double stranded target DNA, e.g., a target gene is replaced by the newly synthesized strand, and the nucleotide edit(s) are incorporated in the region of the double stranded target DNA, e.g., a target gene. In some embodiments, the newly synthesized DNA strand replaces the editing target sequence in the double stranded target DNA, e.g., a target gene, wherein the editing target sequence (or the endogenous sequence complementary to the editing target sequence on the target strand of the target gene) comprises a mutation compared to a wild-type sequence of the same gene, wherein incorporation of the one or more intended nucleotide edits corrects the mutation.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end. In some embodiments, the gRNA core comprises modified nucleotides as compared to a wild-type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild-type RNA sequences in the nexus and/or the bulge regions. In some embodiments, the gRNA core does not include long stretches of A-T pairs, for example, a GUUUU-AAAAC pairing element.

In some embodiments, the gRNA core comprises the sequence:

(SEQ ID NO: 301)
GUUUGAGAGCUAGAAAUAGCAAGUUUAAAUAAGGCUAGUCCGUUAUCAACU
UGAAAAAGUGGGACCGAGUCGGUCC,
or
(SEQ ID NO: 298)
GUUUAAGAGCUAUGCUGGAAACAGCAUAGCAAGUUUAAAUAAGGCUAGUCC
GUUAUCAACUUGAAAAAGUGGCACCGAGUCGGUGC.

In some embodiments, the gRNA core comprises the sequence

(SEQ ID NO: 299)
GUUUUAGAGCUAGAAAUAGCAAGUUAAAAUAAGGCUAGUCCGUUAUCAACU
UGAAAAAGUGGCACCGAGUCGGUGC.

In some embodiments, the gRNA core comprises the sequence

(SEQ ID NO: 300)
GUUUUAGAGCUAGAAAUAGCAAGUUAAAAUAAGGCUAGUCCGUUAUCAACU
UGAAAAAGUGGCACCGAGUCGGUGC.

Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

The 3′ end sequence and the 5′ end sequence of a PEgRNA can be any one of the functional components of the PEgRNA and can comprise any sequence known in the art. In some embodiments, the PEgRNA comprises an extension arm at the 3′ end. For example, the PEgRNA may comprise the structure, from 5′ to 3′: a spacer, a gRNA core, an editing template (e.g, RTT), and a PBS. In some embodiments, the PEgRNA comprises a gRNA core at the 3′ end. For example, the PEgRNA may comprise the structure, from 5′ to 3′: an editing template (e.g., RTT), a PBS, a spacer, and a gRNA core. In some embodiments, the PEgRNA comprises a specific nucleotide sequence at the 3′ end. In some embodiments, the three 3′ most nucleotides of the PEgRNA are 5′-UUU-3′ In some embodiments, the four 3′ most nucleotides of the PEgRNA are 5′-UUUU-3′. In some embodiments, the three 3′ most nucleotides of the PEgRNA are not 5′-UUU-3′. In some embodiments, the four 3′ most nucleotides of the PEgRNA are not 5′-UUUU-3′. In some embodiments, the PEgRNA does not comprise two consecutive uracils in the three 3′ most nucleotides. In some embodiments, the PEgRNA does not comprise two consecutive uracils in the four 3′ most nucleotides. In some embodiments, the PEgRNA does not comprise a uracil in the four 3′ most nucleotides. In some embodiments, the PEgRNA does not comprise a uracil in the three 3′ most nucleotides. In some embodiments, the PEgRNA is chemically synthesized.

In some embodiments, a prime editing system or composition further comprises a nick guide polynucleotide, such as a nick guide RNA (ngRNA). Without wishing to be bound by any particular theory, the non-edit strand of a double stranded target DNA in the double stranded target DNA, e.g., a target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, the ngRNA is a guide RNA which contains a variable spacer sequence and a guide RNA scaffold or core region that interacts with the DNA binding domain, e.g. Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of double stranded target DNA, e.g., a target gene. A prime editing system or complex comprising a ngRNA may be referred to as a “PE3” prime editing system, PE3 prime editing compositions or PE3 prime editing complex.

In some embodiments, the ng search target sequence is located on the non-target strand, within 10 nucleotides to 100 nucleotides of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system maybe referred to as a “PE3b” prime editing system or composition. In some embodiments, the ngRNA comprises a spacer sequence that matches only the edit strand after incorporation of the nucleotide edits, but not the endogenous double stranded target DNA, e.g., a target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be structure guided modifications. In some embodiments, a chemical modification is at the 5′ end and/or the 3′ end of a PEgRNA. In some embodiments, a chemical modification is at the 5′ end and/or the 3′ end of a ngRNA.

In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. The gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′ phosphorothioate (MS), or a 2′-O-methyl 3′ thioPACE (MSP), or any combination thereof. In some embodiments, a chemical modification to a PEgRNA or ngRNA comprises a nucleotide sugar modification. In some embodiments, the chemical modification comprises a 2′O-C1-4 alkyl modification. In some embodiments, the chemical modification comprises a 2′-O-C1-3 alkyl modification. In some embodiments, the chemical modification comprises a 2′-O-methyl (2′-OMe), 2′-deoxy (2′-H), a, for example, 2′-fluoro (2′-F), 2′-methoxyethyl (2′-MOE), 2′-amino (“2′-NH2”), or 2′-arabinosyl (“2′-arabino”), 2′-F-arabinosyl (“2′-F-arabino”) modification. In some embodiments, the chemical modification comprises a locked nucleic acid (LNA) modification. In some embodiments, a chemically modification to a PEgRNA and/or ngRNA comprises an internucleotide linkage modification. In some embodiments, the internucleotide linkage is a phosphorothioate (“PS”), phosphonocarboxylate (P(CH2)nCOOR), phosphoroacetate (PACE), (P(CH2COO—)) thiophosphonocarboxylate ((S)P(CH2)nCOOR), thiophosphonoacetate (thioPACE), ((S)P(CH2COO—)), alkylphosphonate (P(C1-3alkyl) such as methylphosphonate—P(CH3), boranophosphonate (P(BH3)), or phosphorodithioate (P(S)2) modification. In some embodiments, the chemically modified PEgRNA or ngRNA is a 2′-O-methyl (M) RNA, a 2′-O-methyl 3′ phosphorothioate (MS) RNA, a 3′ thioPACE RNA, a 2′-O-methyl 3′ thioPACE (MSP) RNA, a 2′-F RNA, or a RNA having any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

In some embodiments, the PEgRNA comprises the sequence of 5′-mXmXmXmXmX-[rest of spacer sequence-gRNA core—rest of extension arm sequence]-mXmXmXmXmX-3′, wherein X is any nucleotide, wherein the “rest of spacer sequence” represent the unmodified nucleotides of the spacer sequence, wherein the “rest of extension arm sequence” represent the unmodified nucleotides of the extension arm sequence. As used herein in the context of a PEgRNA sequence or guide RNA sequence chemical modification, “m” stands for a 2′-O-methyl modification.

In some embodiments, the PEgRNA comprises the sequence of 5′-mX*mX*mX*mX*mX*-[rest of spacer sequence-gRNA core—rest of extension arm sequence]-mX*mX*mX*mX*mX*-3′, wherein X is any nucleotide, wherein the “rest of spacer sequence” represent the unmodified nucleotides of the spacer sequence, wherein the “rest of extension arm sequence” represent the unmodified nucleotides of the extension arm sequence. As used herein in the context of a PEgRNA sequence or guide RNA sequence chemical modification, “*” stands for a phosphorothioate linkage.

In some embodiments, the PEgRNA comprises the sequence of 5′-mXmXmXmX-[rest of spacer sequence-gRNA core—rest of extension arm sequence]-mXmXmXmX-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mX*mX*mX*mX*-[rest of spacer sequence-gRNA core—rest of extension arm sequence]-mX*mX*mX*mX*-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mXmXmXmXmX-[rest of spacer sequence-gRNA core—rest of extension arm sequence]-mXmXmXmXmX-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mX*mX*mX*-rest of spacer sequence-gRNA core—rest of extension arm sequence]-mX*mX*mX*-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mXmX-[rest of spacer sequence-gRNA core—rest of extension arm sequence]-mXmX-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mX*mX*-[rest of spacer sequence-gRNA core—rest of extension arm sequence]-mX*mX*-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mX-[rest of spacer sequence-gRNA core—rest of extension arm sequence]-mX-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

In some embodiments, the PEgRNA comprises the sequence of 5′-mX*-[rest of spacer sequence-gRNA core—rest of extension arm sequence]-mX*-3′, wherein X is any nucleotide, wherein the “rest of spacer sequence” represent the unmodified nucleotides of the spacer sequence, wherein the “rest of extension arm sequence” represent the unmodified nucleotides of the extension arm sequence.

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs.

Components of a prime editing composition may be combined to form a complex for prime editing, or may be kept separately, e.g., for administration purposes. In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein. In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA. In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding aN-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding aN-terminal half of a prime editor fusion protein and an intein-N (ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA can be delivered sequentially.

### Polynucleotides Encoding Prime Editor Components

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized for improved expression. Codon optimization can refer to engineering a polynucleotide sequence for enhanced expression in a host cell of interest, by replacing at least one codon (e.g., about or more than about 1, 2, 3, 4, 5, 10, 15, 20, 25, 50, or more codons) of the native polynucleotide sequence with codons that are more frequently or most frequently used in the genes of that host cell while maintaining the native amino acid sequence. In some embodiments, codon optimization engineers a polynucleotide sequence for enhanced expression by altering GC content of the polynucleotide sequence to increase mRNA stability in the host cell.

In some embodiments, codon optimization minimizes tandem repeat codons or tandem repeat nucleobase runs that may impair gene construction or expression. Codon optimization may also include customizing transcriptional and translational control regions, inserting or removing protein trafficking sequences, removing or adding post translation modification sites in encoded proteins (e.g., glycosylation sites), adding, removing or shuffling protein domains, inserting or deleting restriction sites, and/or modifying ribosome binding sites and mRNA degradation sites to enhance expression and proper folding of the prime editor polypeptide in the host cell.

In some embodiments, a polynucleotide encoding a prime editor polypeptide, e.g., a DNA sequence or mRNA sequence, is codon optimized, e.g., for expression in a cell of a specific species. Various species exhibit particular bias for certain codons of a particular amino acid. In some embodiments, the polynucleotide can be optimized for increased expression in cells of a specific species, using a codon usage table. Codon usage tables are readily available to those skilled in the art, for example, in Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as GeneArt (Life Technologies), or DNA2.0 (Menlo Park, CA).

In some embodiments, a polynucleotide encoding a prime editor polypeptide, e.g., a DNA sequence or mRNA sequence, is codon optimized for expression in a desired cell from specific species, e.g., in bacterial cell, plant cell, insect cell, or mammalian cell. In some embodiments, the codon optimization is for expression in a eukaryotic cell. In some embodiments, the codon optimization is for expression in a mammalian cell. In some embodiments, the codon optimization is for expression in a human cell. In some embodiments, a polynucleotide encoding a prime editor polypeptide is codon optimized for expression in a desire cell type. In some embodiments, the codon optimization is for expression in a hematopoietic stem cell (HSC). In some embodiments, the codon optimization is for expression in a CD34′HSC. In some embodiments, the codon optimization is for expression in a human hematopoietic stem cell (HSC). In some embodiments, the codon optimization is for expression in a human CD34′ HSC. In some embodiments, the codon optimization is for expression in a human CD34⁺ hematopoietic stem progenitor cell (HSPC). In some embodiments, the codon optimization is for expression in hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells, hematopoietic stem progenitor cells), muscle cells and precursors of these somatic cell types. In some embodiments, the codon optimization is for expression in primary hepatocytes. In some embodiments, the codon optimization is for expression in pluripotent stem cells (iPSCs). In some embodiments, the codon optimization is for expression in neurons. In some embodiments, the codon optimization is for expression in basal ganglia the codon optimization is for expression in epithelial cells from lung, liver, stomach, or intestine the codon optimization is for expression in retinal cells.

In some embodiments, codon optimization engineers a polynucleotide sequence for enhanced expression by altering secondary structure to enhance expression in the host cell. “Secondary structure” refers to the three-dimensional form of local segments of a biopolymer, such as a polynucleotide. In some embodiments, a secondary structure may be formed in a polynucleotide molecule, e.g., a DNA or an RNA molecule. In some embodiments, a secondary structure in a polynucleotide is formed by base pairing of complementary nucleotide sequences within a single polynucleotide molecule. In some embodiments, a secondary structure in a polynucleotide comprises one or more double-stranded regions through base pairing of complementary nucleotide sequences within a single polynucleotide molecule. In some embodiments, the secondary structure of a polynucleotide, e.g., a DNA or mRNA, comprises a hairpin, a stem, a loop, a tetraloop, a pseudoknot, a stem-loop, or any combination thereof. In some embodiments, when a polynucleotide contains an altered secondary structure as compared to a reference polynucleotide, the polynucleotide has a reduced or increased degree of secondary structure compared to the reference polynucleotide. Degree of secondary structure can be measured by the percentage of nucleotides of a polynucleotide that form complementary base pairs within the same polynucleotide.

In some embodiments, an optimized polynucleotide sequence, e.g., a mRNA encoding a prime editor fusion protein, exhibits an increased degree of secondary structure compared to a reference polynucleotide sequence, e.g., an unaltered reference mRNA encoding a PE protein. In some embodiments, a reference sequence is a wild-type polynucleotide sequence encoding all or a portion of a prime editor protein. In some embodiments, a reference sequence is a polynucleotide sequence encoding a functional variant of all or a portion of a prime editor protein, the reference sequence being altered from the wild type polynucleotide sequence only to encode one or more amino acid substitutions in of the functional variant. In some embodiments, a codon optimized polynucleotide sequence exhibits a reduced degree of secondary structure compared to a reference polynucleotide sequence. In some embodiments, a codon optimized polynucleotide comprises a reduced number of inverted repeat motifs compared to a reference polynucleotide sequence. In some embodiments, a codon optimized polynucleotide sequence exhibits an increased degree of secondary structure compared to a reference polynucleotide sequence. In some embodiments, a codon optimized polynucleotide comprises an increased number of inverted repeat motifs compared to a reference polynucleotide sequence.

In some embodiments, a codon optimized polynucleotide exhibits an altered degree of secondary structure in a specific portion as compared to a reference polynucleotide sequence. In some embodiments, a codon optimized polynucleotide exhibits a reduced degree of secondary structure in a specific portion as compared to a reference polynucleotide sequence. In some embodiments, the codon optimized polynucleotide exhibits an altered degree of secondary structure in an open reading frame (ORF) compared to a reference polynucleotide sequence. In some embodiments, the codon optimized polynucleotide exhibits a reduced degree of secondary structure in a ribosome binding site at the 5′ region of an ORF compared to a reference polynucleotide sequence. In some embodiments, the codon optimized polynucleotide exhibits a reduced degree of secondary structure at the N terminus of the ORF compared to a reference polynucleotide sequence. In some embodiments, the codon optimized polynucleotide exhibits a reduced degree of secondary structure at the C terminus of the ORF compared to a reference polynucleotide sequence. In some embodiments, a codon optimized polynucleotide sequence exhibits an increased secondary structure in a specific portion as compared to a reference polynucleotide sequence. In some embodiments, the codon optimized polynucleotide exhibits an increased degree of secondary structure in an open reading frame (ORF) compared to a reference polynucleotide sequence. In some embodiments, the codon optimized polynucleotide exhibits an increased degree of secondary structure at the N terminus of the ORF compared to a reference polynucleotide sequence. In some embodiments, the codon optimized polynucleotide exhibits an increased degree of secondary structure at the C terminus of the ORF compared to a reference polynucleotide sequence. In some embodiments, the codon optimized polynucleotide (e.g., mRNA) that encodes a prime editor polypeptide exhibits an increased degree of secondary structure compared to a reference coding sequence, e.g., of a SpCas9 or a M-MLV RT. In some embodiments, the codon optimized polynucleotide (e.g., mRNA) that encodes a prime editor polypeptide exhibits an increased secondary structure in an open reading frame (ORF) compared to the reference coding sequence, e.g., of a SpCas9 or a M-MLV RT. In some embodiments, the codon optimized polynucleotide (e.g., mRNA) that encodes a prime editor polypeptide exhibits secondary structure(s) that increase stability of the polynucleotide. In some embodiments, the codon optimized polynucleotide (e.g., mRNA) that encodes a prime editor polypeptide exhibits secondary structure(s) that increase initiation of polypeptide synthesis at or from an initiation codon. In some embodiments, the codon optimized polynucleotide (e.g., mRNA) that encodes a prime editor polypeptide exhibits secondary structure(s) that inhibit or reduce of the amount of polypeptide translated from any ORF within the polynucleotide other than the full ORF, thereby increasing translational fidelity of the prime editor polypeptide. In some embodiments, the secondary structure improves stability of the polynucleotide, e.g., mRNA, or a mRNA encoded by the polynucleotide. In some embodiments, the secondary structure improves thermostability of the polynucleotide, e.g., mRNA, or a mRNA encoded by the polynucleotide.

Optimized polynucleotides that encode prime editor polypeptide or components are provided.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.) Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants, and the like, as suited to the particular dosage form desired.

### Methods of Editing

The methods and compositions disclosed herein can be used to edit a double stranded target DNA, e.g., a target gene of interest by prime editing.

In some embodiments, the prime editing method comprises contacting a double stranded target DNA, e.g., a target gene, with a PEgRNA and a prime editor (PE) polypeptide described herein. In some embodiments, the double stranded target DNA, e.g., a target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a double stranded target DNA, e.g., a target gene.

In some embodiments, contacting the double stranded target DNA, e.g., a target gene with the prime editing composition results in binding of the PEgRNA to a target strand of the double stranded target DNA, e.g., a target gene. In some embodiments, contacting the double stranded target DNA, e.g., a target gene with the prime editing composition results in binding of the PEgRNA to a search target sequence on the target strand of the double stranded target DNA, e.g., a target gene upon contacting with the PEgRNA. In some embodiments, contacting the double stranded target DNA, e.g., a target gene with the prime editing composition results in binding of a spacer sequence of the PEgRNA to a search target sequence with the search target sequence on the target strand of the double stranded target DNA, e.g., a target gene upon said contacting of the PEgRNA.

In some embodiments, contacting the double stranded target DNA, e.g., a target gene with the prime editing composition results in binding of the prime editor to the double stranded target DNA, e.g., a target gene, e.g., the double stranded target DNA, e.g., a target gene, upon the contacting of the PE composition with the double stranded target DNA, e.g., a target gene. In some embodiments, the DNA binding domain of the PE associates with the PEgRNA. In some embodiments, the PE binds the double stranded target DNA, e.g., a target gene, directed by the PEgRNA. Accordingly, in some embodiments, the contacting of the double stranded target DNA, e.g., a target gene result in binding of a DNA binding domain of a prime editor of the double stranded target DNA, e.g., a target gene, directed by the PEgRNA.

In some embodiments, contacting the double stranded target DNA, e.g., a target gene with the prime editing composition results in a nick in an edit strand of the double stranded target DNA, e.g., a target gene, by the prime editor upon contacting with the double stranded target DNA, e.g., a target gene, thereby generating a nicked on the edit strand of the double stranded target DNA, e.g., a target gene. In some embodiments, contacting the double stranded target DNA, e.g., a target gene with the prime editing composition results in a single-stranded DNA comprising a free 3′ end at the nick site of the edit strand of the double stranded target DNA, e.g., a target gene. In some embodiments, contacting the double stranded target DNA, e.g., a target gene with the prime editing composition results in a nick in the edit strand of the double stranded target DNA, e.g., a target gene by a DNA binding domain of the prime editor, thereby generating a single-stranded DNA comprising a free 3 end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase

In some embodiments, contacting the double stranded target DNA, e.g., a target gene with the prime editing composition results in hybridization of the PEgRNA with the 3′ end of the nicked single-stranded DNA, thereby priming DNA polymerization by a DNA polymerase domain of the prime editor.

In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the double stranded target DNA, e.g., a target gene with a DNA polymerase, e.g., a reverse transcriptase, as a part of a prime editor fusion protein or prime editing complex (in cis), or as a separate protein (in trans).

In some embodiments, contacting the double stranded target DNA, e.g., a target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the double stranded target DNA, e.g., a target gene. In some embodiments, the intended nucleotide edits are incorporated in the double stranded target DNA, e.g., a target gene, by excision of the 5′ single stranded DNA of the edit strand of the double stranded target DNA, e.g., a target gene generated at the nick site and DNA repair. In some embodiments, the intended nucleotide edits are incorporated in the double stranded target DNA, e.g., a target gene by excision of the editing target sequence and DNA repair. In some embodiments, excision of the 5′ single stranded DNA of the edit strand generated at the nick site is by a flap endonuclease. In some embodiments, the flap nuclease is FEN 1. In some embodiments, the method further comprises contacting the double stranded target DNA, e.g., a target gene with a flap endonuclease. In some embodiments, the flap endonuclease is provided as a part of a prime editor fusion protein. In some embodiments, the flap endonuclease is provided in trans.

In some embodiments, contacting the double stranded target DNA, e.g., a target gene with the prime editing composition generates a mismatched heteroduplex comprising the edit strand of the double stranded target DNA, e.g., a target gene that comprises the edited single stranded DNA, and the unedited target strand of the double stranded target DNA, e.g., a target gene. Without being bound by theory, the endogenous DNA repair and replication may resolve the mismatched edited DNA to incorporate the nucleotide change(s) to form the desired edited double stranded target DNA, e.g., a target gene.

In some embodiments, the method further comprises contacting the double stranded target DNA, e.g., a target gene, with a nick guide (ngRNA) disclosed herein. In some embodiments, the ngRNA comprises a spacer that binds a second search target sequence on the edit strand of the double stranded target DNA, e.g., a target gene. In some embodiments, the contacted ngRNA directs the PE to introduce a nick in the target strand of the double stranded target DNA, e.g., a target gene. In some embodiments, the nick on the target strand (non-edit strand) results in endogenous DNA repair machinery to use the edit strand to repair the non-edit strand, thereby incorporating the intended nucleotide edit in both strand of the double stranded target DNA, e.g., a target gene and modifying the double stranded target DNA, e.g., a target gene. In some embodiments, the ngRNA comprises a spacer sequence that is complementary to, and may hybridize with, the second search target sequence on the edit strand only after the intended nucleotide edit(s) are incorporated in the edit strand of the double stranded target DNA, e.g., a target gene.

In some embodiments, the double stranded target DNA, e.g., a target gene is contacted by the ngRNA, the PEgRNA, and the PE simultaneously. In some embodiments, the ngRNA, the PEgRNA, and the PE form a complex when they contact the double stranded target DNA, e.g., a target gene. In some embodiments, the double stranded target DNA, e.g., a target gene is contacted with the ngRNA, the PEgRNA, and the prime editor sequentially. In some embodiments, the double stranded target DNA, e.g., a target gene is contacted with the ngRNA and/or the PEgRNA after contacting the double stranded target DNA, e.g., a target gene with the PE. In some embodiments, the double stranded target DNA, e.g., a target gene is contacted with the ngRNA and/or the PEgRNA before contacting the double stranded target DNA, e.g., a target gene with the prime editor.

In some embodiments, the double stranded target DNA, e.g., a target gene, is in a cell. Accordingly, also provided herein are methods of modifying a cell.

In some embodiments, the prime editing method comprises introducing a PEgRNA, a prime editor, and/or a ngRNA into the cell that has the double stranded target DNA, e.g., a target gene. In some embodiments, the prime editing method comprises introducing into the cell that has the double stranded target DNA, e.g., a target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a hepatocyte. In some embodiments, the cell is a human hepatocyte. In some embodiments, the cell is a primary human hepatocyte derived from an induced human pluripotent stem cell (iPSC). In some embodiments, the cell is a hematopoietic stem cell (HSC). In some embodiments, the cell is a human HSC. In some embodiments, the cell is a human CD34⁺ HSC. In some embodiments, the codon optimization is for expression in a human CD34⁺ hematopoietic stem progenitor cell (HSPC).

In some embodiments, the double stranded target DNA, e.g., a target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the double stranded target DNA, e.g., a target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the double stranded target DNA, e.g., a target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the double stranded target DNA, e.g., a target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the double stranded target DNA, e.g., a target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the double stranded target DNA, e.g., a target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the double stranded target DNA, e.g., a target gene in sufficient number of cells such that the disease or disorder is treated, prevented or ameliorated.

In some embodiments, editing efficiency of the prime editing compositions and method described herein can be measured by calculating the percentage of edited double stranded target DNA, e.g., a target gene in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a double stranded target DNA, e.g., a target gene to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. In some embodiments, the prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing in a primary cell relative to a suitable control primary cell.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing in a hepatocyte relative to a corresponding control hepatocyte. In some embodiments, the hepatocyte is a human hepatocyte.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing in a hematopoietic stem cell (HSC) relative to a corresponding control HSC. In some embodiments, the HSC is a human HSC.

In some embodiments, the methods disclosed herein having an increased editing efficiency by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300% or more compared to prime editing with a prime editor having the sequence of SEQ ID NO: 77 and/or encoded by SEQ ID NO: 78. In some embodiments, the methods disclosed herein having an increased editing efficiency by at least 5%, at least 10%, at least 15%, at least 20%, at least 25%, at least 30%, at least 35%, at least 400%, at least 45%, at least 50%, at least 55%, at least 600%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300% or more compared to prime editing with a prime editor having the sequence of SEQ ID NO: 77 and/or encoded by SEQ ID NO: 78. In some embodiments, the increased editing efficiency is in a human cell. In some embodiments, the increased editing efficiency is in a primary cell. In some embodiments, the increased editing efficiency is in a human primary cell. In some embodiments, the increased editing efficiency is in a progenitor cell. In some embodiments, the increased editing efficiency is in a human progenitor cell. In some embodiments, the increased editing efficiency is in a hepatocyte. In some embodiments, the increased editing efficiency is in a human hepatocyte. In some embodiments, the increased editing efficiency is in a primary human hepatocyte derived from an induced human pluripotent stem cell (iPSC). In some embodiments, the increased editing efficiency is in a hematopoietic stem cell (HSC). In some embodiments, the increased editing efficiency is in a primary cell. In some embodiments, the increased editing efficiency is in a human CD34+ HSC.

In some embodiments, the prime editing compositions provided herein are capable of incorporating one or more intended nucleotide edits without generating a significant proportion of indels. The term “indel(s)”, as used herein, refers to the insertion or deletion of a nucleotide base within a polynucleotide, for example, a double stranded target DNA, e.g., a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a double stranded target DNA, e.g., a target gene.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 1% in a target cell, e.g., a human HSC. In some embodiments, the prime editing methods disclosed herein have an editing efficiency of at least about 1% and an indel frequency of less than 0.5% in a target cell, a human HSC. In some embodiments, the prime editing methods disclosed herein have an editing efficiency of at least about 1% and an indel frequency of less than 0.1% in a target cell, e.g., a human HSC. In some embodiments, the prime editing methods disclosed herein have an editing efficiency of at least about 5% and an indel frequency of less than 1% in a target cell, e.g. a human HSC. In some embodiments, the prime editing methods disclosed herein have an editing efficiency of at least about 5% and an indel frequency of less than 0.5% in a target cell, e.g., a human HSC. In some embodiments, the prime editing methods disclosed herein have an editing efficiency of at least about 5% and an indel frequency of less than 0.1% in a target cell, e.g., a human HSC.

In some embodiments, the prime editing methods disclosed herein have an editing efficiency of at least about 7.5% and an indel frequency of less than 1% in a target cell, e.g., a human HSC. In some embodiments, the prime editing methods disclosed herein have an editing efficiency of at least about 7.5% and an indel frequency of less than 0.5% in a target cell, e.g., a human HSC. In some embodiments, the prime editing methods disclosed herein have an editing efficiency of at least about 7.5% and an indel frequency of less than 0.1% in a target cell, e.g., a human HSC.

In some embodiments, the prime editing methods disclosed herein have an editing efficiency of at least about 10% and an indel frequency of less than 1% in a target cell, e.g., a human HSC. In some embodiments, the prime editing methods disclosed herein have an editing efficiency of at least about 10% and an indel frequency of less than 0.5% in a target cell, e.g., a human HSC. In some embodiments, the prime editing methods disclosed herein have an editing efficiency of at least about 10% and an indel frequency of less than 0.1% in a target cell, e.g., a human HSC.

In some embodiments, the prime editing methods disclosed herein have an editing efficiency of at least about 15% and an indel frequency of less than 1% in a target cell, e.g., a human HSC. In some embodiments, the prime editing methods disclosed herein have an editing efficiency of at least about 15% and an indel frequency of less than 0.5% in a target cell, e.g., a human HSC. In some embodiments, the prime editing methods disclosed herein have an editing efficiency of at least about 15% and an indel frequency of less than 0.1% in a target cell, e.g., a human HSC.

In some embodiments, the prime editing methods disclosed herein have an editing efficiency of at least about 20% and an indel frequency of less than 1% in a target cell, e.g., a human HSC. In some embodiments, the prime editing methods disclosed herein have an editing efficiency of at least about 20% and an indel frequency of less than 0.5% in a target cell, e.g., a human HSC. In some embodiments, the prime editing methods disclosed herein have an editing efficiency of at least about 20% and an indel frequency of less than 0.1% in a target cell, e.g., a human HSC.

In some embodiments, the prime editing methods disclosed herein have an editing efficiency of at least about 30% and an indel frequency of less than 1% in a target cell, e.g., a human HSC. In some embodiments, the prime editing methods disclosed herein have an editing efficiency of at least about 30% and an indel frequency of less than 0.5% in a target cell, e.g., a human HSC. In some embodiments, the prime editing methods disclosed herein have an editing efficiency of at least about 30% and an indel frequency of less than 0.1% in a target cell, e.g., a human HSC.

In some embodiments, the prime editing methods disclosed herein have an editing efficiency of at least about 40% and an indel frequency of less than 1% in a target cell, e.g., a human HSC. In some embodiments, the prime editing methods disclosed herein have an editing efficiency of at least about 40% and an indel frequency of less than 0.5% in a target cell, e.g., a human HSC. In some embodiments, the prime editing methods disclosed herein have an editing efficiency of at least about 40% and an indel frequency of less than 0.1% in a target cell, e.g., a human HSC.

In some embodiments, the prime editing methods disclosed herein have an editing efficiency of at least about 50% and an indel frequency of less than 1% in a target cell, e.g., a human HSC. In some embodiments, the prime editing methods disclosed herein have an editing efficiency of at least about 50% and an indel frequency of less than 0.5% in a target cell, e.g., a human HSC. In some embodiments, the prime editing methods disclosed herein have an editing efficiency of at least about 50% and an indel frequency of less than 0.1% in a target cell, e.g., a human HSC.

In some embodiments, the prime editing methods disclosed herein have an editing efficiency of at least about 60% and an indel frequency of less than 1% in a target cell, e.g., a human HSC. In some embodiments, the prime editing methods disclosed herein have an editing efficiency of at least about 60% and an indel frequency of less than 0.5% in a target cell, e.g., a human HSC. In some embodiments, the prime editing methods disclosed herein have an editing efficiency of at least about 60% and an indel frequency of less than 0.1% in a target cell, e.g., a human HSC.

In some embodiments, the prime editing methods disclosed herein have an editing efficiency of at least about 70% and an indel frequency of less than 1% in a target cell, e.g., a human HSC. In some embodiments, the prime editing methods disclosed herein have an editing efficiency of at least about 70% and an indel frequency of less than 0.5% in a target cell, e.g., a human HSC. In some embodiments, the prime editing methods disclosed herein have an editing efficiency of at least about 70% and an indel frequency of less than 0.1% in a target cell, e.g., a human HSC.

In some embodiments, the prime editing methods disclosed herein have an editing efficiency of at least about 80% and an indel frequency of less than 1% in a target cell, e.g., a human HSC. In some embodiments, the prime editing methods disclosed herein have an editing efficiency of at least about 80% and an indel frequency of less than 0.5% in a target cell, e.g., a human HSC. In some embodiments, the prime editing methods disclosed herein have an editing efficiency of at least about 80% and an indel frequency of less than 0.1% in a target cell, e.g., a human HSC.

In some embodiments, the prime editing methods disclosed herein have an editing efficiency of at least about 90% and an indel frequency of less than 1% in a target cell, e.g., a human HSC. In some embodiments, the prime editing methods disclosed herein have an editing efficiency of at least about 90% and an indel frequency of less than 0.5% in a target cell, e.g., a human HSC. In some embodiments, the prime editing methods disclosed herein have an editing efficiency of at least about 90% and an indel frequency of less than 0.1% in a target cell, e.g., a human HSC.

In some embodiments, the prime editing methods disclosed herein have an editing efficiency of at least about 95% and an indel frequency of less than 1% in a target cell, e.g., a human cell. In some embodiments, the prime editing methods disclosed herein have an editing efficiency of at least about 95% and an indel frequency of less than 0.5% in a target cell, e.g., a human cell. In some embodiments, the prime editing methods disclosed herein have an editing efficiency of at least about 95% and an indel frequency of less than 0.1% in a target cell, e.g., a human cell.

In some embodiments, the prime editing methods disclosed herein have an editing efficiency of at least about 1% and an indel frequency of less than 10% in a population of target cells, e.g., a population of human cells, such as a human stem cell. In some embodiments, the prime editing methods disclosed herein have an editing efficiency of at least about 1% and an indel frequency of less than 7.5% in a population of target cells. In some embodiments, the prime editing methods disclosed herein have an editing efficiency of at least about 1% and an indel frequency of less than 5% in a population of target cells. In some embodiments, the prime editing methods disclosed herein have an editing efficiency of at least about 1% and an indel frequency of less than 2.5% in a population of target cells. In some embodiments, the prime editing methods disclosed herein have an editing efficiency of at least about 1% and an indel frequency of less than 1% in a population of target cells. In some embodiments, the prime editing methods disclosed herein have an editing efficiency of at least about 1% and an indel frequency of less than 0.5% in a population of target cells. In some embodiments, the prime editing methods disclosed herein have an editing efficiency of at least about 1% and an indel frequency of less than 0.1% in a population of target cells.

In some embodiments, the prime editing methods disclosed herein have an editing efficiency of at least about 5% and an indel frequency of less than 10% in a population of target cells. In some embodiments, the prime editing methods disclosed herein have an editing efficiency of at least about 5% and an indel frequency of less than 7.5% in a population of target cells. In some embodiments, the prime editing methods disclosed herein have an editing efficiency of at least about 5% and an indel frequency of less than 5% in a population of target cells. In some embodiments, the prime editing methods disclosed herein have an editing efficiency of at least about 5% and an indel frequency of less than 2.5% in a population of target cells. In some embodiments, the prime editing methods disclosed herein have an editing efficiency of at least about 5% and an indel frequency of less than 1% in a population of target cells. In some embodiments, the prime editing methods disclosed herein have an editing efficiency of at least about 5% and an indel frequency of less than 0.5% in a population of target cells. In some embodiments, the prime editing methods disclosed herein have an editing efficiency of at least about 5% and an indel frequency of less than 0.1% in a population of target cells.

In some embodiments, the prime editing methods disclosed herein have an editing efficiency of at least about 7.5% and an indel frequency of less than 10% in a population of target cells. In some embodiments, the prime editing methods disclosed herein have an editing efficiency of at least about 7.5% and an indel frequency of less than 7.5% in a population of target cells. In some embodiments, the prime editing methods disclosed herein have an editing efficiency of at least about 7.5% and an indel frequency of less than 5% in a population of target cells. In some embodiments, the prime editing methods disclosed herein have an editing efficiency of at least about 7.5% and an indel frequency of less than 2.5% in a population of target cells. In some embodiments, the prime editing methods disclosed herein have an editing efficiency of at least about 7.5% and an indel frequency of less than 1% in a population of target cells. In some embodiments, the prime editing methods disclosed herein have an editing efficiency of at least about 7.5% and an indel frequency of less than 0.5% in a population of target cells. In some embodiments, the prime editing methods disclosed herein have an editing efficiency of at least about 7.5% and an indel frequency of less than 0.1% in a population of target cells.

In some embodiments, the prime editing methods disclosed herein have an editing efficiency of at least about 10% and an indel frequency of less than 10% in a population of target cell. In some embodiments, the prime editing methods disclosed herein have an editing efficiency of at least about 10% and an indel frequency of less than 7.5% in a population of target cells. In some embodiments, the prime editing methods disclosed herein have an editing efficiency of at least about 10% and an indel frequency of less than 5% in a population of target cells. In some embodiments, the prime editing methods disclosed herein have an editing efficiency of at least about 10% and an indel frequency of less than 2.5% in a population of target cells. In some embodiments, the prime editing methods disclosed herein have an editing efficiency of at least about 10% and an indel frequency of less than 1% in a population of target cells. In some embodiments, the prime editing methods disclosed herein have an editing efficiency of at least about 10% and an indel frequency of less than 0.5% in a population of target cells. In some embodiments, the prime editing methods disclosed herein have an editing efficiency of at least about 10% and an indel frequency of less than 0.1% in a population of target cells.

In some embodiments, the prime editing methods disclosed herein have an editing efficiency of at least about 15% and an indel frequency of less than 10% in a population of target cells. In some embodiments, the prime editing methods disclosed herein have an editing efficiency of at least about 15% and an indel frequency of less than 7.5% in a population of target cells. In some embodiments, the prime editing methods disclosed herein have an editing efficiency of at least about 15% and an indel frequency of less than 5% in a population of target cells. In some embodiments, the prime editing methods disclosed herein have an editing efficiency of at least about 15% and an indel frequency of less than 2.5% in a population of target cells. In some embodiments, the prime editing methods disclosed herein have an editing efficiency of at least about 15% and an indel frequency of less than 1% in a population of target cells. In some embodiments, the prime editing methods disclosed herein have an editing efficiency of at least about 15% and an indel frequency of less than 0.5% in a population of target cells. In some embodiments, the prime editing methods disclosed herein have an editing efficiency of at least about 15% and an indel frequency of less than 0.1% in a population of target cells.

In some embodiments, the prime editing methods disclosed herein have an editing efficiency of at least about 20% and an indel frequency of less than 10% in a population of target cells. In some embodiments, the prime editing methods disclosed herein have an editing efficiency of at least about 20% and an indel frequency of less than 7.5% in a population of target cells. In some embodiments, the prime editing methods disclosed herein have an editing efficiency of at least about 20% and an indel frequency of less than 5% in a population of target cells. In some embodiments, the prime editing methods disclosed herein have an editing efficiency of at least about 20% and an indel frequency of less than 2.5% in a population of target cells. In some embodiments, the prime editing methods disclosed herein have an editing efficiency of at least about 20% and an indel frequency of less than 1% in a population of target cells, e.g., a population of human stem cell. In some embodiments, the prime editing methods disclosed herein have an editing efficiency of at least about 20% and an indel frequency of less than 0.5% in a population of target cells. In some embodiments, the prime editing methods disclosed herein have an editing efficiency of at least about 20% and an indel frequency of less than 0.1% in a population of target cells.

In some embodiments, the prime editing methods disclosed herein have an editing efficiency of at least about 30% and an indel frequency of less than 10% in a population of target cells. In some embodiments, the prime editing methods disclosed herein have an editing efficiency of at least about 30% and an indel frequency of less than 7.5% in a population of target cells. In some embodiments, the prime editing methods disclosed herein have an editing efficiency of at least about 30% and an indel frequency of less than 5% in a population of target cells. In some embodiments, the prime editing methods disclosed herein have an editing efficiency of at least about 30% and an indel frequency of less than 2.5% in a population of target cells. In some embodiments, the prime editing methods disclosed herein have an editing efficiency of at least about 30% and an indel frequency of less than 1% in a population of target cells. In some embodiments, the prime editing methods disclosed herein have an editing efficiency of at least about 30% and an indel frequency of less than 0.5% in a population of target cells. In some embodiments, the prime editing methods disclosed herein have an editing efficiency of at least about 30% and an indel frequency of less than 0.1% in a population of target cells.

In some embodiments, the prime editing methods disclosed herein have an editing efficiency of at least about 40% and an indel frequency of less than 10% in a population of target cells. In some embodiments, the prime editing methods disclosed herein have an editing efficiency of at least about 40% and an indel frequency of less than 7.5% in a population of target cells. In some embodiments, the prime editing methods disclosed herein have an editing efficiency of at least about 40% and an indel frequency of less than 5% in a population of target cells. In some embodiments, the prime editing methods disclosed herein have an editing efficiency of at least about 40% and an indel frequency of less than 2.5% in a population of target cells. In some embodiments, the prime editing methods disclosed herein have an editing efficiency of at least about 40% and an indel frequency of less than 1% in a population of target cells. In some embodiments, the prime editing methods disclosed herein have an editing efficiency of at least about 40% and an indel frequency of less than 0.5% in a population of target cells. In some embodiments, the prime editing methods disclosed herein have an editing efficiency of at least about 40% and an indel frequency of less than 0.1% in a population of target cells.

In some embodiments, the prime editing methods disclosed herein have an editing efficiency of at least about 50% and an indel frequency of less than 10% in a population of target cells. In some embodiments, the prime editing methods disclosed herein have an editing efficiency of at least about 50% and an indel frequency of less than 7.5% in a population of target cells. In some embodiments, the prime editing methods disclosed herein have an editing efficiency of at least about 50% and an indel frequency of less than 5% in a population of target cells. In some embodiments, the prime editing methods disclosed herein have an editing efficiency of at least about 50% and an indel frequency of less than 2.5% in a population of target cells. In some embodiments, the prime editing methods disclosed herein have an editing efficiency of at least about 50% and an indel frequency of less than 1% in a population of target cells. In some embodiments, the prime editing methods disclosed herein have an editing efficiency of at least about 50% and an indel frequency of less than 0.5% in a population of target cells. In some embodiments, the prime editing methods disclosed herein have an editing efficiency of at least about 50% and an indel frequency of less than 0.1% in a population of target cells.

In some embodiments, the prime editing methods disclosed herein have an editing efficiency of at least about 60% and an indel frequency of less than 10% in a population of target cells. In some embodiments, the prime editing methods disclosed herein have an editing efficiency of at least about 60% and an indel frequency of less than 7.5% in a population of target cells. In some embodiments, the prime editing methods disclosed herein have an editing efficiency of at least about 60% and an indel frequency of less than 5% in a population of target cells. In some embodiments, the prime editing methods disclosed herein have an editing efficiency of at least about 60% and an indel frequency of less than 2.5% in a population of target cells. In some embodiments, the prime editing methods disclosed herein have an editing efficiency of at least about 60% and an indel frequency of less than 1% in a population of target cells. In some embodiments, the prime editing methods disclosed herein have an editing efficiency of at least about 60% and an indel frequency of less than 0.5% in a population of target cells. In some embodiments, the prime editing methods disclosed herein have an editing efficiency of at least about 60% and an indel frequency of less than 0.1% in a population of target cells.

In some embodiments, the prime editing methods disclosed herein have an editing efficiency of at least about 70% and an indel frequency of less than 10% in a population of target cells. In some embodiments, the prime editing methods disclosed herein have an editing efficiency of at least about 70% and an indel frequency of less than 7.5% in a population of target cells. In some embodiments, the prime editing methods disclosed herein have an editing efficiency of at least about 70% and an indel frequency of less than 5% in a population of target cells. In some embodiments, the prime editing methods disclosed herein have an editing efficiency of at least about 70% and an indel frequency of less than 2.5% in a population of target cells. In some embodiments, the prime editing methods disclosed herein have an editing efficiency of at least about 70% and an indel frequency of less than 1% in a population of target cells. In some embodiments, the prime editing methods disclosed herein have an editing efficiency of at least about 70% and an indel frequency of less than 0.5% in a population of target cells. In some embodiments, the prime editing methods disclosed herein have an editing efficiency of at least about 70% and an indel frequency of less than 0.1% in a population of target cells.

In some embodiments, the prime editing methods disclosed herein have an editing efficiency of at least about 80% and an indel frequency of less than 10% in a population of target cells. In some embodiments, the prime editing methods disclosed herein have an editing efficiency of at least about 80% and an indel frequency of less than 7.5% in a population of target cells. In some embodiments, the prime editing methods disclosed herein have an editing efficiency of at least about 80% and an indel frequency of less than 5% in a population of target cells. In some embodiments, the prime editing methods disclosed herein have an editing efficiency of at least about 80% and an indel frequency of less than 2.5% in a population of target cells. In some embodiments, the prime editing methods disclosed herein have an editing efficiency of at least about 80% and an indel frequency of less than 1% in a population of target cells. In some embodiments, the prime editing methods disclosed herein have an editing efficiency of at least about 80% and an indel frequency of less than 0.5% in a population of target cells. In some embodiments, the prime editing methods disclosed herein have an editing efficiency of at least about 80% and an indel frequency of less than 0.1% in a population of target cells.

In some embodiments, the prime editing methods disclosed herein have an editing efficiency of at least about 90% and an indel frequency of less than 10% in a population of target cells. In some embodiments, the prime editing methods disclosed herein have an editing efficiency of at least about 90% and an indel frequency of less than 7.5% in a population of target cells. In some embodiments, the prime editing methods disclosed herein have an editing efficiency of at least about 90% and an indel frequency of less than 5% in a population of target cells. In some embodiments, the prime editing methods disclosed herein have an editing efficiency of at least about 90% and an indel frequency of less than 2.5% in a population of target cells. In some embodiments, the prime editing methods disclosed herein have an editing efficiency of at least about 90% and an indel frequency of less than 1% in a population of target cells. In some embodiments, the prime editing methods disclosed herein have an editing efficiency of at least about 90% and an indel frequency of less than 0.5% in a population of target cells. In some embodiments, the prime editing methods disclosed herein have an editing efficiency of at least about 90% and an indel frequency of less than 0.1% in a population of target cells.

In some embodiments, the prime editing methods disclosed herein have an editing efficiency of at least about 95% and an indel frequency of less than 10% in a population of target cells. In some embodiments, the prime editing methods disclosed herein have an editing efficiency of at least about 95% and an indel frequency of less than 7.5% in a population of target cells. In some embodiments, the prime editing methods disclosed herein have an editing efficiency of at least about 95% and an indel frequency of less than 5% in a population of target cells. In some embodiments, the prime editing methods disclosed herein have an editing efficiency of at least about 95% and an indel frequency of less than 2.5% in a population of target cells. In some embodiments, the prime editing methods disclosed herein have an editing efficiency of at least about 95% and an indel frequency of less than 1% in a population of target cells. In some embodiments, the prime editing methods disclosed herein have an editing efficiency of at least about 95% and an indel frequency of less than 0.5% in a population of target cells. In some embodiments, the prime editing methods disclosed herein have an editing efficiency of at least about 95% and an indel frequency of less than 0.1% in a population of target cells.

In some embodiments, the target gene is in a target cell. Accordingly, in one aspect provided herein is a method of editing a target cell comprising a double stranded target DNA (e.g., a target gene) that encoded a polypeptide, wherein the double stranded target DNA comprises one or more mutations relative to the wild-type double stranded DNA (e.g., wild-type gene). In some embodiments, the methods of the present disclosure comprise introducing a prime editing composition comprising a PEgRNA, a prime editor polypeptide, a ngRNA, and/or a polynucleotide encoding the PEgRNA, the prime editor polypeptide, or the ngRNA into the target cell that has the target gene to edit the target gene, thereby generating an edited cell. In some embodiments, a target cell is a cell disclosed herein. In some embodiments, the target cell is a mammalian cell. In some embodiments, the target cell is a human cell.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a double stranded target DNA, e.g., a target gene to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a double stranded target DNA, e.g., a target gene, to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the double stranded target DNA, e.g., a target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a double stranded target DNA, e.g., a target gene (e.g., a nucleic acid within the genome of a cell) to a prime editing composition.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, the prime editing compositions (e.g., PEgRNAs and prime editors as described herein) and prime editing methods disclosed herein can be used to edit a double stranded target DNA, e.g., a target gene. In some embodiments, the double stranded target DNA, e.g., a target gene, comprises a mutation compared to a wild-type sequence of the same gene. In some embodiments, the mutation is associated with a genetic disease or disorder. In some embodiments, the mutation is in a coding region of the double stranded target DNA, e.g., a target gene. In some embodiments, the mutation is in an exon of the double stranded target DNA, e.g., a target gene. In some embodiments, the prime editing method comprises contacting a double stranded target DNA, e.g., a target gene, with a prime editing composition comprising a prime editor, a PEgRNA, and/or a ngRNA. In some embodiments, contacting the double stranded target DNA, e.g., a target gene, with the prime editing composition results in incorporation of one or more intended nucleotide edits in the double stranded target DNA, e.g. a target gene. In some embodiments, the incorporation is in a region of the double stranded target DNA, e.g., a target gene, that corresponds to an editing target sequence in the target gene. In some embodiments, the one or more intended nucleotide edits comprises a single nucleotide substitution, an insertion, a deletion, or any combination thereof, compared to the endogenous sequence of the double stranded target DNA, e.g., a target gene. In some embodiments, incorporation of the one or more intended nucleotide edits results in replacement of one or more mutations with a DNA sequence that encodes a corresponding wild-type protein. In some embodiments, incorporation of the one or more intended nucleotide edits results in replacement of the one or more mutations with the corresponding wild-type gene sequence. In some embodiments, incorporation of the one more intended nucleotide edits results in correction of a mutation in the double stranded target DNA, e.g., a target gene. In some embodiments, the double stranded target DNA, e.g., a target gene, comprises an editing template sequence that contains the mutation. In some embodiments, contacting the double stranded target DNA, e.g., a target gene, with the prime editing composition results in incorporation of one or more intended nucleotide edits in the double stranded target DNA, e.g., a target gene, which corrects the mutation in the editing target sequence (or a double stranded region comprising the editing target sequence and the complementary sequence to the editing target sequence on a target strand) in the double stranded target DNA, e.g., a target gene. In some embodiments, incorporation of the one or more intended nucleotide edits in the double stranded target DNA, e.g., a target gene, that comprises one or more mutations, restores wild-type expression and function of a protein encoded by the target gene. In some embodiments, expression and/or function of the protein encoded by the target gene may be measured when expressed in a target cell. In some embodiments, incorporation of the one or more intended nucleotide edits in the double stranded target DNA, e.g., a target gene, leads to a fold change in a level of the target gene expression and/or a fold change in a level of the functional protein encoded by the target gene. In some embodiments, a change in the level of the target gene expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the double stranded target DNA, e.g., a target gene, that comprises one or more mutations, restores wild-type expression of the functional protein encoded by the target gene by at least 10%, 20%, 30%, 40%, 50%, 60%, 70%, 80%, 90%, 95%, 99% or more as compared to wild-type expression of the corresponding protein in a suitable control cell that comprises a wild-type target gene.

In some embodiments, an expression increase can be measured by a functional assay. In some embodiments, protein expression can be measured using a protein assay. In some embodiments, protein expression can be measured using antibody testing. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

In some embodiments, the target gene comprises one or more mutations associated with a genetic disease or disorder. Accordingly, in some embodiments, provided herein are methods for treatment of a subject diagnosed with a disease associated with or caused by one or more pathogenic mutations that can be corrected by prime editing.

In some embodiments, provided herein are methods for treating a genetic disease that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the double stranded target DNA, e.g., a target gene, in the subject. In some embodiments, administration of the prime editing composition results in correction of one or more pathogenic mutations, e.g., point mutations, insertions, or deletions, associated with a disease in the subject. In some embodiments, the double stranded target DNA, e.g., a target gene comprises an editing target sequence that contains the pathogenic mutation. In some embodiments, administration of the prime editing composition results in incorporation of one or more intended nucleotide edits in the double stranded target DNA, e.g., a target gene that corrects the pathogenic mutation in the editing target sequence (or a double stranded region comprising the editing target sequence and the complementary sequence to the editing target sequence on a target strand) of the double stranded target DNA, e.g., a target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the target gene having pathogenic mutation(s) in a subject, e.g., a human subject, suffering from, having, susceptible to, or at risk for the disease. Identifying a subject in need of such treatment can be in the judgment of a subject or a health care professional and can be subjective (e.g. opinion) or objective (e.g. measurable by a test or diagnostic method).

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection or infusion or transfusion, transplantation (e.g., allogeneic hematopoietic stem cell transplantation (HSCT) using cells that have been contacted with a prime editing complex as described herein) to a subject. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. The cells may be contacted ex vivo with any approach described herein or known in the art. For example, in some embodiments, one or more target cells are contacted with one or more components of a prime editing composition ex vivo by electroporation. In some embodiments, one or more target cells are contacted with one or more components of a prime editing composition ex vivo by a LNP comprising the prime editing composition or components thereof. In some embodiments, one or more target cells are contacted with one or more components of a prime editing composition ex vivo, wherein one or more components of the prime editing composition is associated with a cell penetrating peptide. In some embodiments, the ex v/vo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are hepatocytes. In some embodiments, the edited cells are primary human cells. In some embodiments, the edited cells are human progenitor cells. In some embodiments, the edited cells are human stem cells. In some embodiments, the edited cells are human hepatocytes. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

In some embodiments, the target gene to be edited in a subject is a SERPINA1 gene. In some embodiments, the SERPINA1 gene comprises a mutation associated with Alpha-1-antitrypsin (A1AT) deficiency. In some embodiments, the SERPINA1 gene comprises a mutation that encodes an adenine (A) instead of a guanine (G) in the A1AT encoded by the SERPINA1 gene compared to a wild type A1AT. In some embodiments, provided herein is a prime editing composition comprising a prime editor and a PEgRNA, wherein the PEgRNA is capable of directing the prime editor to correct the mutation associated with A1AT deficiency in a SERPINA1 gene. In some embodiments, the PEgRNA comprises an editing template that comprises an intended nucleotide edit, and wherein incorporation of the intended nucleotide edit in the SERPINA1 gene corrects the mutation in the SERPINA1 gene associated with A1AT deficiency. In some embodiments, the editing template comprises a wild type sequence of a wild type SERPINA1 gene. Accordingly, in some embodiments, provided herein are methods of correcting a mutation associated with A1AT deficiency in a SERPINA1 gene. In some embodiments, the method comprises contacting the SERPINA1 gene with a PEgRNA and a prime editor, wherein the PEgRNA directs the prime editor to incorporate an intended nucleotide edit in the SERPINA1 gene, thereby correcting the mutation associated with A1AT deficiency in the SERPINA1 gene. In some embodiments, the SERPINA1 gene is in a cell. Accordingly, in some embodiments, the method comprises introducing into the cell comprising the SERPINA1 gene with a PEgRNA and a prime editor, wherein the PEgRNA directs the prime editor to incorporate an intended nucleotide edit in the SERPINA1 gene, thereby correcting the mutation associated with A1AT deficiency in the SERPINA1 gene. In some embodiments, the method comprises introducing into the cell comprising the SERPINA1 gene with a PEgRNA and a polynucleotide encoding the prime editor, wherein upon expression of the prime editor, the PEgRNA directs the prime editor to incorporate an intended nucleotide edit in the SERPINA1 gene, thereby correcting the mutation associated with A1AT deficiency in the SERPINA1 gene. In some embodiments, the cell is a liver cell. In some embodiments, the cell is in vivo. In some embodiments, the cell is ex vivo. In some embodiments, the PEgRNA and the prime editor are introduced into the cell simultaneously. In some embodiments, the PEgRNA and the polynucleotide encoding the prime editor are introduced into the cell simultaneously. In some embodiments, the PEgRNA and the prime editor are introduced into the cell sequentially, for example, the PEgRNA may be introduced prior to or after introduction of the prime editor. In some embodiments, the PEgRNA and the polynucleotide encoding the prime editor are introduced into the cell sequentially, for example, the PEgRNA may be introduced prior to or after introduction of the polynucleotide encoding the prime editor.

Accordingly, in some embodiments, provided herein is a method of treating A1AT deficiency, wherein the method comprises administering to a subject in need thereof a PEgRNA and a prime editor or a polynucleotide encoding the prime editor, wherein the PEgRNA directs the prime editor to incorporate the intended nucleotide edit in a SERPINA1 gene in the subject, thereby correcting a mutation in the SERPINA1 gene and treating A1AT deficiency. In some embodiments, the method of treating A1AT deficiency comprises introducing a PEgRNA and a prime editor or a polynucleotide encoding the prime editor to a cell or a population of cells to correct a mutation associated with A1AT deficiency in a SERPINA1 gene, and subsequently administering the edited cell or the edited population of cells to a subject in need thereof. In some embodiments, the cell or the population of cells are obtained from the subject in need thereof prior to editing. In some embodiments, the cell or the population of cells are obtained from a donor prior to editing. In some embodiments, the cell or the population of cells are hematopoietic stem cells. In some embodiments, the PEgRNA and the prime editor are administered simultaneously. In some embodiments, the PEgRNA and the polynucleotide encoding the prime editor are administered simultaneously. In some embodiments, the PEgRNA and the prime editor are administered sequentially, for example, the PEgRNA may be administered prior to or after administration of the prime editor. In some embodiments, the PEgRNA and the polynucleotide encoding the prime editor are administered sequentially, for example, the PEgRNA may be administered prior to or after administration of the polynucleotide encoding the prime editor.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

### Delivery

Prime editing compositions described herein can be delivered to a cellular environment with any approach known in the art. Components of a prime editing composition can be delivered to a cell by the same mode or different modes. For example, in some embodiments, a prime editor or components thereof (e.g., a DNA binding domain or a DNA polymerase domain) can be delivered as a polypeptide or a polynucleotide (DNA or RNA) encoding the polypeptide or as a ribonucleoprotein (RNP) complex. In some embodiments, a PEgRNA can be delivered directly as an RNA or as a DNA encoding the PEgRNA or as an RNA complexed to the PE protein as an RNP complex. In some embodiments, components of a prime editing composition can be delivered as a combination of DNA and RNA. In some embodiments, components of a prime editor composition can be delivered as a combination of polynucleotide e.g., DNA, or RNA, and protein.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA binding domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, HI promoter).

In some embodiments, the polynucleotide encoding one or more prime editing composition components is a part of, or is encoded by, a vector (e.g., a plasmid vector or a viral vector). In some embodiments, the vector is a viral vector. In some embodiments, the vector is a non-viral vector. In some embodiments, delivery is in vivo, in vitro, ex vivo, or in situ.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a prime editor-encoding mRNA, can be delivered in the form of RNA. In some embodiments, one or more components of the prime editing system that are RNAs is produced by direct chemical synthesis or may be transcribed in vitro from a DNA. In some embodiments, an mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a double stranded target DNA, e.g., a target gene, or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, electroporation, microinjection, biolistics, virosomes, liposomes, immunoliposomes, cell penetrating peptides, polycation or lipid nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), Simian Immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector. In some embodiments, the AAV is a recombinant AAV (rAAV).

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and y2 cells or PA317 cells (e.g, for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins. In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size. A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 302). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 396), and octa-arginine (SEQ ID NO: 395). The nona-arginine (R9) (SEQ ID NO: 396) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell.

### EXAMPLES

While several experimental Examples are contemplated, these Examples are intended to be non-limiting.

### Example 1. Prime Editing of the α-1 Antitrypsin (A1AT) Pathogenic Z-Mutation (E342K) in Human Primary Fibroblast Cells

α-1 antitrypsin deficiency may result from the presence of one or more mutations in the SERPINA1 gene. The most common mutation is the E342K mutation, which results from a single nucleotide polymorphism (SNP) where a guanine (G) is replaced by an adenine (A). Prime editing was employed to correct the A1AT E342K genomic locus.

Human primary fibroblast cells containing the A1AT gene with the pathogenic E342K G>A SNP were utilized for various prime editing correction. These cells were transfected with mRNA encoding the prime editor designated HRB-314, which employs, from N-terminus to C-terminus, an SpCas9 nickase with the H840A mutation, the SGGS-(EAAAK)₄-SGGS (SEQ ID NO: 277) linker, and the G504X reverse transcriptase. The prime editor HRB-314, including the G504X RT, are further described in WO2023283092A1, incorporated herein by reference. The cells were also transfected with one or several pegRNAs to correct the E342K mutation. All of the pegRNAs employed the same spacer sequence (UCCCCUCCAGGCCGUGCAUA, SEQ ID NO: 1), but varied in primer binding site (PBS) and RT template (RTT) length.

The various PBS sequences employed were GCACGGC (SEQ ID NO: 2), GCACGGCC (SEQ ID NO: 3), GCACGGCCU (SEQ ID NO: 4), GCACGGCCUG (SEQ ID NO: 5), GCACGGCCUGG (SEQ ID NO: 6), GCACGGCCUGGA (SEQ ID NO: 7), GCACGGCCUGGAG (SEQ ID NO: 8), GCACGGCCUGGAGG (SEQ ID NO: 9), or GCACGGCCUGGAGGG (SEQ ID NO: 10).

The various RTT (i.e., editing template) sequences employed were

(SEQ ID NO: 11)
GCAGCUUCAGUCCCUUUCUCGUCGAUGGUCAGCACAGCCUUAU,
(SEQ ID NO: 12)
CAGCUUCAGUCCCUUUCUCGUCGAUGGUCAGCACAGCCUUAU,
(SEQ ID NO: 13)
AGCUUCAGUCCCUUUCUCGUCGAUGGUCAGCACAGCCUUAU,
(SEQ ID NO: 14)
GCUUCAGUCCCUUUCUCGUCGAUGGUCAGCACAGCCUUAU,
(SEQ ID NO: 15)
CUUCAGUCCCUUUCUCGUCGAUGGUCAGCACAGCCUUAU,
(SEQ ID NO: 16)
UUCAGUCCCUUUCUCGUCGAUGGUCAGCACAGCCUUAU,
(SEQ ID NO: 17)
UCAGUCCCUUUCUCGUCGAUGGUCAGCACAGCCUUAU,
(SEQ ID NO: 18)
CAGUCCCUUUCUCGUCGAUGGUCAGCACAGCCUUAU,
(SEQ ID NO: 19)
AGUCCCUUUCUCGUCGAUGGUCAGCACAGCCUUAU,
(SEQ ID NO: 20)
GUCCCUUUCUCGUCGAUGGUCAGCACAGCCUUAU,
(SEQ ID NO: 21)
UCCCUUUCUCGUCGAUGGUCAGCACAGCCUUAU,
(SEQ ID NO: 22)
CCCUUUCUCGUCGAUGGUCAGCACAGCCUUAU,
(SEQ ID NO: 23)
CCUUUCUCGUCGAUGGUCAGCACAGCCUUAU,
(SEQ ID NO: 24)
CUUUCUCGUCGAUGGUCAGCACAGCCUUAU,
or
(SEQ ID NO: 25)
UUUCUCGUCGAUGGUCAGCACAGCCUUAU.

mRNAs encoding the prime editors were introduced into the cells by lipofection, using MessengerMax™ lipid reagent (ThermoFisher). 1 μg of prime editor mRNA and 750 ng of pegRNA were used for each well.

For each of the prime editor-encoding mRNAs, two technical replicates were examined. 3 days post lipofection, genomic DNA was harvested and sequenced using Illumina NGS as described above to measure prime editing efficiency and indel frequencies.

The pegRNA employed are recited in Table 1 below:

TABLE 1
Sequences of the pegRNA. “m” corresponds
to a 2′-O-Methyl ribose modification and “s”
correspond to a phosphorothioate modification.

| Peg | | |
|---|---|---|
| RNA | | SEQ ID |
| ID | Full sequence | Nos. |
| gRN | mUsmCsmCsCCUCCAGGCCGUGCAUAGUUUUAGAGCUAG | 79 |
| A52 | AAAUAGCAAGUUAAAAUAAGGCUAGUCCGUUAUCAACU | |
| 70 | UGAAAAAGUGGCACCGAGUCGGUGCUUUCUCGUCGAUG | |
| | GUCAGCACAGCCUUAUGCACGGCCUGGAGGmUsmUsmUs | |
| | U | |
| gRN | mUsmCsmCsCCUCCAGGCCGUGCAUAGUUUUAGAGCUAG | 80 |
| A52 | AAAUAGCAAGUUAAAAUAAGGCUAGUCCGUUAUCAACU | |
| 71 | UGAAAAAGUGGCACCGAGUCGGUGCUUUCUCGUCGAUG | |
| | GUCAGCACAGCCUUAUGCACGGCCUGGAGGGmUsmUsmU | |
| | SU | |
| gRN | mUsmCsmCsCCUCCAGGCCGUGCAUAGUUUUAGAGCUAG | 81 |
| A52 | AAAUAGCAAGUUAAAAUAAGGCUAGUCCGUUAUCAACU | |
| 72 | UGAAAAAGUGGCACCGAGUCGGUGCUUUCUCGUCGAUG | |
| | GUCAGCACAGCCUUAUGCACGGCCUGmUsmUsmUsU | |
| gRN | mUsmCsmCsCCUCCAGGCCGUGCAUAGUUUUAGAGCUAG | 82 |
| A52 | AAAUAGCAAGUUAAAAUAAGGCUAGUCCGUUAUCAACU | |
| 73 | UGAAAAAGUGGCACCGAGUCGGUGCUUUCUCGUCGAUG | |
| | GUCAGCACAGCCUUAUGCACGGCCUGGAGmUsmUsmUsU | |
| gRN | mUsmCsmCsCCUCCAGGCCGUGCAUAGUUUUAGAGCUAG | 83 |
| A52 | AAAUAGCAAGUUAAAAUAAGGCUAGUCCGUUAUCAACU | |
| 74 | UGAAAAAGUGGCACCGAGUCGGUGCUUUCUCGUCGAUG | |
| | GUCAGCACAGCCUUAUGCACGGCCUGGAmUsmUsmUsU | |
| gRN | mUsmCsmCsCCUCCAGGCCGUGCAUAGUUUUAGAGCUAG | 84 |
| A52 | AAAUAGCAAGUUAAAAUAAGGCUAGUCCGUUAUCAACU | |
| 75 | UGAAAAAGUGGCACCGAGUCGGUGCUUUCUCGUCGAUG | |
| | GUCAGCACAGCCUUAUGCACGGCCUGGmUsmUsmUsU | |
| gRN | mUsmCsmCsCCUCCAGGCCGUGCAUAGUUUUAGAGCUAG | 85 |
| A52 | AAAUAGCAAGUUAAAAUAAGGCUAGUCCGUUAUCAACU | |
| 76 | UGAAAAAGUGGCACCGAGUCGGUGCCUUUCUCGUCGAU | |
| | GGUCAGCACAGCCUUAUGCACGGCCUGGAGGmUsmUsmU | |
| | SU | |
| gRN | mUsmCsmCsCCUCCAGGCCGUGCAUAGUUUUAGAGCUAG | 86 |
| A52 | AAAUAGCAAGUUAAAAUAAGGCUAGUCCGUUAUCAACU | |
| 77 | UGAAAAAGUGGCACCGAGUCGGUGCCUUUCUCGUCGAU | |
| | GGUCAGCACAGCCUUAUGCACGGCCUGGAGGGmUsmUsm | |
| | UsU | |
| gRN | mUsmCsmCsCCUCCAGGCCGUGCAUAGUUUUAGAGCUAG | 87 |
| A52 | AAAUAGCAAGUUAAAAUAAGGCUAGUCCGUUAUCAACU | |
| 78 | UGAAAAAGUGGCACCGAGUCGGUGCUUUCUCGUCGAUG | |
| | GUCAGCACAGCCUUAUGCACGGCCUmUsmUsmUsU | |
| gRN | mUsmCsmCsCCUCCAGGCCGUGCAUAGUUUUAGAGCUAG | 88 |
| A52 | AAAUAGCAAGUUAAAAUAAGGCUAGUCCGUUAUCAACU | |
| 79 | UGAAAAAGUGGCACCGAGUCGGUGCCUUUCUCGUCGAU | |
| | GGUCAGCACAGCCUUAUGCACGGCCUGmUsmUsmUsU | |
| gRN | mUsmCsmCsCCUCCAGGCCGUGCAUAGUUUUAGAGCUAG | 89 |
| A52 | AAAUAGCAAGUUAAAAUAAGGCUAGUCCGUUAUCAACU | |
| 80 | UGAAAAAGUGGCACCGAGUCGGUGCCUUUCUCGUCGAU | |
| | GGUCAGCACAGCCUUAUGCACGGCCUGGAGmUsmUsmUs | |
| | U | |
| gRN | mUsmCsmCsCCUCCAGGCCGUGCAUAGUUUUAGAGCUAG | 90 |
| A52 | AAAUAGCAAGUUAAAAUAAGGCUAGUCCGUUAUCAACU | |
| 81 | UGAAAAAGUGGCACCGAGUCGGUGCCUUUCUCGUCGAU | |
| | GGUCAGCACAGCCUUAUGCACGGCCUGGAmUsmUsmUsU | |
| gRN | mUsmCsmCsCCUCCAGGCCGUGCAUAGUUUUAGAGCUAG | 91 |
| A52 | AAAUAGCAAGUUAAAAUAAGGCUAGUCCGUUAUCAACU | |
| 82 | UGAAAAAGUGGCACCGAGUCGGUGCCUUUCUCGUCGAU | |
| | GGUCAGCACAGCCUUAUGCACGGCCUGGmUsmUsmUsU | |
| gRN | mUsmCsmCsCCUCCAGGCCGUGCAUAGUUUUAGAGCUAG | 92 |
| A52 | AAAUAGCAAGUUAAAAUAAGGCUAGUCCGUUAUCAACU | |
| 83 | UGAAAAAGUGGCACCGAGUCGGUGCUUUCUCGUCGAUG | |
| | GUCAGCACAGCCUUAUGCACGGCCmUsmUsmUsU | |
| gRN | mUsmCsmCsCCUCCAGGCCGUGCAUAGUUUUAGAGCUAG | 93 |
| A52 | AAAUAGCAAGUUAAAAUAAGGCUAGUCCGUUAUCAACU | |
| 84 | UGAAAAAGUGGCACCGAGUCGGUGCCCUUUCUCGUCGA | |
| | UGGUCAGCACAGCCUUAUGCACGGCCUGGAGGmUsmUsm | |
| | UsU | |
| gRN | mUsmCsmCsCCUCCAGGCCGUGCAUAGUUUUAGAGCUAG | 94 |
| A52 | AAAUAGCAAGUUAAAAUAAGGCUAGUCCGUUAUCAACU | |
| 85 | UGAAAAAGUGGCACCGAGUCGGUGCCCUUUCUCGUCGA | |
| | UGGUCAGCACAGCCUUAUGCACGGCCUGGAGGGmUsmUs | |
| | mUsU | |
| gRN | mUsmCsmCsCCUCCAGGCCGUGCAUAGUUUUAGAGCUAG | 95 |
| A52 | AAAUAGCAAGUUAAAAUAAGGCUAGUCCGUUAUCAACU | |
| 86 | UGAAAAAGUGGCACCGAGUCGGUGCCUUUCUCGUCGAU | |
| | GGUCAGCACAGCCUUAUGCACGGCCUmUsmUsmUsU | |
| gRN | mUsmCsmCsCCUCCAGGCCGUGCAUAGUUUUAGAGCUAG | 96 |
| A52 | AAAUAGCAAGUUAAAAUAAGGCUAGUCCGUUAUCAACU | |
| 87 | UGAAAAAGUGGCACCGAGUCGGUGCCCUUUCUCGUCGA | |
| | UGGUCAGCACAGCCUUAUGCACGGCCUGmUsmUsmUsU | |
| gRN | mUsmCsmCsCCUCCAGGCCGUGCAUAGUUUUAGAGCUAG | 97 |
| A52 | AAAUAGCAAGUUAAAAUAAGGCUAGUCCGUUAUCAACU | |
| 88 | UGAAAAAGUGGCACCGAGUCGGUGCCCUUUCUCGUCGA | |
| | UGGUCAGCACAGCCUUAUGCACGGCCUGGAGmUsmUsmU | |
| | SU | |
| gRN | mUsmCsmCsCCUCCAGGCCGUGCAUAGUUUUAGAGCUAG | 98 |
| A52 | AAAUAGCAAGUUAAAAUAAGGCUAGUCCGUUAUCAACU | |
| 89 | UGAAAAAGUGGCACCGAGUCGGUGCCCUUUCUCGUCGA | |
| | UGGUCAGCACAGCCUUAUGCACGGCCUGGAmUsmUsmUs | |
| | U | |
| gRN | mUsmCsmCsCCUCCAGGCCGUGCAUAGUUUUAGAGCUAG | 99 |
| A52 | AAAUAGCAAGUUAAAAUAAGGCUAGUCCGUUAUCAACU | |
| 90 | UGAAAAAGUGGCACCGAGUCGGUGCCCUUUCUCGUCGA | |
| | UGGUCAGCACAGCCUUAUGCACGGCCUGGmUsmUsmUsU | |
| gRN | mUsmCsmCsCCUCCAGGCCGUGCAUAGUUUUAGAGCUAG | 100 |
| A52 | AAAUAGCAAGUUAAAAUAAGGCUAGUCCGUUAUCAACU | |
| 91 | UGAAAAAGUGGCACCGAGUCGGUGCUUUCUCGUCGAUG | |
| | GUCAGCACAGCCUUAUGCACGGCmUsmUsmUsU | |
| gRN | mUsmCsmCsCCUCCAGGCCGUGCAUAGUUUUAGAGCUAG | 101 |
| A52 | AAAUAGCAAGUUAAAAUAAGGCUAGUCCGUUAUCAACU | |
| 92 | UGAAAAAGUGGCACCGAGUCGGUGCCUUUCUCGUCGAU | |
| | GGUCAGCACAGCCUUAUGCACGGCCmUsmUsmUsU | |
| gRN | mUsmCsmCsCCUCCAGGCCGUGCAUAGUUUUAGAGCUAG | 102 |
| A52 | AAAUAGCAAGUUAAAAUAAGGCUAGUCCGUUAUCAACU | |
| 93 | UGAAAAAGUGGCACCGAGUCGGUGCCCUUUCUCGUCGA | |
| | UGGUCAGCACAGCCUUAUGCACGGCCUmUsmUsmUsU | |
| gRN | mUsmCsmCsCCUCCAGGCCGUGCAUAGUUUUAGAGCUAG | 103 |
| A52 | AAAUAGCAAGUUAAAAUAAGGCUAGUCCGUUAUCAACU | |
| 94 | UGAAAAAGUGGCACCGAGUCGGUGCAGCUUCAGUCCCU | |
| | UUCUCGUCGAUGGUCAGCACAGCCUUAUGCACGGCCUG | |
| | GAGGmUsmUsmUsU | |
| gRN | mUsmCsmCsCCUCCAGGCCGUGCAUAGUUUUAGAGCUAG | 104 |
| A52 | AAAUAGCAAGUUAAAAUAAGGCUAGUCCGUUAUCAACU | |
| 95 | UGAAAAAGUGGCACCGAGUCGGUGCUCCCUUUCUCGUC | |
| | GAUGGUCAGCACAGCCUUAUGCACGGCCUGGAGGmUsmU | |
| | smUsU | |
| gRN | mUsmCsmCsCCUCCAGGCCGUGCAUAGUUUUAGAGCUAG | 105 |
| A52 | AAAUAGCAAGUUAAAAUAAGGCUAGUCCGUUAUCAACU | |
| 96 | UGAAAAAGUGGCACCGAGUCGGUGCAGCUUCAGUCCCU | |
| | UUCUCGUCGAUGGUCAGCACAGCCUUAUGCACGGCCUG | |
| | GAGGGmUsmUsmUsU | |
| gRN | mUsmCsmCsCCUCCAGGCCGUGCAUAGUUUUAGAGCUAG | 106 |
| A52 | AAAUAGCAAGUUAAAAUAAGGCUAGUCCGUUAUCAACU | |
| 97 | UGAAAAAGUGGCACCGAGUCGGUGCGUCCCUUUCUCGU | |
| | CGAUGGUCAGCACAGCCUUAUGCACGGCCUGGAGGmUsm | |
| | UsmUsU | |
| gRN | mUsmCsmCsCCUCCAGGCCGUGCAUAGUUUUAGAGCUAG | 107 |
| A52 | AAAUAGCAAGUUAAAAUAAGGCUAGUCCGUUAUCAACU | |
| 98 | UGAAAAAGUGGCACCGAGUCGGUGCUCCCUUUCUCGUC | |
| | GAUGGUCAGCACAGCCUUAUGCACGGCCUGGAGmUsmUs | |
| | mUsU | |
| gRN | mUsmCsmCsCCUCCAGGCCGUGCAUAGUUUUAGAGCUAG | 108 |
| A52 | AAAUAGCAAGUUAAAAUAAGGCUAGUCCGUUAUCAACU | |
| 99 | UGAAAAAGUGGCACCGAGUCGGUGCUUCAGUCCCUUUC | |
| | UCGUCGAUGGUCAGCACAGCCUUAUGCACGGCCUGGAG | |
| | GmUsmUsmUsU | |
| gRN | mUsmCsmCsCCUCCAGGCCGUGCAUAGUUUUAGAGCUAG | 109 |
| A53 | AAAUAGCAAGUUAAAAUAAGGCUAGUCCGUUAUCAACU | |
| 00 | UGAAAAAGUGGCACCGAGUCGGUGCGCAGCUUCAGUCC | |
| | CUUUCUCGUCGAUGGUCAGCACAGCCUUAUGCACGGCCU | |
| | GGAGGmUsmUsmUsU | |
| gRN | mUsmCsmCsCCUCCAGGCCGUGCAUAGUUUUAGAGCUAG | 110 |
| A53 | AAAUAGCAAGUUAAAAUAAGGCUAGUCCGUUAUCAACU | |
| 01 | UGAAAAAGUGGCACCGAGUCGGUGCUUCAGUCCCUUUC | |
| | UCGUCGAUGGUCAGCACAGCCUUAUGCACGGCCUGGAG | |
| | GGmUsmUsmUsU | |
| gRN | mUsmCsmCsCCUCCAGGCCGUGCAUAGUUUUAGAGCUAG | 111 |
| A53 | AAAUAGCAAGUUAAAAUAAGGCUAGUCCGUUAUCAACU | |
| 02 | UGAAAAAGUGGCACCGAGUCGGUGCAGUCCCUUUCUCG | |
| | UCGAUGGUCAGCACAGCCUUAUGCACGGCCUGGAGGmUs | |
| | mUsmUsU | |
| gRN | mUsmCsmCsCCUCCAGGCCGUGCAUAGUUUUAGAGCUAG | 112 |
| A53 | AAAUAGCAAGUUAAAAUAAGGCUAGUCCGUUAUCAACU | |
| 03 | UGAAAAAGUGGCACCGAGUCGGUGCUCCCUUUCUCGUC | |
| | GAUGGUCAGCACAGCCUUAUGCACGGCCUGGAmUsmUsm | |
| | UsU | |
| gRN | mUsmCsmCsCCUCCAGGCCGUGCAUAGUUUUAGAGCUAG | 113 |
| A53 | AAAUAGCAAGUUAAAAUAAGGCUAGUCCGUUAUCAACU | |
| 04 | UGAAAAAGUGGCACCGAGUCGGUGCAGCUUCAGUCCCU | |
| | UUCUCGUCGAUGGUCAGCACAGCCUUAUGCACGGCCUG | |
| | mUsmUsmUsU | |
| gRN | mUsmCsmCsCCUCCAGGCCGUGCAUAGUUUUAGAGCUAG | 114 |
| A53 | AAAUAGCAAGUUAAAAUAAGGCUAGUCCGUUAUCAACU | |
| 05 | UGAAAAAGUGGCACCGAGUCGGUGCGUCCCUUUCUCGU | |
| | CGAUGGUCAGCACAGCCUUAUGCACGGCCUGGAGGGmUs | |
| | mUsmUsU | |
| gRN | mUsmCsmCsCCUCCAGGCCGUGCAUAGUUUUAGAGCUAG | 115 |
| A53 | AAAUAGCAAGUUAAAAUAAGGCUAGUCCGUUAUCAACU | |
| 06 | UGAAAAAGUGGCACCGAGUCGGUGCGCUUCAGUCCCUU | |
| | UCUCGUCGAUGGUCAGCACAGCCUUAUGCACGGCCUGG | |
| | AGGmUsmUsmUsU | |
| gRN | mUsmCsmCsCCUCCAGGCCGUGCAUAGUUUUAGAGCUAG | 116 |
| A53 | AAAUAGCAAGUUAAAAUAAGGCUAGUCCGUUAUCAACU | |
| 07 | UGAAAAAGUGGCACCGAGUCGGUGCGUCCCUUUCUCGU | |
| | CGAUGGUCAGCACAGCCUUAUGCACGGCCUGGAGmUsmU | |
| | smUsU | |
| gRN | mUsmCsmCsCCUCCAGGCCGUGCAUAGUUUUAGAGCUAG | 117 |
| A53 | AAAUAGCAAGUUAAAAUAAGGCUAGUCCGUUAUCAACU | |
| 08 | UGAAAAAGUGGCACCGAGUCGGUGCGCAGCUUCAGUCC | |
| | CUUUCUCGUCGAUGGUCAGCACAGCCUUAUGCACGGCCU | |
| | GGAGGGmUsmUsmUsU | |
| gRN | mUsmCsmCsCCUCCAGGCCGUGCAUAGUUUUAGAGCUAG | 118 |
| A53 | AAAUAGCAAGUUAAAAUAAGGCUAGUCCGUUAUCAACU | |
| 09 | UGAAAAAGUGGCACCGAGUCGGUGCAGCUUCAGUCCCU | |
| | UUCUCGUCGAUGGUCAGCACAGCCUUAUGCACGGCCUG | |
| | GAGmUsmUsmUsU | |
| gRN | mUsmCsmCsCCUCCAGGCCGUGCAUAGUUUUAGAGCUAG | 119 |
| A53 | AAAUAGCAAGUUAAAAUAAGGCUAGUCCGUUAUCAACU | |
| 10 | UGAAAAAGUGGCACCGAGUCGGUGCAGCUUCAGUCCCU | |
| | UUCUCGUCGAUGGUCAGCACAGCCUUAUGCACGGCCUG | |
| | GAmUsmUsmUsU | |
| gRN | mUsmCsmCsCCUCCAGGCCGUGCAUAGUUUUAGAGCUAG | 120 |
| A53 | AAAUAGCAAGUUAAAAUAAGGCUAGUCCGUUAUCAACU | |
| 11 | UGAAAAAGUGGCACCGAGUCGGUGCUCCCUUUCUCGUC | |
| | GAUGGUCAGCACAGCCUUAUGCACGGCCUGmUsmUsmUs | |
| | U | |
| gRN | mUsmCsmCsCCUCCAGGCCGUGCAUAGUUUUAGAGCUAG | 121 |
| A53 | AAAUAGCAAGUUAAAAUAAGGCUAGUCCGUUAUCAACU | |
| 12 | UGAAAAAGUGGCACCGAGUCGGUGCGUCCCUUUCUCGU | |
| | CGAUGGUCAGCACAGCCUUAUGCACGGCCUGmUsmUsmU | |
| | SU | |
| gRN | mUsmCsmCsCCUCCAGGCCGUGCAUAGUUUUAGAGCUAG | 122 |
| A53 | AAAUAGCAAGUUAAAAUAAGGCUAGUCCGUUAUCAACU | |
| 13 | UGAAAAAGUGGCACCGAGUCGGUGCUCCCUUUCUCGUC | |
| | GAUGGUCAGCACAGCCUUAUGCACGGCCUGGAGGGmUsm | |
| | UsmUsU | |
| gRN | mUsmCsmCsCCUCCAGGCCGUGCAUAGUUUUAGAGCUAG | 123 |
| A53 | AAAUAGCAAGUUAAAAUAAGGCUAGUCCGUUAUCAACU | |
| 14 | UGAAAAAGUGGCACCGAGUCGGUGCGUCCCUUUCUCGU | |
| | CGAUGGUCAGCACAGCCUUAUGCACGGCCUGGAmUsmUs | |
| | mUsU | |
| gRN | mUsmCsmCsCCUCCAGGCCGUGCAUAGUUUUAGAGCUAG | 124 |
| A53 | AAAUAGCAAGUUAAAAUAAGGCUAGUCCGUUAUCAACU | |
| 15 | UGAAAAAGUGGCACCGAGUCGGUGCUUCAGUCCCUUUC | |
| | UCGUCGAUGGUCAGCACAGCCUUAUGCACGGCCUGGAG | |
| | mUsmUsmUsU | |
| gRN | mUsmCsmCsCCUCCAGGCCGUGCAUAGUUUUAGAGCUAG | 125 |
| A53 | AAAUAGCAAGUUAAAAUAAGGCUAGUCCGUUAUCAACU | |
| 16 | UGAAAAAGUGGCACCGAGUCGGUGCUCCCUUUCUCGUC | |
| | GAUGGUCAGCACAGCCUUAUGCACGGCCUGGmUsmUsmU | |
| | SU | |
| gRN | mUsmCsmCsCCUCCAGGCCGUGCAUAGUUUUAGAGCUAG | 126 |
| A53 | AAAUAGCAAGUUAAAAUAAGGCUAGUCCGUUAUCAACU | |
| 17 | UGAAAAAGUGGCACCGAGUCGGUGCGCUUCAGUCCCUU | |
| | UCUCGUCGAUGGUCAGCACAGCCUUAUGCACGGCCUGG | |
| | AGGGmUsmUsmUsU | |
| gRN | mUsmCsmCsCCUCCAGGCCGUGCAUAGUUUUAGAGCUAG | 127 |
| A53 | AAAUAGCAAGUUAAAAUAAGGCUAGUCCGUUAUCAACU | |
| 18 | UGAAAAAGUGGCACCGAGUCGGUGCCCUUUCUCGUCGA | |
| | UGGUCAGCACAGCCUUAUGCACGGCCmUsmUsmUsU | |
| gRN | mUsmCsmCsCCUCCAGGCCGUGCAUAGUUUUAGAGCUAG | 128 |
| A53 | AAAUAGCAAGUUAAAAUAAGGCUAGUCCGUUAUCAACU | |
| 19 | UGAAAAAGUGGCACCGAGUCGGUGCGUCCCUUUCUCGU | |
| | CGAUGGUCAGCACAGCCUUAUGCACGGCCUGGmUsmUsm | |
| | UsU | |
| gRN | mUsmCsmCsCCUCCAGGCCGUGCAUAGUUUUAGAGCUAG | 129 |
| A53 | AAAUAGCAAGUUAAAAUAAGGCUAGUCCGUUAUCAACU | |
| 20 | UGAAAAAGUGGCACCGAGUCGGUGCAGUCCCUUUCUCG | |
| | UCGAUGGUCAGCACAGCCUUAUGCACGGCCUGGAGGGm | |
| | UsmUsmUsU | |
| gRN | mUsmCsmCsCCUCCAGGCCGUGCAUAGUUUUAGAGCUAG | 130 |
| A53 | AAAUAGCAAGUUAAAAUAAGGCUAGUCCGUUAUCAACU | |
| 21 | UGAAAAAGUGGCACCGAGUCGGUGCAGUCCCUUUCUCG | |
| | UCGAUGGUCAGCACAGCCUUAUGCACGGCCUGGAGmUsm | |
| | UsmUsU | |
| gRN | mUsmCsmCsCCUCCAGGCCGUGCAUAGUUUUAGAGCUAG | 131 |
| A53 | AAAUAGCAAGUUAAAAUAAGGCUAGUCCGUUAUCAACU | |
| 22 | UGAAAAAGUGGCACCGAGUCGGUGCUUCAGUCCCUUUC | |
| | UCGUCGAUGGUCAGCACAGCCUUAUGCACGGCCUGmUsm | |
| | UsmUsU | |
| gRN | mUsmCsmCsCCUCCAGGCCGUGCAUAGUUUUAGAGCUAG | 132 |
| A53 | AAAUAGCAAGUUAAAAUAAGGCUAGUCCGUUAUCAACU | |
| 23 | UGAAAAAGUGGCACCGAGUCGGUGCAGCUUCAGUCCCU | |
| | UUCUCGUCGAUGGUCAGCACAGCCUUAUGCACGGCCUG | |
| | GmUsmUsmUsU | |
| gRN | mUsmCsmCsCCUCCAGGCCGUGCAUAGUUUUAGAGCUAG | 133 |
| A53 | AAAUAGCAAGUUAAAAUAAGGCUAGUCCGUUAUCAACU | |
| 24 | UGAAAAAGUGGCACCGAGUCGGUGCUUCAGUCCCUUUC | |
| | UCGUCGAUGGUCAGCACAGCCUUAUGCACGGCCUGGAm | |
| | UsmUsmUsU | |
| gRN | mUsmCsmCsCCUCCAGGCCGUGCAUAGUUUUAGAGCUAG | 134 |
| A53 | AAAUAGCAAGUUAAAAUAAGGCUAGUCCGUUAUCAACU | |
| 25 | UGAAAAAGUGGCACCGAGUCGGUGCUCCCUUUCUCGUC | |
| | GAUGGUCAGCACAGCCUUAUGCACGGCCUmUsmUsmUsU | |
| gRN | mUsmCsmCsCCUCCAGGCCGUGCAUAGUUUUAGAGCUAG | 135 |
| A53 | AAAUAGCAAGUUAAAAUAAGGCUAGUCCGUUAUCAACU | |
| 26 | UGAAAAAGUGGCACCGAGUCGGUGCGCAGCUUCAGUCC | |
| | CUUUCUCGUCGAUGGUCAGCACAGCCUUAUGCACGGCCU | |
| | GmUsmUsmUsU | |
| gRN | mUsmCsmCsCCUCCAGGCCGUGCAUAGUUUUAGAGCUAG | 136 |
| A53 | AAAUAGCAAGUUAAAAUAAGGCUAGUCCGUUAUCAACU | |
| 27 | UGAAAAAGUGGCACCGAGUCGGUGCAGUCCCUUUCUCG | |
| | UCGAUGGUCAGCACAGCCUUAUGCACGGCCUGmUsmUsm | |
| | UsU | |
| gRN | mUsmCsmCsCCUCCAGGCCGUGCAUAGUUUUAGAGCUAG | 137 |
| A53 | AAAUAGCAAGUUAAAAUAAGGCUAGUCCGUUAUCAACU | |
| 28 | UGAAAAAGUGGCACCGAGUCGGUGCCUUUCUCGUCGAU | |
| | GGUCAGCACAGCCUUAUGCACGGCmUsmUsmUsU | |
| gRN | mUsmCsmCsCCUCCAGGCCGUGCAUAGUUUUAGAGCUAG | 138 |
| A53 | AAAUAGCAAGUUAAAAUAAGGCUAGUCCGUUAUCAACU | |
| 29 | UGAAAAAGUGGCACCGAGUCGGUGCAGUCCCUUUCUCG | |
| | UCGAUGGUCAGCACAGCCUUAUGCACGGCCUGGAmUsmU | |
| | smUsU | |
| gRN | mUsmCsmCsCCUCCAGGCCGUGCAUAGUUUUAGAGCUAG | 139 |
| A53 | AAAUAGCAAGUUAAAAUAAGGCUAGUCCGUUAUCAACU | |
| 30 | UGAAAAAGUGGCACCGAGUCGGUGCUCAGUCCCUUUCU | |
| | CGUCGAUGGUCAGCACAGCCUUAUGCACGGCCUGGAGG | |
| | mUsmUsmUsU | |
| gRN | mUsmCsmCsCCUCCAGGCCGUGCAUAGUUUUAGAGCUAG | 140 |
| A53 | AAAUAGCAAGUUAAAAUAAGGCUAGUCCGUUAUCAACU | |
| 31 | UGAAAAAGUGGCACCGAGUCGGUGCGCUUCAGUCCCUU | |
| | UCUCGUCGAUGGUCAGCACAGCCUUAUGCACGGCCUGG | |
| | AGmUsmUsmUsU | |
| gRN | mUsmCsmCsCCUCCAGGCCGUGCAUAGUUUUAGAGCUAG | 141 |
| A53 | AAAUAGCAAGUUAAAAUAAGGCUAGUCCGUUAUCAACU | |
| 32 | UGAAAAAGUGGCACCGAGUCGGUGCGCAGCUUCAGUCC | |
| | CUUUCUCGUCGAUGGUCAGCACAGCCUUAUGCACGGCCU | |
| | GGAGmUsmUsmUsU | |
| gRN | mUsmCsmCsCCUCCAGGCCGUGCAUAGUUUUAGAGCUAG | 142 |
| A53 | AAAUAGCAAGUUAAAAUAAGGCUAGUCCGUUAUCAACU | |
| 33 | UGAAAAAGUGGCACCGAGUCGGUGCGCAGCUUCAGUCC | |
| | CUUUCUCGUCGAUGGUCAGCACAGCCUUAUGCACGGCCU | |
| | GGAmUsmUsmUsU | |
| gRN | mUsmCsmCsCCUCCAGGCCGUGCAUAGUUUUAGAGCUAG | 143 |
| A53 | AAAUAGCAAGUUAAAAUAAGGCUAGUCCGUUAUCAACU | |
| 34 | UGAAAAAGUGGCACCGAGUCGGUGCUUCAGUCCCUUUC | |
| | UCGUCGAUGGUCAGCACAGCCUUAUGCACGGCCUGGmUs | |
| | mUsmUsU | |
| gRN | mUsmCsmCsCCUCCAGGCCGUGCAUAGUUUUAGAGCUAG | 144 |
| A53 | AAAUAGCAAGUUAAAAUAAGGCUAGUCCGUUAUCAACU | |
| 35 | UGAAAAAGUGGCACCGAGUCGGUGCAGUCCCUUUCUCG | |
| | UCGAUGGUCAGCACAGCCUUAUGCACGGCCUGGmUsmUs | |
| | mUsU | |
| gRN | mUsmCsmCsCCUCCAGGCCGUGCAUAGUUUUAGAGCUAG | 145 |
| A53 | AAAUAGCAAGUUAAAAUAAGGCUAGUCCGUUAUCAACU | |
| 36 | UGAAAAAGUGGCACCGAGUCGGUGCGCUUCAGUCCCUU | |
| | UCUCGUCGAUGGUCAGCACAGCCUUAUGCACGGCCUGm | |
| | UsmUsmUsU | |
| gRN | mUsmCsmCsCCUCCAGGCCGUGCAUAGUUUUAGAGCUAG | 146 |
| A53 | AAAUAGCAAGUUAAAAUAAGGCUAGUCCGUUAUCAACU | |
| 37 | UGAAAAAGUGGCACCGAGUCGGUGCGCUUCAGUCCCUU | |
| | UCUCGUCGAUGGUCAGCACAGCCUUAUGCACGGCCUGG | |
| | AmUsmUsmUsU | |
| gRN | mUsmCsmCsCCUCCAGGCCGUGCAUAGUUUUAGAGCUAG | 147 |
| A53 | AAAUAGCAAGUUAAAAUAAGGCUAGUCCGUUAUCAACU | |
| 38 | UGAAAAAGUGGCACCGAGUCGGUGCAGCUUCAGUCCCU | |
| | UUCUCGUCGAUGGUCAGCACAGCCUUAUGCACGGCCUm | |
| | UsmUsmUsU | |
| gRN | mUsmCsmCsCCUCCAGGCCGUGCAUAGUUUUAGAGCUAG | 148 |
| A53 | AAAUAGCAAGUUAAAAUAAGGCUAGUCCGUUAUCAACU | |
| 39 | UGAAAAAGUGGCACCGAGUCGGUGCGCAGCUUCAGUCC | |
| | CUUUCUCGUCGAUGGUCAGCACAGCCUUAUGCACGGCCU | |
| | GGmUsmUsmUsU | |
| gRN | mUsmCsmCsCCUCCAGGCCGUGCAUAGUUUUAGAGCUAG | 149 |
| A53 | AAAUAGCAAGUUAAAAUAAGGCUAGUCCGUUAUCAACU | |
| 40 | UGAAAAAGUGGCACCGAGUCGGUGCUCAGUCCCUUUCU | |
| | CGUCGAUGGUCAGCACAGCCUUAUGCACGGCCUGGAGG | |
| | GmUsmUsmUsU | |
| gRN | mUsmCsmCsCCUCCAGGCCGUGCAUAGUUUUAGAGCUAG | 150 |
| A53 | AAAUAGCAAGUUAAAAUAAGGCUAGUCCGUUAUCAACU | |
| 41 | UGAAAAAGUGGCACCGAGUCGGUGCUCAGUCCCUUUCU | |
| | CGUCGAUGGUCAGCACAGCCUUAUGCACGGCCUGGAGm | |
| | UsmUsmUsU | |
| gRN | mUsmCsmCsCCUCCAGGCCGUGCAUAGUUUUAGAGCUAG | 151 |
| A53 | AAAUAGCAAGUUAAAAUAAGGCUAGUCCGUUAUCAACU | |
| 42 | UGAAAAAGUGGCACCGAGUCGGUGCGUCCCUUUCUCGU | |
| | CGAUGGUCAGCACAGCCUUAUGCACGGCCUmUsmUsmUs | |
| | U | |
| gRN | mUsmCsmCsCCUCCAGGCCGUGCAUAGUUUUAGAGCUAG | 152 |
| A53 | AAAUAGCAAGUUAAAAUAAGGCUAGUCCGUUAUCAACU | |
| 43 | UGAAAAAGUGGCACCGAGUCGGUGCUUCAGUCCCUUUC | |
| | UCGUCGAUGGUCAGCACAGCCUUAUGCACGGCCUmUsmU | |
| | smUsU | |
| gRN | mUsmCsmCsCCUCCAGGCCGUGCAUAGUUUUAGAGCUAG | 153 |
| A53 | AAAUAGCAAGUUAAAAUAAGGCUAGUCCGUUAUCAACU | |
| 44 | UGAAAAAGUGGCACCGAGUCGGUGCGCUUCAGUCCCUU | |
| | UCUCGUCGAUGGUCAGCACAGCCUUAUGCACGGCCUGG | |
| | mUsmUsmUsU | |
| gRN | mUsmCsmCsCCUCCAGGCCGUGCAUAGUUUUAGAGCUAG | 154 |
| A53 | AAAUAGCAAGUUAAAAUAAGGCUAGUCCGUUAUCAACU | |
| 45 | UGAAAAAGUGGCACCGAGUCGGUGCUCAGUCCCUUUCU | |
| | CGUCGAUGGUCAGCACAGCCUUAUGCACGGCCUGGAmUs | |
| | mUsmUsU | |
| gRN | mUsmCsmCsCCUCCAGGCCGUGCAUAGUUUUAGAGCUAG | 155 |
| A53 | AAAUAGCAAGUUAAAAUAAGGCUAGUCCGUUAUCAACU | |
| 46 | UGAAAAAGUGGCACCGAGUCGGUGCUCAGUCCCUUUCU | |
| | CGUCGAUGGUCAGCACAGCCUUAUGCACGGCCUGmUsmU | |
| | smUsU | |
| gRN | mUsmCsmCsCCUCCAGGCCGUGCAUAGUUUUAGAGCUAG | 156 |
| A53 | AAAUAGCAAGUUAAAAUAAGGCUAGUCCGUUAUCAACU | |
| 47 | UGAAAAAGUGGCACCGAGUCGGUGCAGUCCCUUUCUCG | |
| | UCGAUGGUCAGCACAGCCUUAUGCACGGCCUmUsmUsmU | |
| | SU | |
| gRN | mUsmCsmCsCCUCCAGGCCGUGCAUAGUUUUAGAGCUAG | 157 |
| A53 | AAAUAGCAAGUUAAAAUAAGGCUAGUCCGUUAUCAACU | |
| 48 | UGAAAAAGUGGCACCGAGUCGGUGCUCCCUUUCUCGUC | |
| | GAUGGUCAGCACAGCCUUAUGCACGGCCmUsmUsmUsU | |
| gRN | mUsmCsmCsCCUCCAGGCCGUGCAUAGUUUUAGAGCUAG | 158 |
| A53 | AAAUAGCAAGUUAAAAUAAGGCUAGUCCGUUAUCAACU | |
| 49 | UGAAAAAGUGGCACCGAGUCGGUGCUCAGUCCCUUUCU | |
| | CGUCGAUGGUCAGCACAGCCUUAUGCACGGCCUGGmUsm | |
| | UsmUsU | |
| gRN | mUsmCsmCsCCUCCAGGCCGUGCAUAGUUUUAGAGCUAG | 159 |
| A53 | AAAUAGCAAGUUAAAAUAAGGCUAGUCCGUUAUCAACU | |
| 50 | UGAAAAAGUGGCACCGAGUCGGUGCCUUCAGUCCCUUU | |
| | CUCGUCGAUGGUCAGCACAGCCUUAUGCACGGCCUGGA | |
| | GGmUsmUsmUsU | |
| gRN | mUsmCsmCsCCUCCAGGCCGUGCAUAGUUUUAGAGCUAG | 160 |
| A53 | AAAUAGCAAGUUAAAAUAAGGCUAGUCCGUUAUCAACU | |
| 51 | UGAAAAAGUGGCACCGAGUCGGUGCGCAGCUUCAGUCC | |
| | CUUUCUCGUCGAUGGUCAGCACAGCCUUAUGCACGGCCU | |
| | mUsmUsmUsU | |
| gRN | mUsmCsmCsCCUCCAGGCCGUGCAUAGUUUUAGAGCUAG | 161 |
| A53 | AAAUAGCAAGUUAAAAUAAGGCUAGUCCGUUAUCAACU | |
| 52 | UGAAAAAGUGGCACCGAGUCGGUGCCUUCAGUCCCUUU | |
| | CUCGUCGAUGGUCAGCACAGCCUUAUGCACGGCCUGGA | |
| | GGGmUsmUsmUsU | |
| gRN | mUsmCsmCsCCUCCAGGCCGUGCAUAGUUUUAGAGCUAG | 162 |
| A53 | AAAUAGCAAGUUAAAAUAAGGCUAGUCCGUUAUCAACU | |
| 53 | UGAAAAAGUGGCACCGAGUCGGUGCGCUUCAGUCCCUU | |
| | UCUCGUCGAUGGUCAGCACAGCCUUAUGCACGGCCUmUs | |
| | mUsmUsU | |
| gRN | mUsmCsmCsCCUCCAGGCCGUGCAUAGUUUUAGAGCUAG | 163 |
| A53 | AAAUAGCAAGUUAAAAUAAGGCUAGUCCGUUAUCAACU | |
| 54 | UGAAAAAGUGGCACCGAGUCGGUGCGUCCCUUUCUCGU | |
| | CGAUGGUCAGCACAGCCUUAUGCACGGCCmUsmUsmUsU | |
| gRN | mUsmCsmCsCCUCCAGGCCGUGCAUAGUUUUAGAGCUAG | 164 |
| A53 | AAAUAGCAAGUUAAAAUAAGGCUAGUCCGUUAUCAACU | |
| 55 | UGAAAAAGUGGCACCGAGUCGGUGCCCUUUCUCGUCGA | |
| | UGGUCAGCACAGCCUUAUGCACGGCmUsmUsmUsU | |
| gRN | mUsmCsmCsCCUCCAGGCCGUGCAUAGUUUUAGAGCUAG | 165 |
| A53 | AAAUAGCAAGUUAAAAUAAGGCUAGUCCGUUAUCAACU | |
| 56 | UGAAAAAGUGGCACCGAGUCGGUGCUCAGUCCCUUUCU | |
| | CGUCGAUGGUCAGCACAGCCUUAUGCACGGCCUmUsmUs | |
| | mUsU | |
| gRN | mUsmCsmCsCCUCCAGGCCGUGCAUAGUUUUAGAGCUAG | 166 |
| A53 | AAAUAGCAAGUUAAAAUAAGGCUAGUCCGUUAUCAACU | |
| 57 | UGAAAAAGUGGCACCGAGUCGGUGCAGCUUCAGUCCCU | |
| | UUCUCGUCGAUGGUCAGCACAGCCUUAUGCACGGCCmUs | |
| | mUsmUsU | |
| gRN | mUsmCsmCsCCUCCAGGCCGUGCAUAGUUUUAGAGCUAG | 167 |
| A53 | AAAUAGCAAGUUAAAAUAAGGCUAGUCCGUUAUCAACU | |
| 58 | UGAAAAAGUGGCACCGAGUCGGUGCCUUCAGUCCCUUU | |
| | CUCGUCGAUGGUCAGCACAGCCUUAUGCACGGCCUGGA | |
| | GmUsmUsmUsU | |
| gRN | mUsmCsmCsCCUCCAGGCCGUGCAUAGUUUUAGAGCUAG | 168 |
| A53 | AAAUAGCAAGUUAAAAUAAGGCUAGUCCGUUAUCAACU | |
| 59 | UGAAAAAGUGGCACCGAGUCGGUGCCAGUCCCUUUCUC | |
| | GUCGAUGGUCAGCACAGCCUUAUGCACGGCCUGGAGGG | |
| | mUsmUsmUsU | |
| gRN | mUsmCsmCsCCUCCAGGCCGUGCAUAGUUUUAGAGCUAG | 169 |
| A53 | AAAUAGCAAGUUAAAAUAAGGCUAGUCCGUUAUCAACU | |
| 60 | UGAAAAAGUGGCACCGAGUCGGUGCCAGUCCCUUUCUC | |
| | GUCGAUGGUCAGCACAGCCUUAUGCACGGCCUGGAGGm | |
| | UsmUsmUsU | |
| gRN | mUsmCsmCsCCUCCAGGCCGUGCAUAGUUUUAGAGCUAG | 170 |
| A53 | AAAUAGCAAGUUAAAAUAAGGCUAGUCCGUUAUCAACU | |
| 61 | UGAAAAAGUGGCACCGAGUCGGUGCUUCAGUCCCUUUC | |
| | UCGUCGAUGGUCAGCACAGCCUUAUGCACGGCCmUsmUs | |
| | mUsU | |
| gRN | mUsmCsmCsCCUCCAGGCCGUGCAUAGUUUUAGAGCUAG | 171 |
| A53 | AAAUAGCAAGUUAAAAUAAGGCUAGUCCGUUAUCAACU | |
| 62 | UGAAAAAGUGGCACCGAGUCGGUGCCAGCUUCAGUCCC | |
| | UUUCUCGUCGAUGGUCAGCACAGCCUUAUGCACGGCCU | |
| | GGAGGGmUsmUsmUsU | |
| gRN | mUsmCsmCsCCUCCAGGCCGUGCAUAGUUUUAGAGCUAG | 172 |
| A53 | AAAUAGCAAGUUAAAAUAAGGCUAGUCCGUUAUCAACU | |
| 63 | UGAAAAAGUGGCACCGAGUCGGUGCAGUCCCUUUCUCG | |
| | UCGAUGGUCAGCACAGCCUUAUGCACGGCCmUsmUsmUs | |
| | U | |
| gRN | mUsmCsmCsCCUCCAGGCCGUGCAUAGUUUUAGAGCUAG | 173 |
| A53 | AAAUAGCAAGUUAAAAUAAGGCUAGUCCGUUAUCAACU | |
| 64 | UGAAAAAGUGGCACCGAGUCGGUGCCAGCUUCAGUCCC | |
| | UUUCUCGUCGAUGGUCAGCACAGCCUUAUGCACGGCCU | |
| | GGAGGmUsmUsmUsU | |
| gRN | mUsmCsmCsCCUCCAGGCCGUGCAUAGUUUUAGAGCUAG | 174 |
| A53 | AAAUAGCAAGUUAAAAUAAGGCUAGUCCGUUAUCAACU | |
| 65 | UGAAAAAGUGGCACCGAGUCGGUGCCUUCAGUCCCUUU | |
| | CUCGUCGAUGGUCAGCACAGCCUUAUGCACGGCCUGGA | |
| | mUsmUsmUsU | |
| gRN | mUsmCsmCsCCUCCAGGCCGUGCAUAGUUUUAGAGCUAG | 175 |
| A53 | AAAUAGCAAGUUAAAAUAAGGCUAGUCCGUUAUCAACU | |
| 66 | UGAAAAAGUGGCACCGAGUCGGUGCCUUCAGUCCCUUU | |
| | CUCGUCGAUGGUCAGCACAGCCUUAUGCACGGCCUGmUs | |
| | mUsmUsU | |
| gRN | mUsmCsmCsCCUCCAGGCCGUGCAUAGUUUUAGAGCUAG | 176 |
| A53 | AAAUAGCAAGUUAAAAUAAGGCUAGUCCGUUAUCAACU | |
| 67 | UGAAAAAGUGGCACCGAGUCGGUGCCUUCAGUCCCUUU | |
| | CUCGUCGAUGGUCAGCACAGCCUUAUGCACGGCCUGGm | |
| | UsmUsmUsU | |
| gRN | mUsmCsmCsCCUCCAGGCCGUGCAUAGUUUUAGAGCUAG | 177 |
| A53 | AAAUAGCAAGUUAAAAUAAGGCUAGUCCGUUAUCAACU | |
| 68 | UGAAAAAGUGGCACCGAGUCGGUGCCAGUCCCUUUCUC | |
| | GUCGAUGGUCAGCACAGCCUUAUGCACGGCCUGGAGmUs | |
| | mUsmUsU | |
| gRN | mUsmCsmCsCCUCCAGGCCGUGCAUAGUUUUAGAGCUAG | 178 |
| A53 | AAAUAGCAAGUUAAAAUAAGGCUAGUCCGUUAUCAACU | |
| 69 | UGAAAAAGUGGCACCGAGUCGGUGCCAGUCCCUUUCUC | |
| | GUCGAUGGUCAGCACAGCCUUAUGCACGGCCUGmUsmUs | |
| | mUsU | |
| gRN | mUsmCsmCsCCUCCAGGCCGUGCAUAGUUUUAGAGCUAG | 179 |
| A53 | AAAUAGCAAGUUAAAAUAAGGCUAGUCCGUUAUCAACU | |
| 70 | UGAAAAAGUGGCACCGAGUCGGUGCGCUUCAGUCCCUU | |
| | UCUCGUCGAUGGUCAGCACAGCCUUAUGCACGGCCmUsm | |
| | UsmUsU | |
| gRN | mUsmCsmCsCCUCCAGGCCGUGCAUAGUUUUAGAGCUAG | 180 |
| A53 | AAAUAGCAAGUUAAAAUAAGGCUAGUCCGUUAUCAACU | |
| 71 | UGAAAAAGUGGCACCGAGUCGGUGCGCAGCUUCAGUCC | |
| | CUUUCUCGUCGAUGGUCAGCACAGCCUUAUGCACGGCC | |
| | mUsmUsmUsU | |
| gRN | mUsmCsmCsCCUCCAGGCCGUGCAUAGUUUUAGAGCUAG | 181 |
| A53 | AAAUAGCAAGUUAAAAUAAGGCUAGUCCGUUAUCAACU | |
| 72 | UGAAAAAGUGGCACCGAGUCGGUGCCAGUCCCUUUCUC | |
| | GUCGAUGGUCAGCACAGCCUUAUGCACGGCCUGGAmUsm | |
| | UsmUsU | |
| gRN | mUsmCsmCsCCUCCAGGCCGUGCAUAGUUUUAGAGCUAG | 182 |
| A53 | AAAUAGCAAGUUAAAAUAAGGCUAGUCCGUUAUCAACU | |
| 73 | UGAAAAAGUGGCACCGAGUCGGUGCCAGCUUCAGUCCC | |
| | UUUCUCGUCGAUGGUCAGCACAGCCUUAUGCACGGCCU | |
| | GmUsmUsmUsU | |
| gRN | mUsmCsmCsCCUCCAGGCCGUGCAUAGUUUUAGAGCUAG | 183 |
| A53 | AAAUAGCAAGUUAAAAUAAGGCUAGUCCGUUAUCAACU | |
| 74 | UGAAAAAGUGGCACCGAGUCGGUGCCAGUCCCUUUCUC | |
| | GUCGAUGGUCAGCACAGCCUUAUGCACGGCCUGGmUsmU | |
| | smUsU | |
| gRN | mUsmCsmCsCCUCCAGGCCGUGCAUAGUUUUAGAGCUAG | 184 |
| A53 | AAAUAGCAAGUUAAAAUAAGGCUAGUCCGUUAUCAACU | |
| 75 | UGAAAAAGUGGCACCGAGUCGGUGCUCAGUCCCUUUCU | |
| | CGUCGAUGGUCAGCACAGCCUUAUGCACGGCCmUsmUsm | |
| | UsU | |
| gRN | mUsmCsmCsCCUCCAGGCCGUGCAUAGUUUUAGAGCUAG | 185 |
| A53 | AAAUAGCAAGUUAAAAUAAGGCUAGUCCGUUAUCAACU | |
| 76 | UGAAAAAGUGGCACCGAGUCGGUGCCAGCUUCAGUCCC | |
| | UUUCUCGUCGAUGGUCAGCACAGCCUUAUGCACGGCCU | |
| | GGAmUsmUsmUsU | |
| gRN | mUsmCsmCsCCUCCAGGCCGUGCAUAGUUUUAGAGCUAG | 186 |
| A53 | AAAUAGCAAGUUAAAAUAAGGCUAGUCCGUUAUCAACU | |
| 77 | UGAAAAAGUGGCACCGAGUCGGUGCCAGCUUCAGUCCC | |
| | UUUCUCGUCGAUGGUCAGCACAGCCUUAUGCACGGCCU | |
| | GGAGmUsmUsmUsU | |
| gRN | mUsmCsmCsCCUCCAGGCCGUGCAUAGUUUUAGAGCUAG | 187 |
| A53 | AAAUAGCAAGUUAAAAUAAGGCUAGUCCGUUAUCAACU | |
| 78 | UGAAAAAGUGGCACCGAGUCGGUGCUCCCUUUCUCGUC | |
| | GAUGGUCAGCACAGCCUUAUGCACGGCmUsmUsmUsU | |
| gRN | mUsmCsmCsCCUCCAGGCCGUGCAUAGUUUUAGAGCUAG | 188 |
| A53 | AAAUAGCAAGUUAAAAUAAGGCUAGUCCGUUAUCAACU | |
| 79 | UGAAAAAGUGGCACCGAGUCGGUGCCAGCUUCAGUCCC | |
| | UUUCUCGUCGAUGGUCAGCACAGCCUUAUGCACGGCCU | |
| | GGmUsmUsmUsU | |
| gRN | mUsmCsmCsCCUCCAGGCCGUGCAUAGUUUUAGAGCUAG | 189 |
| A53 | AAAUAGCAAGUUAAAAUAAGGCUAGUCCGUUAUCAACU | |
| 80 | UGAAAAAGUGGCACCGAGUCGGUGCGUCCCUUUCUCGU | |
| | CGAUGGUCAGCACAGCCUUAUGCACGGCmUsmUsmUsU | |
| gRN | mUsmCsmCsCCUCCAGGCCGUGCAUAGUUUUAGAGCUAG | 190 |
| A53 | AAAUAGCAAGUUAAAAUAAGGCUAGUCCGUUAUCAACU | |
| 81 | UGAAAAAGUGGCACCGAGUCGGUGCCUUCAGUCCCUUU | |
| | CUCGUCGAUGGUCAGCACAGCCUUAUGCACGGCCUmUsm | |
| | UsmUsU | |
| gRN | mUsmCsmCsCCUCCAGGCCGUGCAUAGUUUUAGAGCUAG | 191 |
| A53 | AAAUAGCAAGUUAAAAUAAGGCUAGUCCGUUAUCAACU | |
| 82 | UGAAAAAGUGGCACCGAGUCGGUGCCAGUCCCUUUCUC | |
| | GUCGAUGGUCAGCACAGCCUUAUGCACGGCCUmUsmUsm | |
| | UsU | |
| gRN | mUsmCsmCsCCUCCAGGCCGUGCAUAGUUUUAGAGCUAG | 192 |
| A53 | AAAUAGCAAGUUAAAAUAAGGCUAGUCCGUUAUCAACU | |
| 83 | UGAAAAAGUGGCACCGAGUCGGUGCAGUCCCUUUCUCG | |
| | UCGAUGGUCAGCACAGCCUUAUGCACGGCmUsmUsmUsU | |
| gRN | mUsmCsmCsCCUCCAGGCCGUGCAUAGUUUUAGAGCUAG | 193 |
| A53 | AAAUAGCAAGUUAAAAUAAGGCUAGUCCGUUAUCAACU | |
| 84 | UGAAAAAGUGGCACCGAGUCGGUGCAGCUUCAGUCCCU | |
| | UUCUCGUCGAUGGUCAGCACAGCCUUAUGCACGGCmUsm | |
| | UsmUsU | |
| gRN | mUsmCsmCsCCUCCAGGCCGUGCAUAGUUUUAGAGCUAG | 194 |
| A53 | AAAUAGCAAGUUAAAAUAAGGCUAGUCCGUUAUCAACU | |
| 85 | UGAAAAAGUGGCACCGAGUCGGUGCUUCAGUCCCUUUC | |
| | UCGUCGAUGGUCAGCACAGCCUUAUGCACGGCmUsmUsm | |
| | UsU | |
| gRN | mUsmCsmCsCCUCCAGGCCGUGCAUAGUUUUAGAGCUAG | 195 |
| A53 | AAAUAGCAAGUUAAAAUAAGGCUAGUCCGUUAUCAACU | |
| 86 | UGAAAAAGUGGCACCGAGUCGGUGCUUCUCGUCGAUGG | |
| | UCAGCACAGCCUUAUGCACGGCCUGGAGGmUsmUsmUsU | |
| gRN | mUsmCsmCsCCUCCAGGCCGUGCAUAGUUUUAGAGCUAG | 196 |
| A53 | AAAUAGCAAGUUAAAAUAAGGCUAGUCCGUUAUCAACU | |
| 87 | UGAAAAAGUGGCACCGAGUCGGUGCUUCUCGUCGAUGG | |
| | UCAGCACAGCCUUAUGCACGGCCUGGAGGGmUsmUsmUs | |
| | U | |
| gRN | mUsmCsmCsCCUCCAGGCCGUGCAUAGUUUUAGAGCUAG | 197 |
| A53 | AAAUAGCAAGUUAAAAUAAGGCUAGUCCGUUAUCAACU | |
| 88 | UGAAAAAGUGGCACCGAGUCGGUGCCAGCUUCAGUCCC | |
| | UUUCUCGUCGAUGGUCAGCACAGCCUUAUGCACGGCCU | |
| | mUsmUsmUsU | |
| gRN | mUsmCsmCsCCUCCAGGCCGUGCAUAGUUUUAGAGCUAG | 198 |
| A53 | AAAUAGCAAGUUAAAAUAAGGCUAGUCCGUUAUCAACU | |
| 89 | UGAAAAAGUGGCACCGAGUCGGUGCCAGUCCCUUUCUC | |
| | GUCGAUGGUCAGCACAGCCUUAUGCACGGCCmUsmUsmU | |
| | SU | |
| gRN | mUsmCsmCsCCUCCAGGCCGUGCAUAGUUUUAGAGCUAG | 199 |
| A53 | AAAUAGCAAGUUAAAAUAAGGCUAGUCCGUUAUCAACU | |
| 90 | UGAAAAAGUGGCACCGAGUCGGUGCUUCUCGUCGAUGG | |
| | UCAGCACAGCCUUAUGCACGGCCUGGAGmUsmUsmUsU | |
| gRN | mUsmCsmCsCCUCCAGGCCGUGCAUAGUUUUAGAGCUAG | 200 |
| A53 | AAAUAGCAAGUUAAAAUAAGGCUAGUCCGUUAUCAACU | |
| 91 | UGAAAAAGUGGCACCGAGUCGGUGCUUCUCGUCGAUGG | |
| | UCAGCACAGCCUUAUGCACGGCCUGmUsmUsmUsU | |
| gRN | mUsmCsmCsCCUCCAGGCCGUGCAUAGUUUUAGAGCUAG | 201 |
| A53 | AAAUAGCAAGUUAAAAUAAGGCUAGUCCGUUAUCAACU | |
| 92 | UGAAAAAGUGGCACCGAGUCGGUGCCUUCAGUCCCUUU | |
| | CUCGUCGAUGGUCAGCACAGCCUUAUGCACGGCCmUsmU | |
| | smUsU | |
| gRN | mUsmCsmCsCCUCCAGGCCGUGCAUAGUUUUAGAGCUAG | 202 |
| A53 | AAAUAGCAAGUUAAAAUAAGGCUAGUCCGUUAUCAACU | |
| 93 | | |
| | UGAAAAAGUGGCACCGAGUCGGUGCUUCUCGUCGAUGG | |
| | UCAGCACAGCCUUAUGCACGGCCUGGAmUsmUsmUsU | |
| gRN | mUsmCsmCsCCUCCAGGCCGUGCAUAGUUUUAGAGCUAG | 203 |
| A53 | AAAUAGCAAGUUAAAAUAAGGCUAGUCCGUUAUCAACU | |
| 94 | UGAAAAAGUGGCACCGAGUCGGUGCUUCUCGUCGAUGG | |
| | UCAGCACAGCCUUAUGCACGGCCUGGmUsmUsmUsU | |
| gRN | mUsmCsmCsCCUCCAGGCCGUGCAUAGUUUUAGAGCUAG | 204 |
| A53 | AAAUAGCAAGUUAAAAUAAGGCUAGUCCGUUAUCAACU | |
| 95 | UGAAAAAGUGGCACCGAGUCGGUGCCCCUUUCUCGUCG | |
| | AUGGUCAGCACAGCCUUAUGCACGGCCUGGAGmUsmUsm | |
| | UsU | |
| gRN | mUsmCsmCsCCUCCAGGCCGUGCAUAGUUUUAGAGCUAG | 205 |
| A53 | AAAUAGCAAGUUAAAAUAAGGCUAGUCCGUUAUCAACU | |
| 96 | UGAAAAAGUGGCACCGAGUCGGUGCCCCUUUCUCGUCG | |
| | AUGGUCAGCACAGCCUUAUGCACGGCCUGGAGGmUsmUs | |
| | mUsU | |
| gRN | mUsmCsmCsCCUCCAGGCCGUGCAUAGUUUUAGAGCUAG | 206 |
| A53 | AAAUAGCAAGUUAAAAUAAGGCUAGUCCGUUAUCAACU | |
| 97 | UGAAAAAGUGGCACCGAGUCGGUGCGCAGCUUCAGUCC | |
| | CUUUCUCGUCGAUGGUCAGCACAGCCUUAUGCACGGCm | |
| | UsmUsmUsU | |
| gRN | mUsmCsmCsCCUCCAGGCCGUGCAUAGUUUUAGAGCUAG | 207 |
| A53 | AAAUAGCAAGUUAAAAUAAGGCUAGUCCGUUAUCAACU | |
| 98 | UGAAAAAGUGGCACCGAGUCGGUGCGCUUCAGUCCCUU | |
| | UCUCGUCGAUGGUCAGCACAGCCUUAUGCACGGCmUsmU | |
| | smUsU | |
| gRN | mUsmCsmCsCCUCCAGGCCGUGCAUAGUUUUAGAGCUAG | 208 |
| A53 | AAAUAGCAAGUUAAAAUAAGGCUAGUCCGUUAUCAACU | |
| 99 | UGAAAAAGUGGCACCGAGUCGGUGCUCAGUCCCUUUCU | |
| | CGUCGAUGGUCAGCACAGCCUUAUGCACGGCmUsmUsmU | |
| | SU | |
| gRN | mUsmCsmCsCCUCCAGGCCGUGCAUAGUUUUAGAGCUAG | 209 |
| A54 | AAAUAGCAAGUUAAAAUAAGGCUAGUCCGUUAUCAACU | |
| 00 | UGAAAAAGUGGCACCGAGUCGGUGCCCCUUUCUCGUCG | |
| | AUGGUCAGCACAGCCUUAUGCACGGCCUmUsmUsmUsU | |
| gRN | mUsmCsmCsCCUCCAGGCCGUGCAUAGUUUUAGAGCUAG | 210 |
| A54 | AAAUAGCAAGUUAAAAUAAGGCUAGUCCGUUAUCAACU | |
| 01 | UGAAAAAGUGGCACCGAGUCGGUGCCCCUUUCUCGUCG | |
| | AUGGUCAGCACAGCCUUAUGCACGGCCUGGAmUsmUsmU | |
| | SU | |
| gRN | mUsmCsmCsCCUCCAGGCCGUGCAUAGUUUUAGAGCUAG | 211 |
| A54 | AAAUAGCAAGUUAAAAUAAGGCUAGUCCGUUAUCAACU | |
| 02 | UGAAAAAGUGGCACCGAGUCGGUGCCAGCUUCAGUCCC | |
| | UUUCUCGUCGAUGGUCAGCACAGCCUUAUGCACGGCCm | |
| | UsmUsmUsU | |
| gRN | mUsmCsmCsCCUCCAGGCCGUGCAUAGUUUUAGAGCUAG | 212 |
| A54 | AAAUAGCAAGUUAAAAUAAGGCUAGUCCGUUAUCAACU | |
| 03 | UGAAAAAGUGGCACCGAGUCGGUGCCCCUUUCUCGUCG | |
| | AUGGUCAGCACAGCCUUAUGCACGGCCUGmUsmUsmUsU | |
| gRN | mUsmCsmCsCCUCCAGGCCGUGCAUAGUUUUAGAGCUAG | 213 |
| A54 | AAAUAGCAAGUUAAAAUAAGGCUAGUCCGUUAUCAACU | |
| 04 | UGAAAAAGUGGCACCGAGUCGGUGCUUCUCGUCGAUGG | |
| | UCAGCACAGCCUUAUGCACGGCCUmUsmUsmUsU | |
| gRN | mUsmCsmCsCCUCCAGGCCGUGCAUAGUUUUAGAGCUAG | 214 |
| A54 | AAAUAGCAAGUUAAAAUAAGGCUAGUCCGUUAUCAACU | |
| 05 | UGAAAAAGUGGCACCGAGUCGGUGCCCCUUUCUCGUCG | |
| | AUGGUCAGCACAGCCUUAUGCACGGCCUGGmUsmUsmUs | |
| | U | |
| gRN | mUsmCsmCsCCUCCAGGCCGUGCAUAGUUUUAGAGCUAG | 215 |
| A54 | AAAUAGCAAGUUAAAAUAAGGCUAGUCCGUUAUCAACU | |
| 06 | UGAAAAAGUGGCACCGAGUCGGUGCCCCUUUCUCGUCG | |
| | AUGGUCAGCACAGCCUUAUGCACGGCCUGGAGGGmUsmU | |
| | smUsU | |
| gRN | mUsmCsmCsCCUCCAGGCCGUGCAUAGUUUUAGAGCUAG | 216 |
| A54 | AAAUAGCAAGUUAAAAUAAGGCUAGUCCGUUAUCAACU | |
| 07 | UGAAAAAGUGGCACCGAGUCGGUGCCCCUUUCUCGUCG | |
| | AUGGUCAGCACAGCCUUAUGCACGGCCmUsmUsmUsU | |
| gRN | mUsmCsmCsCCUCCAGGCCGUGCAUAGUUUUAGAGCUAG | 217 |
| A54 | AAAUAGCAAGUUAAAAUAAGGCUAGUCCGUUAUCAACU | |
| 08 | UGAAAAAGUGGCACCGAGUCGGUGCCAGUCCCUUUCUC | |
| | GUCGAUGGUCAGCACAGCCUUAUGCACGGCmUsmUsmUs | |
| | U | |
| gRN | mUsmCsmCsCCUCCAGGCCGUGCAUAGUUUUAGAGCUAG | 218 |
| A54 | AAAUAGCAAGUUAAAAUAAGGCUAGUCCGUUAUCAACU | |
| 09 | UGAAAAAGUGGCACCGAGUCGGUGCCUUCAGUCCCUUU | |
| | CUCGUCGAUGGUCAGCACAGCCUUAUGCACGGCmUsmUs | |
| | mUsU | |
| gRN | mUsmCsmCsCCUCCAGGCCGUGCAUAGUUUUAGAGCUAG | 219 |
| A54 | AAAUAGCAAGUUAAAAUAAGGCUAGUCCGUUAUCAACU | |
| 10 | UGAAAAAGUGGCACCGAGUCGGUGCUUCUCGUCGAUGG | |
| | UCAGCACAGCCUUAUGCACGGCCmUsmUsmUsU | |
| gRN | mUsmCsmCsCCUCCAGGCCGUGCAUAGUUUUAGAGCUAG | 220 |
| A54 | AAAUAGCAAGUUAAAAUAAGGCUAGUCCGUUAUCAACU | |
| 11 | UGAAAAAGUGGCACCGAGUCGGUGCUCUCGUCGAUGGU | |
| | CAGCACAGCCUUAUGCACGGCCUGGAGGGmUsmUsmUsU | |
| gRN | mUsmCsmCsCCUCCAGGCCGUGCAUAGUUUUAGAGCUAG | 221 |
| A54 | AAAUAGCAAGUUAAAAUAAGGCUAGUCCGUUAUCAACU | |
| 12 | UGAAAAAGUGGCACCGAGUCGGUGCUCUCGUCGAUGGU | |
| | CAGCACAGCCUUAUGCACGGCCUGGAGGmUsmUsmUsU | |
| gRN | mUsmCsmCsCCUCCAGGCCGUGCAUAGUUUUAGAGCUAG | 222 |
| A54 | AAAUAGCAAGUUAAAAUAAGGCUAGUCCGUUAUCAACU | |
| 13 | UGAAAAAGUGGCACCGAGUCGGUGCCAGCUUCAGUCCC | |
| | UUUCUCGUCGAUGGUCAGCACAGCCUUAUGCACGGCmUs | |
| | mUsmUsU | |
| gRN | mUsmCsmCsCCUCCAGGCCGUGCAUAGUUUUAGAGCUAG | 223 |
| A54 | AAAUAGCAAGUUAAAAUAAGGCUAGUCCGUUAUCAACU | |
| 14 | UGAAAAAGUGGCACCGAGUCGGUGCUCUCGUCGAUGGU | |
| | CAGCACAGCCUUAUGCACGGCCUGmUsmUsmUsU | |
| gRN | mUsmCsmCsCCUCCAGGCCGUGCAUAGUUUUAGAGCUAG | 224 |
| A54 | AAAUAGCAAGUUAAAAUAAGGCUAGUCCGUUAUCAACU | |
| 15 | UGAAAAAGUGGCACCGAGUCGGUGCUCUCGUCGAUGGU | |
| | CAGCACAGCCUUAUGCACGGCCUGGAGmUsmUsmUsU | |
| gRN | mUsmCsmCsCCUCCAGGCCGUGCAUAGUUUUAGAGCUAG | 225 |
| A54 | AAAUAGCAAGUUAAAAUAAGGCUAGUCCGUUAUCAACU | |
| 16 | UGAAAAAGUGGCACCGAGUCGGUGCCCCUUUCUCGUCG | |
| | AUGGUCAGCACAGCCUUAUGCACGGCmUsmUsmUsU | |
| gRN | mUsmCsmCsCCUCCAGGCCGUGCAUAGUUUUAGAGCUAG | 226 |
| A54 | AAAUAGCAAGUUAAAAUAAGGCUAGUCCGUUAUCAACU | |
| 17 | UGAAAAAGUGGCACCGAGUCGGUGCUCUCGUCGAUGGU | |
| | CAGCACAGCCUUAUGCACGGCCUGGAmUsmUsmUsU | |
| gRN | mUsmCsmCsCCUCCAGGCCGUGCAUAGUUUUAGAGCUAG | 227 |
| A54 | AAAUAGCAAGUUAAAAUAAGGCUAGUCCGUUAUCAACU | |
| 18 | UGAAAAAGUGGCACCGAGUCGGUGCUCUCGUCGAUGGU | |
| | CAGCACAGCCUUAUGCACGGCCUGGmUsmUsmUsU | |
| gRN | mUsmCsmCsCCUCCAGGCCGUGCAUAGUUUUAGAGCUAG | 228 |
| A54 | AAAUAGCAAGUUAAAAUAAGGCUAGUCCGUUAUCAACU | |
| 19 | UGAAAAAGUGGCACCGAGUCGGUGCUUCUCGUCGAUGG | |
| | UCAGCACAGCCUUAUGCACGGCmUsmUsmUsU | |
| gRN | mUsmCsmCsCCUCCAGGCCGUGCAUAGUUUUAGAGCUAG | 229 |
| A54 | AAAUAGCAAGUUAAAAUAAGGCUAGUCCGUUAUCAACU | |
| 20 | UGAAAAAGUGGCACCGAGUCGGUGCUCUCGUCGAUGGU | |
| | CAGCACAGCCUUAUGCACGGCCUmUsmUsmUsU | |
| gRN | mUsmCsmCsCCUCCAGGCCGUGCAUAGUUUUAGAGCUAG | 230 |
| A54 | AAAUAGCAAGUUAAAAUAAGGCUAGUCCGUUAUCAACU | |
| 21 | UGAAAAAGUGGCACCGAGUCGGUGCUCUCGUCGAUGGU | |
| | CAGCACAGCCUUAUGCACGGCCmUsmUsmUsU | |
| gRN | mUsmCsmCsCCUCCAGGCCGUGCAUAGUUUUAGAGCUAG | 231 |
| A54 | AAAUAGCAAGUUAAAAUAAGGCUAGUCCGUUAUCAACU | |
| 22 | UGAAAAAGUGGCACCGAGUCGGUGCUCUCGUCGAUGGU | |
| | CAGCACAGCCUUAUGCACGGCmUsmUsmUsU | |

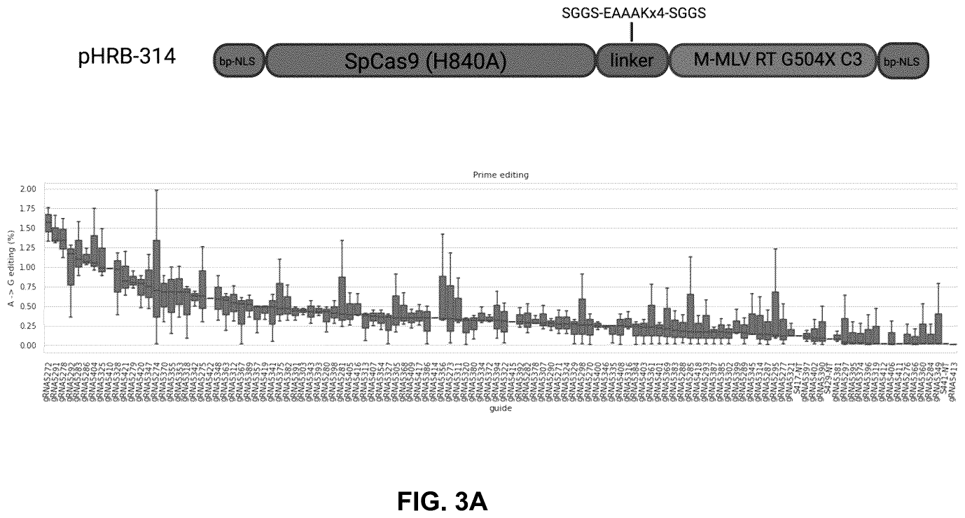

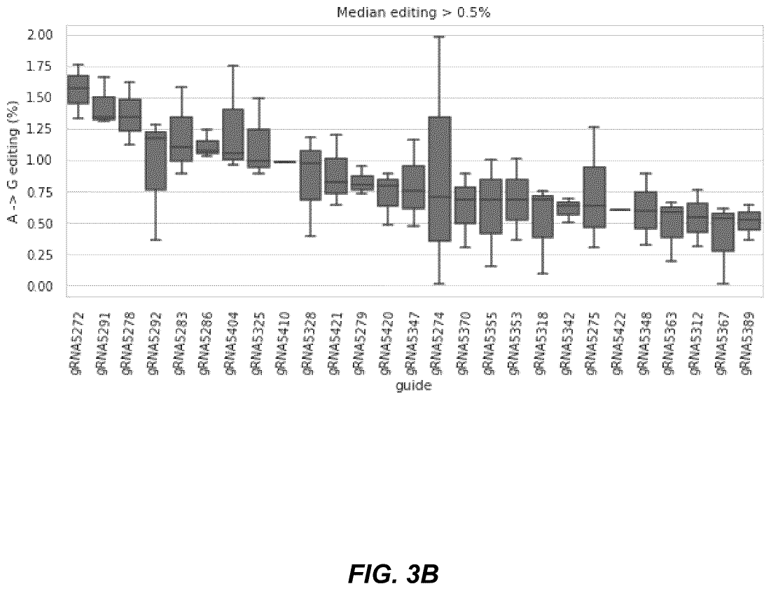

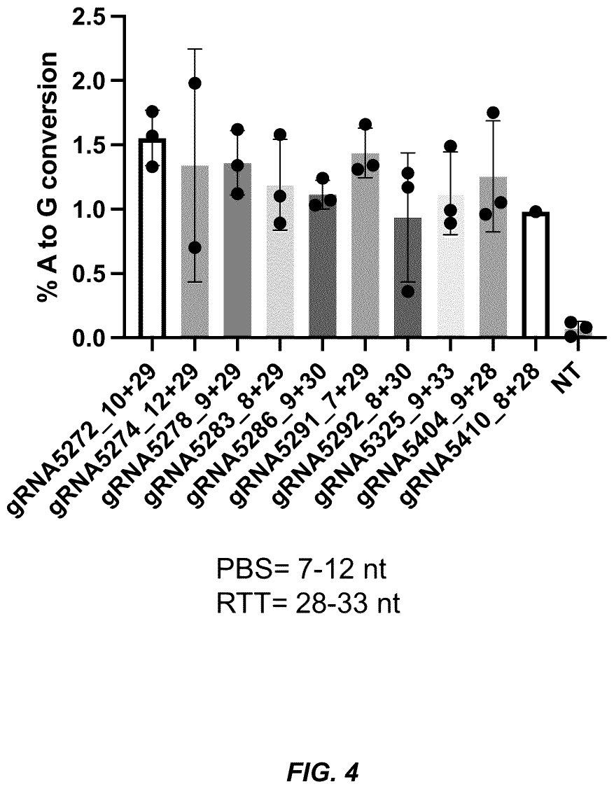

As shown in FIG. 3A, many of the tested pegRNAs, when paired with the prime editor, were capable of correcting the E342K mutation. Among the pegRNAs tested, the median prime editing was above 0.5% (FIG. 3B). Several pegRNAs led to an editing efficiency of ˜1% (FIG. 4). These were selected for further experiments.

### Example 2: Optimization of Prime Editor Architectures to Screen for Prime Editing Efficiency of the α-1 Antitrypsin (A1AT) Pathogenic Z-Mutation (E342K) in Human Primary Fibroblast Cells

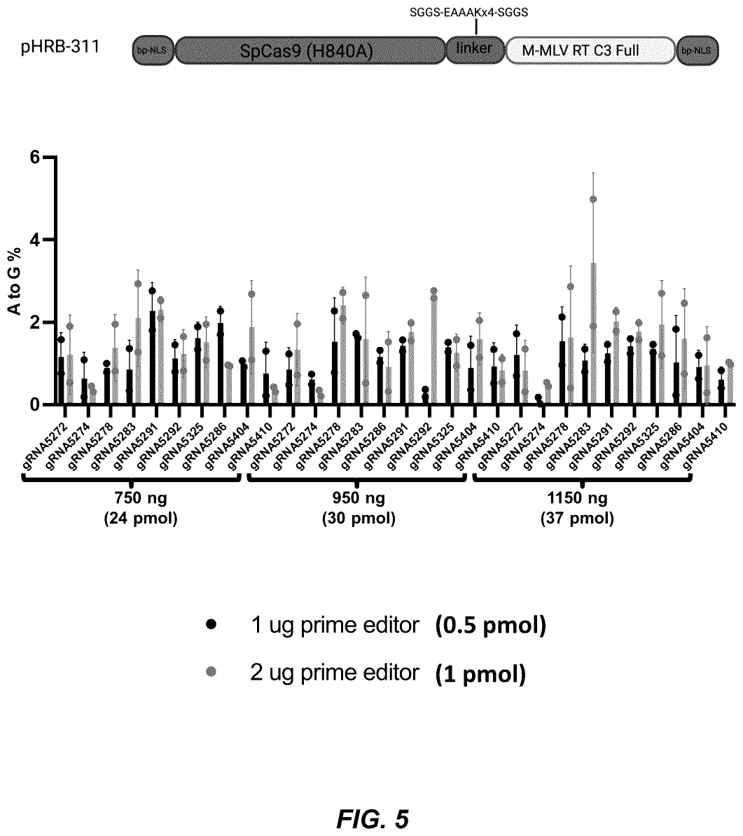

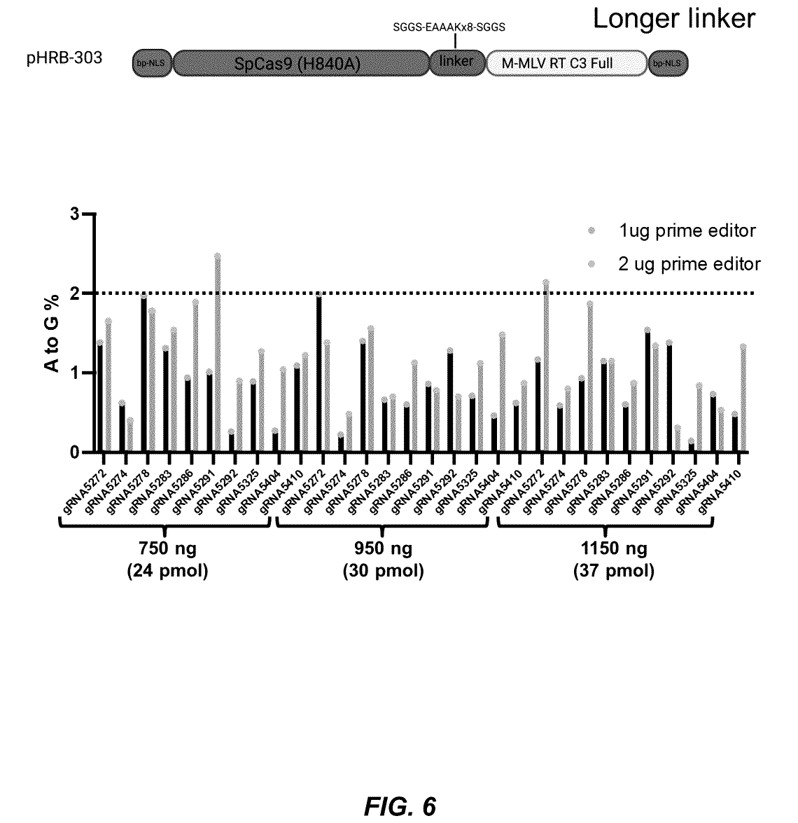

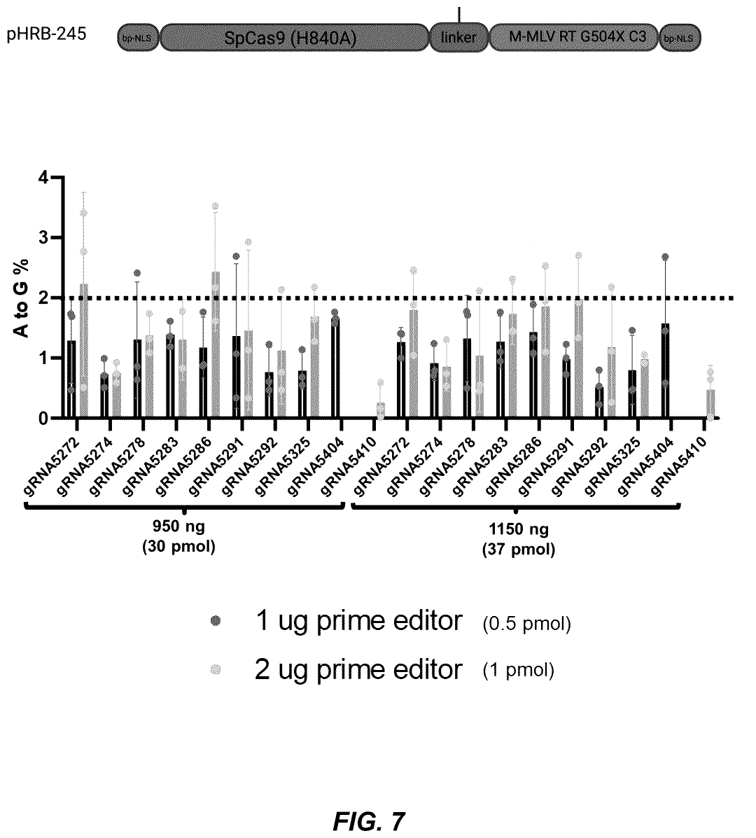

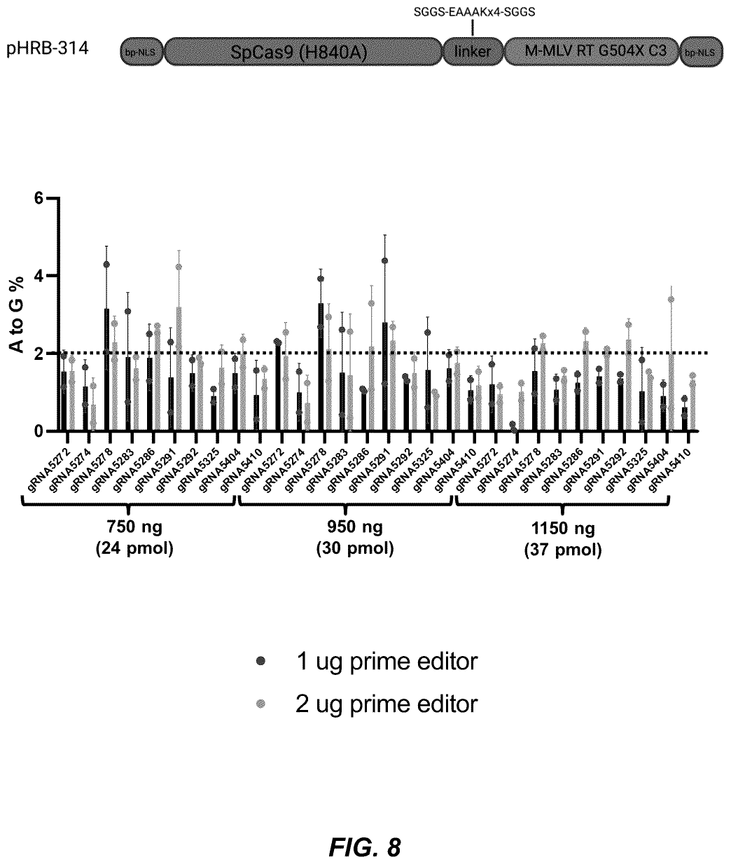

A series of optimized prime editors were screened using different transfection conditions to test their editing efficiency. First, several prime editor variants were assessed using the PE2 prime editing strategy with 1 μg vs 2 μg of the prime editor and 750, 950, or 1150 ng of the pegRNA. The pHRB-311 prime editor employing the SGGS-(EAAAK)₄-SGGS (SEQ ID NO: 277) linker and the full length RT (FIG. 5), the pHRB-303 employing a longer linker, the SGGS-(EAAAK)₈-SGGS (SEQ ID NO: 281) linker, the and the full length RT (FIG. 6), the pHRB-245 prime editor employing the XTEN linker and the G504X RT (FIG. 7), and the pHRB-314 prime editor employing the SGGS-(EAAAK)₄-SGGS (SEQ ID NO: 277) linker and the G504X RT (FIG. 8) were all tested under several transfection conditions.

Across all prime editors and varied pegRNA amounts, robust editing on A1AT E342K was observed. The pHRB-314 prime editor led to an increase in editing efficiency on A1AT E342K upwards of 2%. Thus, this prime editor was carried forward for future experiments.

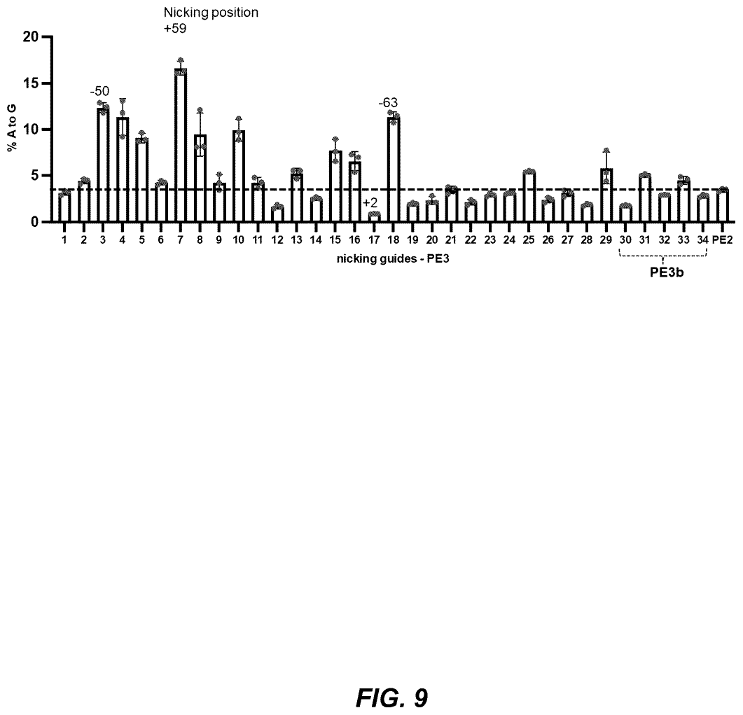

The pHRB-314 prime editor was then evaluated using the PE3 or PE3b strategies to compare its efficiency using the PE2 strategy. HEK293T cells containing the A1AT gene with the pathogenic E342K G>A SNP were transfected with the pHRB-314 prime editor using 34 nicking guides (FIG. 9). The PE3 approach improved efficiency from −3% up to 17% (the 17% improvement was observed using nicking guide #7).

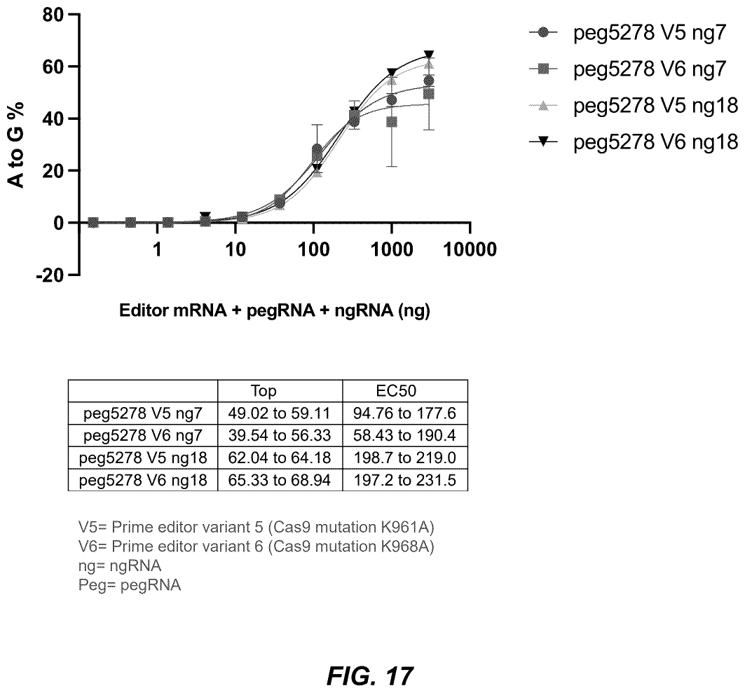

From the nicking guides screen performed in FIG. 9, two candidate nicking guides with high A-to-G editing efficiency were selected for additional testing with two different optimized prime editors (described further in Example 3) to see if editing efficiency could be further improved. Prime editor variant 5 (with a Cas9 mutation K961A; “V5”) or prime editor 6 (with a Cas9 mutation K968A; “V6”), pegRNA 5278 (see Table 1), along with either nicking guide #7 or nicking guide #18 were transfected in human primary fibroblast cell line containing the A1AT gene with the pathogenic E342K G>A SNP (HPF E342K). As shown in FIG. 17 (top of figure), a dose-response curve of the percent editing efficiency of A-to-G correction increased up to 59% and 69% for nicking guide #7 and #18, respectively, when paired with the V5 and V6 optimized prime editors. The EC50 values obtained from the dose response curve were also tabulated and reported in FIG. 17 (bottom of figure). Lower EC50 values were obtained between 58-190 ng when utilizing the combination of prime editor variant 6, pegRNA 5278, and nick guide #7.

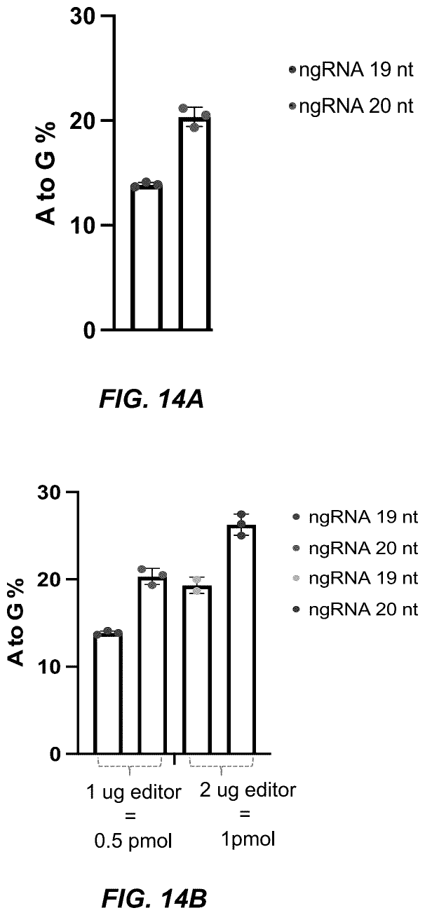

Subsequently, two nicking guides were designed to contain either a 19 or 20 nucleotide spacer region. These nicking guides were called, “gRNA8721 nick7 (20 nt spacer)” and “gRNA8722 nick7 (19 nt spacer).” As shown in FIG. 14, these nicking guide RNA (called “ngRNA 19 nt” or ngRNA 20 nt”; 0.25 ug [6.6 pmol]) were transfected along with prime editor V5 mRNA (“editor” (1 ug [0.5 pmol] or 2 ug [1 pmol]) and pegRNA pegRNA5278 (1 ug [21 pmol]) to correct the pathogenic G>A SNP in the BPF E342A cell line.

The addition of the longer nicking guide improved the prime editing efficiency of an A-to-G correction from 13% to 20% (FIG. 14A). Doubling the amount of prime editor mRNA from 1 ug (0.5 μmol) to 2 ug (1 μmol) further improved the editing efficiency of an A-to-G correction from 20% to 27% (FIG. 14B). Collectively, these results confirm that addition of nicking guide with a 20-nucleotide spacer corresponds with better overall editing efficiency when compared to utilizing a nicking guide with a 19-nucleotide spacer.

The ngRNA spacer sequences and full length ngRNA with chemical modifications used are recited in Table 2 and Table 3 below.

TABLE 2
ngRNA spacer sequences.

| # | | | SEQ |
|---|---|---|---|
| Nicking | Nicking | | ID |
| Guide | Position | Spacer | Nos. |
| 1 | 24 | GUCAGCACAGCCUUAUGCA | 27 |
| 2 | 18 | GAAAGGGACUGAAGCUGCU | 28 |
| 3 | −50 | CCUCGGGGGGGAUAGACAU | 29 |
| 4 | 82 | UGAUCCCAGGCCUCGAGCA | 30 |
| 5 | 70 | ACGUUGUAAGGCUGAUCCC | 31 |
| 6 | 19 | AAAGGGACUGAAGCUGCUG | 32 |
| 7 | 59 | GAAGCAGAGACACGUUGUA | 26 |
| 8 | −30 | GGUAUGGCCUCUAAAAACA | 33 |
| 9 | 61 | CCCAUGUCUAUCCCCCCCG | 34 |
| 10 | 91 | GCCUCGAGCAAGGCUCACG | 35 |
| 11 | −66 | GGUUUGUUGAACUUGACCU | 36 |
| 12 | 34 | CCUUAUGCACGGCCUGGAG | 37 |
| 13 | 17 | AGAAAGGGACUGAAGCUGC | 38 |
| 14 | 29 | CACAGCCUUAUGCACGGCC | 39 |
| 15 | −45 | GGGGGGAUAGACAUGGGUA | 40 |
| 16 | −65 | GUUUGUUGAACUUGACCUC | 41 |
| 17 | 2 | UGCUGACCAUCGACAAGAA | 42 |
| 18 | −63 | UUGUUGAACUUGACCUCGG | 43 |
| 19 | 33 | GCCUUAUGCACGGCCUGGA | 44 |
| 20 | −64 | UUUGUUGAACUUGACCUCG | 45 |
| 21 | −61 | GUUGAACUUGACCUCGGGG | 46 |
| 22 | −35 | CUCUGCUUCUCUCCCCUCC | 47 |
| 23 | −75 | UGAGCCUUGCUCGAGGCCU | 48 |
| 24 | 32 | AGCCUUAUGCACGGCCUGG | 49 |
| 25 | −51 | ACCUCGGGGGGGAUAGACA | 50 |
| 26 | 3 | UCAGUCCCUUUCUUGUCGA | 51 |
| 27 | −62 | UGUUGAACUUGACCUCGGG | 52 |
| 28 | −23 | CCCCUCCAGGCCGUGCAUA | 53 |
| 29 | −76 | GUGAGCCUUGCUCGAGGCC | 54 |
| 30 | 3 | GCUGACCAUCGACAAGAAA | 55 |
| 31 | 34 | GCUGGGGCCAUGUUUUUAG | 56 |
| 32 | 2 | UGCUGACCAUCGACGAGAA | 57 |
| 33 | 3 | UCAGUCCCUUUCUCGUCGA | 58 |
| 34 | 3 | GCUGACCAUCGACGAGAAA | 59 |

TABLE 3
Full length ngRNA for editing A1AT E342K cells.

| # | | |
|---|---|---|
| Nicking | | SEQ ID |
| Guide | Sequence | Nos. |
| 1 | mG*mU*mC*rArGrCrArCrArGrCrCrUrUrArUrGrCrArGrUr | 232 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 2 | mG*mA*mA*rArGrGrGrArCrUrGrArArGrCrUrGrCrUrGrU | 233 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 3 | mC*mC*mU*rCrGrGrGrGrGrGrGrArUrArGrArCrArUrGrU | 234 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 4 | mU*mG*mA*rUrCrCrCrArGrGrCrCrUrCrGrArGrCrArGrUr | 235 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 5 | mA*mC*mG*rUrUrGrUrArArGrGrCrUrGrArUrCrCrCrGrUr | 236 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 6 | mA*mA*mA*rGrGrGrArCrUrGrArArGrCrUrGrCrUrGrGrU | 237 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 7 | mG*mA*mA*rGrCrArGrArGrArCrArCrGrUrUrGrUrArGrU | 238 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 8 | mG*mG*mU*rArUrGrGrCrCrUrCrUrArArArArArCrArGrU | 239 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 9 | mC*mC*mC*rArUrGrUrCrUrArUrCrCrCrCrCrCrCrGrGrUr | 240 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 10 | mG*mC*mC*rUrCrGrArGrCrArArGrGrCrUrCrArCrGrGrUr | 241 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 11 | mG*mG*mU*rUrUrGrUrUrGrArArCrUrUrGrArCrCrUrGrU | 242 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 12 | mC*mC*mU*rUrArUrGrCrArCrGrGrCrCrUrGrGrArGrGrUr | 243 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 13 | mA*mG*mA*rArArGrGrGrArCrUrGrArArGrCrUrGrCrGrU | 244 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 14 | mC*mA*mC*rArGrCrCrUrUrArUrGrCrArCrGrGrCrCrGrUr | 245 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 15 | mG*mG*mG*rGrGrGrArUrArGrArCrArUrGrGrGrUrArGrU | 246 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 16 | mG*mU*mU*rUrGrUrUrGrArArCrUrUrGrArCrCrUrCrGrU | 247 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 17 | mU*mG*mC*rUrGrArCrCrArUrCrGrArCrArArGrArArGrUr | 248 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 18 | mU*mU*mG*rUrUrGrArArCrUrUrGrArCrCrUrCrGrGrGrU | 249 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 19 | mG*mC*mC*rUrUrArUrGrCrArCrGrGrCrCrUrGrGrArGrUr | 250 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 20 | mU*mU*mU*rGrUrUrGrArArCrUrUrGrArCrCrUrCrGrGrU | 251 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 21 | mG*mU*mU*rGrArArCrUrUrGrArCrCrUrCrGrGrGrGrGrU | 252 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 22 | mC*mU*mC*rUrGrCrUrUrCrUrCrUrCrCrCrCrUrCrCrGrUr | 253 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 23 | mU*mG*mA*rGrCrCrUrUrGrCrUrCrGrArGrGrCrCrUrGrUr | 254 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 24 | mA*mG*mC*rCrUrUrArUrGrCrArCrGrGrCrCrUrGrGrGrUr | 255 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 25 | mA*mC*mC*rUrCrGrGrGrGrGrGrGrArUrArGrArCrArGrU | 256 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 26 | mU*mC*mA*rGrUrCrCrCrUrUrUrCrUrUrGrUrCrGrArGrUr | 257 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 27 | mU*mG*mU*rUrGrArArCrUrUrGrArCrCrUrCrGrGrGrGrU | 258 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 28 | mC*mC*mC*rCrUrCrCrArGrGrCrCrGrUrGrCrArUrArGrUr | 259 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 29 | mG*mU*mG*rArGrCrCrUrUrGrCrUrCrGrArGrGrCrCrGrUr | 260 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 30 | mG*mC*mU*rGrArCrCrArUrCrGrArCrArArGrArArArGrUr | 261 |
| | UrUrUrArGrArGrCrUrArGrArArArUrArGrCrArArGrUrUrA | |
| | ArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 31 | mG*mC*mU*rGrGrGrGrCrCrArUrGrUrUrUrUrUrArGrGrU | 262 |
| | rUrUrUrArGrArGrCrUrArGrArArArUrArGrCrArArGrUrUr | |
| | ArArArArUrArArGrGrCrUrArGrUrCrCrGrUrUrArUrCrArA | |
| | rCrUrUrGrArArArArArGrUrGrGrCrArCrCrGrArGrUrCrGrG | |
| | rUrGrCmU*mU*mU*rU | |
| 32 | mU*mG*mC*rUrGrArCrCrArUrCrGrArCrGrArGrArArGrUr | 263 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 33 | mU*mC*mA*rGrUrCrCrCrUrUrUrCrUrCrGrUrCrGrArGrUr | 264 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| 34 | mG*mC*mU*rGrArCrCrArUrCrGrArCrGrArGrArArArGrUr | 265 |
| | UrUrUrArGrArGrCrUrArGrArArArUrArGrCrArArGrUrUrA | |
| | rArArArUrArArGrGrCrUrArGrUrCrCrGrUrUrArUrCrArArC | |
| | rUrUrGrArArArArArGrUrGrGrCrArCrCrGrArGrUrCrGrGr | |
| | UrGrCmU*mU*mU*rU | |
| ngRNA7 | mAsmGsmAsAGCAGAGACACGUUGUAGUUUUAGAGC | 266 |
| gRNA8721 | UAGAAAUAGCAAGUUAAAAUAAGGCUAGUCCGUUA | |
| | UCAACUUGAAAAAGUGGCACCGAGUCGGUGCmUsm | |
| | UsmUsU | |
| gRNA87 | gsasasGCAGAGACACGUUGUAGUUUUAGAgccggcggaaa | 267 |
| 22 nick7 | cgccggcAAGUUAAAAUAAGGCUAGUCCGUUAUCAacu | |
| (19 nt | ugaaaaaguggcaccgagucggugcusususu | |
| spacer) | | |
| ngRNA1 | mUsmUsmUsGUUGAACUUGACCUCGGGUUUUAGAGC | 268 |
| 8 = | UAGAAAUAGCAAGUUAAAAUAAGGCUAGUCCGUUA | |
| gRNA7824 | UCAACUUGAAAAAGUGGCACCGAGUCGGUGCmUsm | |
| | UsmUsU | |
| n/a | wherein “m” corresponds to a 2'-O-methyl | |
| | modification, “s” and “*” corresponds to | |
| | a phosphorothioate internucleotide | |
| | modification, and “r” corresponds | |
| | to an unmodified ribose nucleotide | |

### Example 3. Introducing Mutations in the nCas9 to Alter Prime Editing Efficiency of PegRNAs Containing Silent Mutations in the RTT

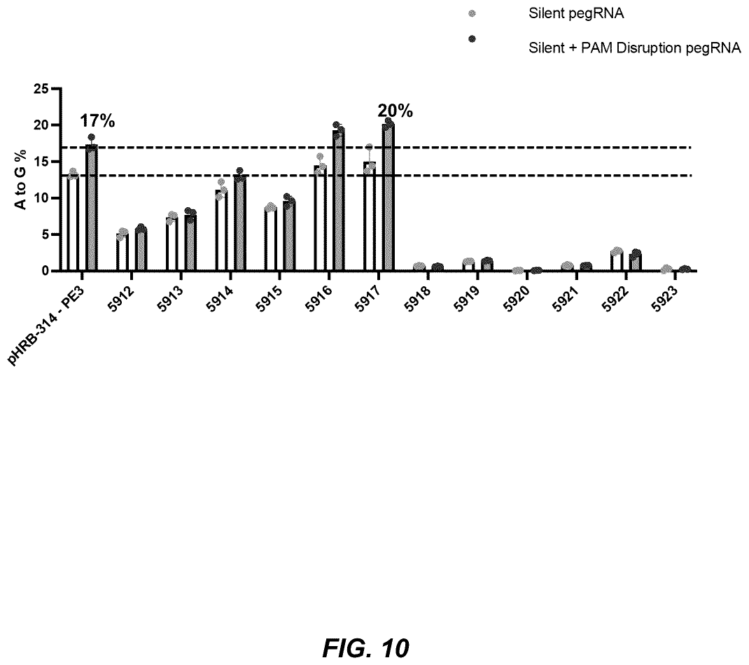

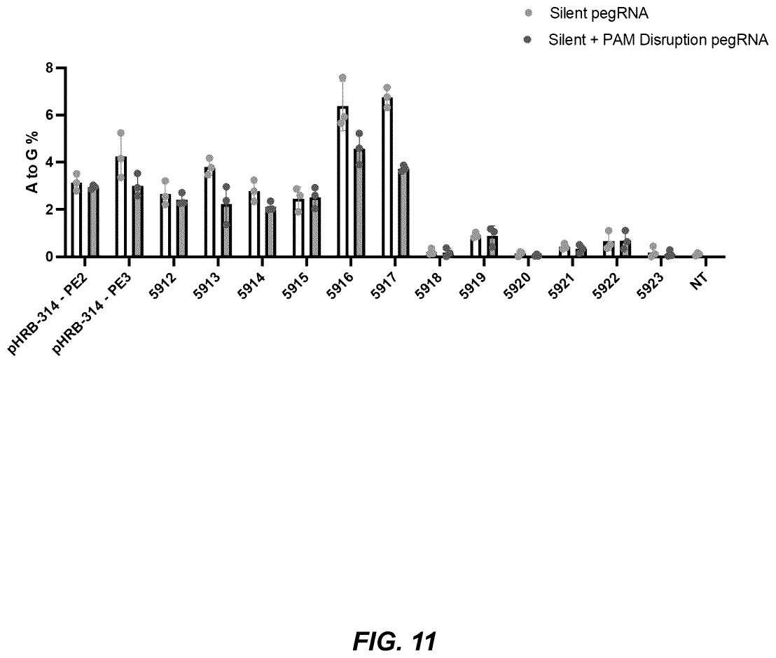

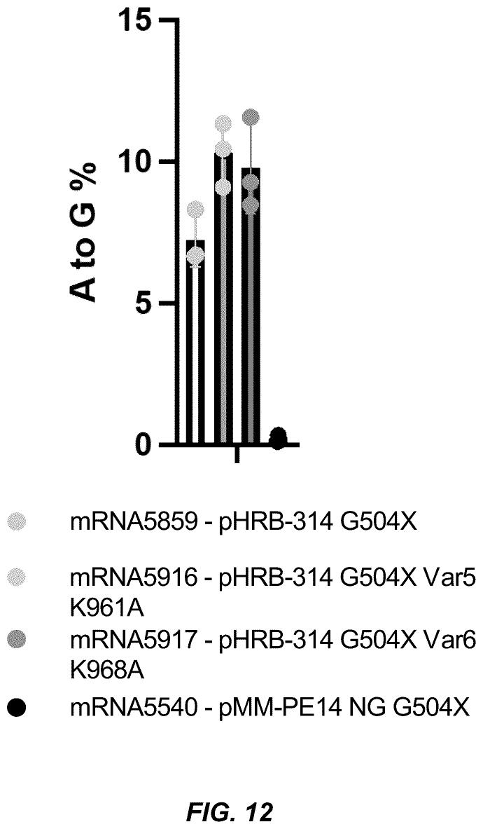

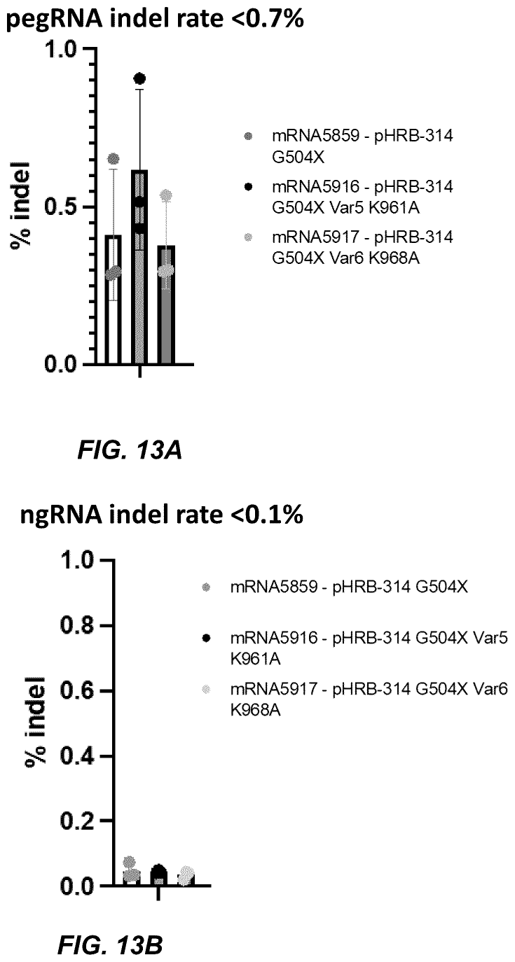

One, two, three, or more mutations were introduced into either the HNH or RuvC domain of the Cas9, resulting in 12 prime editor variants. These variants were also tested along with the pegRNAs containing silent mutations in the RTT and pegRNAs containing both silent mutations and silent PAM disruptions in the RTT. PAM disruption mutations are mutations in the RTT that are done to incorporate silent mutations into the PAM domain and prevent further cutting by Cas9 once an edit is incorporated. Screening was done using the PE3 prime editing strategy and using nicking guide #7 which has previously shown improved editing efficiency in HEK293T cells harboring cells with the A1AT E342K mutation. Only variants 5916 (K961A; prime editor variant 5) and 5917 (K968A) showed an improvement as compared to pHRB-314 from 17% to 20% (FIG. 10 and FIG. 11). Prime editor variants 5 and 6 were further tested for editing efficiency. As shown in FIG. 12, variants 5 and 6 performed better than the original editor HRB-314. Indel rates for the pegRNA and ngRNA were low for all tested prime editors as well (FIG. 13A and FIG. 13B).

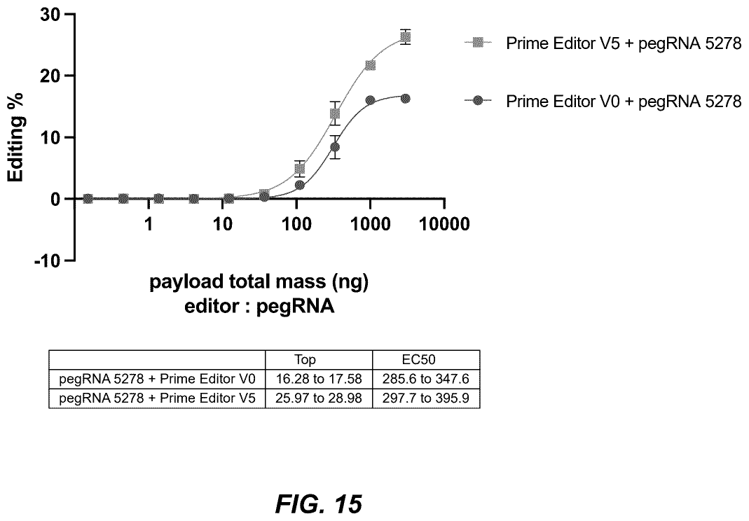

Subsequently, the A-to-G editing efficiency of optimized prime editor variant 5 (prime editor variant 5; mRNA 5916; SEQ ID NO: 65 in Table 4; added at 4 ng) was compared with parental control (prime editor V0) when transfected along with escalating concentrations of pegRNA 5278 (added at 2-10 ng; see Table 1) in a HepG2 E342K cell line. FIG. 15 shows the A-to-G editing efficiency plotted as a dose response curve (FIG. 15, top of figure) and the EC50 values were reported in table format (FIG. 15, bottom of figure). The results show that this optimized prime editor had greater editing efficiency than the parental control.

The variants harboring mutations in nicking Cas9 (nCas9) are recited in Table 4 below:

TABLE 4
Mutations in nCas9 variants.

| Variant # & | |
|---|---|
| Mutation(s) | Sequence |
| Variant 1 | DKKYSIGLDIGTNSVGWAVITDEYKVPSKKFKVLGNTDRH |
| (mRNA 5912); | SIKKNLIGALLFDSGETAEATRLKRTARRRYTRRKNRICY |
| R765A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 61 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMAAENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 2 | DKKYSIGLDIGTNSVGWAVITDEYKVPSKKFKVLGNTDRH |
| (mRNA 5913); | SIKKNLIGALLFDSGETAEATRLKRTARRRYTRRKNRICY |
| K848A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 62 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLADDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHILFTLTNLGA |
| | PAAFKYFDTTIDRKRYTSTKEVLDATLIHQSITGLYETRI |
| | DLSQLGGD |
| Variant 3 | DKKYSIGLDIGTNSVGWAVITDEYKVPSKKFKVLGNTDRH |
| (mRNA 5914); | SIKKNLIGALLFDSGETAEATRLKRTARRRYTRRKNRICY |
| K855A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 63 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNAVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 4 | DKKYSIGLDIGTNSVGWAVITDEYKVPSKKFKVLGNTDRH |
| (mRNA 5915); | SIKKNLIGALLFDSGETAEATRLKRTARRRYTRRKNRICY |
| K959A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 64 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLASK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 5 | DKKYSIGLDIGTNSVGWAVITDEYKVPSKKFKVLGNTDRH |
| (mRNA 5916); | SIKKNLIGALLFDSGETAEATRLKRTARRRYTRRKNRICY |
| K961A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 65 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSA |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 6 | DKKYSIGLDIGTNSVGWAVITDEYKVPSKKFKVLGNTDRH |
| (mRNA 5917); | SIKKNLIGALLFDSGETAEATRLKRTARRRYTRRKNRICY |
| K968A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 66 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLASK |
| | LVSDFRADFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 7 | DKKYSIGLDIGTNSVGWAVITDEYKVPSKKFKVLGNTDRH |
| (mRNA 5918); | SIKKNLIGALLFDSGETAEATRLKRTARRRYTRRKNRICY |
| K974A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 67 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYAVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 8 | DKKYSIGLDIGTNSVGWAVITDEYKVPSKKFKVLGNTDRH |
| (mRNA 5919); | SIKKNLIGALLFDSGETAEATRLKRTARRRYTRRKNRICY |
| R976A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 68 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVAEINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 9 | DKKYSIGLDIGTNSVGWAVITDEYKVPSKKFKVLGNTDRH |
| (mRNA 5920); | SIKKNLIGALLFDSGETAEATRLKRTARRRYTRRKNRICY |
| R765A/K848A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 69 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMAAENQTTQKGQKNSRERMKRIEEGIKELGSQILKEHPV |
| | ENTQLQNEKLYLYYLQNGRDMYVDQELDINRLSDYDVDAI |
| | VPQSFLADDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 10 | DKKYSIGLDIGTNSVGWAVITDEYKVPSKKFKVLGNTDRH |
| (mRNA 5921); | SIKKNLIGALLFDSGETAEATRLKRTARRRYTRRKNRICY |
| K974A/R976A | LQEIFSNEMAKVDDSFFHRLEESFLVEEDKKHERHPIFGN |
| SEQ ID NO: | IVDEVAYHEKYPTIYHLRKKLVDSTDKADLRLIYLALAHM |
| 70 | IKFRGHFLIEGDLNPDNSDVDKLFIQLVQTYNQLFEENPI |
| | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYAVAEINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 11 | DKKYSIGLDIGTNSVGWAVITDEYKVPSKKFKVLGNTDRH |
| (mRNA 5922); | SIKKNLIGALLFDSGETAEATRLKRTARRRYTRRKNRICY |
| R765A/K848A | LQEIFSNEMAKVDDSFFHRLEESFLVEEDKKHERHPIFGN |
| /K855A | IVDEVAYHEKYPTIYHLRKKLVDSTDKADLRLIYLALAHM |
| SEQ ID NO: | IKFRGHFLIEGDLNPDNSDVDKLFIQLVQTYNQLFEENPI |
| 71 | NASGVDAKAILSARLSKSRRLENLIAQLPGEKKNGLFGNL |
| | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVJ |
| | EMAAENQTTQKGQKNSRERMKRIEEGIKELGSQILKEHPV |
| | ENTQLQNEKLYLYYLQNGRDMYVDQELDINRLSDYDVDAI |
| | VPQSFLADDSIDNAVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLKSK |
| | LVSDFRKDFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |
| Variant 12 | DKKYSIGLDIGTNSVGWAVITDEYKVPSKKFKVLGNTDRH |
| (mRNA 5923); | SIKKNLIGALLFDSGETAEATRLKRTARRRYTRRKNRICY |
| K959A/ | LQEIFSNEMAKVDDSFFHRLEESFLVEEDKKHERHPIFGN |
| K961A/ | IVDEVAYHEKYPTIYHLRKKLVDSTDKADLRLIYLALAHM |
| K968A | IKFRGHFLIEGDLNPDNSDVDKLFIQLVQTYNQLFEENPI |
| SEQ ID NO: | NASGVDAKAILSARLSKSRRLENLIAQLPGEKKNGLFGNL |
| 72 | IALSLGLTPNFKSNFDLAEDAKLQLSKDTYDDDLDNLLAQ |
| | IGDQYADLFLAAKNLSDAILLSDILRVNTEITKAPLSASM |
| | IKRYDEHHQDLTLLKALVRQQLPEKYKEIFFDQSKNGYAG |
| | YIDGGASQEEFYKFIKPILEKMDGTEELLVKLNREDLLRK |
| | QRTFDNGSIPHQIHLGELHAILRRQEDFYPFLKDNREKIE |
| | KILTFRIPYYVGPLARGNSRFAWMTRKSEETITPWNFEEV |
| | VDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKII |
| | KDKDFLDNEENEDILEDIVLTLTLFEDREMIEERLKTYAH |
| | LFDDKVMKQLKRRRYTGWGRLSRKLINGIRDKQSGKTILD |
| | FLKSDGFANRNFMQLIHDDSLTFKEDIQKAQVSGQGDSLH |
| | EHIANLAGSPAIKKGILQTVKVVDELVKVMGRHKPENIVI |
| | EMARENQTTQKGQKNSRERMKRIEEGIKELGSQILKEHPV |
| | ENTQLQNEKLYLYYLQNGRDMYVDQELDINRLSDYDVDAI |
| | VPQSFLKDDSIDNKVLTRSDKNRGKSDNVPSEEVVKKMKN |
| | YWRQLLNAKLITQRKFDNLTKAERGGLSELDKAGFIKRQL |
| | VETRQITKHVAQILDSRMNTKYDENDKLIREVKVITLASA |
| | LVSDFRADFQFYKVREINNYHHAHDAYLNAVVGTALIKKY |
| | PKLESEFVYGDYKVYDVRKMIAKSEQEIGKATAKYFFYSN |
| | IMNFFKTEITLANGEIRKRPLIETNGETGEIVWDKGRDFA |
| | TVRKVLSMPQVNIVKKTEVQTGGFSKESILPKRNSDKLIA |
| | RKKDWDPKKYGGFDSPTVAYSVLVVAKVEKGKSKKLKSVK |
| | ELLGITIMERSSFEKNPIDFLEAKGYKEVKKDLIIKLPKY |
| | SLFELENGRKRMLASAGELQKGNELALPSKYVNFLYLASH |
| | YEKLKGSPEDNEQKQLFVEQHKHYLDEIIEQISEFSKRVI |
| | LADANLDKVLSAYNKHRDKPIREQAENIIHLFTLTNLGAP |
| | AAFKYFDTTIDRKRYTSTKEVLDATLIHQSITGLYETRID |
| | LSQLGGD |

Mutation position numbering in the variants is relative to the sequence recited below, which is the Cas9 component of the prime editor HRB-314:

(SEQ ID NO: 60)
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD.

An additional Cas9 sequence is recited below, harboring the H840A mutation, which is present in Variants 1-12:

(SEQ ID NO: 76)
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD.

These prime editor variants were also tested along with the PegRNAs containing silent mutations in the RTU and PegRNAs containing both silent mutations and silent PAM disruptions in the RTU using the sane experimental design as above (PE3 prime editing strategy and using nicking guide #7) in human primary fibroblast cells. The same variants 5916 (K961A) and 5917 (K968A) also showed an improvement as compared to pHRB-314 from 5% to 7% (FIG. 10 and FIG. 11). In contrast, pegRNA with the silent D341D+PAM disruption mutations did not show an improved editing efficiency when compared to the silent mutation D341D alone.

The editing template and PBS for the silent D341D mutation pegRNAs are recited below in Table 5. Each silent D341D mutation pegRNA employed the same spacer sequence of UCCCCUCCAGGCCGUGCAUA (SEQ ID NO: 1).

TABLE 5
Silent D341D mutation pegRNAs

| Silent | | |
|---|---|---|
| mutation | Editing template | |
| pegRNA | sequence | PBS sequence |
| gRNA6361 | UUUCUCAUCGAUGGU | GCACGGCCUG |
| | CAGCACAGCCUUAU | (SEQ ID NO: 5) |
| | (SEQ ID NO: 283) | |
| gRNA6362 | UUUCUCAUCGAUGGU | GCACGGCCU |
| | CAGCACAGCCUUAU | (SEQ ID NO: 4) |
| | (SEQ ID NO: 283) | |
| gRNA6363 | UUUCUCAUCGAUGGU | GCACGGCC |
| | CAGCACAGCCUUAU | (SEQ ID NO: 3) |
| | (SEQ ID NO: 283) | |
| gRNA6364 | UUUCUCAUCGAUGGU | GCACGGC (SEQ |
| | CAGCACAGCCUUAU | ID NO: 2) |
| | (SEQ ID NO: 283) | |
| gRNA6365 | CUUUCUCAUCGAUGG | GCACGGCC |
| | UCAGCACAGCCUUAU | (SEQ ID NO: 3) |
| | (SEQ ID NO: 284) | |

The prime editor amino acid sequence, and prime editor mRNA sequence employed are recited below:

| SEQ ID | Sequence |
|---|---|
| PEAv2 amino acid | MKRTADGSEFESPKKKRKVDKKYSIGLDIGTNSVGWAVITD |
| (also designated | EYKVPSKKFKVLGNTDRHSIKKNLIGALLFDSGETAEATRL |
| as “PEAG504X”) | KRTARRRYTRRKNRICYLQEIFSNEMAKVDDSFFHRLEESFL |
| | VEEDKKHERHPIFGNIVDEVAYHEKYPTIYHLRKKLVDSTD |
| | KADLRLIYLALAHMIKFRGHFLIEGDLNPDNSDVDKLFIQLV |
| | QTYNQLFEENPINASGVDAKAILSARLSKSRRLENLIAQLPG |
| | EKKNGLFGNLIALSLGLTPNFKSNFDLAEDAKLQLSKDTYD |
| | DDLDNLLAQIGDQYADLFLAAKNLSDAILLSDILRVNTEITK |
| | APLSASMIKRYDEHHQDLTLLKALVRQQLPEKYKEIFFDQS |
| | KNGYAGYIDGGASQEEFYKFIKPILEKMDGTEELLVKLNRE |
| | DLLRKQRTFDNGSIPHQIHLGELHAILRRQEDFYPFLKDNRE |
| | KIEKILTFRIPYYVGPLARGNSRFAWMTRKSEETITPWNFEE |
| | VVDKGASAQSFIERMTNFDKNLPNEKVLPKHSLLYEYFTVY |
| | NELTKVKYVTEGMRKPAFLSGEQKKAIVDLLFKTNRKVTV |
| | KQLKEDYFKKIECFDSVEISGVEDRFNASLGTYHDLLKIIKD |
| | KDFLDNEENEDILEDIVLTLTLFEDREMIEERLKTYAHLFDD |
| | KVMKQLKRRRYTGWGRLSRKLINGIRDKQSGKTILDFLKSD |
| | GFANRNFMQLIHDDSLTFKEDIQKAQVSGQGDSLHEHIANL |
| | AGSPAIKKGILQTVKVVDELVKVMGRHKPENIVIEMARENQ |
| | TTQKGQKNSRERMKRIEEGIKELGSQILKEHPVENTQLQNE |
| | KLYLYYLQNGRDMYVDQELDINRLSDYDVDAIVPQSFLKD |
| | DSIDNKVLTRSDKNRGKSDNVPSEEVVKKMKNYWRQLLN |
| | AKLITQRKFDNLTKAERGGLSELDKAGFIKRQLVETRQITKH |
| | VAQILDSRMNTKYDENDKLIREVKVITLKSKLVSDFRKDFQ |
| | FYKVREINNYHHAHDAYLNAVVGTALIKKYPKLESEFVYG |
| | DYKVYDVRKMIAKSEQEIGKATAKYFFYSNIMNFFKTEITL |
| | ANGEIRKRPLIETNGETGEIVWDKGRDFATVRKVLSMPQVN |
| | IVKKTEVQTGGFSKESILPKRNSDKLIARKKDWDPKKYGGF |
| | DSPTVAYSVLVVAKVEKGKSKKLKSVKELLGITIMERSSFE |
| | KNPIDFLEAKGYKEVKKDLIIKLPKYSLFELENGRKRMLASA |
| | GELQKGNELALPSKYVNFLYLASHYEKLKGSPEDNEQKQLF |
| | VEQHKHYLDEIIEQISEFSKRVILADANLDKVLSAYNKHRDK |
| | PIREQAENIIHLFTLTNLGAPAAFKYFDTTIDRKRYTSTKEVL |
| | DATLIHQSITGLYETRIDLSQLGGDSGGSEAAAKEAAAKEA |
| | AAKEAAAKSGGSTLNIEDEYRLHETSKEPDVSLGSTWLSDF |
| | PQAWAETGGMGLAVRQAPLIIPLKATSTPVSIKQYPMSQEA |
| | RLGIKPHIQRLLDQGILVPCQSPWNTPLLPVKKPGTNDYRPV |
| | QDLREVNKRVEDIHPTVPNPYNLLSGLPPSHQWYTVLDLKD |
| | AFFCLRLHPTSQPLFAFEWRDPEMGISGQLTWTRLPQGFKN |
| | SPTLFNEALHRDLADFRIQHPDLILLQYVDDLLLAATSELDC |
| | QQGTRALLQTLGNLGYRASAKKAQICQKQVKYLGYLLKEG |
| | QRWLTEARKETVMGQPTPKTPRQLREFLGKAGFCRLFIPGF |
| | AEMAAPLYPLTKPGTLFNWGPDQQKAYQEIKQALLTAPAL |
| | GLPDLTKPFELFVDEKQGYAKGVLTQKLGPWRRPVAYLSK |
| | KLDPVAAGWPPCLRMVAAIAVLTKDAGKLTMGQPLVILAP |
| | HAVEALVKQPPDRWLSNARMTHYQALLLDTDRVQFGPVV |
| | ALNPATLLPLPEEGLQHNCLDILAEAHGGGSKRTADGSEFEP |
| | KKKRKV (SEQ ID NO: 307) |
| PEAv2 mRNA open | AUGAAACGGACAGCCGACGGAAGCGAGUUCGAGUCACC |
| reading frame | AAAGAAGAAGCGGAAAGUCGACAAGAAGUACAGCAUCG |
| (also designated | GCCUGGACAUCGGCACCAACUCUGUGGGCUGGGCCGUG |
| as “PEAG504X”) | AUCACCGACGAGUACAAGGUGCCCAGCAAGAAAUUCAA |
| | GGUGCUGGGCAACACCGACCGGCACAGCAUCAAGAAGA |
| | ACCUGAUCGGAGCCCUGCUGUUCGACAGCGGCGAAACA |
| | GCCGAGGCCACCCGGCUGAAGAGAACCGCCAGAAGAAG |
| | AUACACCAGACGGAAGAACCGGAUCUGCUAUCUGCAAG |
| | AGAUCUUCAGCAACGAGAUGGCCAAGGUGGACGACAGC |
| | UUCUUCCACAGACUGGAAGAGUCCUUCCUGGUGGAAGA |
| | GGAUAAGAAGCACGAGCGGCACCCCAUCUUCGGCAACA |
| | UCGUGGACGAGGUGGCCUACCACGAGAAGUACCCCACC |
| | AUCUACCACCUGAGAAAGAAACUGGUGGACAGCACCGA |
| | CAAGGCCGACCUGCGGCUGAUCUAUCUGGCCCUGGCCC |
| | ACAUGAUCAAGUUCCGGGGCCACUUCCUGAUCGAGGGC |
| | GACCUGAACCCCGACAACAGCGACGUGGACAAGCUGUU |
| | CAUCCAGCUGGUGCAGACCUACAACCAGCUGUUCGAGG |
| | AAAACCCCAUCAACGCCAGCGGCGUGGACGCCAAGGCC |
| | AUCCUGUCUGCCAGACUGAGCAAGAGCAGACGGCUGGA |
| | AAAUCUGAUCGCCCAGCUGCCCGGCGAGAAGAAGAAUG |
| | GCCUGUUCGGAAACCUGAUUGCCCUGAGCCUGGGCCUG |
| | ACCCCCAACUUCAAGAGCAACUUCGACCUGGCCGAGGA |
| | UGCCAAACUGCAGCUGAGCAAGGACACCUACGACGACG |
| | ACCUGGACAACCUGCUGGCCCAGAUCGGCGACCAGUAC |
| | GCCGACCUGUUUCUGGCCGCCAAGAACCUGUCCGACGC |
| | CAUCCUGCUGAGCGACAUCCUGAGAGUGAACACCGAGA |
| | UCACCAAGGCCCCCCUGAGCGCCUCUAUGAUCAAGAGA |
| | UACGACGAGCACCACCAGGACCUGACCCUGCUGAAAGC |
| | UCUCGUGCGGCAGCAGCUGCCUGAGAAGUACAAAGAGA |
| | UUUUCUUCGACCAGAGCAAGAACGGCUACGCCGGCUAC |
| | AUUGACGGCGGAGCCAGCCAGGAAGAGUUCUACAAGUU |
| | CAUCAAGCCCAUCCUGGAAAAGAUGGACGGCACCGAGG |
| | AACUGCUCGUGAAGCUGAACAGAGAGGACCUGCUGCGG |
| | AAGCAGCGGACCUUCGACAACGGCAGCAUCCCCCACCA |
| | GAUCCACCUGGGAGAGCUGCACGCCAUUCUGCGGCGGC |
| | AGGAAGAUUUUUACCCAUUCCUGAAGGACAACCGGGAA |
| | AAGAUCGAGAAGAUCCUGACCUUCCGCAUCCCCUACUA |
| | CGUGGGCCCUCUGGCCAGGGGAAACAGCAGAUUCGCCU |
| | GGAUGACCAGAAAGAGCGAGGAAACCAUCACCCCCUGG |
| | AACUUCGAGGAAGUGGUGGACAAGGGCGCUUCCGCCCA |
| | GAGCUUCAUCGAGCGGAUGACCAACUUCGAUAAGAACC |
| | UGCCCAACGAGAAGGUGCUGCCCAAGCACAGCCUGCUG |
| | UACGAGUACUUCACCGUGUAUAACGAGCUGACCAAAGU |
| | GAAAUACGUGACCGAGGGAAUGAGAAAGCCCGCCUUCC |
| | UGAGCGGCGAGCAGAAAAAGGCCAUCGUGGACCUGCUG |
| | UUCAAGACCAACCGGAAAGUGACCGUGAAGCAGCUGAA |
| | AGAGGACUACUUCAAGAAAAUCGAGUGCUUCGACUCCG |
| | UGGAAAUCUCCGGCGUGGAAGAUCGGUUCAACGCCUCC |
| | CUGGGCACAUACCACGAUCUGCUGAAAAUUAUCAAGGA |
| | CAAGGACUUCCUGGACAAUGAGGAAAACGAGGACAUUC |
| | UGGAAGAUAUCGUGCUGACCCUGACACUGUUUGAGGAC |
| | AGAGAGAUGAUCGAGGAACGGCUGAAAACCUAUGCCCA |
| | CCUGUUCGACGACAAAGUGAUGAAGCAGCUGAAGCGGC |
| | GGAGAUACACCGGCUGGGGCAGGCUGAGCCGGAAGCUG |
| | AUCAACGGCAUCCGGGACAAGCAGUCCGGCAAGACAAU |
| | CCUGGAUUUCCUGAAGUCCGACGGCUUCGCCAACAGAA |
| | ACUUCAUGCAGCUGAUCCACGACGACAGCCUGACCUUU |
| | AAAGAGGACAUCCAGAAAGCCCAGGUGUCCGGCCAGGG |
| | CGAUAGCCUGCACGAGCACAUUGCCAAUCUGGCCGGCA |
| | GCCCCGCCAUUAAGAAGGGCAUCCUGCAGACAGUGAAG |
| | GUGGUGGACGAGCUCGUGAAAGUGAUGGGCCGGCACAA |
| | GCCCGAGAACAUCGUGAUCGAAAUGGCCAGAGAGAACC |
| | AGACCACCCAGAAGGGACAGAAGAACAGCCGCGAGAGA |
| | AUGAAGCGGAUCGAAGAGGGCAUCAAAGAGCUGGGCAG |
| | CCAGAUCCUGAAAGAACACCCCGUGGAAAACACCCAGC |
| | UGCAGAACGAGAAGCUGUACCUGUACUACCUGCAGAAU |
| | GGGCGGGAUAUGUACGUGGACCAGGAACUGGACAUCAA |
| | CCGGCUGUCCGACUACGAUGUGGACGCUAUCGUGCCUC |
| | AGAGCUUUCUGAAGGACGACUCCAUCGACAACAAGGUG |
| | CUGACCAGAAGCGACAAGAACCGGGGCAAGAGCGACAA |
| | CGUGCCCUCCGAAGAGGUCGUGAAGAAGAUGAAGAACU |
| | ACUGGCGGCAGCUGCUGAACGCCAAGCUGAUUACCCAG |
| | AGAAAGUUCGACAAUCUGACCAAGGCCGAGAGAGGCGG |
| | CCUGAGCGAACUGGAUAAGGCCGGCUUCAUCAAGAGAC |
| | AGCUGGUGGAAACCCGGCAGAUCACAAAGCACGUGGCA |
| | CAGAUCCUGGACUCCCGGAUGAACACUAAGUACGACGA |
| | GAAUGACAAGCUGAUCCGGGAAGUGAAAGUGAUCACCC |
| | UGAAGUCCAAGCUGGUGUCCGAUUUCCGGAAGGAUUUC |
| | CAGUUUUACAAAGUGCGCGAGAUCAACAACUACCACCA |
| | CGCCCACGACGCCUACCUGAACGCCGUCGUGGGAACCG |
| | CCCUGAUCAAAAAGUACCCUAAGCUGGAAAGCGAGUUC |
| | GUGUACGGCGACUACAAGGUGUACGACGUGCGGAAGAU |
| | GAUCGCCAAGAGCGAGCAGGAAAUCGGCAAGGCUACCG |
| | CCAAGUACUUCUUCUACAGCAACAUCAUGAACUUUUUC |
| | AAGACCGAGAUUACCCUGGCCAACGGCGAGAUCCGGAA |
| | GCGGCCUCUGAUCGAGACAAACGGCGAAACCGGGGAGA |
| | UCGUGUGGGAUAAGGGCCGGGAUUUUGCCACCGUGCGG |
| | AAAGUGCUGAGCAUGCCCCAAGUGAAUAUCGUGAAAAA |
| | GACCGAGGUGCAGACAGGCGGCUUCAGCAAAGAGUCUA |
| | UCCUGCCCAAGAGGAACAGCGAUAAGCUGAUCGCCAGA |
| | AAGAAGGACUGGGACCCUAAGAAGUACGGCGGCUUCGA |
| | CAGCCCCACCGUGGCCUAUUCUGUGCUGGUGGUGGCCA |
| | AAGUGGAAAAGGGCAAGUCCAAGAAACUGAAGAGUGU |
| | GAAAGAGCUGCUGGGGAUCACCAUCAUGGAAAGAAGCA |
| | GCUUCGAGAAGAAUCCCAUCGACUUUCUGGAAGCCAAG |
| | GGCUACAAAGAAGUGAAAAAGGACCUGAUCAUCAAGCU |
| | GCCUAAGUACUCCCUGUUCGAGCUGGAAAACGGCCGGA |
| | AGAGAAUGCUGGCCUCUGCCGGCGAACUGCAGAAGGGA |
| | AACGAACUGGCCCUGCCCUCCAAAUAUGUGAACUUCCU |
| | GUACCUGGCCAGCCACUAUGAGAAGCUGAAGGGCUCCC |
| | CCGAGGAUAAUGAGCAGAAACAGCUGUUUGUGGAACAG |
| | CACAAGCACUACCUGGACGAGAUCAUCGAGCAGAUCAG |
| | CGAGUUCUCCAAGAGAGUGAUCCUGGCCGACGCUAAUC |
| | UGGACAAAGUGCUGUCCGCCUACAACAAGCACCGGGAU |
| | AAGCCCAUCAGAGAGCAGGCCGAGAAUAUCAUCCACCU |
| | GUUUACCCUGACCAAUCUGGGAGCCCCUGCCGCCUUCA |
| | AGUACUUUGACACCACCAUCGACCGGAAGAGGUACACC |
| | AGCACCAAAGAGGUGCUGGACGCCACCCUGAUCCACCA |
| | GAGCAUCACCGGCCUGUACGAGACACGGAUCGACCUGU |
| | CUCAGCUGGGAGGUGACUCCGGCGGCAGCGAGGCCGCC |
| | GCCAAGGAAGCCGCCGCCAAGGAAGCCGCUGCCAAGGA |
| | GGCCGCUGCUAAAAGCGGCGGAUCUACCCUGAACAUCG |
| | AGGACGAGUACAGGCUGCACGAGACCAGCAAGGAGCCC |
| | GACGUGAGCCUGGGCAGCACCUGGCUGAGCGAUUUCCC |
| | UCAGGCUUGGGCCGAGACCGGCGGCAUGGGCCUGGCCG |
| | UGCGGCAGGCCCCCCUGAUUAUCCCCCUGAAGGCCACC |
| | AGCACCCCCGUGAGCAUCAAGCAGUACCCAAUGUCCCA |
| | GGAGGCCAGGCUGGGCAUCAAGCCUCACAUCCAGAGGC |
| | UGCUGGACCAGGGCAUCCUGGUGCCAUGCCAGUCCCCC |
| | UGGAACACCCCUCUGCUGCCCGUGAAGAAGCCUGGCAC |
| | CAACGACUACCGGCCCGUGCAGGACCUGAGAGAAGUGA |
| | ACAAGCGGGUGGAGGACAUCCACCCAACCGUGCCCAAC |
| | CCUUACAACCUGCUGUCCGGCCUGCCCCCCAGCCACCAG |
| | UGGUACACCGUGCUGGACCUGAAGGACGCCUUCUUCUG |
| | CCUGAGACUGCACCCCACCUCUCAGCCCCUGUUCGCCUU |
| | CGAGUGGCGCGACCCCGAGAUGGGCAUCAGCGGCCAGC |
| | UGACCUGGACCAGACUGCCACAGGGCUUUAAGAAUAGC |
| | CCAACCCUGUUUAACGAGGCCCUGCACAGGGACCUGGC |
| | CGACUUCAGGAUCCAGCACCCCGACCUGAUUCUGCUGC |
| | AGUACGUGGACGACCUGCUGCUGGCCGCUACCAGCGAG |
| | CUGGACUGCCAGCAGGGCACCAGAGCCCUGCUGCAGAC |
| | CCUGGGCAACCUGGGCUACAGAGCCAGCGCCAAGAAGG |
| | CCCAGAUCUGUCAGAAGCAGGUGAAGUAUCUGGGCUAC |
| | CUGCUGAAGGAAGGCCAGAGAUGGCUGACCGAGGCCAG |
| | AAAGGAGACUGUGAUGGGCCAGCCCACCCCCAAGACCC |
| | CCAGGCAGCUGCGGGAGUUCCUGGGCAAGGCCGGCUUU |
| | UGCAGACUGUUUAUCCCUGGCUUCGCCGAGAUGGCCGC |
| | CCCACUGUACCCUCUGACCAAGCCUGGCACCCUGUUUA |
| | ACUGGGGCCCCGACCAGCAGAAGGCCUACCAGGAGAUC |
| | AAGCAGGCCCUGCUGACCGCCCCCGCCCUGGGCCUGCCC |
| | GACCUGACCAAGCCUUUCGAGCUGUUCGUGGACGAGAA |
| | GCAGGGAUACGCCAAAGGCGUGCUGACCCAGAAGCUGG |
| | GCCCCUGGCGGAGGCCCGUGGCCUACCUGAGCAAAAAA |
| | CUGGACCCUGUGGCCGCCGGCUGGCCCCCAUGCCUGCG |
| | GAUGGUGGCCGCCAUCGCUGUGCUGACCAAGGACGCCG |
| | GCAAGCUGACCAUGGGCCAGCCCCUGGUGAUCCUGGCC |
| | CCUCACGCCGUGGAGGCUCUGGUGAAGCAGCCUCCAGA |
| | CAGGUGGCUGUCCAACGCCAGGAUGACCCACUACCAGG |
| | CCCUGCUGCUGGACACCGACCGGGUGCAGUUCGGCCCU |
| | GUGGUGGCCCUGAACCCCGCCACCCUGCUGCCUCUGCCA |
| | GAGGAGGGCCUGCAGCACAACUGCCUGGACAUCCUGGC |
| | CGAGGCCCACGGCGGCGGCUCCAAACGCACCGCCGACG |
| | GGAGCGAGUUCGAGCCCAAGAAGAAGAGGAAAGUCUAA |
| | (SEQ ID NO: 308) |

### Example 4. Prime Editing Efficiency Utilizing Chemically Modified LNA pegRNA

Locked nucleic acids (LNA) and analogs thereof have a methylene (—CH2-) bridge between 2′ and 4′ carbons of the ribose sugar. The methylene bridging locks the flexible parental furanose ring restricting the nucleic acid into a rigid structure with a 3′ carbon endo conformation. Advantages of introducing and utilizing LNA nucleotides into RNA architecture include improving duplex stability and enhanced binding affinity to complementary sequences. Accordingly, this Example tests whether LNA nucleic acids can be designed and utilized in pegRNA architectures (i.e., LNA pegRNA).

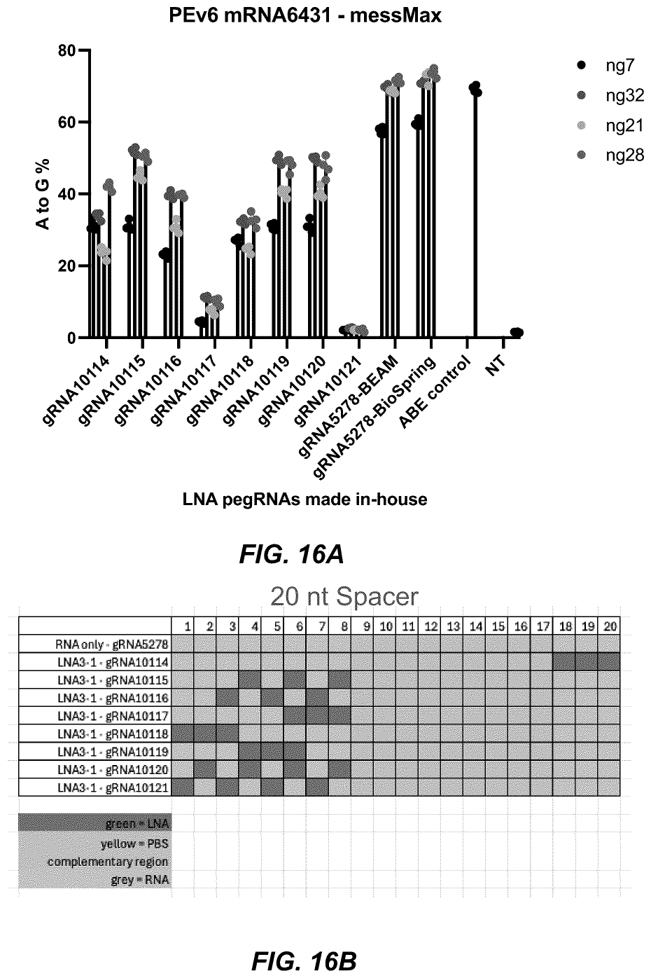

Chemically modified pegRNA 5278 as shown in Table 1 was used as the parental pegRNA design which further include LNA nucleosides. FIG. 16B shows a spatial map of the eight LNA pegRNA designs with respect to the position of the LNA nucleoside in the 20-nucleotide spacer region as well as in relation to the PBS complementary region. Table 7 below shows the pegRNA full sequence with “1” corresponding to a LNA nucleic acid.

As shown in FIG. 16A, a HepG2 E342K cell line was transfected with a prime editor ((prime editor V6 (PEv6 mRNA6431)), a nicking guide (either nicking guide (ng) #7, 32, 21, or 28), and either one of the eight LNA pegRNAs designed in FIG. 15B and shown in Table 7 or controls and the percent of A-to-G correction was measured. The LNA pegRNAs did not outperform the editing efficiency of chemically modified parental pegRNA 5278. Further, pegRNA 5278 controls synthesized at two distinct facilities (labeled “gRNA5278-BEAM” or “gRNA5278-BioSpring”) had the same editing efficiency of over 70%. In FIG. 16A, utilizing ng #21, 28, and 32 outperformed utilizing ng #7. Further, in data not shown, the indel rate for experimental groups utilizing ng #21, 28, and 32 (which nick on the on-target strand) were lower than the indel rate for experimental groups utilizing ng #7.

TABLE 7
LNA substituted pegRNA; “m” corresponds to a 2′-O-Methyl ribose
modification; “s” corresponds to a phosphorothioate modification;
“I” corresponds to a LNA modification

| Peg | | |
|---|---|---|
| RNA | | |
| ID | Full sequence | SEQ ID Nos. |
| gRNA | mUsmCsmCsCCUCCAGGCCGUGClAlUlAGUUUUAGAGC | 269 |
| 10114 | UAGAAAUAGCAAGUUAAAAUAAGGCUAGUCCGUUA | |
| | UCAACUUGAAAAAGUGGCACCGAGUCGGUGCUUUCU | |
| | CGUCGAUGGUCAGCACAGCCUUAUGCACGGCCUmUs | |
| | mUsmUsU | |
| gRNA | mUsmCsmCslCClUClCAGGCCGUGCAUAGUUUUAGAGC | 270 |
| 10115 | UAGAAAUAGCAAGUUAAAAUAAGGCUAGUCCGUUA | |
| | UCAACUUGAAAAAGUGGCACCGAGUCGGUGCUUUCU | |
| | CGUCGAUGGUCAGCACAGCCUUAUGCACGGCCUmUs | |
| | mUsmUsU | |
| gRNA | mUsmCslCsClCUlCCAGGCCGUGCAUAGUUUUAGAGCU | 271 |
| 10116 | AGAAAUAGCAAGUUAAAAUAAGGCUAGUCCGUUAU | |
| | CAACUUGAAAAAGUGGCACCGAGUCGGUGCUUUCUC | |
| | GUCGAUGGUCAGCACAGCCUUAUGCACGGCCUmUsm | |
| | UsmUsU | |
| gRNA | mUsmCsmCsCClUlClCAGGCCGUGCAUAGUUUUAGAGC | 272 |
| 10117 | UAGAAAUAGCAAGUUAAAAUAAGGCUAGUCCGUUA | |
| | UCAACUUGAAAAAGUGGCACCGAGUCGGUGCUUUCU | |
| | CGUCGAUGGUCAGCACAGCCUUAUGCACGGCCUmUs | |
| | mUsmUsU | |
| gRNA | lUslCslCsCCUCCAGGCCGUGCAUAGUUUUAGAGCUAG | 273 |
| 10118 | AAAUAGCAAGUUAAAAUAAGGCUAGUCCGUUAUCA | |
| | ACUUGAAAAAGUGGCACCGAGUCGGUGCUUUCUCGU | |
| | CGAUGGUCAGCACAGCCUUAUGCACGGCCUmUsmUs | |
| | mUsU | |
| gRNA | mUsmCsmCslClClUCCAGGCCGUGCAUAGUUUUAGAGC | 274 |
| 10119 | UAGAAAUAGCAAGUUAAAAUAAGGCUAGUCCGUUA | |
| | UCAACUUGAAAAAGUGGCACCGAGUCGGUGCUUUCU | |
| | CGUCGAUGGUCAGCACAGCCUUAUGCACGGCCUmUs | |
| | mUsmUsU | |
| gRNA | mUslCsmCslCClUClCAGGCCGUGCAUAGUUUUAGAGC | 275 |
| 10120 | UAGAAAUAGCAAGUUAAAAUAAGGCUAGUCCGUUA | |
| | UCAACUUGAAAAAGUGGCACCGAGUCGGUGCUUUCU | |
| | CGUCGAUGGUCAGCACAGCCUUAUGCACGGCCUmUs | |
| | mUsmUsU | |
| gRNA | lUsmCslCsClCUlCCAGGCCGUGCAUAGUUUUAGAGCU | 276 |
| 10121 | AGAAAUAGCAAGUUAAAAUAAGGCUAGUCCGUUAU | |
| | CAACUUGAAAAAGUGGCACCGAGUCGGUGCUUUCUC | |
| | GUCGAUGGUCAGCACAGCCUUAUGCACGGCCUmUsm | |
| | UsmUsU | |

### Example 5. Nuclear Localization Signal

The ability of a prime editor system components to localize efficiently in the nucleus is critical to the efficiency of the prime editing system. Therefore, this Example tests the design and addition of nuclear localization signal (NLS) sequences to pegRNA.

Chemically modified pegRNA 5278 described in previous examples was used as the base architecture to which the NLS sequence was added. Table 8 below sets forth the sequence and corresponding pegRNA identifier (“gRNA9120”).

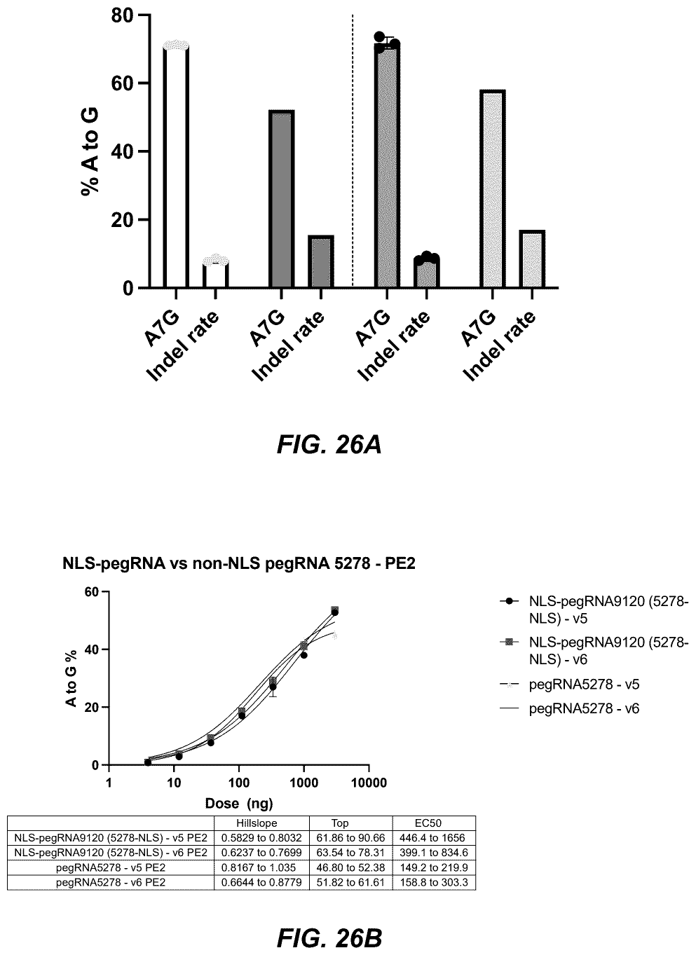

As shown in FIG. 26A, the percentage of editing efficiency as reported as an A-to-G percent correction (A7G) along with the indel rate in HepG2 E342K cells upon transfection with a prime editor variant (“PE2 V5”; “PE3 V5” “PE2 V6”; “PE3 V6”) and the designed pegRNA containing a nuclear localization signal (NLS) “gRNA9120” described herein reached as high as 70%. The highest editing efficiency was seen when gRNA9120 was paired with the PE2 V5 or the PE2 V6 prime editors. To further analyze the editing efficiency, gRNA9120 was compared with the parental pegRNA 5278 that did not have the NLS signal. As shown in FIG. 26B, the dose response curve for the A-to-G correction versus the dosing (ng) upon addition of escalating concentrations of prime editor and pegRNA demonstrates that pegRNA 5278 is more potent than gRNA9120. The corresponding EC50 values (FIG. 26B table) for experimental groups utilizing pegRNA 5278 are about 150-300 ng whereas experimental groups utilizing gRNA9120 are about 400-1600 ng.

TABLE 8
NLS pegRNA sequence

| PegRNA | |
|---|---|
| ID | Full sequence |
| gRNA9120 | MUsmCsmCsCCUCCAGGCCGUGCAUAGUUUUAGAGCUAGAAAUA |
| sequence | GCAAGUUAAAAUAAGGCUAGUCCGUUAUCAACUUGAAAAAGUG |
| (also | GCACCGAGUCGGUGCUUUCUCGUCGAUGGUCAGCACAGCCUUA |
| referred to | UGCACGGCCUmUsmUsmUsU (SEQ ID NO: 87)-NHC6-CrossL- |
| as NLS- | CysLysArgThrAlaAspGlySerGluPheGluSerProLysLysLysArgLysVal |
| pegRNA5278) | (SEQ ID NO: 309) |

### Example 6. LNP Encapsulated Prime Editing System Delivery Results in Editing of Cells In Vitro and In Vivo

Lipid nanoparticles (LNPs) can be used to encapsulate the prime editing components to enhance RNA stability, delivery efficiency, and editing potency. This Example demonstrates the ability to design, test, and optimize LNP delivery systems which encapsulate the prime editing components for in vitro and in vivo editing.

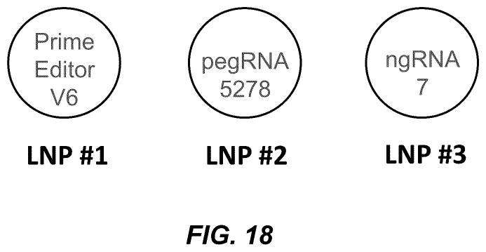

As shown in FIG. 18, one envisaged LNP delivery system described herein is for each separate prime editing component of the prime editing system (prime editor mRNA, pegRNA, and ngRNA) to be split and encapsulated as cargo into three LNPs (depicted as LNP #1, 2, and 3; note however, the chemical composition of the LNP can be the same with only the cargo changing (i.e., the prime editing components are different).

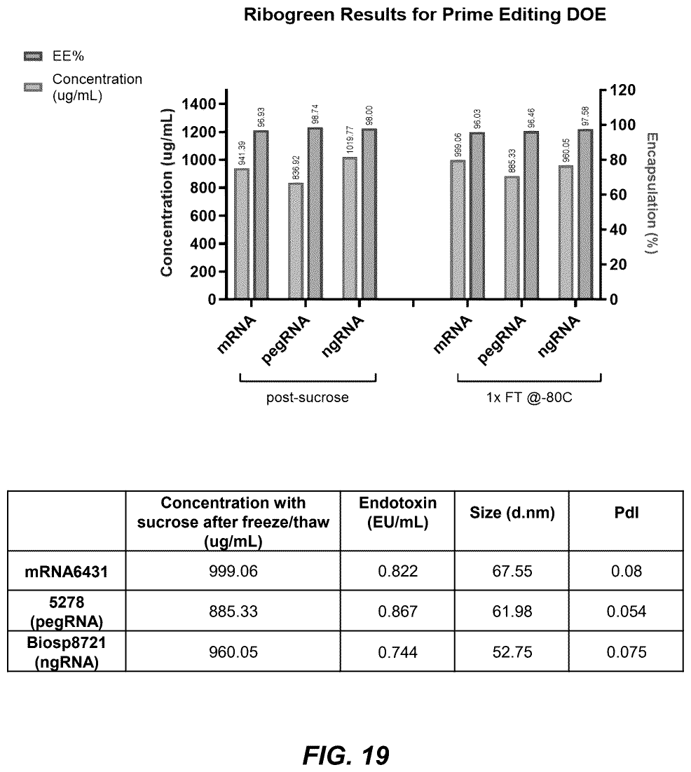

The stability of the LNP formulation after suspension in a sucrose-containing buffer or after one freeze thaw (lx FT) cycle after storage at −80° C. is shown in FIG. 19. The left y-axis displays the RNA concentration (ug/mL) and the right y-axis displays the percent of LNP encapsulation (FIG. 19; top of figure). The corresponding table reporting the concentration (ug/mL), endotoxin (EU/mL), LNP diameter (d.nm), and polydispersity index (PdI) values for each experimental group is shown at the bottom of FIG. 19. Accordingly, prime editor components were successfully encapsulated into LNPs and were stable when subjected to freeze/thaw conditions.

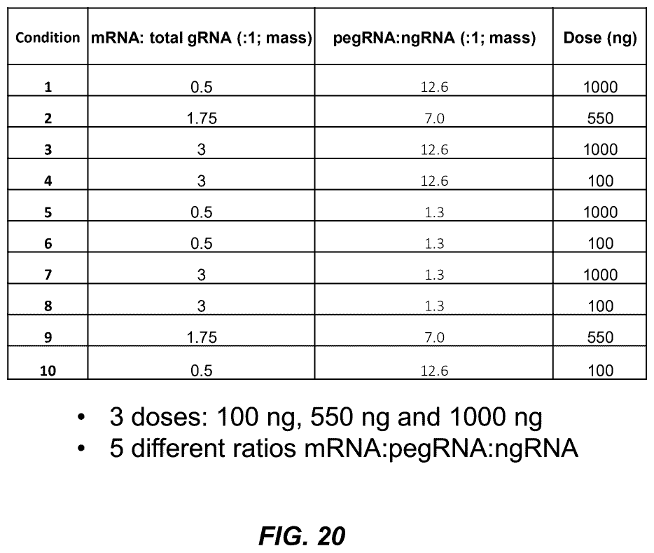

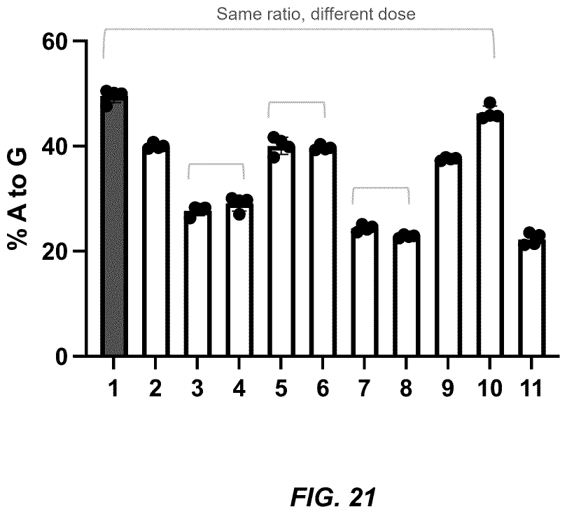

Subsequently, to probe the most effective LNP delivery conditions, several doses and LNP mass ratios were designed as conditions as shown in FIG. 20. In total, ten distinct conditions of LNP mass ratios (PE mRNA: pegRNA; ngRNA; middle columns) and dosing (100, 550, and 1000 ng; last right column) for the LNP encapsulated prime editing components were tested in an initial in vitro screen using a HepG2 cell line containing the A1AT gene with the pathogenic E342K G>A SNP. As shown in FIG. 21, the prime editing efficiency of an A-to-G correction with the ten distinct experimental groups (condition #1-10) of LNP encapsulated prime editing components (PEv6 6431+pegRNA 5278+ngRNA #7) show that the dosing parameter had little to no impact on the editing efficiency. For example, the paired conditions with bars connecting the conditions shown in FIG. 21 (experimental condition group #: 1 and 10; 3 and 4; and 7 and 8) indicate that those experimental groups had the same mass ratio and different dose considerations but resulted in a comparable percentage of A-to-G correction efficiency. Therefore, it was confirmed that the LNP RNA payload mass ratio parameter not the dosing parameter had the biggest impact on editing efficiency.

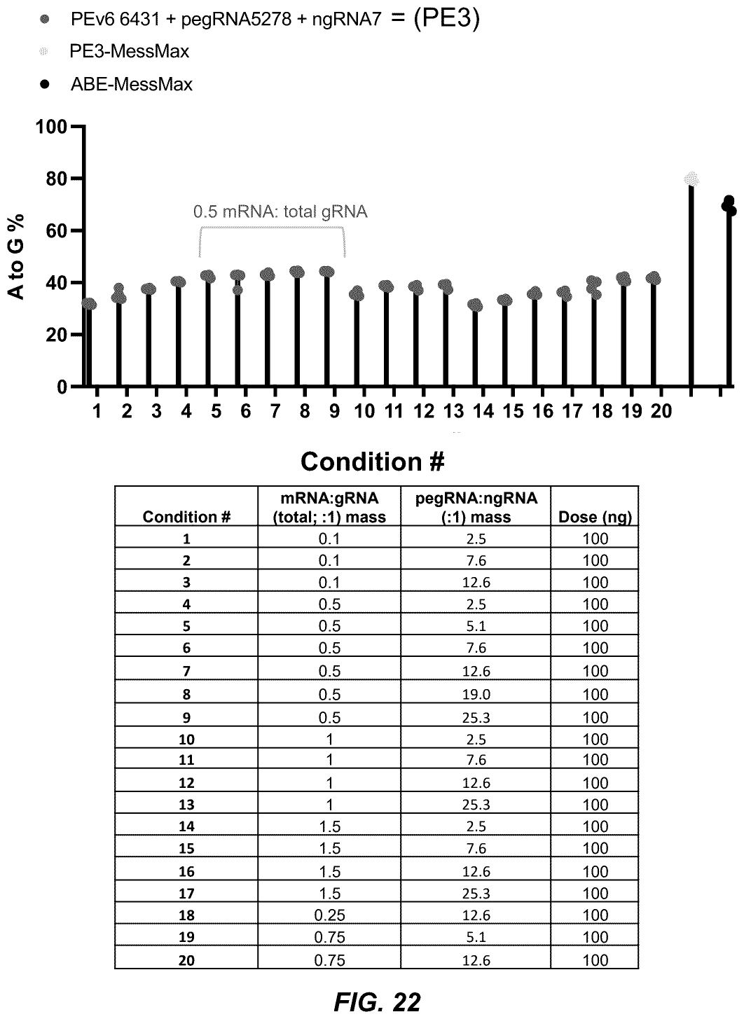

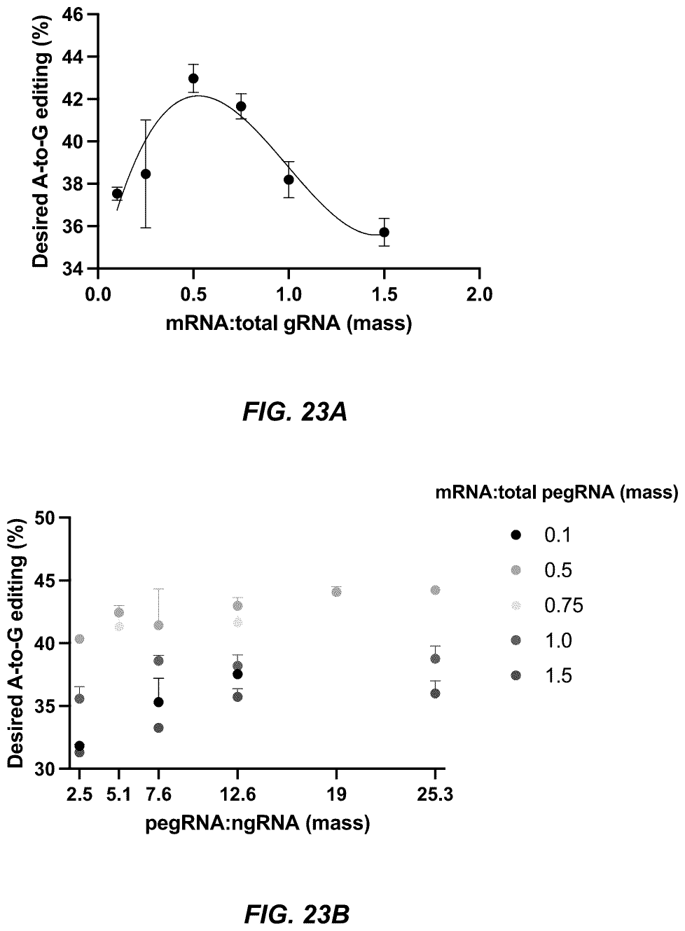

Next, a second round of in vitro screening utilizing the same experimental set-up as FIG. 21 was performed. Based on the results of the first screen, the second screen focused on varying the RNA mass ratio parameter while maintaining the dose amount as shown in conditions labeled 1-20 on the bottom table of FIG. 22. The FIG. 22 graph shows that the editing efficiency of conditions #5-9 were the highest of all experimental conditions tested. Conditions #5-9 also all have a relative ratio of 0.5 PE mRNA to 1 total gRNA. To further analyze what the best mass ratios were from the experimental conditions tested in FIG. 22, the data was replotted with the x-axis displaying escalating mass ratio conditions of mRNA: total gRNA as shown in FIG. 23A and pegRNA: ngRNA as shown in FIG. 23B. From this study, the best mass ratio selected to proceed to down stream in vivo testing was: 0.5 PE mRNA: 0.95 pegRNA: 0.05 ngRNA.

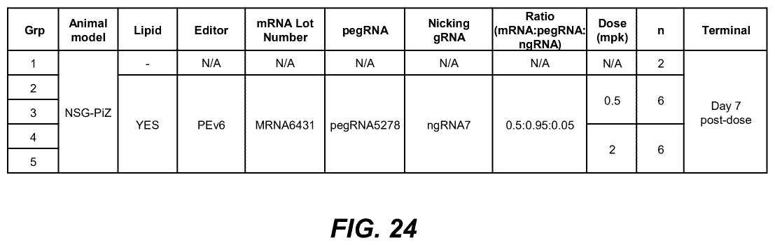

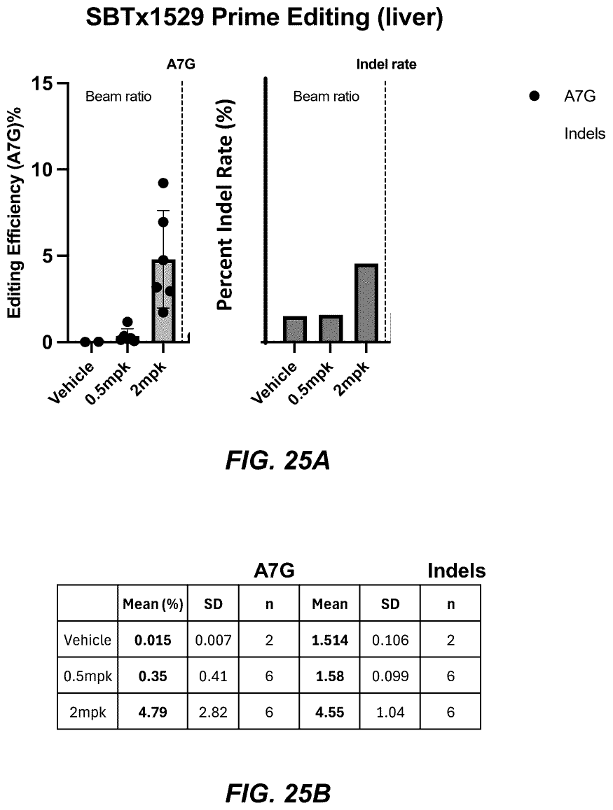

The table displayed in FIG. 24 shows the experimental set-up for the in vivo administration in of LNP encapsulated prime editing components (mRNA PEv6; pegRNA5278; ngRNA #7) at mass ratios of 0.5 PE mRNA: 0.95 pegRNA5278: 0.05 ngRNA7 at a 0.5 or 2 milligrams per kilogram (mpk) dose in NSG-PiZ mice (mice which express mutant SERPINA1 (E342K) mutation). As shown in FIG. 25, there was detectable E342K correction by prime editing by all experimental groups observed in livers of NSG-PiZ mice dosed with LNP formulations. The highest correction efficiency was about 5% and observed in mice administered the 2 mpk dose for the 0.5 PE mRNA: 0.95 pegRNA: 0.05 ngRNA ratio.

### Example 7. In-Laid, Circularity Permutated, or Variant Linker Prime Editor Designs Editing Efficiency in in Human Primary Fibroblast Cells

This example screens prime editing systems utilizing prime editor variants along with different pegRNA designs described in Table 1.

The prime editing components utilized in this example are the mRNA prime editor variants shown in Table 9 and the pegRNA designs shown in Table 10. The naming of some of the prime editor designs with the linker sequences are shown in Table 11 and 12, respectively.

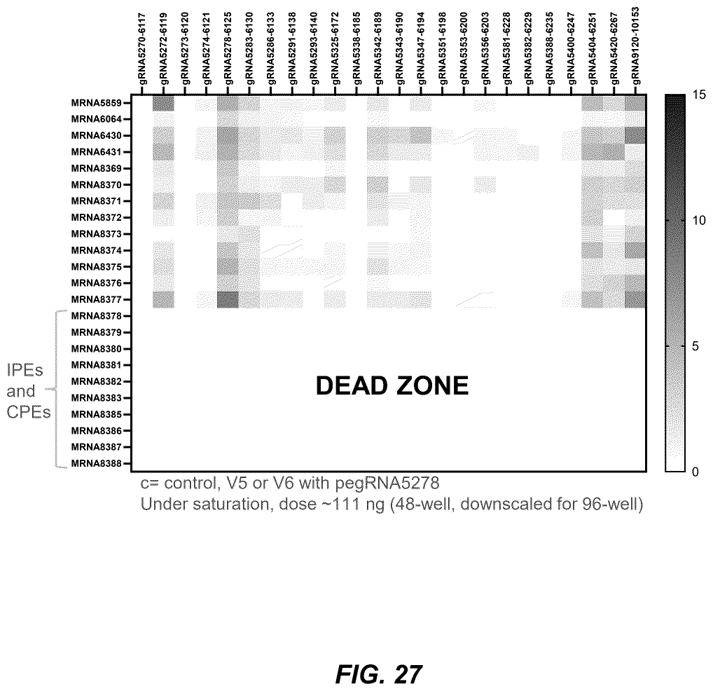

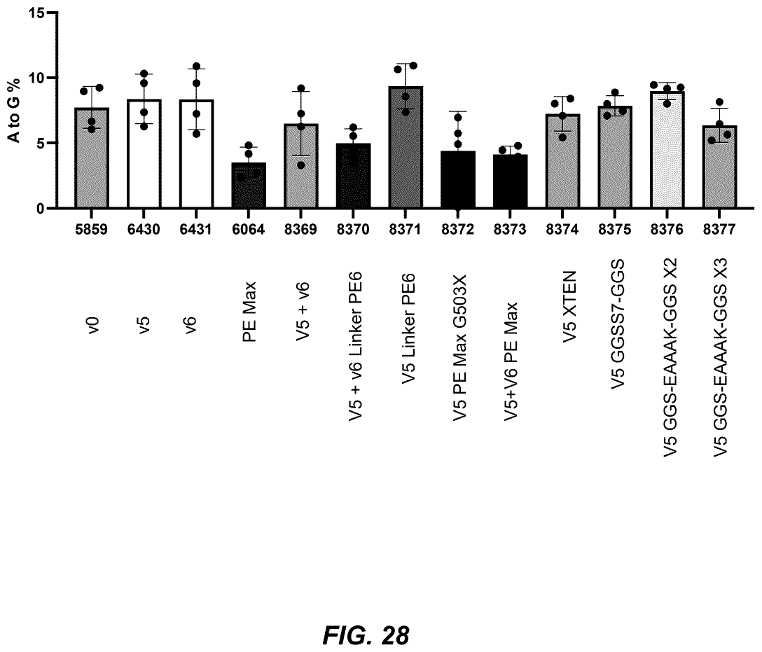

A heat map displaying a scale of 0-15% editing efficiency of a A-to-G correction upon transfecting a HepG2 E342K cell line harboring the G>A SNP with prime editor and pegRNA variants is shown in FIG. 27. The heat map displays a panel of prime editor variants matrixed with a panel of pegRNA variants where one prime editor (labeled on the y-axis; see also Table 9) was added along with one pegRNA (labeled across the x-axis; see also Table 10) in one well at a dose of about 111 ng in a 96-well format. The editing efficiency of prime editor variants with an in-laid or circularly permutated design matrixed with a panel of pegRNA variants was not detectable (see FIG. 27 “dead zone” label). Accordingly, the subsequent testing focused on testing the prime editing efficiency of prime editors with various linkers as shown in FIG. 28. As shown in FIG. 28, prime editor variants with different linkers modulated the amount of editing efficiency by at most 5%.

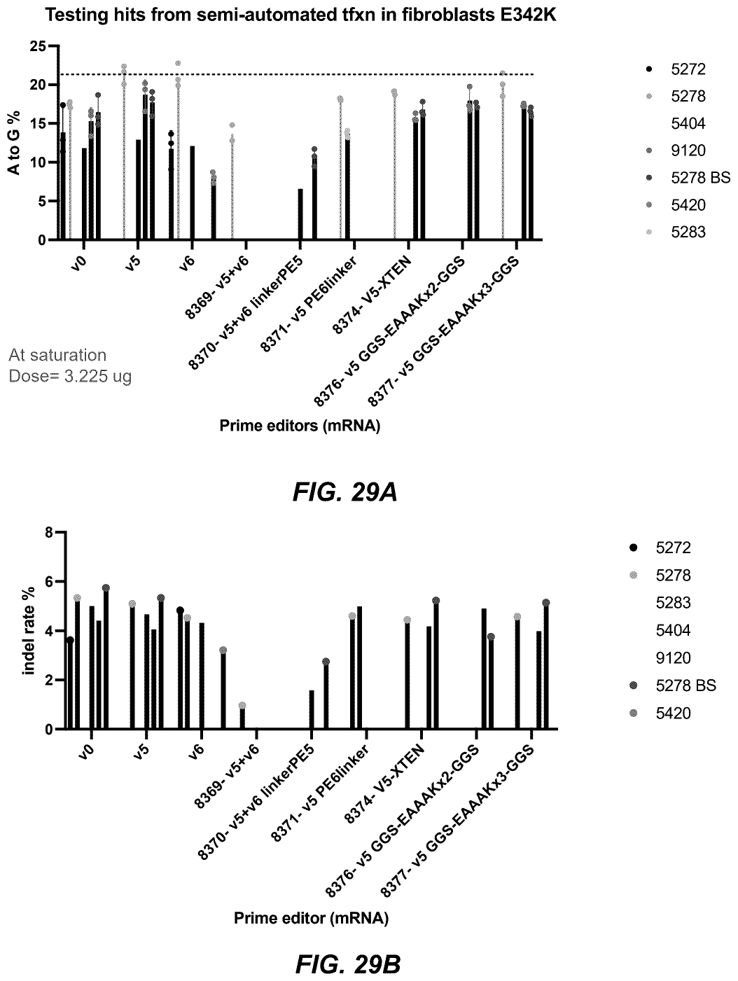

As shown in FIG. 29A and FIG. 29B, the prime editor V5 and prime editor V6 along with pegRNA 5278 remained the most optimal reagents for AMAT E342K correction achieving up to 22% editing efficiency if nicking guide #7 was also included.

TABLE 9
mRNA Prime editor variant designs

| Prime Editor | mRNA# |
|---|---|
| v0 | pHRB-314 |
| | (5859) |
| v5 | mRNA6430 |
| v6 | mRNA6431 |
| 32 PEMax | mRNA6064 |
| 33 v5 + v6 | mRNA8369 |
| 34 v5 + v6 linkerPE6 | mRNA8370 |
| 35-v5 PE6linker | mRNA8371 |
| 36 PEMax G503X v5 | mRNA8372 |
| 37 PEMax V5 + v6 | mRNA8373 |
| 39 v5 XTEN | mRNA8374 |
| 41 v5 GGSS7-GGS (SEQ ID NO: 303) | mRNA8375 |
| 43 v5 GGS-EAAAKx2-GGS (SEQ ID NO: 305) | mRNA8376 |
| 44 v5 GGS-EAAAKx3-GGS (SEQ ID NO: 306) | mRNA8377 |
| 31-IPE01 | mRNA8378 |
| 31-IPE02 | mRNA8379 |
| 31-IPE03 | mRNA8380 |
| 31-IPE04 | mRNA8381 |
| 31-CP01 | mRNA8382 |
| 31-CP02 | mRNA8383 |
| 31-CP03 | MRNA8385 |
| 31-CP04 | MRNA8386 |
| 31-CP05 | MRNA8387 |
| 31-CP06 | MRNA8388 |

IPE = in-laid prime editors; CP = circularly permutated prime editors; G503X = MLV reverse transcriptase truncation (version without the RNAseH domain); PE6 linker; V5 = n Cas9 single point mutation K961A; V6 = nCas9 single point mutation K968A

TABLE 10
pegRNAs with variable PBS and RTT lengths tested

| pegRNA | PBS | RTT | Protospacer + | terminal | total |
|---|---|---|---|---|---|
| number | length | Length | scaffold | Us | length |
| gRNA5291 | 7 | 29 | 96 | 4 | 136 |
| gRNA5283 | 8 | 29 | 96 | 4 | 137 |
| gRNA5420 | 9 | 27 | 96 | 4 | 136 |
| gRNA5404 | 9 | 28 | 96 | 4 | 137 |
| gRNA5278* | 9 | 29 | 96 | 4 | 138 |
| gRNA5286 | 9 | 30 | 96 | 4 | 139 |
| gRNA5293 | 9 | 31 | 96 | 4 | 140 |
| gRNA5400 | 9 | 32 | 96 | 4 | 141 |
| gRNA5325 | 9 | 33 | 96 | 4 | 142 |
| gRNA5342 | 9 | 34 | 96 | 4 | 143 |
| gRNA5347 | 9 | 35 | 96 | 4 | 144 |
| gRNA5382 | 9 | 36 | 96 | 4 | 145 |
| gRNA5356 | 9 | 37 | 96 | 4 | 146 |
| gRNA5343 | 9 | 38 | 96 | 4 | 147 |
| gRNA5381 | 9 | 39 | 96 | 4 | 148 |
| gRNA5353 | 9 | 40 | 96 | 4 | 149 |
| gRNA5338 | 9 | 41 | 96 | 4 | 150 |
| gRNA5388 | 9 | 42 | 96 | 4 | 151 |
| gRNA5351 | 9 | 43 | 96 | 4 | 152 |
| gRNA5272 | 10 | 29 | 96 | 4 | 139 |
| gRNA5274 | 12 | 29 | 96 | 4 | 141 |
| gRNA5273 | 13 | 29 | 96 | 4 | 142 |
| gRNA5270 | 14 | 29 | 96 | 4 | 143 |

TABLE 11
Inlaid and Circularly Permutated Prime Editor Designs

Inlaid Prime Editors (IPEs)
pMM-PE31_v5_pVLM123_IPE01; 28aa linker
pMM-PE31_v5_pVLM123_IPE02; 18aa linker
pMM-PE31_v5_pVLM123_IPE03; 13aa linker
pMM-PE31_v5_pVLM123_IPE04; 8aa linker
Circularly permuted Prime Editors (CPEs)
pMM-PE31_v5_pVLM123_CP1; 8aa linker (C)
pMM-PE31_v5_pVLM123_CP2; 18aa linker (C)
pMM-PE31_v5_pVLM123_CP3; 28aa linker (C)
pMM-PE31_v5_pVLM123_CP4; 8aa linker (N)
pMM-PE31_v5_pVLM123_CP5; 18aa linker (N)
pMM-PE31_v5_pVLM123_CP6; 28aa linker (N)

TABLE 12
Linker sequences utilized in prime editor designs

| Linker Name | Amino Acid Sequence |
|---|---|
| 28 aa | SGGSEAAAKEAAAKEAAAKEAAAKSGGS |
| | (SEQ ID NO: 277) |
| 18 aa | SGGSEAAAKEAAAKSGGS (SEQ ID NO: 278) |
| 13 aa | SGGSEAAAKSGGS (SEQ ID NO: 279) |
| 8 aa | SGGS (SEQ ID NO: 280) |

1. A prime editing guide RNA (PEgRNA) comprising:
a spacer that is complementary to a search target sequence on a first strand of a human SERPINA1 gene,
a primer binding site sequence (PBS) at least partially complementary to the spacer,
an editing template that comprises a region of complementarity to an editing target sequence on a second strand of the SERPINA1 gene, and
a gRNA core that associates with a prime editor comprising a DNA binding domain and a DNA polymerase domain,
wherein the PBS comprises or consists of the sequence GCACGGC (SEQ ID NO: 2) and the editing template comprises or consists of the sequence
(SEQ ID NO: 25)
UUUCUCGUCGAUGGUCAGCACAGCCUUAU

2. The PEgRNA of claim 1, wherein
the spacer sequence comprises or consists of the sequence
(SEQ ID NO: 1)
UCCCCUCCAGGCCGUGCAUA;
the gRNA core is between the spacer and the editing template;
the editing template comprises an intended nucleotide edit compared to the SERPINA1 gene;
the PEgRNA guides the prime editor to incorporate the intended nucleotide edit into the SERPINA1 gene when contacted with the SERPINA1 gene;
the prime editor synthesizes a single stranded DNA encoded by the editing template, wherein the single stranded DNA replaces the editing target sequence and results in incorporation of the intended nucleotide edit into a region corresponding to the editing target in the SERPINA1 gene;
the search target sequence is complementary to a protospacer sequence in the SERPINA1 gene, and wherein the protospacer sequence is adjacent to a search target adjacent motif (PAM) in the SERPINA1 gene;
the PAM comprises NGG;
the PEgRNA results in incorporation of the intended nucleotide edit in the PAM when contacted with the SERPINA1 gene;
wherein the PBS is about 2 to about 20 base pairs in length;
the PBS is about 8 to about 16 base pairs in length;
the PBS comprises or consists of the sequence GCACGGCC (SEQ ID NO: 3), GCACGGCCU (SEQ ID NO: 4), GCACGGCCUG (SEQ ID NO: 5), GCACGGCCUGG (SEQ ID NO: 6), GCACGGCCUGGA (SEQ ID NO: 7), GCACGGCCUGGAG (SEQ ID NO: 8), GCACGGCCUGGAGG (SEQ ID NO: 9), or GCACGGCCUGGAGGG (SEQ ID NO: 10);
wherein the editing template is about 4 to 30 base pairs in length;
the editing template is about 10 to 30 base pairs in length; and/or
the editing template comprises or consists of the sequence
(SEQ ID NO: 11)
GCAGCUUCAGUCCCUUUCUCGUCGAUGGUCAGCACAGCCUUAU,
(SEQ ID NO: 12)
CAGCUUCAGUCCCUUUCUCGUCGAUGGUCAGCACAGCCUUAU,
(SEQ ID NO: 13)
AGCUUCAGUCCCUUUCUCGUCGAUGGUCAGCACAGCCUUAU,
(SEQ ID NO: 14)
GCUUCAGUCCCUUUCUCGUCGAUGGUCAGCACAGCCUUAU,
(SEQ ID NO: 15)
CUUCAGUCCCUUUCUCGUCGAUGGUCAGCACAGCCUUAU,
(SEQ ID NO: 16)
UUCAGUCCCUUUCUCGUCGAUGGUCAGCACAGCCUUAU,
(SEQ ID NO: 17)
UCAGUCCCUUUCUCGUCGAUGGUCAGCACAGCCUUAU,
(SEQ ID NO: 18)
CAGUCCCUUUCUCGUCGAUGGUCAGCACAGCCUUAU,
(SEQ ID NO: 19)
AGUCCCUUUCUCGUCGAUGGUCAGCACAGCCUUAU,
(SEQ ID NO: 20)
GUCCCUUUCUCGUCGAUGGUCAGCACAGCCUUAU,
(SEQ ID NO: 21)
UCCCUUUCUCGUCGAUGGUCAGCACAGCCUUAU,
(SEQ ID NO: 22)
CCCUUUCUCGUCGAUGGUCAGCACAGCCUUAU,
(SEQ ID NO: 23)
CCUUUCUCGUCGAUGGUCAGCACAGCCUUAU,
(SEQ ID NO: 24)
CUUUCUCGUCGAUGGUCAGCACAGCCUUAU,
or
(SEQ ID NO: 25)
UUUCUCGUCGAUGGUCAGCACAGCCUUAU;
the PEgRNA results in incorporation of intended nucleotide edit about 0 to 27 base pairs downstream of the 5′ end of the PAM when contacted with the SERPINA1 gene;
the intended nucleotide edit comprises a single nucleotide substitution compared to the region corresponding to the editing target in the SERPINA1 gene;
the intended nucleotide edit comprise an insertion compared to the region corresponding to the editing target in the SERPINA1 gene;
the intended nucleotide edit comprises a deletion compared to the region corresponding to the editing target in the SERPINA1 gene;
the editing target sequence comprises a mutation associated with alpha-1 antitrypsin deficiency (A1AD);
the editing template comprises a wild type SERPINA1 gene sequence;
the PEgRNA results in correction of the mutation when contacted with the SERPINA1 gene;
the PEgRNA comprises or consists of any one of the sequences recited in Table 1;
the PEgRNA comprises at least one chemical modification;
the at least one chemical modification is selected from the group consisting of a 2′-O-methyl (2′-OMe) modification, a 2′-deoxy (2′-H) modification, a 2′-fluoro (2′-F) modification, a 2′-methoxyethyl (2′-MOE) modification, a 2′-amino (2′—NH2) modification, a 2′-arabinosyl (2′-arabino) modification, a 2′-F-arabinosyl (2′-F-arabino) modification, and a locked nucleic acid (LNA) modification;
the at least one chemical modification comprises an internucleotide linkage modification;
the at least one internucleotide linkage modification comprises a phosphonoacetate (PACE) modification; and/or
the PEgRNA comprises or consists of any one of the sequences recited in Table 1.

3.-28. (canceled)

29. A PEgRNA system comprising the PEgRNA according to claim 1 and further comprising a nick guide RNA (ngRNA), wherein the ngRNA comprises an ng spacer that is complementary to a second search target sequence in the SERPINA1 gene.

30. The PEgRNA system of claim 29, wherein the second search target sequence is on the second strand of the SERPINA1 gene, optionally wherein
the ngRNA comprises a spacer sequence selected from the group consisting of:
(SEQ ID NO: 26)
GAAGCAGAGACACGUUGUA,
(SEQ ID NO: 27)
GUCAGCACAGCCUUAUGCA,
(SEQ ID NO: 28)
GAAAGGGACUGAAGCUGCU,
(SEQ ID NO: 29)
CCUCGGGGGGGAUAGACAU,
(SEQ ID NO: 30)
UGAUCCCAGGCCUCGAGCA,
(SEQ ID NO: 31)
ACGUUGUAAGGCUGAUCCC,
(SEQ ID NO: 32)
AAAGGGACUGAAGCUGCUG,
(SEQ ID NO: 33)
GGUAUGGCCUCUAAAAACA,
(SEQ ID NO: 34)
CCCAUGUCUAUCCCCCCCG,
(SEQ ID NO: 35)
GCCUCGAGCAAGGCUCACG,
(SEQ ID NO: 36)
GGUUUGUUGAACUUGACCU,
(SEQ ID NO: 37)
CCUUAUGCACGGCCUGGAG,
(SEQ ID NO: 38)
AGAAAGGGACUGAAGCUGC,
(SEQ ID NO: 39)
CACAGCCUUAUGCACGGCC,
(SEQ ID NO: 40)
GGGGGGAUAGACAUGGGUA,
(SEQ ID NO: 41)
GUUUGUUGAACUUGACCUC,
(SEQ ID NO: 42)
UGCUGACCAUCGACAAGAA,
(SEQ ID NO: 43)
UUGUUGAACUUGACCUCGG,
(SEQ ID NO: 44)
GCCUUAUGCACGGCCUGGA,
(SEQ ID NO: 45)
UUUGUUGAACUUGACCUCG,
(SEQ ID NO: 46)
GUUGAACUUGACCUCGGGG,
(SEQ ID NO: 47)
CUCUGCUUCUCUCCCCUCC,
(SEQ ID NO: 48)
UGAGCCUUGCUCGAGGCCU,
(SEQ ID NO: 49)
AGCCUUAUGCACGGCCUGG,
(SEQ ID NO: 50)
ACCUCGGGGGGGAUAGACA,
(SEQ ID NO: 51)
UCAGUCCCUUUCUUGUCGA,
(SEQ ID NO: 52)
UGUUGAACUUGACCUCGGG,
(SEQ ID NO: 53)
CCCCUCCAGGCCGUGCAUA,
(SEQ ID NO: 54)
GUGAGCCUUGCUCGAGGCC,
(SEQ ID NO: 55)
GCUGACCAUCGACAAGAAA,
(SEQ ID NO: 56)
GCUGGGGCCAUGUUUUUAG,
(SEQ ID NO: 57)
UGCUGACCAUCGACGAGAA,
(SEQ ID NO: 58)
UCAGUCCCUUUCUCGUCGA,
and/or
(SEQ ID NO: 59)
GCUGACCAUCGACGAGAAA.

31. (canceled)

32. A PEgRNA system comprising the PEgRNA according to claim 1, and a ngRNA wherein the ngRNA comprises an ng spacer that is complementary to a second search target sequence in the SERPINA1 gene.

33. A prime editing complex comprising: (i) the PEgRNA of claim 1 or a PEgRNA system comprising the PEgRNA according to claim 1, and a the ngRNA wherein the ngRNA comprises an ng spacer that is complementary to a second search target sequence in the SERPINA1 gene; and (ii) a prime editor comprising a DNA binding domain and a DNA polymerase domain.

34. The prime editing complex of claim 33, wherein
the DNA binding domain is a CRISPR associated (Cas) protein domain;
the Cas protein domain has nickase activity;
the Cas protein domain is a Cas9;
the Cas9 comprises a mutation in an HNH domain;
the Cas9 comprises a H840A mutation in the HNH domain;
the Cas9 comprises the sequence of SEQ ID NO: 60;
the Cas9 comprises a mutation at one or more amino acids positions of R765, K848, K855, K959, K961, K968, K974, or R976 relative to SEQ ID NO: 60;
the Cas9 comprises one or more mutations of R765A, K848A, K855A, K959A, K961A, K968A, K974A, or R976A relative to SEQ ID NO: 60;
the Cas9 comprises or consists of any one of the sequences of SEQ ID NO: 61 to 72;
the DNA polymerase domain is a reverse transcriptase;
the reverse transcriptase is a retrovirus reverse transcriptase; and/or
the reverse transcriptase is a Moloney murine leukemia virus (M-MLV) reverse transcriptase.

35.-45. (canceled)

46. The prime editing complex of claim 34, wherein the reverse transcriptase comprises the sequence of SEQ ID NO: 75 (TLNIEDEYRLHETSKEPDVSLGSTWLSDFPQAWAETGGMGLAVRQAPLIIPLKATSTPVS IKQYPMSQEARLGIKPHIQRLLDQGILVPCQSPWNTPLLPVKKPGTNDYRPVQDLREVN KRVEDIHPTVPNPYNLLSGLPPSHQWYTVLDLKDAFFCLRLHPTSQPLFAFEWRDPEMGI SGQLTWTRLPQGFKNSPTLFNEALHRDLADFRIQHPDLILLQYVDDLLLAATSELDCQQ GTRALLQTLGNLGYRASAKKAQICQKQVKYLGYLLKEGQRWLTEARKETVMGQPTPK TPRQLREFLGKAGFCRLFIPGFAEMAAPLYPLTKPGTLFNWGPDQQKAYQEIKQALLTA PALGLPDLTKPFELFVDEKQGYAKGVLTQKLGPWRRPVAYLSKKLDPVAAGWPPCLR MVAAIAVLTKDAGKLTMGQPLVILAPHAVEALVKQPPDRWLSNARMTHYQALLLDTD RVQFGPVVALNPATLLPLPEEGLQHNCLDILAEAHGGGSKRTADGSEFE), optionally wherein
the DNA polymerase and the DNA binding domain are fused or linked to form a fusion protein,
the DNA polymerase and the programmable DNA binding domain are linked by a linker comprising an amino acid sequence of SGGSEAAAKEAAAKEAAAKEAAAKSGGS (SEQ ID NO: 277),
the fusion protein comprises the sequence of SEQ ID NO: 77
(MKRTADGSEFESPKKKRKVDKKYSIGLDIGTNSVGWAVITDEYKVPSKKFKVLGNTDR
HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYY
LQNGRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQ
SITGLYETRIDLSQLGGDSGGSEAAAKEAAAKEAAAKEAAAKSGGSTLNIEDEYRLHETS
KEPDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPTVPNPY
NLLSGLPPSHQWYTVLDLKDAFFCLRLHPTSQPLFAFEWRDPEMGISGQLTWTRLPQGF
KNSPTLFNEALHRDLADFRIQHPDLILLQYVDDLLLAATSELDCQQGTRALLQTLGNLG
YRASAKKAQICQKQVKYLGYLLKEGQRWLTEARKETVMGQPTPKTPRQLREFLGKAGF
CRLFIPGFAEMAAPLYPLTKPGTLFNWGPDQQKAYQEIKQALLTAPALGLPDLTKPFELF
VDEKQGYAKGVLTQKLGPWRRPVAYLSKKLDPVAAGWPPCLRMVAAIAVLTKDAGK
LTMGQPLVILAPHAVEALVKQPPDRWLSNARMTHYQALLLDTDRVQFGPVVALNPATL
LPLPEEGLQHNCLDILAEAHGGGSKRTADGSEFE);
the fusion protein comprises a nuclear localization signal (NLS);
the NLS comprises an amino acid sequence of PKKKRKV (SEQ ID NO: 282),
the fusion protein is encoded by the polynucleotide sequence:
(SEQ ID NO: 78)
ATGAAACGGACAGCCGACGGAAGCGAGTTCGAGTCACCAAAGAAGAAGCGGAAAG
TCGACAAGAAGTACAGCATCGGCCTGGACATCGGCACCAACTCTGTGGGCTGGGCC
GTGATCACCGACGAGTACAAGGTGCCCAGCAAGAAATTCAAGGTGCTGGGCAACAC
CGACCGGCACAGCATCAAGAAGAACCTGATCGGAGCCCTGCTGTTCGACAGCGGCG
AAACAGCCGAGGCCACCCGGCTGAAGAGAACCGCCAGAAGAAGATACACCAGACG
GAAGAACCGGATCTGCTATCTGCAAGAGATCTTCAGCAACGAGATGGCCAAGGTGG
ACGACAGCTTCTTCCACAGACTGGAAGAGTCCTTCCTGGTGGAAGAGGATAAGAAG
CACGAGCGGCACCCCATCTTCGGCAACATCGTGGACGAGGTGGCCTACCACGAGAA
GTACCCCACCATCTACCACCTGAGAAAGAAACTGGTGGACAGCACCGACAAGGCCG
ACCTGCGGCTGATCTATCTGGCCCTGGCCCACATGATCAAGTTCCGGGGCCACTTCC
TGATCGAGGGCGACCTGAACCCCGACAACAGCGACGTGGACAAGCTGTTCATCCAG
CTGGTGCAGACCTACAACCAGCTGTTCGAGGAAAACCCCATCAACGCCAGCGGCGT
GGACGCCAAGGCCATCCTGTCTGCCAGACTGAGCAAGAGCAGACGGCTGGAAAATC
TGATCGCCCAGCTGCCCGGCGAGAAGAAGAATGGCCTGTTCGGAAACCTGATTGCC
CTGAGCCTGGGCCTGACCCCCAACTTCAAGAGCAACTTCGACCTGGCCGAGGATGC
CAAACTGCAGCTGAGCAAGGACACCTACGACGACGACCTGGACAACCTGCTGGCCC
AGATCGGCGACCAGTACGCCGACCTGTTTCTGGCCGCCAAGAACCTGTCCGACGCC
ATCCTGCTGAGCGACATCCTGAGAGTGAACACCGAGATCACCAAGGCCCCCCTGAG
CGCCTCTATGATCAAGAGATACGACGAGCACCACCAGGACCTGACCCTGCTGAAAG
CTCTCGTGCGGCAGCAGCTGCCTGAGAAGTACAAAGAGATTTTCTTCGACCAGAGCA
AGAACGGCTACGCCGGCTACATTGACGGCGGAGCCAGCCAGGAAGAGTTCTACAAG
TTCATCAAGCCCATCCTGGAAAAGATGGACGGCACCGAGGAACTGCTCGTGAAGCT
GAACAGAGAGGACCTGCTGCGGAAGCAGCGGACCTTCGACAACGGCAGCATCCCCC
ACCAGATCCACCTGGGAGAGCTGCACGCCATTCTGCGGCGGCAGGAAGATTTTTAC
CCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAA
AGAGCGAGGAAACCATCACCCCCTGGAACTTCGAGGAAGTGGTGGACAAGGGCGCT
TCCGCCCAGAGCTTCATCGAGCGGATGACCAACTTCGATAAGAACCTGCCCAACGA
GAAGGTGCTGCCCAAGCACAGCCTGCTGTACGAGTACTTCACCGTGTATAACGAGCT
GACCAAAGTGAAATACGTGACCGAGGGAATGAGAAAGCCCGCCTTCCTGAGCGGCG
AGCAGAAAAAGGCCATCGTGGACCTGCTGTTCAAGACCAACCGGAAAGTGACCGTG
AAGCAGCTGAAAGAGGACTACTTCAAGAAAATCGAGTGCTTCGACTCCGTGGAAAT
CTCCGGCGTGGAAGATCGGTTCAACGCCTCCCTGGGCACATACCACGATCTGCTGAA
AATTATCAAGGACAAGGACTTCCTGGACAATGAGGAAAACGAGGACATTCTGGAAG
ATATCGTGCTGACCCTGACACTGTTTGAGGACAGAGAGATGATCGAGGAACGGCTG
AAAACCTATGCCCACCTGTTCGACGACAAAGTGATGAAGCAGCTGAAGCGGCGGAG
ATACACCGGCTGGGGCAGGCTGAGCCGGAAGCTGATCAACGGCATCCGGGACAAGC
AGTCCGGCAAGACAATCCTGGATTTCCTGAAGTCCGACGGCTTCGCCAACAGAAAC
TTCATGCAGCTGATCCACGACGACAGCCTGACCTTTAAAGAGGACATCCAGAAAGC
CCAGGTGTCCGGCCAGGGCGATAGCCTGCACGAGCACATTGCCAATCTGGCCGGCA
GCCCCGCCATTAAGAAGGGCATCCTGCAGACAGTGAAGGTGGTGGACGAGCTCGTG
AAAGTGATGGGCCGGCACAAGCCCGAGAACATCGTGATCGAAATGGCCAGAGAGA
ACCAGACCACCCAGAAGGGACAGAAGAACAGCCGCGAGAGAATGAAGCGGATCGA
AGAGGGCATCAAAGAGCTGGGCAGCCAGATCCTGAAAGAACACCCCGTGGAAAAC
ACCCAGCTGCAGAACGAGAAGCTGTACCTGTACTACCTGCAGAATGGGCGGGATAT
GTACGTGGACCAGGAACTGGACATCAACCGGCTGTCCGACTACGATGTGGACGCTA
TCGTGCCTCAGAGCTTTCTGAAGGACGACTCCATCGACAACAAGGTGCTGACCAGA
AGCGACAAGAACCGGGGCAAGAGCGACAACGTGCCCTCCGAAGAGGTCGTGAAGA
AGATGAAGAACTACTGGCGGCAGCTGCTGAACGCCAAGCTGATTACCCAGAGAAAG
TTCGACAATCTGACCAAGGCCGAGAGAGGCGGCCTGAGCGAACTGGATAAGGCCGG
CTTCATCAAGAGACAGCTGGTGGAAACCCGGCAGATCACAAAGCACGTGGCACAGA
TCCTGGACTCCCGGATGAACACTAAGTACGACGAGAATGACAAGCTGATCCGGGAA
GTGAAAGTGATCACCCTGAAGTCCAAGCTGGTGTCCGATTTCCGGAAGGATTTCCAG
TTTTACAAAGTGCGCGAGATCAACAACTACCACCACGCCCACGACGCCTACCTGAA
CGCCGTCGTGGGAACCGCCCTGATCAAAAAGTACCCTAAGCTGGAAAGCGAGTTCG
TGTACGGCGACTACAAGGTGTACGACGTGCGGAAGATGATCGCCAAGAGCGAGCAG
GAAATCGGCAAGGCTACCGCCAAGTACTTCTTCTACAGCAACATCATGAACTTTTTC
AAGACCGAGATTACCCTGGCCAACGGCGAGATCCGGAAGCGGCCTCTGATCGAGAC
AAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGACA
GGCGGCTTCAGCAAAGAGTCTATCCTGCCCAAGAGGAACAGCGATAAGCTGATCGC
CAGAAAGAAGGACTGGGACCCTAAGAAGTACGGCGGCTTCGACAGCCCCACCGTGG
CCTATTCTGTGCTGGTGGTGGCCAAAGTGGAAAAGGGCAAGTCCAAGAAACTGAAG
AGTGTGAAAGAGCTGCTGGGGATCACCATCATGGAAAGAAGCAGCTTCGAGAAGAA
TCCCATCGACTTTCTGGAAGCCAAGGGCTACAAAGAAGTGAAAAAGGACCTGATCA
TCAAGCTGCCTAAGTACTCCCTGTTCGAGCTGGAAAACGGCCGGAAGAGAATGCTG
GCCTCTGCCGGCGAACTGCAGAAGGGAAACGAACTGGCCCTGCCCTCCAAATATGT
GAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATC
GAGCAGATCAGCGAGTTCTCCAAGAGAGTGATCCTGGCCGACGCTAATCTGGACAA
AGTGCTGTCCGCCTACAACAAGCACCGGGATAAGCCCATCAGAGAGCAGGCCGAGA
ATATCATCCACCTGTTTACCCTGACCAATCTGGGAGCCCCTGCCGCCTTCAAGTACTT
TGACACCACCATCGACCGGAAGAGGTACACCAGCACCAAAGAGGTGCTGGACGCCA
CCCTGATCCACCAGAGCATCACCGGCCTGTACGAGACACGGATCGACCTGTCTCAGC
TGGGAGGTGACTCCGGCGGCAGCGAGGCCGCCGCCAAGGAAGCCGCCGCCAAGGA
AGCCGCTGCCAAGGAGGCCGCTGCTAAAAGCGGCGGATCTACCCTGAACATCGAGG
ACGAGTACAGGCTGCACGAGACCAGCAAGGAGCCCGACGTGAGCCTGGGCAGCAC
CTGGCTGAGCGATTTCCCTCAGGCTTGGGCCGAGACCGGCGGCATGGGCCTGGCCGT
GCGGCAGGCCCCCCTGATTATCCCCCTGAAGGCCACCAGCACCCCCGTGAGCATCA
AGCAGTACCCAATGTCCCAGGAGGCCAGGCTGGGCATCAAGCCTCACATCCAGAGG
CTGCTGGACCAGGGCATCCTGGTGCCATGCCAGTCCCCCTGGAACACCCCTCTGCTG
CCCGTGAAGAAGCCTGGCACCAACGACTACCGGCCCGTGCAGGACCTGAGAGAAGT
GAACAAGCGGGTGGAGGACATCCACCCAACCGTGCCCAACCCTTACAACCTGCTGT
CCGGCCTGCCCCCCAGCCACCAGTGGTACACCGTGCTGGACCTGAAGGACGCCTTCT
TCTGCCTGAGACTGCACCCCACCTCTCAGCCCCTGTTCGCCTTCGAGTGGCGCGACC
CCGAGATGGGCATCAGCGGCCAGCTGACCTGGACCAGACTGCCACAGGGCTTTAAG
AATAGCCCAACCCTGTTTAACGAGGCCCTGCACAGGGACCTGGCCGACTTCAGGAT
CCAGCACCCCGACCTGATTCTGCTGCAGTACGTGGACGACCTGCTGCTGGCCGCTAC
CAGCGAGCTGGACTGCCAGCAGGGCACCAGAGCCCTGCTGCAGACCCTGGGCAACC
TGGGCTACAGAGCCAGCGCCAAGAAGGCCCAGATCTGTCAGAAGCAGGTGAAGTAT
CTGGGCTACCTGCTGAAGGAAGGCCAGAGATGGCTGACCGAGGCCAGAAAGGAGA
CTGTGATGGGCCAGCCCACCCCCAAGACCCCCAGGCAGCTGCGGGAGTTCCTGGGC
AAGGCCGGCTTTTGCAGACTGTTTATCCCTGGCTTCGCCGAGATGGCCGCCCCACTG
TACCCTCTGACCAAGCCTGGCACCCTGTTTAACTGGGGCCCCGACCAGCAGAAGGCC
TACCAGGAGATCAAGCAGGCCCTGCTGACCGCCCCCGCCCTGGGCCTGCCCGACCT
GACCAAGCCTTTCGAGCTGTTCGTGGACGAGAAGCAGGGATACGCCAAAGGCGTGC
TGACCCAGAAGCTGGGCCCCTGGCGGAGGCCCGTGGCCTACCTGAGCAAAAAACTG
GACCCTGTGGCCGCCGGCTGGCCCCCATGCCTGCGGATGGTGGCCGCCATCGCTGTG
CTGACCAAGGACGCCGGCAAGCTGACCATGGGCCAGCCCCTGGTGATCCTGGCCCC
TCACGCCGTGGAGGCTCTGGTGAAGCAGCCTCCAGACAGGTGGCTGTCCAACGCCA
GGATGACCCACTACCAGGCCCTGCTGCTGGACACCGACCGGGTGCAGTTCGGCCCT
GTGGTGGCCCTGAACCCCGCCACCCTGCTGCCTCTGCCAGAGGAGGGCCTGCAGCA
CAACTGCCTGGACATCCTGGCCGAGGCCCACGGCGGCGGCTCCAAACGCACCGCCG
ACGGGAGCGAGTTCGAGCCCAAGAAGAAGAGGAAAGTCTAA;
and/or
the polynucleotide sequence is an mRNA.

47.-53. (canceled)

54. A PEgRNA system comprising:
i) a prime editing guide RNA (PEgRNA) comprising:
a spacer that is complementary to a search target sequence on a first strand of a human SERPINA1 gene,
a primer binding site sequence (PBS) at least partially complementary to the spacer,
an editing template that comprises a region of complementarity to an editing target sequence on a second strand of the SERPINA1 gene, and
a gRNA core that associates with a prime editor comprising a DNA binding domain and a DNA polymerase domain; and
ii) a nick guide RNA (ngRNA), wherein the ngRNA comprises an ng spacer that is complementary to a second search target sequence in the SERPINA1 gene,
wherein the ngRNA comprises a spacer sequence selected from the group consisting of:
(SEQ ID NO: [[##]]26)
GAAGCAGAGACACGUUGUA,
(SEQ ID NO: [[##]]27)
GUCAGCACAGCCUUAUGCA,
(SEQ ID NO: [[##]]28)
GAAAGGGACUGAAGCUGCU,
(SEQ ID NO: [[##]]29)
CCUCGGGGGGGAUAGACAU,
(SEQ ID NO: [[##]]30)
UGAUCCCAGGCCUCGAGCA,
(SEQ ID NO: [[##]]31)
ACGUUGUAAGGCUGAUCCC,
(SEQ ID NO: [[##]]32)
AAAGGGACUGAAGCUGCUG,
(SEQ ID NO: [[##]]33)
GGUAUGGCCUCUAAAAACA,
(SEQ ID NO: [[##]]34)
CCCAUGUCUAUCCCCCCCG,
(SEQ ID NO: [[##]]35)
GCCUCGAGCAAGGCUCACG,
(SEQ ID NO: [[##]]36)
GGUUUGUUGAACUUGACCU,
(SEQ ID NO: [[##]]37)
CCUUAUGCACGGCCUGGAG,
(SEQ ID NO: [[##]]38)
AGAAAGGGACUGAAGCUGC,
(SEQ ID NO: [[##]]39)
CACAGCCUUAUGCACGGCC,
(SEQ ID NO: [[##]]40)
GGGGGGAUAGACAUGGGUA,
(SEQ ID NO: [[##]]41)
GUUUGUUGAACUUGACCUC,
(SEQ ID NO: [[##]]42)
UGCUGACCAUCGACAAGAA,
(SEQ ID NO: [[##]]43)
UUGUUGAACUUGACCUCGG,
(SEQ ID NO: [[##]]44)
GCCUUAUGCACGGCCUGGA,
(SEQ ID NO: [[##]]45)
UUUGUUGAACUUGACCUCG,
(SEQ ID NO: [[##]]46)
GUUGAACUUGACCUCGGGG,
(SEQ ID NO: [[##]]47)
CUCUGCUUCUCUCCCCUCC,
(SEQ ID NO: [[##]]48)
UGAGCCUUGCUCGAGGCCU,
(SEQ ID NO: [[##]]49)
AGCCUUAUGCACGGCCUGG,
(SEQ ID NO: [[##]]50)
ACCUCGGGGGGGAUAGACA,
(SEQ ID NO: [[##]]51)
UCAGUCCCUUUCUUGUCGA,
(SEQ ID NO: [[##]]52)
UGUUGAACUUGACCUCGGG,
(SEQ ID NO: [[##]]53)
CCCCUCCAGGCCGUGCAUA,
(SEQ ID NO: [[##]]54)
GUGAGCCUUGCUCGAGGCC,
(SEQ ID NO: [[##]]55)
GCUGACCAUCGACAAGAAA,
(SEQ ID NO: [[##]]56)
GCUGGGGCCAUGUUUUUAG,
(SEQ ID NO: [[##]]57)
UGCUGACCAUCGACGAGAA,
(SEQ ID NO: [[##]]58)
UCAGUCCCUUUCUCGUCGA,
and/or
(SEQ ID NO: [[##]]59)
GCUGACCAUCGACGAGAAA.

55. The PEgRNA system of claim 54, wherein
the PBS comprises or consists of the sequence GCACGGC (SEQ ID NO: 2) and the editing template comprises or consists of the sequence UUUCUCGUCGAUGGUCAGCACAGCCUUAU (SEQ ID NO: 25); and/or,
the spacer sequence comprises or consists of the sequence
(SEQ ID NO: 1)
UCCCCUCCAGGCCGUGCAUA

56. (canceled)

57. A prime editing guide RNA (PEgRNA) comprising:
a spacer that is complementary to a search target sequence on a first strand of a human SERPINA1 gene,
a primer binding site sequence (PBS) at least partially complementary to the spacer,
an editing template that comprises a region of complementarity to an editing target sequence on a second strand of the SERPINA1 gene, and
a gRNA core that associates with a prime editor comprising a DNA binding domain and a DNA polymerase domain,
wherein the editing template comprises or consists of the sequence
(SEQ ID NO: [[##]]283)
UUUCUCAUCGAUGGUCAGCACAGCCUUAU
or
(SEQ ID NO: [[##]]284)
CUUUCUCAUCGAUGGUCAGCACAGCCUUAU.

58. The PEgRNA of claim 57, wherein
the PBS comprises or consists of the sequence GCACGGC (SEQ ID NO: 2); and/or,
the spacer sequence comprises or consists of the sequence
(SEQ ID NO: 1)
UCCCCUCCAGGCCGUGCAUA

59. (canceled)

60. A lipid nanoparticle (LNP) or ribonucleoprotein (RNP) comprising the prime editing complex of claim 33, or a component thereof.

61. A lipid nanoparticle (LNP) composition comprising i) the PEgRNA of claim 1, ii) a ngRNA, and iii) a polynucleotide encoding the prime editor.

62. The LNP composition of claim 61, wherein
the PERNA, the ngRNA, and the polynucleotide encoding the prime editor are each encapsulated in separate LNPs;
the PEgRNA, the ngRNA, and the polynucleotide encoding the prime editor are encapsulated in a single LNP
the mass ratio of the polynucleotide encoding the prime editor to the combination of the PERNA and the ngRNA (total gRNA content) is about 0.1:1 to about 3.0:1;
the mass ratio of the polynucleotide encoding the prime editor to the combination of the PERNA and the ngRNA (total gRNA content) is about 0.5:1;
the mass ratio of the PERNA to the ngRNA is about 1:1 to about 25:1; and/or
the mass ratio of the PEgRNA to the ngRNA is about 19:1.

63.-67. (canceled)

68. A polynucleotide encoding the PEgRNA of claim 1 optionally wherein
the polynucleotide is a mRNA;
the polynucleotide is operably linked to a regulatory element; and/or
the regulatory element is an inducible regulatory element.

69.-71. (canceled)

72. A vector comprising the polynucleotide of claim 68, optionally wherein the vector is an AAV vector.

73. (canceled)

74. An isolated cell comprising the PERNA of claim 1, the PEgRNA system comprising the PEgRNA of claim 1, the prime editing complex comprising the PEgRNA of claim 1, the LNP or RNP comprising the PEgRNA of claim 1, the polynucleotide encoding the PERNA of claim 1, or the vector comprising a polynucleotide encoding the PEgRNA of claim 1, optionally wherein
the cell is a human cell;
the cell is a hepatocyte, a hepatic stellate cell, a Kupffer cell, or a liver sinusoidal endothelial cell; and/or
wherein the cell is a hepatocyte or a hepatic stellate cell.

75.-77. (canceled)

78. A pharmaceutical composition comprising (i) the PEgRNA of of claim 1, the PEgRNA system comprising the PEgRNA of claim 1, the prime editing complex comprising the PEgRNA of claim 1, the LNP or RNP comprising the PERNA of claim 1, the polynucleotide encoding the PERNA of claim 1, the vector of claim comprising a polynucleotide encoding the PEgRNA, or the cell comprising PEgRNA of claim 1; and (ii) a pharmaceutically acceptable carrier.

79. A method for
editing a SERPINA1 gene, the method comprising contacting the SERPINA1 gene with (i) the PERNA of claim 1 or the PEgRNA system comprising a PEgRNA of claim 1 and (ii) a prime editor comprising a DNA binding domain and a DNA polymerase domain, wherein the PEgRNA or the PERNA system directs the prime editor to incorporate the intended nucleotide edit in the SERPINA1 gene, thereby editing the SERPINA1 gene;
editing an SERPINA1 gene, the method comprising contacting the SERPINA1 gene with the prime editing complex comprising a PEgRNA of claim 1, wherein the PERNA directs the prime editor to incorporate the intended nucleotide edit in the SERPINA1 gene, thereby editing the SERPINA1 gene,
optionally wherein
the method comprising contacting a cell with the LNP composition comprising a PEgRNA of claim 1;
the prime editor synthesizes a single stranded DNA encoded by the editing template, wherein the single stranded DNA replaces the editing target sequence and results in incorporation of the intended nucleotide edit into a region corresponding to the editing target in the SERPINA1 gene;
the SERPINA1 gene is in a cell;
the cell is a mammalian cell;
the cell is a human cell;
the cell is a hepatocyte or a hepatic stellate cell;
the cell is in a subject;
the subject is a human;
the cell is from a subject having A1AD;
further comprising administering the cell to the subject after incorporation of the intended nucleotide edit;
a cell or a population of cells is generated by said method; and/or
treating A1AD in a subject in need thereof, the method comprising administering to the subject (i) the PEgRNA of claim 1 or the PEgRNA system comprising a PEgRNA of claim 1 and (ii) a prime editor comprising a DNA binding domain and a DNA polymerase domain, wherein the PERNA directs the prime editor to incorporate the intended nucleotide edit in the SERPINA1 gene in the subject, thereby treating A1AD in the subject optionally wherein the subject is a human;
the method comprising administering to the subject the prime editing complex comprising a PEgRNA of claim 1, the LNP or RNP comprising a PEgRNA of claim 1, or the pharmaceutical composition comprising a PERNA of claim 1, wherein the PERNA directs the prime editor to incorporate the intended nucleotide edit in the SERPINA1 gene in the subject, thereby treating A1AD in the subject optionally wherein the subject is a human; and/or
treating A1AD in a subject in need thereof, the method comprising administering to the subject the LNP composition comprising a PEgRNA of claim 1, thereby treating A1AD in the subject, optionally wherein the subject is a human.

80.-96. (canceled)


SEQUENCE LISTING (396 sequences)

SEQ ID NO: 1 - RNA, 20 nt, synthetic construct
  source 1..20: mol_type = other RNA
tcccctccaggccgtgcata

SEQ ID NO: 2-4 - skipped (3 entries)

SEQ ID NO: 5 - RNA, 10 nt, synthetic construct
  source 1..10: mol_type = other RNA
gcacggcctg

SEQ ID NO: 6 - RNA, 11 nt, synthetic construct
  source 1..11: mol_type = other RNA
gcacggcctgg

SEQ ID NO: 7 - RNA, 12 nt, synthetic construct
  source 1..12: mol_type = other RNA
gcacggcctgga

SEQ ID NO: 8 - RNA, 13 nt, synthetic construct
  source 1..13: mol_type = other RNA
gcacggcctggag

SEQ ID NO: 9 - RNA, 14 nt, synthetic construct
  source 1..14: mol_type = other RNA
gcacggcctggagg

SEQ ID NO: 10 - RNA, 15 nt, synthetic construct
  source 1..15: mol_type = other RNA
gcacggcctggaggg

SEQ ID NO: 11 - RNA, 43 nt, synthetic construct
  source 1..43: mol_type = other RNA
gcagcttcagtccctttctcgtcgatggtcagcacagccttat

SEQ ID NO: 12 - RNA, 42 nt, synthetic construct
  source 1..42: mol_type = other RNA
cagcttcagtccctttctcgtcgatggtcagcacagccttat

SEQ ID NO: 13 - RNA, 41 nt, synthetic construct
  source 1..41: mol_type = other RNA
agcttcagtccctttctcgtcgatggtcagcacagccttat

SEQ ID NO: 14 - RNA, 40 nt, synthetic construct
  source 1..40: mol_type = other RNA
gcttcagtccctttctcgtcgatggtcagcacagccttat

SEQ ID NO: 15 - RNA, 39 nt, synthetic construct
  source 1..39: mol_type = other RNA
cttcagtccctttctcgtcgatggtcagcacagccttat

SEQ ID NO: 16 - RNA, 38 nt, synthetic construct
  source 1..38: mol_type = other RNA
ttcagtccctttctcgtcgatggtcagcacagccttat

SEQ ID NO: 17 - RNA, 37 nt, synthetic construct
  source 1..37: mol_type = other RNA
tcagtccctttctcgtcgatggtcagcacagccttat

SEQ ID NO: 18 - RNA, 36 nt, synthetic construct
  source 1..36: mol_type = other RNA
cagtccctttctcgtcgatggtcagcacagccttat

SEQ ID NO: 19 - RNA, 35 nt, synthetic construct
  source 1..35: mol_type = other RNA
agtccctttctcgtcgatggtcagcacagccttat

SEQ ID NO: 20 - RNA, 34 nt, synthetic construct
  source 1..34: mol_type = other RNA
gtccctttctcgtcgatggtcagcacagccttat

SEQ ID NO: 21 - RNA, 33 nt, synthetic construct
  source 1..33: mol_type = other RNA
tccctttctcgtcgatggtcagcacagccttat

SEQ ID NO: 22 - RNA, 32 nt, synthetic construct
  source 1..32: mol_type = other RNA
ccctttctcgtcgatggtcagcacagccttat

SEQ ID NO: 23 - RNA, 31 nt, synthetic construct
  source 1..31: mol_type = other RNA
cctttctcgtcgatggtcagcacagccttat

SEQ ID NO: 24 - RNA, 30 nt, synthetic construct
  source 1..30: mol_type = other RNA
ctttctcgtcgatggtcagcacagccttat

SEQ ID NO: 25 - RNA, 29 nt, synthetic construct
  source 1..29: mol_type = other RNA
tttctcgtcgatggtcagcacagccttat

SEQ ID NO: 26 - RNA, 19 nt, synthetic construct
  source 1..19: mol_type = other RNA
gaagcagagacacgttgta

SEQ ID NO: 27 - RNA, 19 nt, synthetic construct
  source 1..19: mol_type = other RNA
gtcagcacagccttatgca

SEQ ID NO: 28 - RNA, 19 nt, synthetic construct
  source 1..19: mol_type = other RNA
gaaagggactgaagctgct

SEQ ID NO: 29 - RNA, 19 nt, synthetic construct
  source 1..19: mol_type = other RNA
cctcgggggggatagacat

SEQ ID NO: 30 - RNA, 19 nt, synthetic construct
  source 1..19: mol_type = other RNA
tgatcccaggcctcgagca

SEQ ID NO: 31 - RNA, 19 nt, synthetic construct
  source 1..19: mol_type = other RNA
acgttgtaaggctgatccc

SEQ ID NO: 32 - RNA, 19 nt, synthetic construct
  source 1..19: mol_type = other RNA
aaagggactgaagctgctg

SEQ ID NO: 33 - RNA, 19 nt, synthetic construct
  source 1..19: mol_type = other RNA
ggtatggcctctaaaaaca

SEQ ID NO: 34 - RNA, 19 nt, synthetic construct
  source 1..19: mol_type = other RNA
cccatgtctatcccccccg

SEQ ID NO: 35 - RNA, 19 nt, synthetic construct
  source 1..19: mol_type = other RNA
gcctcgagcaaggctcacg

SEQ ID NO: 36 - RNA, 19 nt, synthetic construct
  source 1..19: mol_type = other RNA
ggtttgttgaacttgacct

SEQ ID NO: 37 - RNA, 19 nt, synthetic construct
  source 1..19: mol_type = other RNA
ccttatgcacggcctggag

SEQ ID NO: 38 - RNA, 19 nt, synthetic construct
  source 1..19: mol_type = other RNA
agaaagggactgaagctgc

SEQ ID NO: 39 - RNA, 19 nt, synthetic construct
  source 1..19: mol_type = other RNA
cacagccttatgcacggcc

SEQ ID NO: 40 - RNA, 19 nt, synthetic construct
  source 1..19: mol_type = other RNA
ggggggatagacatgggta

SEQ ID NO: 41 - RNA, 19 nt, synthetic construct
  source 1..19: mol_type = other RNA
gtttgttgaacttgacctc

SEQ ID NO: 42 - RNA, 19 nt, synthetic construct
  source 1..19: mol_type = other RNA
tgctgaccatcgacaagaa

SEQ ID NO: 43 - RNA, 19 nt, synthetic construct
  source 1..19: mol_type = other RNA
ttgttgaacttgacctcgg

SEQ ID NO: 44 - RNA, 19 nt, synthetic construct
  source 1..19: mol_type = other RNA
gccttatgcacggcctgga

SEQ ID NO: 45 - RNA, 19 nt, synthetic construct
  source 1..19: mol_type = other RNA
tttgttgaacttgacctcg

SEQ ID NO: 46 - RNA, 19 nt, synthetic construct
  source 1..19: mol_type = other RNA
gttgaacttgacctcgggg

SEQ ID NO: 47 - RNA, 19 nt, synthetic construct
  source 1..19: mol_type = other RNA
ctctgcttctctcccctcc

SEQ ID NO: 48 - RNA, 19 nt, synthetic construct
  source 1..19: mol_type = other RNA
tgagccttgctcgaggcct

SEQ ID NO: 49 - RNA, 19 nt, synthetic construct
  source 1..19: mol_type = other RNA
agccttatgcacggcctgg

SEQ ID NO: 50 - RNA, 19 nt, synthetic construct
  source 1..19: mol_type = other RNA
acctcgggggggatagaca

SEQ ID NO: 51 - RNA, 19 nt, synthetic construct
  source 1..19: mol_type = other RNA
tcagtccctttcttgtcga

SEQ ID NO: 52 - RNA, 19 nt, synthetic construct
  source 1..19: mol_type = other RNA
tgttgaacttgacctcggg

SEQ ID NO: 53 - RNA, 19 nt, synthetic construct
  source 1..19: mol_type = other RNA
cccctccaggccgtgcata

SEQ ID NO: 54 - RNA, 19 nt, synthetic construct
  source 1..19: mol_type = other RNA
gtgagccttgctcgaggcc

SEQ ID NO: 55 - RNA, 19 nt, synthetic construct
  source 1..19: mol_type = other RNA
gctgaccatcgacaagaaa

SEQ ID NO: 56 - RNA, 19 nt, synthetic construct
  source 1..19: mol_type = other RNA
gctggggccatgtttttag

SEQ ID NO: 57 - RNA, 19 nt, synthetic construct
  source 1..19: mol_type = other RNA
tgctgaccatcgacgagaa

SEQ ID NO: 58 - RNA, 19 nt, synthetic construct
  source 1..19: mol_type = other RNA
tcagtccctttctcgtcga

SEQ ID NO: 59 - RNA, 19 nt, synthetic construct
  source 1..19: mol_type = other RNA
gctgaccatcgacgagaaa

SEQ ID NO: 60 - protein, 1368 aa, synthetic construct [sequence omitted]

SEQ ID NO: 61 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMAAENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 62 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 63 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 64 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLASKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 65 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSALVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 66 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLASKLVSDFRADFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 67 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYAVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 68 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 69 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMAAENQTTQKGQKNSRERMKRIEEGIKELGSQILKEHPVENTQLQNEKLYLYYLQNGRDMYVDQELDINRLSDYDVDAIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 70 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYAVAEINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMAAENQTTQKGQKNSRERMKRIEEGIKELGSQILKEHPVENTQLQNEKLYLYYLQNGRDMYVDQELDINRLSDYDVDAIVPQSFLADDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 72 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLASALVSDFRADFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 73 - protein, 677 aa, unidentified
  source 1..677: note = Moloney murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 74 - protein, 677 aa, unidentified
  source 1..677: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 75 - protein, 517 aa, unidentified
  source 1..517: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGGGSKRTADGSEFE

SEQ ID NO: 76 - protein, 1368 aa, synthetic construct [sequence omitted]

SEQ ID NO: 77 - protein, 1931 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSEAAAKEAAAKEAAAKEAAAKSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGGGSKRTADGSEFE

SEQ ID NO: 78 - DNA, 5817 bp, synthetic construct
  source 1..5817: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggcagcgaggccgccgccaaggaagccgccgccaaggaagccgctgccaaggaggccgctgctaaaagcggcggatctaccctgaacatcgaggacgagtacaggctgcacgagaccagcaaggagcccgacgtgagcctgggcagcacctggctgagcgatttccctcaggcttgggccgagaccggcggcatgggcctggccgtgcggcaggcccccctgattatccccctgaaggccaccagcacccccgtgagcatcaagcagtacccaatgtcccaggaggccaggctgggcatcaagcctcacatccagaggctgctggaccagggcatcctggtgccatgccagtccccctggaacacccctctgctgcccgtgaagaagcctggcaccaacgactaccggcccgtgcaggacctgagagaagtgaacaagcgggtggaggacatccacccaaccgtgcccaacccttacaacctgctgtccggcctgccccccagccaccagtggtacaccgtgctggacctgaaggacgccttcttctgcctgagactgcaccccacctctcagcccctgttcgccttcgagtggcgcgaccccgagatgggcatcagcggccagctgacctggaccagactgccacagggctttaagaatagcccaaccctgtttaacgaggccctgcacagggacctggccgacttcaggatccagcaccccgacctgattctgctgcagtacgtggacgacctgctgctggccgctaccagcgagctggactgccagcagggcaccagagccctgctgcagaccctgggcaacctgggctacagagccagcgccaagaaggcccagatctgtcagaagcaggtgaagtatctgggctacctgctgaaggaaggccagagatggctgaccgaggccagaaaggagactgtgatgggccagcccacccccaagacccccaggcagctgcgggagttcctgggcaaggccggcttttgcagactgtttatccctggcttcgccgagatggccgccccactgtaccctctgaccaagcctggcaccctgtttaactggggccccgaccagcagaaggcctaccaggagatcaagcaggccctgctgaccgcccccgccctgggcctgcccgacctgaccaagcctttcgagctgttcgtggacgagaagcagggatacgccaaaggcgtgctgacccagaagctgggcccctggcggaggcccgtggcctacctgagcaaaaaactggaccctgtggccgccggctggcccccatgcctgcggatggtggccgccatcgctgtgctgaccaaggacgccggcaagctgaccatgggccagcccctggtgatcctggcccctcacgccgtggaggctctggtgaagcagcctccagacaggtggctgtccaacgccaggatgacccactaccaggccctgctgctggacaccgaccgggtgcagttcggccctgtggtggccctgaaccccgccaccctgctgcctctgccagaggagggcctgcagcacaactgcctggacatcctggccgaggcccacggcggcggctccaaacgcaccgccgacgggagcgagttcgagcccaagaagaagaggaaagtctaa

SEQ ID NO: 79 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctggaggtttt

SEQ ID NO: 80 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctggagggtttt

SEQ ID NO: 81 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctgtttt

SEQ ID NO: 82 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctggagtttt

SEQ ID NO: 83 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctggatttt

SEQ ID NO: 84 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctggtttt

SEQ ID NO: 85 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctggaggtttt

SEQ ID NO: 86 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctggagggtttt

SEQ ID NO: 87 - RNA, 138 nt, synthetic construct
  source 1..138: mol_type = other RNA
  modified_base order(1..3,135..137): mod_base = OTHER; note = 2-Prime-O-methyl ribonucleotides
  modified_base order(1..4,135..138): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggccttttt

SEQ ID NO: 88 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctgtttt

SEQ ID NO: 89 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctggagtttt

SEQ ID NO: 90 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctggatttt

SEQ ID NO: 91 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctggtttt

SEQ ID NO: 92 - RNA, 137 nt, synthetic construct
  source 1..137: mol_type = other RNA
  modified_base order(1..3,134..136): mod_base = OTHER; note = 2-Prime-O-methyl ribonucleotides
  modified_base order(1..4,134..137): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggcctttt

SEQ ID NO: 93 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctggaggtttt

SEQ ID NO: 94 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctggagggtttt

SEQ ID NO: 95 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggccttttt

SEQ ID NO: 96 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctgtttt

SEQ ID NO: 97 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctggagtttt

SEQ ID NO: 98 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctggatttt

SEQ ID NO: 99 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctggtttt

SEQ ID NO: 100 - RNA, 136 nt, synthetic construct
  source 1..136: mol_type = other RNA
  modified_base order(1..3,133..135): mod_base = OTHER; note = 2-Prime-O-methyl ribonucleotides
  modified_base order(1..4,133..136): mod_base = OTHER; note = Phosphorothioate linkages
tcccctccaggccgtgcatagttttagagctagaaatagcaagttaaaataaggctagtccgttatcaacttgaaaaagtggcaccgagtcggtgctttctcgtcgatggtcagcacagccttatgcacggctttt

SEQ ID NO: 101 - RNA, 138 nt, synthetic construct
  source 1..138: mol_type = other RNA
  modified_base order(1..3,135..137): mod_base = OTHER; note = 2-Prime-O-methyl ribonucleotides
  modified_base order(1..4,135..138): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggcctttt

SEQ ID NO: 102 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggccttttt

SEQ ID NO: 103 - RNA, 155 nt, synthetic construct
  source 1..155: mol_type = other RNA
  modified_base order(1..3,152..154): mod_base = OTHER; note = 2-Prime-O-methyl ribonucleotides
  modified_base order(1..4,152..155): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctggaggtttt

SEQ ID NO: 104 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctggaggtttt

SEQ ID NO: 105 - RNA, 156 nt, synthetic construct
  source 1..156: mol_type = other RNA
  modified_base order(1..3,153..155): mod_base = OTHER; note = 2-Prime-O-methyl ribonucleotides
  modified_base order(1..4,153..156): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctggagggtttt

SEQ ID NO: 106 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctggaggtttt

SEQ ID NO: 107 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctggagtttt

SEQ ID NO: 108 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctggaggtttt

SEQ ID NO: 109 - RNA, 157 nt, synthetic construct
  source 1..157: mol_type = other RNA
  modified_base order(1..3,154..156): mod_base = OTHER; note = 2-Prime-O-methyl ribonucleotides
  modified_base order(1..4,154..157): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctggaggtttt

SEQ ID NO: 110 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctggagggtttt

SEQ ID NO: 111 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctggaggtttt

SEQ ID NO: 112 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctggatttt

SEQ ID NO: 113 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctgtttt

SEQ ID NO: 114 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctggagggtttt

SEQ ID NO: 115 - RNA, 154 nt, synthetic construct
  source 1..154: mol_type = other RNA
  modified_base order(1..3,151..153): mod_base = OTHER; note = 2-Prime-O-methyl ribonucleotides
  modified_base order(1..4,151..154): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctggaggtttt

SEQ ID NO: 116 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctggagtttt

SEQ ID NO: 117 - RNA, 158 nt, synthetic construct
  source 1..158: mol_type = other RNA
  modified_base order(1..3,155..157): mod_base = OTHER; note = 2-Prime-O-methyl ribonucleotides
  modified_base order(1..4,155..158): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctggagggtttt

SEQ ID NO: 118 - RNA, 154 nt, synthetic construct
  source 1..154: mol_type = other RNA
  modified_base order(1..3,151..153): mod_base = OTHER; note = 2-Prime-O-methyl ribonucleotides
  modified_base order(1..4,151..154): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctggagtttt

SEQ ID NO: 119 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctggatttt

SEQ ID NO: 120 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctgtttt

SEQ ID NO: 121 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctgtttt

SEQ ID NO: 122 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctggagggtttt

SEQ ID NO: 123 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctggatttt

SEQ ID NO: 124 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctggagtttt

SEQ ID NO: 125 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctggtttt

SEQ ID NO: 126 - RNA, 155 nt, synthetic construct
  source 1..155: mol_type = other RNA
  modified_base order(1..3,152..154): mod_base = OTHER; note = 2-Prime-O-methyl ribonucleotides
  modified_base order(1..4,152..155): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctggagggtttt

SEQ ID NO: 127 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggcctttt

SEQ ID NO: 128 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctggtttt

SEQ ID NO: 129 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctggagggtttt

SEQ ID NO: 130 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctggagtttt

SEQ ID NO: 131 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctgtttt

SEQ ID NO: 132 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctggtttt

SEQ ID NO: 133 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctggatttt

SEQ ID NO: 134 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggccttttt

SEQ ID NO: 135 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctgtttt

SEQ ID NO: 136 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctgtttt

SEQ ID NO: 137 - RNA, 137 nt, synthetic construct
  source 1..137: mol_type = other RNA
  modified_base order(1..3,134..136): mod_base = OTHER; note = 2-Prime-O-methyl ribonucleotides
  modified_base order(1..4,134..137): mod_base = OTHER; note = Phosphorothioate linkages
tcccctccaggccgtgcatagttttagagctagaaatagcaagttaaaataaggctagtccgttatcaacttgaaaaagtggcaccgagtcggtgcctttctcgtcgatggtcagcacagccttatgcacggctttt

SEQ ID NO: 138 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctggatttt

SEQ ID NO: 139 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctggaggtttt

SEQ ID NO: 140 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctggagtttt

SEQ ID NO: 141 - RNA, 156 nt, synthetic construct
  source 1..156: mol_type = other RNA
  modified_base order(1..3,153..155): mod_base = OTHER; note = 2-Prime-O-methyl ribonucleotides
  modified_base order(1..4,153..156): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctggagtttt

SEQ ID NO: 142 - RNA, 155 nt, synthetic construct
  source 1..155: mol_type = other RNA
  modified_base order(1..3,152..154): mod_base = OTHER; note = 2-Prime-O-methyl ribonucleotides
  modified_base order(1..4,152..155): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctggatttt

SEQ ID NO: 143 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctggtttt

SEQ ID NO: 144 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctggtttt

SEQ ID NO: 145 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctgtttt

SEQ ID NO: 146 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctggatttt

SEQ ID NO: 147 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggccttttt

SEQ ID NO: 148 - RNA, 154 nt, synthetic construct
  source 1..154: mol_type = other RNA
  modified_base order(1..3,151..153): mod_base = OTHER; note = 2-Prime-O-methyl ribonucleotides
  modified_base order(1..4,151..154): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctggtttt

SEQ ID NO: 149 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctggagggtttt

SEQ ID NO: 150 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctggagtttt

SEQ ID NO: 151 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggccttttt

SEQ ID NO: 152 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggccttttt

SEQ ID NO: 153 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctggtttt

SEQ ID NO: 154 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctggatttt

SEQ ID NO: 155 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctgtttt

SEQ ID NO: 156 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggccttttt

SEQ ID NO: 157 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggcctttt

SEQ ID NO: 158 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctggtttt

SEQ ID NO: 159 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctggaggtttt

SEQ ID NO: 160 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggccttttt

SEQ ID NO: 161 - RNA, 154 nt, synthetic construct
  source 1..154: mol_type = other RNA
  modified_base order(1..3,151..153): mod_base = OTHER; note = 2-Prime-O-methyl ribonucleotides
  modified_base order(1..4,151..154): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctggagggtttt

SEQ ID NO: 162 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggccttttt

SEQ ID NO: 163 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggcctttt

SEQ ID NO: 164 - RNA, 138 nt, synthetic construct
  source 1..138: mol_type = other RNA
  modified_base order(1..3,135..137): mod_base = OTHER; note = 2-Prime-O-methyl ribonucleotides
  modified_base order(1..4,135..138): mod_base = OTHER; note = Phosphorothioate linkages
tcccctccaggccgtgcatagttttagagctagaaatagcaagttaaaataaggctagtccgttatcaacttgaaaaagtggcaccgagtcggtgccctttctcgtcgatggtcagcacagccttatgcacggctttt

SEQ ID NO: 165 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggccttttt

SEQ ID NO: 166 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggcctttt

SEQ ID NO: 167 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctggagtttt

SEQ ID NO: 168 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctggagggtttt

SEQ ID NO: 169 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctggaggtttt

SEQ ID NO: 170 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggcctttt

SEQ ID NO: 171 - RNA, 157 nt, synthetic construct
  source 1..157: mol_type = other RNA
  modified_base order(1..3,154..156): mod_base = OTHER; note = 2-Prime-O-methyl ribonucleotides
  modified_base order(1..4,154..157): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctggagggtttt

SEQ ID NO: 172 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggcctttt

SEQ ID NO: 173 - RNA, 156 nt, synthetic construct
  source 1..156: mol_type = other RNA
  modified_base order(1..3,153..155): mod_base = OTHER; note = 2-Prime-O-methyl ribonucleotides
  modified_base order(1..4,153..156): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctggaggtttt

SEQ ID NO: 174 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctggatttt

SEQ ID NO: 175 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctgtttt

SEQ ID NO: 176 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctggtttt

SEQ ID NO: 177 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctggagtttt

SEQ ID NO: 178 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctgtttt

SEQ ID NO: 179 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggcctttt

SEQ ID NO: 180 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggcctttt

SEQ ID NO: 181 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctggatttt

SEQ ID NO: 182 - RNA, 152 nt, synthetic construct
  source 1..152: mol_type = other RNA
  modified_base order(1..3,149..151): mod_base = OTHER; note = 2-Prime-O-methyl ribonucleotides
  modified_base order(1..4,149..152): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctgtttt

SEQ ID NO: 183 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctggtttt

SEQ ID NO: 184 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggcctttt

SEQ ID NO: 185 - RNA, 154 nt, synthetic construct
  source 1..154: mol_type = other RNA
  modified_base order(1..3,151..153): mod_base = OTHER; note = 2-Prime-O-methyl ribonucleotides
  modified_base order(1..4,151..154): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctggatttt

SEQ ID NO: 186 - RNA, 155 nt, synthetic construct
  source 1..155: mol_type = other RNA
  modified_base order(1..3,152..154): mod_base = OTHER; note = 2-Prime-O-methyl ribonucleotides
  modified_base order(1..4,152..155): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctggagtttt

SEQ ID NO: 187 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgctccctttctcgtcgatggtcagcacagccttatgcacggctttt

SEQ ID NO: 188 - RNA, 153 nt, synthetic construct
  source 1..153: mol_type = other RNA
  modified_base order(1..3,150..152): mod_base = OTHER; note = 2-Prime-O-methyl ribonucleotides
  modified_base order(1..4,150..153): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctggtttt

SEQ ID NO: 189 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgtccctttctcgtcgatggtcagcacagccttatgcacggctttt

SEQ ID NO: 190 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggccttttt

SEQ ID NO: 191 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggccttttt

SEQ ID NO: 192 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagtccctttctcgtcgatggtcagcacagccttatgcacggctttt

SEQ ID NO: 193 - RNA, 148 nt, synthetic construct
  source 1..148: mol_type = other RNA
  modified_base order(1..3,145..147): mod_base = OTHER; note = 2-Prime-O-methyl ribonucleotides
  modified_base order(1..4,145..148): mod_base = OTHER; note = Phosphorothioate linkages
tcccctccaggccgtgcatagttttagagctagaaatagcaagttaaaataaggctagtccgttatcaacttgaaaaagtggcaccgagtcggtgcagcttcagtccctttctcgtcgatggtcagcacagccttatgcacggctttt

SEQ ID NO: 194 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttcagtccctttctcgtcgatggtcagcacagccttatgcacggctttt

SEQ ID NO: 195 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctggaggtttt

SEQ ID NO: 196 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctggagggtttt

SEQ ID NO: 197 - RNA, 151 nt, synthetic construct
  source 1..151: mol_type = other RNA
  modified_base order(1..3,148..150): mod_base = OTHER; note = 2-Prime-O-methyl ribonucleotides
  modified_base order(1..4,148..151): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggccttttt

SEQ ID NO: 198 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggcctttt

SEQ ID NO: 199 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctggagtttt

SEQ ID NO: 200 - RNA, 138 nt, synthetic construct
  source 1..138: mol_type = other RNA
  modified_base order(1..3,135..137): mod_base = OTHER; note = 2-Prime-O-methyl ribonucleotides
  modified_base order(1..4,135..138): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctgtttt

SEQ ID NO: 201 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggcctttt

SEQ ID NO: 202 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctggatttt

SEQ ID NO: 203 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctggtttt

SEQ ID NO: 204 - RNA, 145 nt, synthetic construct
  source 1..145: mol_type = other RNA
  modified_base order(1..3,142..144): mod_base = OTHER; note = 2-Prime-O-methyl ribonucleotides
  modified_base order(1..4,142..145): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctggagtttt

SEQ ID NO: 205 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctggaggtttt

SEQ ID NO: 206 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcagcttcagtccctttctcgtcgatggtcagcacagccttatgcacggctttt

SEQ ID NO: 207 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgcgcttcagtccctttctcgtcgatggtcagcacagccttatgcacggctttt

SEQ ID NO: 208 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgctcagtccctttctcgtcgatggtcagcacagccttatgcacggctttt

SEQ ID NO: 209 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggccttttt

SEQ ID NO: 210 - RNA, 144 nt, synthetic construct
  source 1..144: mol_type = other RNA
  modified_base order(1..3,141..143): mod_base = OTHER; note = 2-Prime-O-methyl ribonucleotides
  modified_base order(1..4,141..144): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctggatttt

SEQ ID NO: 211 - RNA, 150 nt, synthetic construct
  source 1..150: mol_type = other RNA
  modified_base order(1..3,147..149): mod_base = OTHER; note = 2-Prime-O-methyl ribonucleotides
  modified_base order(1..4,147..150): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggcctttt

SEQ ID NO: 212 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctgtttt

SEQ ID NO: 213 - RNA, 137 nt, synthetic construct
  source 1..137: mol_type = other RNA
  modified_base order(1..3,134..136): mod_base = OTHER; note = 2-Prime-O-methyl ribonucleotides
  modified_base order(1..4,134..137): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggccttttt

SEQ ID NO: 214 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctggtttt

SEQ ID NO: 215 - RNA, 147 nt, synthetic construct
  source 1..147: mol_type = other RNA
  modified_base order(1..3,144..146): mod_base = OTHER; note = 2-Prime-O-methyl ribonucleotides
  modified_base order(1..4,144..147): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctggagggtttt

SEQ ID NO: 216 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggcctttt

SEQ ID NO: 217 - RNA, 143 nt, synthetic construct
  source 1..143: mol_type = other RNA
  modified_base order(1..3,140..142): mod_base = OTHER; note = 2-Prime-O-methyl ribonucleotides
  modified_base order(1..4,140..143): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagtccctttctcgtcgatggtcagcacagccttatgcacggctttt

SEQ ID NO: 218 - RNA, 146 nt, synthetic construct
  source 1..146: mol_type = other RNA
  modified_base order(1..3,143..145): mod_base = OTHER; note = 2-Prime-O-methyl ribonucleotides
  modified_base order(1..4,143..146): mod_base = OTHER; note = Phosphorothioate linkages
tcccctccaggccgtgcatagttttagagctagaaatagcaagttaaaataaggctagtccgttatcaacttgaaaaagtggcaccgagtcggtgccttcagtccctttctcgtcgatggtcagcacagccttatgcacggctttt

SEQ ID NO: 219 - RNA, 136 nt, synthetic construct
  source 1..136: mol_type = other RNA
  modified_base order(1..3,133..135): mod_base = OTHER; note = 2-Prime-O-methyl ribonucleotides
  modified_base order(1..4,133..136): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggcctttt

SEQ ID NO: 220 - RNA, 142 nt, synthetic construct
  source 1..142: mol_type = other RNA
  modified_base order(1..3,139..141): mod_base = OTHER; note = 2-Prime-O-methyl ribonucleotides
  modified_base order(1..4,139..142): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctggagggtttt

SEQ ID NO: 221 - RNA, 141 nt, synthetic construct
  source 1..141: mol_type = other RNA
  modified_base order(1..3,138..140): mod_base = OTHER; note = 2-Prime-O-methyl ribonucleotides
  modified_base order(1..4,138..141): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctggaggtttt

SEQ ID NO: 222 - RNA, 149 nt, synthetic construct
  source 1..149: mol_type = other RNA
  modified_base order(1..3,146..148): mod_base = OTHER; note = 2-Prime-O-methyl ribonucleotides
  modified_base order(1..4,146..149): mod_base = OTHER; note = Phosphorothioate linkages
tcccctccaggccgtgcatagttttagagctagaaatagcaagttaaaataaggctagtccgttatcaacttgaaaaagtggcaccgagtcggtgccagcttcagtccctttctcgtcgatggtcagcacagccttatgcacggctttt

SEQ ID NO: 223 - RNA, 137 nt, synthetic construct
  source 1..137: mol_type = other RNA
  modified_base order(1..3,134..136): mod_base = OTHER; note = 2-Prime-O-methyl ribonucleotides
  modified_base order(1..4,134..137): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctgtttt

SEQ ID NO: 224 - RNA, 140 nt, synthetic construct
  source 1..140: mol_type = other RNA
  modified_base order(1..3,137..139): mod_base = OTHER; note = 2-Prime-O-methyl ribonucleotides
  modified_base order(1..4,137..140): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctggagtttt

SEQ ID NO: 225 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgcccctttctcgtcgatggtcagcacagccttatgcacggctttt

SEQ ID NO: 226 - RNA, 139 nt, synthetic construct
  source 1..139: mol_type = other RNA
  modified_base order(1..3,136..138): mod_base = OTHER; note = 2-Prime-O-methyl ribonucleotides
  modified_base order(1..4,136..139): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctggatttt

SEQ ID NO: 227 - RNA, 138 nt, synthetic construct
  source 1..138: mol_type = other RNA
  modified_base order(1..3,135..137): mod_base = OTHER; note = 2-Prime-O-methyl ribonucleotides
  modified_base order(1..4,135..138): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctggtttt

SEQ ID NO: 228 - RNA, 135 nt, synthetic construct
  source 1..135: mol_type = other RNA
  modified_base order(1..3,132..134): mod_base = OTHER; note = 2-Prime-O-methyl ribonucleotides
  modified_base order(1..4,132..135): mod_base = OTHER; note = Phosphorothioate linkages
tcccctccaggccgtgcatagttttagagctagaaatagcaagttaaaataaggctagtccgttatcaacttgaaaaagtggcaccgagtcggtgcttctcgtcgatggtcagcacagccttatgcacggctttt

SEQ ID NO: 229 - RNA, 136 nt, synthetic construct
  source 1..136: mol_type = other RNA
  modified_base order(1..3,133..135): mod_base = OTHER; note = 2-Prime-O-methyl ribonucleotides
  modified_base order(1..4,133..136): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggccttttt

SEQ ID NO: 230 - RNA, 135 nt, synthetic construct
  source 1..135: mol_type = other RNA
  modified_base order(1..3,132..134): mod_base = OTHER; note = 2-Prime-O-methyl ribonucleotides
  modified_base order(1..4,132..135): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggcctttt

SEQ ID NO: 231 - RNA, 134 nt, synthetic construct
  source 1..134: mol_type = other RNA
  modified_base order(1..3,131..133): mod_base = OTHER; note = 2-Prime-O-methyl ribonucleotides
  modified_base order(1..4,131..134): mod_base = OTHER; note = Phosphorothioate linkages
tcccctccaggccgtgcatagttttagagctagaaatagcaagttaaaataaggctagtccgttatcaacttgaaaaagtggcaccgagtcggtgctctcgtcgatggtcagcacagccttatgcacggctttt

SEQ ID NO: 232 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gtcagcacagccttatgcagttttagagctagaaatagcaagttaaaataaggctagtccgttatcaacttgaaaaagtggcaccgagtcggtgctttt

SEQ ID NO: 233 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gaaagggactgaagctgctgttttagagctagaaatagcaagttaaaataaggctagtccgttatcaacttgaaaaagtggcaccgagtcggtgctttt

SEQ ID NO: 234 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
cctcgggggggatagacatgttttagagctagaaatagcaagttaaaataaggctagtccgttatcaacttgaaaaagtggcaccgagtcggtgctttt

SEQ ID NO: 235 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tgatcccaggcctcgagcagttttagagctagaaatagcaagttaaaataaggctagtccgttatcaacttgaaaaagtggcaccgagtcggtgctttt

SEQ ID NO: 236 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
acgttgtaaggctgatcccgttttagagctagaaatagcaagttaaaataaggctagtccgttatcaacttgaaaaagtggcaccgagtcggtgctttt

SEQ ID NO: 237 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
aaagggactgaagctgctggttttagagctagaaatagcaagttaaaataaggctagtccgttatcaacttgaaaaagtggcaccgagtcggtgctttt

SEQ ID NO: 238 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gaagcagagacacgttgtagttttagagctagaaatagcaagttaaaataaggctagtccgttatcaacttgaaaaagtggcaccgagtcggtgctttt

SEQ ID NO: 239 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ggtatggcctctaaaaacagttttagagctagaaatagcaagttaaaataaggctagtccgttatcaacttgaaaaagtggcaccgagtcggtgctttt

SEQ ID NO: 240 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
cccatgtctatcccccccggttttagagctagaaatagcaagttaaaataaggctagtccgttatcaacttgaaaaagtggcaccgagtcggtgctttt

SEQ ID NO: 241 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gcctcgagcaaggctcacggttttagagctagaaatagcaagttaaaataaggctagtccgttatcaacttgaaaaagtggcaccgagtcggtgctttt

SEQ ID NO: 242 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ggtttgttgaacttgacctgttttagagctagaaatagcaagttaaaataaggctagtccgttatcaacttgaaaaagtggcaccgagtcggtgctttt

SEQ ID NO: 243 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ccttatgcacggcctggaggttttagagctagaaatagcaagttaaaataaggctagtccgttatcaacttgaaaaagtggcaccgagtcggtgctttt

SEQ ID NO: 244 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
agaaagggactgaagctgcgttttagagctagaaatagcaagttaaaataaggctagtccgttatcaacttgaaaaagtggcaccgagtcggtgctttt

SEQ ID NO: 245 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
cacagccttatgcacggccgttttagagctagaaatagcaagttaaaataaggctagtccgttatcaacttgaaaaagtggcaccgagtcggtgctttt

SEQ ID NO: 246 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ggggggatagacatgggtagttttagagctagaaatagcaagttaaaataaggctagtccgttatcaacttgaaaaagtggcaccgagtcggtgctttt

SEQ ID NO: 247 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gtttgttgaacttgacctcgttttagagctagaaatagcaagttaaaataaggctagtccgttatcaacttgaaaaagtggcaccgagtcggtgctttt

SEQ ID NO: 248 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tgctgaccatcgacaagaagttttagagctagaaatagcaagttaaaataaggctagtccgttatcaacttgaaaaagtggcaccgagtcggtgctttt

SEQ ID NO: 249 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ttgttgaacttgacctcgggttttagagctagaaatagcaagttaaaataaggctagtccgttatcaacttgaaaaagtggcaccgagtcggtgctttt

SEQ ID NO: 250 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gccttatgcacggcctggagttttagagctagaaatagcaagttaaaataaggctagtccgttatcaacttgaaaaagtggcaccgagtcggtgctttt

SEQ ID NO: 251 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tttgttgaacttgacctcggttttagagctagaaatagcaagttaaaataaggctagtccgttatcaacttgaaaaagtggcaccgagtcggtgctttt

SEQ ID NO: 252 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gttgaacttgacctcgggggttttagagctagaaatagcaagttaaaataaggctagtccgttatcaacttgaaaaagtggcaccgagtcggtgctttt

SEQ ID NO: 253 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
ctctgcttctctcccctccgttttagagctagaaatagcaagttaaaataaggctagtccgttatcaacttgaaaaagtggcaccgagtcggtgctttt

SEQ ID NO: 254 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tgagccttgctcgaggcctgttttagagctagaaatagcaagttaaaataaggctagtccgttatcaacttgaaaaagtggcaccgagtcggtgctttt

SEQ ID NO: 255 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
agccttatgcacggcctgggttttagagctagaaatagcaagttaaaataaggctagtccgttatcaacttgaaaaagtggcaccgagtcggtgctttt

SEQ ID NO: 256 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
acctcgggggggatagacagttttagagctagaaatagcaagttaaaataaggctagtccgttatcaacttgaaaaagtggcaccgagtcggtgctttt

SEQ ID NO: 257 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tcagtccctttcttgtcgagttttagagctagaaatagcaagttaaaataaggctagtccgttatcaacttgaaaaagtggcaccgagtcggtgctttt

SEQ ID NO: 258 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tgttgaacttgacctcggggttttagagctagaaatagcaagttaaaataaggctagtccgttatcaacttgaaaaagtggcaccgagtcggtgctttt

SEQ ID NO: 259 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
cccctccaggccgtgcatagttttagagctagaaatagcaagttaaaataaggctagtccgttatcaacttgaaaaagtggcaccgagtcggtgctttt

SEQ ID NO: 260 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gtgagccttgctcgaggccgttttagagctagaaatagcaagttaaaataaggctagtccgttatcaacttgaaaaagtggcaccgagtcggtgctttt

SEQ ID NO: 261 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gctgaccatcgacaagaaagttttagagctagaaatagcaagttaaaataaggctagtccgttatcaacttgaaaaagtggcaccgagtcggtgctttt

SEQ ID NO: 262 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gctggggccatgtttttaggttttagagctagaaatagcaagttaaaataaggctagtccgttatcaacttgaaaaagtggcaccgagtcggtgctttt

SEQ ID NO: 263 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tgctgaccatcgacgagaagttttagagctagaaatagcaagttaaaataaggctagtccgttatcaacttgaaaaagtggcaccgagtcggtgctttt

SEQ ID NO: 264 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
tcagtccctttctcgtcgagttttagagctagaaatagcaagttaaaataaggctagtccgttatcaacttgaaaaagtggcaccgagtcggtgctttt

SEQ ID NO: 265 - RNA, 99 nt, synthetic construct
  source 1..99: mol_type = other RNA
  modified_base order(1..3,96..98): mod_base = OTHER; note = 2-Prime-O-methyl ribonucleotides
  modified_base order(1..4,96..99): mod_base = OTHER; note = Phosphorothioate linkages
gctgaccatcgacgagaaagttttagagctagaaatagcaagttaaaataaggctagtccgttatcaacttgaaaaagtggcaccgagtcggtgctttt

SEQ ID NO: 266 - RNA, 100 nt, synthetic construct
  source 1..100: mol_type = other RNA
  modified_base order(1..3,97..99): mod_base = OTHER; note = 2-Prime-O-methyl ribonucleotides
  modified_base order(1..4,97..100): mod_base = OTHER; note = Phosphorothioate linkages
agaagcagagacacgttgtagttttagagctagaaatagcaagttaaaataaggctagtccgttatcaacttgaaaaagtggcaccgagtcggtgctttt

SEQ ID NO: 267 - RNA, 105 nt, synthetic construct
  source 1..105: mol_type = other RNA
  modified_base order(1..4,102..105): mod_base = OTHER; note = Phosphorothioate linkages
gaagcagagacacgttgtagttttagagccggcggaaacgccggcaagttaaaataaggctagtccgttatcaacttgaaaaagtggcaccgagtcggtgctttt

SEQ ID NO: 268 - RNA, 100 nt, synthetic construct
  source 1..100: mol_type = other RNA
  modified_base order(1..3,97..99): mod_base = OTHER; note = 2-Prime-O-methyl ribonucleotides
  modified_base order(1..4,97..100): mod_base = OTHER; note = Phosphorothioate linkages
tttgttgaacttgacctcgggttttagagctagaaatagcaagttaaaataaggctagtccgttatcaacttgaaaaagtggcaccgagtcggtgctttt

SEQ ID NO: 269 - RNA, 138 nt, synthetic construct; identical to SEQ ID NO: 87
  source 1..138: mol_type = other RNA
  modified_base order(1..3,135..137): mod_base = OTHER; note = 2-Prime-O-methyl ribonucleotides
  modified_base 18..20: mod_base = OTHER; note = Locked nucleic acid
  modified_base order(1..4,135..138): mod_base = OTHER; note = Phosphorothioate linkages

SEQ ID NO: 270 - RNA, 138 nt, synthetic construct; identical to SEQ ID NO: 87
  source 1..138: mol_type = other RNA
  modified_base order(1..3,135..137): mod_base = OTHER; note = 2-Prime-O-methyl ribonucleotides
  modified_base order(4,6,8): mod_base = OTHER; note = Locked nucleic acid
  modified_base order(1..4,135..138): mod_base = OTHER; note = Phosphorothioate linkages

SEQ ID NO: 271 - RNA, 138 nt, synthetic construct; identical to SEQ ID NO: 87
  source 1..138: mol_type = other RNA
  modified_base order(1..2,135..137): mod_base = OTHER; note = 2-Prime-O-methyl ribonucleotides
  modified_base order(3,5,7): mod_base = OTHER; note = Locked nucleic acid
  modified_base order(1..4,135..138): mod_base = OTHER; note = Phosphorothioate linkages

SEQ ID NO: 272 - RNA, 138 nt, synthetic construct; identical to SEQ ID NO: 87
  source 1..138: mol_type = other RNA
  modified_base order(1..3,135..137): mod_base = OTHER; note = 2-Prime-O-methyl ribonucleotides
  modified_base 6..8: mod_base = OTHER; note = Locked nucleic acid
  modified_base order(1..4,135..138): mod_base = OTHER; note = Phosphorothioate linkages

SEQ ID NO: 273 - RNA, 138 nt, synthetic construct; identical to SEQ ID NO: 87
  source 1..138: mol_type = other RNA
  modified_base 135..137: mod_base = OTHER; note = 2-Prime-O-methyl ribonucleotides
  modified_base 1..3: mod_base = OTHER; note = Locked nucleic acid
  modified_base order(1..4,135..138): mod_base = OTHER; note = Phosphorothioate linkages

SEQ ID NO: 274 - RNA, 138 nt, synthetic construct; identical to SEQ ID NO: 87
  source 1..138: mol_type = other RNA
  modified_base order(1..3,135..137): mod_base = OTHER; note = 2-Prime-O-methyl ribonucleotides
  modified_base 4..6: mod_base = OTHER; note = Locked nucleic acid
  modified_base order(1..4,135..138): mod_base = OTHER; note = Phosphorothioate linkages

SEQ ID NO: 275 - RNA, 138 nt, synthetic construct; identical to SEQ ID NO: 87
  source 1..138: mol_type = other RNA
  modified_base order(1,3,135..137): mod_base = OTHER; note = 2-Prime-O-methyl ribonucleotides
  modified_base order(2,4,6,8): mod_base = OTHER; note = Locked nucleic acid
  modified_base order(1..4,135..138): mod_base = OTHER; note = Phosphorothioate linkages

SEQ ID NO: 276 - RNA, 138 nt, synthetic construct; identical to SEQ ID NO: 87
  source 1..138: mol_type = other RNA
  modified_base order(2,135..137): mod_base = OTHER; note = 2-Prime-O-methyl ribonucleotides
  modified_base order(1,3,5,7): mod_base = OTHER; note = Locked nucleic acid
  modified_base order(1..4,135..138): mod_base = OTHER; note = Phosphorothioate linkages

SEQ ID NO: 277 - protein, 28 aa, synthetic construct
SGGSEAAAKEAAAKEAAAKEAAAKSGGS

SEQ ID NO: 278 - protein, 18 aa, synthetic construct
SGGSEAAAKEAAAKSGGS

SEQ ID NO: 279 - protein, 13 aa, synthetic construct
SGGSEAAAKSGGS

SEQ ID NO: 280 - protein, 4 aa, synthetic construct
SGGS

SEQ ID NO: 281 - protein, 48 aa, synthetic construct
SGGSEAAAKEAAAKEAAAKEAAAKEAAAKEAAAKEAAAKEAAAKSGGS

SEQ ID NO: 282 - protein, 7 aa, synthetic construct
PKKKRKV

SEQ ID NO: 283 - RNA, 29 nt, synthetic construct
  source 1..29: mol_type = other RNA
tttctcatcgatggtcagcacagccttat

SEQ ID NO: 284 - RNA, 30 nt, synthetic construct
  source 1..30: mol_type = other RNA
ctttctcatcgatggtcagcacagccttat

SEQ ID NO: 285 - protein, 1386 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 286 - protein, 30 aa, synthetic construct
MDSLLMNRRKFLYQFKNVRWAKGRRETYLC

SEQ ID NO: 287 - protein, 18 aa, synthetic construct
KRTADGSEFESPKKKRKV

SEQ ID NO: 288 - protein, 17 aa, synthetic construct
KRTADGSEFEPKKKRKV

SEQ ID NO: 289 - protein, 4 aa, synthetic construct
GGGS

SEQ ID NO: 290 - protein, 4 aa, synthetic construct
GGSS

SEQ ID NO: 291 - protein, 5 aa, synthetic construct
GGGGS

SEQ ID NO: 292 - protein, 5 aa, synthetic construct
EAAAK

SEQ ID NO: 293 - protein, 16 aa, synthetic construct
SGSETPGTSESATPES

SEQ ID NO: 294 - protein, 32 aa, synthetic construct
SGGSSGGSSGSETPGTSESATPESSGGSSGGS

SEQ ID NO: 295 - protein, 10 aa, synthetic construct
SGGSGGSGGS

SEQ ID NO: 296 - protein, 60 aa, synthetic construct
SGGSSGGSSGSETPGTSESATPESAGSYPYDVPDYAGSAAPAAKKKKLDGSGSGGSSGGS

SEQ ID NO: 297 - protein, 9 aa, synthetic construct
PAAKRVKLD

SEQ ID NO: 298 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 299 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 300 - RNA, 76 nt, synthetic construct; identical to SEQ ID NO: 299
  source 1..76: mol_type = other RNA

SEQ ID NO: 301 - RNA, 76 nt, synthetic construct
  source 1..76: mol_type = other RNA
gtttgagagctagaaatagcaagtttaaataaggctagtccgttatcaacttgaaaaagtgggaccgagtcggtcc

SEQ ID NO: 302 - protein, 16 aa, synthetic construct
RQIKIWFQNRRMKWKK

SEQ ID NO: 303 - protein, 31 aa, synthetic construct
GGSSGGSSGGSSGGSSGGSSGGSSGGSSGGS

SEQ ID NO: 304 - protein, 19 aa, synthetic construct
MKRTADGSEFESPKKKRKV

SEQ ID NO: 305 - protein, 16 aa, synthetic construct
GGSEAAAKEAAAKGGS

SEQ ID NO: 306 - protein, 21 aa, synthetic construct
GGSEAAAKEAAAKEAAAKGGS

SEQ ID NO: 307 - protein, 1938 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSEAAAKEAAAKEAAAKEAAAKSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGGGSKRTADGSEFEPKKKRKV

SEQ ID NO: 308 - RNA, 5817 nt, synthetic construct
  source 1..5817: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggcagcgaggccgccgccaaggaagccgccgccaaggaagccgctgccaaggaggccgctgctaaaagcggcggatctaccctgaacatcgaggacgagtacaggctgcacgagaccagcaaggagcccgacgtgagcctgggcagcacctggctgagcgatttccctcaggcttgggccgagaccggcggcatgggcctggccgtgcggcaggcccccctgattatccccctgaaggccaccagcacccccgtgagcatcaagcagtacccaatgtcccaggaggccaggctgggcatcaagcctcacatccagaggctgctggaccagggcatcctggtgccatgccagtccccctggaacacccctctgctgcccgtgaagaagcctggcaccaacgactaccggcccgtgcaggacctgagagaagtgaacaagcgggtggaggacatccacccaaccgtgcccaacccttacaacctgctgtccggcctgccccccagccaccagtggtacaccgtgctggacctgaaggacgccttcttctgcctgagactgcaccccacctctcagcccctgttcgccttcgagtggcgcgaccccgagatgggcatcagcggccagctgacctggaccagactgccacagggctttaagaatagcccaaccctgtttaacgaggccctgcacagggacctggccgacttcaggatccagcaccccgacctgattctgctgcagtacgtggacgacctgctgctggccgctaccagcgagctggactgccagcagggcaccagagccctgctgcagaccctgggcaacctgggctacagagccagcgccaagaaggcccagatctgtcagaagcaggtgaagtatctgggctacctgctgaaggaaggccagagatggctgaccgaggccagaaaggagactgtgatgggccagcccacccccaagacccccaggcagctgcgggagttcctgggcaaggccggcttttgcagactgtttatccctggcttcgccgagatggccgccccactgtaccctctgaccaagcctggcaccctgtttaactggggccccgaccagcagaaggcctaccaggagatcaagcaggccctgctgaccgcccccgccctgggcctgcccgacctgaccaagcctttcgagctgttcgtggacgagaagcagggatacgccaaaggcgtgctgacccagaagctgggcccctggcggaggcccgtggcctacctgagcaaaaaactggaccctgtggccgccggctggcccccatgcctgcggatggtggccgccatcgctgtgctgaccaaggacgccggcaagctgaccatgggccagcccctggtgatcctggcccctcacgccgtggaggctctggtgaagcagcctccagacaggtggctgtccaacgccaggatgacccactaccaggccctgctgctggacaccgaccgggtgcagttcggccctgtggtggccctgaaccccgccaccctgctgcctctgccagaggagggcctgcagcacaactgcctggacatcctggccgaggcccacggcggcggctccaaacgcaccgccgacgggagcgagttcgagcccaagaagaagaggaaagtctaa

SEQ ID NO: 309 - protein, 19 aa, synthetic construct
CKRTADGSEFESPKKKRKV

SEQ ID NO: 310 - protein, 5 aa, synthetic construct
  VARIANT 1..5: note = The entire sequence of amino acids 1-5 can be repeated one to thirty times
GGGGS

SEQ ID NO: 311 - protein, 4 aa, synthetic construct
  VARIANT 4: note = G residue can be repeated up to twenty six times
GGGG

SEQ ID NO: 312 - protein, 5 aa, synthetic construct
  VARIANT 1..5: note = The entire sequence of amino acids 1-5 can be repeated one to thirty times
EAAAK

SEQ ID NO: 313 - protein, 6 aa, synthetic construct
  VARIANT 1..3: note = GGS residues can be repeated up to twenty eight times
GGSGGS

SEQ ID NO: 314 - protein, 4 aa, synthetic construct
  VARIANT 1..4: note = The entire sequence of amino acids 1-4 can be repeated one to thirty times
SGGS

SEQ ID NO: 315 - protein, 4 aa, synthetic construct
  VARIANT 1..4: note = The entire sequence of amino acids 1-4 can be repeated one to thirty times
GGSS

SEQ ID NO: 316 - protein, 8 aa, synthetic construct
  VARIANT 1: note = X can be any amino acid
  VARIANT 3: note = X can be any amino acid
  VARIANT 5: note = X can be any amino acid
  VARIANT 7: note = X can be any amino acid
  VARIANT 1..2: note = XP residues can be repeated up to twenty six times
XPXPXPXP

SEQ ID NO: 317 - protein, 9 aa, synthetic construct
  VARIANT 1..3: note = GGS residues can be repeated up to seven times
GGSGGSGGS

SEQ ID NO: 318 - protein, 7 aa, synthetic construct
  VARIANT 1..4: note = GGSS residues can be repeated up to fifty times or absent
  VARIANT 5..7: note = GGS residues can be repeated up to fifty times or absent
GGSSGGS

SEQ ID NO: 319 - protein, 7 aa, synthetic construct
  VARIANT 1..3: note = GGS residues can be repeated up to fifty times or absent
  VARIANT 4..7: note = GGSS residues can be repeated up to fifty times or absent
GGSGGSS

SEQ ID NO: 320 - protein, 7 aa, synthetic construct
GGSSGGS

SEQ ID NO: 321 - protein, 11 aa, synthetic construct
GGSSGGSSGGS

SEQ ID NO: 322 - protein, 15 aa, synthetic construct
GGSSGGSSGGSSGGS

SEQ ID NO: 323 - protein, 19 aa, synthetic construct
GGSSGGSSGGSSGGSSGGS

SEQ ID NO: 324 - protein, 23 aa, synthetic construct
GGSSGGSSGGSSGGSSGGSSGGS

SEQ ID NO: 325 - protein, 27 aa, synthetic construct
GGSSGGSSGGSSGGSSGGSSGGSSGGS

SEQ ID NO: 326 - protein, 21 aa, synthetic construct
GGSSGGSSGGSSGGSGGSGGS

SEQ ID NO: 327 - protein, 35 aa, synthetic construct
GGSSGGSSGGSSGGSSGGSSGGSSGGSSGGSSGGS

SEQ ID NO: 328 - protein, 39 aa, synthetic construct
GGSSGGSSGGSSGGSSGGSSGGSSGGSSGGSSGGSSGGS

SEQ ID NO: 329 - protein, 43 aa, synthetic construct
GGSSGGSSGGSSGGSSGGSSGGSSGGSSGGSSGGSSGGSSGGS

SEQ ID NO: 330 - protein, 47 aa, synthetic construct
GGSSGGSSGGSSGGSSGGSSGGSSGGSSGGSSGGSSGGSSGGSSGGS

SEQ ID NO: 331 - protein, 51 aa, synthetic construct
GGSSGGSSGGSSGGSSGGSSGGSSGGSSGGSSGGSSGGSSGGSSGGSSGGS

SEQ ID NO: 332 - protein, 55 aa, synthetic construct
GGSSGGSSGGSSGGSSGGSSGGSSGGSSGGSSGGSSGGSSGGSSGGSSGGSSGGS

SEQ ID NO: 333 - protein, 59 aa, synthetic construct
GGSSGGSSGGSSGGSSGGSSGGSSGGSSGGSSGGSSGGSSGGSSGGSSGGSSGGSSGGS

SEQ ID NO: 334 - protein, 63 aa, synthetic construct
GGSSGGSSGGSSGGSSGGSSGGSSGGSSGGSSGGSSGGSSGGSSGGSSGGSSGGSSGGSSGGS

SEQ ID NO: 335 - protein, 10 aa, synthetic construct
GGSSGGSGGS

SEQ ID NO: 336 - protein, 13 aa, synthetic construct
GGSSGGSGGSGGS

SEQ ID NO: 337 - protein, 16 aa, synthetic construct
GGSSGGSGGSGGSGGS

SEQ ID NO: 338 - protein, 19 aa, synthetic construct
GGSSGGSGGSGGSGGSGGS

SEQ ID NO: 339 - protein, 22 aa, synthetic construct
GGSSGGSGGSGGSGGSGGSGGS

SEQ ID NO: 340 - protein, 25 aa, synthetic construct
GGSSGGSGGSGGSGGSGGSGGSGGS

SEQ ID NO: 341 - protein, 28 aa, synthetic construct
GGSSGGSGGSGGSGGSGGSGGSGGSGGS

SEQ ID NO: 342 - protein, 31 aa, synthetic construct
GGSSGGSGGSGGSGGSGGSGGSGGSGGSGGS

SEQ ID NO: 343 - protein, 8 aa, synthetic construct
GGSSGGSK

SEQ ID NO: 344 - protein, 37 aa, synthetic construct
GGSSGGSGGSGGSGGSGGSGGSGGSGGSGGSGGSGGS

SEQ ID NO: 345 - protein, 40 aa, synthetic construct
GGSSGGSGGSGGSGGSGGSGGSGGSGGSGGSGGSGGSGGS

SEQ ID NO: 346 - protein, 43 aa, synthetic construct
GGSSGGSGGSGGSGGSGGSGGSGGSGGSGGSGGSGGSGGSGGS

SEQ ID NO: 347 - protein, 46 aa, synthetic construct
GGSSGGSGGSGGSGGSGGSGGSGGSGGSGGSGGSGGSGGSGGSGGS

SEQ ID NO: 348 - protein, 49 aa, synthetic construct
GGSSGGSGGSGGSGGSGGSGGSGGSGGSGGSGGSGGSGGSGGSGGSGGS

SEQ ID NO: 349 - protein, 14 aa, synthetic construct
GGSSGGSSGGSGGS

SEQ ID NO: 350 - protein, 28 aa, synthetic construct
GGSSGGSSGGSSGGSSGGSGGSGGSGGS

SEQ ID NO: 351 - protein, 35 aa, synthetic construct
GGSSGGSSGGSSGGSSGGSSGGSGGSGGSGGSGGS

SEQ ID NO: 352 - protein, 42 aa, synthetic construct
GGSSGGSSGGSSGGSSGGSSGGSSGGSGGSGGSGGSGGSGGS

SEQ ID NO: 353 - protein, 49 aa, synthetic construct
GGSSGGSSGGSSGGSSGGSSGGSSGGSSGGSGGSGGSGGSGGSGGSGGS

SEQ ID NO: 354 - protein, 56 aa, synthetic construct
GGSSGGSSGGSSGGSSGGSSGGSSGGSSGGSSGGSGGSGGSGGSGGSGGSGGSGGS

SEQ ID NO: 355 - protein, 63 aa, synthetic construct
GGSSGGSSGGSSGGSSGGSSGGSSGGSSGGSSGGSSGGSGGSGGSGGSGGSGGSGGSGGSGGS

SEQ ID NO: 356 - protein, 44 aa, synthetic construct
GGSSGGSSGGSSGGSSGGSSGGSSGGSSGGSSGGSSGGSSGGSK

SEQ ID NO: 357 - protein, 77 aa, synthetic construct
GGSSGGSSGGSSGGSSGGSSGGSSGGSSGGSSGGSSGGSSGGSSGGSGGSGGSGGSGGSGGSGGSGGSGGSGGSGGS

SEQ ID NO: 358 - protein, 84 aa, synthetic construct
GGSSGGSSGGSSGGSSGGSSGGSSGGSSGGSSGGSSGGSSGGSSGGSSGGSGGSGGSGGSGGSGGSGGSGGSGGSGGSGGSGGS

SEQ ID NO: 359 - protein, 91 aa, synthetic construct
GGSSGGSSGGSSGGSSGGSSGGSSGGSSGGSSGGSSGGSSGGSSGGSSGGSSGGSGGSGGSGGSGGSGGSGGSGGSGGSGGSGGSGGSGGS

SEQ ID NO: 360 - protein, 98 aa, synthetic construct
GGSSGGSSGGSSGGSSGGSSGGSSGGSSGGSSGGSSGGSSGGSSGGSSGGSSGGSSGGSGGSGGSGGSGGSGGSGGSGGSGGSGGSGGSGGSGGSGGS

SEQ ID NO: 361 - protein, 105 aa, synthetic construct
GGSSGGSSGGSSGGSSGGSSGGSSGGSSGGSSGGSSGGSSGGSSGGSSGGSSGGSSGGSSGGSGGSGGSGGSGGSGGSGGSGGSGGSGGSGGSGGSGGSGGSGGS

SEQ ID NO: 362 - protein, 8 aa, synthetic construct
GGSSGGSS

SEQ ID NO: 363 - protein, 12 aa, synthetic construct
GGSSGGSSGGSS

SEQ ID NO: 364 - protein, 16 aa, synthetic construct
GGSSGGSSGGSSGGSS

SEQ ID NO: 365 - protein, 20 aa, synthetic construct
GGSSGGSSGGSSGGSSGGSS

SEQ ID NO: 366 - protein, 24 aa, synthetic construct
GGSSGGSSGGSSGGSSGGSSGGSS

SEQ ID NO: 367 - protein, 28 aa, synthetic construct
GGSSGGSSGGSSGGSSGGSSGGSSGGSS

SEQ ID NO: 368 - protein, 32 aa, synthetic construct
GGSSGGSSGGSSGGSSGGSSGGSSGGSSGGSS

SEQ ID NO: 369 - protein, 36 aa, synthetic construct
GGSSGGSSGGSSGGSSGGSSGGSSGGSSGGSSGGSS

SEQ ID NO: 370 - protein, 28 aa, synthetic construct
  VARIANT 1..4: note = GGSS residues can be repeated up to thirteen times
GGSSGGSSGGSSGGSSGGSSGGSSGGSS

SEQ ID NO: 371 - protein, 12 aa, synthetic construct
  VARIANT 1..4: note = GGSS residues can be repeated up to seventeen times
GGSSGGSSGGSS

SEQ ID NO: 372 - protein, 6 aa, synthetic construct
  VARIANT 1..3: note = GGS residues can be repeated up to eighteen times
GGSGGS

SEQ ID NO: 373 - protein, 8 aa, synthetic construct
SGGSSGGS

SEQ ID NO: 374 - protein, 12 aa, synthetic construct
SGGSSGGSSGGS

SEQ ID NO: 375 - protein, 16 aa, synthetic construct
SGGSSGGSSGGSSGGS

SEQ ID NO: 376 - protein, 20 aa, synthetic construct
SGGSSGGSSGGSSGGSSGGS

SEQ ID NO: 377 - protein, 24 aa, synthetic construct
SGGSSGGSSGGSSGGSSGGSSGGS

SEQ ID NO: 378 - protein, 28 aa, synthetic construct
SGGSSGGSSGGSSGGSSGGSSGGSSGGS

SEQ ID NO: 379 - protein, 32 aa, synthetic construct
SGGSSGGSSGGSSGGSSGGSSGGSSGGSSGGS

SEQ ID NO: 380 - protein, 36 aa, synthetic construct
SGGSSGGSSGGSSGGSSGGSSGGSSGGSSGGSSGGS

SEQ ID NO: 381 - protein, 28 aa, synthetic construct
  VARIANT 1..4: note = SGGS residues can be repeated up to thirteen times
SGGSSGGSSGGSSGGSSGGSSGGSSGGS

SEQ ID NO: 382 - protein, 12 aa, synthetic construct
  VARIANT 1..3: note = SGGS residues can be repeated up to seventeen times
SGGSSGGSSGGS

SEQ ID NO: 383 - protein, 504 aa, unidentified
  source 1..504: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHG

SEQ ID NO: 384 - protein, 15 aa, synthetic construct
EAAAKEAAAKEAAAK

SEQ ID NO: 385 - protein, 20 aa, synthetic construct
EAAAKEAAAKEAAAKEAAAK

SEQ ID NO: 386 - protein, 25 aa, synthetic construct
EAAAKEAAAKEAAAKEAAAKEAAAK

SEQ ID NO: 387 - protein, 30 aa, synthetic construct
EAAAKEAAAKEAAAKEAAAKEAAAKEAAAK

SEQ ID NO: 388 - protein, 35 aa, synthetic construct
EAAAKEAAAKEAAAKEAAAKEAAAKEAAAKEAAAK

SEQ ID NO: 389 - protein, 40 aa, synthetic construct
EAAAKEAAAKEAAAKEAAAKEAAAKEAAAKEAAAKEAAAK

SEQ ID NO: 390 - protein, 45 aa, synthetic construct
EAAAKEAAAKEAAAKEAAAKEAAAKEAAAKEAAAKEAAAKEAAAK

SEQ ID NO: 391 - protein, 35 aa, synthetic construct; identical to SEQ ID NO: 388
  VARIANT 1..5: note = EAAAK residues can be repeated up to thirteen times

SEQ ID NO: 392 - protein, 15 aa, synthetic construct
  VARIANT 1..5: note = EAAAK residues can be repeated up to seventeen times
EAAAKEAAAKEAAAK

SEQ ID NO: 393 - protein, 14 aa, synthetic construct
GGSEAAAKGGSGGS

SEQ ID NO: 394 - protein, 17 aa, synthetic construct
GGSEAAAKGGSGGSGGS

SEQ ID NO: 395 - protein, 8 aa, synthetic construct
RRRRRRRR

SEQ ID NO: 396 - protein, 9 aa, synthetic construct
RRRRRRRRR